SEQUENCE LISTING (47 sequences)

SEQ ID NO: 1 - DNA, 102938 bp, Homo sapiens
gaagggaatgaaactcctcatgggaaatggtcatcgggatgagaaggaaggaaactgagaagaagaaaaaaaatgctaccaagctagagagttcactgatgacattcagatttcagaaagtctaccagaaatcaccacagcagggataggtctgccaggttgctgggaaaaagttactcctagtggatgctggtaaagaggctatctaaagagaaaatgttggctgtattgtattgctggtcattattcccaaccccctcaacatggaaggcagagatcacaaggaaatacaaacgccactgtgacacagttacaagaaaaataatattgttaggtgcccccatcctccacccagctttacacctatctcatggaatcattcaggagtatactgcatgacattctagaaaacacaaaataatgtggctaattttacaaagctaaggattctattcctgtagctgagccagtattcaaaagagatccctggatctgcatattaaaagccaaagctgggctgggcacagtggctcacgcctgtaatcccagcagtttgggaggcagaggcaggtggatcaccaaaggtcaggagttcgagaccagcctggccaaaatggtgaaagctggtttccactaaaaatacaaaaaaattagccgagcgtagcggtgcatgcctgtagtcccagctactcaggagggagaggcagcagaatcacttgaacccaaggggtggaggttgcagtgagctgagatcacgccattgcactccagcctgggtgaaaagagcaaaacgcagtcttaaataaataaataagccaaagctatgaaaatggttgatataagaagactacttggccaagagttagattcccatataaataagcagtttatgcctccattcatttaaaaatatgaatcccacacctctacatgctagacatatatttccagggctgggtatatggtgctgaagaaactaacattcctgcccctgccccttggtgtctttctatgcaggggtcggggtgagagggtcggtaatggtggtagatcagacaataaacaagaaaaataaatagttccagatagtgataagtgctgcaaagaaaatacagcaggaagaggtttggggggtgagattacagaggtaggcctagatctcacccgataagtaggtccttgtgggctctggtcagacttcaaattttgtataaatgcaatggaaaaccactggagggctttcagcaggggaataacatgatctgacgcgattttccaagaccgttctagatgctgtatgttcaaaaggtgggaaagaatatataagctgtgacatcagggaggggccttctccatgagtccaggggaaaggcattccaaagggacaccagggctggtggtggatgaaggaacaatcagagtagaaatttgaaaggcagagccaataggacttgctgatgcattggatgtagggagacaggacaaaagaaaaatcaaaaataactcctaatcttttttttcacatcagctgcagcaatgctcagccaggcctaagtagcggaaagactgtcttaattctattaaaaaacatacacacacaacagatagtcaacaatcagtaagtcagaaaaccctgatgagtaagttggaagggagagggaggaagcagatagaaaaacagattcataatgtcaggaaacggccaaaacctgagggcccggagatgggggtagaaaaatatgaacacacagagttggtttcaacagtctgcaatatcattcggagtgtcccagtggaccttaaatagagcaatggggcctcgggaactggagttcaagtctaataatcagcagcattttcagtagagatagatttgtatccatagtgatgtgctagaggcggagcatcaatctaaagttgtttcctagaaggagagtcttagactcccttgactgtataccaggaaacaaacccaggaaacaaattatatttgagatgggctcaaatatgaaatgggaatattgcacagaatcacataaagaaaaatgggaatgagcagcttgtactcactcatcttaaccaaggaatgaaaggcaattgtgagaaactaggtgtctcaggatggtgctaatactgagaacatccgaaaccgttataatcattatgcatttacaatgtattacacactgcaccaatgtacttgcaatatctcacggatgtctcacgattttcctggaagaaagcacagacttgattaaaagcatggactaggaaaccacactccctggtttcaaatcccaatactgccaccttgggcagctgacatcacctctttgaatctgagtttctctgcctgtaaatttcagaggataatggtagctacttcataggacctctgagaagattaaatgagctcatgtaaataaagtacttagaacagtgcctgggacagagtgatattcaattagtgatagcgactattactatcattccttttttacagggaaagacttagaagctgagagatgcttaagtaacttgtctaatgtcacagggctgaaatgtgacaaagccagggttttaatcacctctgtcctccagacccagtatctatcagataccagataggtcacactggcaagctgtgaaagcgagtctccaaacctgggaccaagtgaactatgacctcaatattcatatccatgagtggtccactcccacactgaatctgcgctggccctgtgacttgatttaaccaacaaaaagtggcagaagtaatgctgtgcctggcagcttctgcttttgtgctcttggaagccctgagccaccatggattaagtctgatccatggagagtgatggagagtccatatggagagaccacgtggagagaaaagaagctcaacctttccagcatgccagctgagcccatctttctagccattcccccaaggcaccagacatattagtggatacatattgaacatttcagccccaactaccctctgactgcatccccatgggacaccaaagcaagaccagcaaaacaactctccaactaagctacagtggtgacaatttattgttttaagcctctatgtttttgttatgcaacaatatgcaactgaaacactaatctataagaaaaagacttattggggacaaaaatgtgtcagtaacacaaggctgaaacctagagtgactgttgcatcccagaagcaagctgtacagaaaataatcatccatgctctggagtcctattaatgagcatataatagtgccaaccacacagtaagaaatcaacaaaaaattagctgctttttttattattaacaagaatacacttattctagatgcaatttaattgcaatgcaaggaatagataaaaatccttagtttttgcttctaacttctccaggaataatgctgcaggaaatttgtatcactttcctttgctttaggaaatttcagtgcacagtattgtaaagacttgattattaaaagatttgacaaggacaaaaaaagttaagaaagagaagatgaccacaactggtctcatactgctcgttaagagtagtgacttcctggctgggcatggtggctcacatctctaatcccagcactttgggaggctgaggcaggtggaccacctgaggtcaggagttcaagaccagcctggccaacatggtgaaaccccgtctctactgaaaacacaatatttagctgggcacagtggtgggcgcctataattccagctactagggaggctgaggcaggagaatcacttgaacccaggaggcagaggttgcagtgcaccgagatcacgccattgcactccagcctgggtagcaaaagggaaactctgtttaaaaaaaaagaaaaaagatagggacttcccaagttactcctgcaaaatggcattgttacagagcaatgtatcatgacaggcattttttgacaaaaaaaaaaaaaattcatttccaattcagacacaaagtagtatgagattttgtctatgcaaagaattttaaacctgtctcactccacaatggtgggagaatattgagttcccagaaataaagcagcagtcaaaggtagcacagacaaagacgctgagcccatgagttacatagaggagtgcagctgtactgcattcatcattttgatgaataaatagataagggaaaagcaaggccttccatagaaataaaggttatttctgtagattactttaaacgtataataaagtccttcagcgaaaaaaatagacattccaacagagatctcagggaacaagagcaacatggaatacccagaagacgatctagattgaacagaatagaatcatcagcagtagaattgagaacaatcaccagacctttggtggaggtggagctgagcactgatctttggatacgttatacatcctgtattgcttcatcgaagcaatggtatagtccagaggtccttgacctgcgggccacggatgggtactagtctgtggcctgttaggaaccaggctgcatagcaggaggtgagcagcaggcaagcgagtgagcgaagattcctctgtatttaaagcttctccgcatcattcgcattattgcctgagctctgtctcctgtcagatcagcggtggcattagattctcatggcagtgccaaccctattgcgaactgcgcatgcgagggatctaggttgcacgctccttaagagaatctaactcctgatgatctgtcactgtctcccatcacccccaggtgggaccctctagttgcaggaaaacaagctcagggctcccactgatcctacaatttggtgagttgtataattatttcattattgttaacaatgtaataataccaaagtacaaaataaatgtaatgcacttgaatcatcccaaaaccattccccatcccaggcgtgtggaaaaattgtcttccatgaaaccattccacggtgacaaaaaggttggggactgctggtttagtccattgaaaatcattccttgtttccataaagatcctcctcttctaccatttgtgatatgtggagttttggatgcatgagccctatctcattattagagtcttgggatttgtcaagagttggcaaaaccaacgatgcagcccactctgatttcctccttgaagaatttatttatttctggctaaaagtgctacgatttactccaggaggctgataattatagaaggcagctgtagtaatgtagaggaagccctaggctttaattcaagagacatagtccctcaacaaacacttgccgaggccctggcgtgaggcaaggcaatgtttaggtacatgaagaattactaaaaaccaaagagatcagagattattgattcatgttttatccaccgtgcccagcatagggctggatgtgtagcaggttttcagtaaatgtttgtttaaccaaattaataatgacactagttagcaaatcaatttccctctctggggttggtatttcacctcctattttaaaatttaaaaaatgtaagtgggttatagcagcacttctcaaaattgaatgtgcataacaatttcccggggatcctgttaaggtacatattccaaaccagtaggctgggaaggaaggcctggaagttgcgtttctaacaaacctccagcattactggcccatggaccacactttgagtagcaatggggcaaatgatctctcagggagaaattttggatacaatgagggatcctccgattctgttttttaattatgaaaaaatagtagatactctaactatggaagggagaaaaagagaggtaaatggggctgggcacggtgcctggggtggatccaggaggttggtagaggcttctcctgggggccagcaggaaaggcccactcttcgtcacaacgcacaaacaaattctttttggtgcctgcaaaaagaatcagcaataatttcgctggctcggaaggctctgtctcttaatgattcttcctgtttttctcagcctgatccaagagaatgagaaaacagccctagtgaaggtcagcggaggtcagagaggtccctggagagggccgctttcagggaaatggagcagatagaggcccaccccttctgcccaccctcatctctgcctaactctagcgctactccttgaccaatccagcccaatcccagtgtatcagtactctgccccaagtcaattccattcaattagacattcattactactgatgtgtgtgccagcatgctagatgccccggatgatgccaggatgtgacactgccagggccctccagttgcccatggtctaatggggaggagttccacagtcacaagaagacgtgttacaatgaagaatgcaatcaaggacacgagaaagagatctgcaagtacaatatgaatttagagaagtgagagaccaaactttacagagaaggggacatctgaaatttaaagatgggtataagctcttcagtaaagatggaaatgagccattccaggtggagggaaagcaggagagacagcacagaggggaaggaagagtggatgctcacgacagaatccagtgtgactggaacaaagggtattttgaaagaatagcctgcaatggagggtctgggaggtgcaggggagattttcatctttagggtcttagtcctccctttctcatctttagagtctgataattggccctcccttcaacatgcatatgcttccctgacccatctttaggatcatacttgcaaaaacagttcaagattaaccacagcaattcatttattccacaatcttttaccaaaaccctaagacacgtgaggcactgtgcaagcataggaaatacagccagggatgagataggtaagttccagagcactggacaaacaatcagtaaagtgagacggtagaaagaacataggaacaggaaagtcctgggtgcaaatatacgtcgtgtgtcatgaacaaaatatttcatatctctgagccctggtttctacctccctgatggggataattatacttattccacggggttattgtagaaattaacctgtggggaaagagcttcatactgtgttgggcacactgtaagcattcgttacccatgtattctttgttcagccataaagagaggcgcctggaataccaaatctccagggtccctttcagctctgatattttgcttgggcagttctaagactctgcggcacctgtaactgtgcccactgaccagggtcctggcacatcccaaggagtgacgctccatccctccctgagcaagaagcttctaccagggtggcctgagatgaggccaatggatcaggcaggcattaatcaggtggccgccctccttggctgtggcgccggcaggccaggcagcgttctggctgcctagcttccccgatcggcttccacaccactccacagacccgttgccatgacagtgaaccccaaggggcacccaggagtggccagagctggagaggaagggttccattgatcgaaatccatctgactcagccttggttgatttcaggatcctggagtgagaacaaacaacaatttaaatctactgaatgcttttttcaatgccagacaacatattaagtgtgtttttccgattctctcatttcatactcacaatggtaacgtgaaatagggatcctcattcccacttaacagaatgagaaactggggatcagagaagaaaataccgatgtcacccacatagtaagtggtaaggccaggacacaactgtgccccccagctccagccccagggatgtatcattccaaagaccctgtgcttttccacaaaccacagtcttccgagccacctgagcctccaggaaagttgatgccactttccccacttgcctgaatgttcctgaggggccaagggggacaggtgcagagagctaggtctctgagtgattcctcagagacaaggccaggtcacccagatgatgggctactcagacagctgccaaaaatatgttctgggattttccaatgtaatcccaatttcaaatattctgcttgtttttctaaagaatgaaaattccaacatactgccaccaaactaatacgtctctcaaatgagctgttgttcatctggaaacagcacaatatttgacaacatctgtcagaccccgtcacaacatttggttctaaagagcacggtccctgtattttgatgcaaggtagcaatgtgcaagctctggccttagatttagggctctgacctgccagaaggaggatatccagaaaccagaggacatcaaccctttttctaacctcagcagctggggaatttgccgaaagaacagcttggcattccttccctgcctttcaccccccttcccatggctactgaactattgagcccaaactctgggaacccatgacatgggaaaatccagctggcttctgtagccccagacatctggattccctaaccccaggcagccagctgagtgaggaggcagcaacacaaggatgcttgggaaagccttcggagggacgtctgccaagtcatctccctcctgaacaaacacatgtcccactatctaggggtcagcagagccatgtttatatgtgcttaggccattcaactgattaattaattagctccctgggtggcctttggccactaaagcttcatcccttgaggcctcagttttcttatctataaaatgttagaattaggcaagttgatgtctaaggttacttcctgccctacgatttcagctttctaccataaaagaacaggatgagcatggacaggatttggctctaatactgtccctgctctgccagcatgggaaagtcccttcccatttctaagcctcagtttccttggggagttaaatgaggggcggactagatgatctctcagtccagagctctgttatctcagaaactggaacacatcttgacatgccctctccctgatcacctgcatgcactgtaacccagcagaccccatctcgacagctccattgccacctactcaatcaggctgcctgctctcacctggaccactgcaatagcctcctaactggtcaagcaacatctgccgcccctccctcatctctatgctggagactaccagtgtgaagtttcttttcaaaacacaaatcagctaggcacaatggctcacgcctgtaatcaatcccagaactttgagaggctgaagcaggaggattacttgagcgtaggagtttgagaccacccgggacaacatagcaagatcccagctctaccaaaaaaaaaaaatttaaattatccaggcatgggggcgtttgtgtgtagtcccagctactcaagaggctgaggcaggaagatgacttgagcccaggaggtcagggctgcagtgagccatgctcatgccactgcactccagcctgagcaacagagtgagaccctgtctcaaaaaaaaaaaaaaaatatatatatatatatatataagatcatgttagcctgctgcttgtatggagttccattgcaattagggtaaacaatagtgcagcttgtgagaccctcacggctaacccagcccacccctcagcaccatgtctcaccaccctcccttcctcaatgtacacaagtcccttggcctttcgcttccttaatagacaagcctcccaagcctcaatctttgttcatgcaatttctttgcctagaatgctgatctgcttcctctctatcccactcccttcttcaacttgtccatttcttcccttcctttagatttcacctcaggcatctctgcttagaaatcctcctgggaactcaccaggctagctcagattccctgggtagactttcccataagaccattgccttccctacagagcattcctgtccatttctgagtgcgttcagggatgacattgctttctctcattcctactcatcccatagtctacactcaataaaccttttcaagatgagggactggatgttctgcccttaggaagctcctaagaagaggatggcaccttttccaaaagccctggcagagaccaagactaaaacagatctgatctaagactgtcaggctcccgaggagacatcctgctgactgctggaaagctctgcccaaggcaagcttgagacttagccaattgctatggctatgctcagagttcattccagcagagggtgctcagaagtccaccctagagcgctctgagctggctgccctcccagccagcaggcagcccggggcaatggctccagagcatcagattaggaggagagctgaagtctgtactgtggaaagacaagatgactagatagattcttctttggaaatgtcttaaagtgccttttcccccaccttctccacccactgacttgcacagatcattcaagtgtaaatgggcctcctccttcaggaagccttcctgtatcacccctgctggactaaggttcctaaatctgcatttctatgcctcctgggcatcccactagcattgtatactactacactgagagaatgtgtttccacacctgtctcctcagccagaatagccttcttcatctcctatcaccagcatccagtgcctagaacacaactatcacttgggaagactctatcattttatagatgaggaaaccaagatccaggagaaagaatgatttgtcccccagggtcacacaagtcactcactggcagggccaggaatctaacccacgtctaccacttcaagctgacacctttccctgttgcatccctgccttcaggaagggatgggaatgaatttgcgataggattcatttatgatcctgtggacaagagcctccaatgactctccggggatgcctaatcacccctttataagaaaggctccaagagcaagatgccggactctcccatggggagagcacctgtagcctgttgtgttgcctgcccaccagctggagaggctgaccctactcagtactgagattaatgaagaatccaacctcagttccagcaggaggtgagcacacgatggcaatccctcccttctctgcacgcagaaagttcatcttccaggaaatggagagttatggttccttcccaccccctacccccagttgatgctttctgagagtcaaccagcatttattaaatgtgccgtctcattcggtcattcaaccccagaccataagtttcttgaagtcaggaaagtatcttaagtttggatcacagaatatattgttctaaagggcctttaggattcaccaggttccagcttctctgtttacacctaggaaatggggtccagagggtgaaagagacttgccatgtgaatgactgcagagccaaggtctgggtcctgctccctggtgtcccacactgatctctctgctccatgtctctggactctggccctgacaagaagcctccttggatccttccaggcctgagagcagagcaggtccctgtgaaggtgtgtctgtccgttgatgggagagagcatgtcaagattcaaactgcaaatctgatcgcctcacttctctgttgaaggactgcagtggcttcaccccaccctcatatgaagttcatccttcaggatgggtcctccgagaccctgaccccgatgtcgtctccagacacggccctgtcactctgagccacagtccctcacttggcacttcccagctcctcccttaccttaccagctcactcagggctgtttcccacctcttcacttggccaactcctgctcttccttcagctgaatgcccagcctcctcctctatgaagccttccctcatgttatcccaccccacacacaacctccaaagcaccctggacttttggatttccattcagctcactgtgcttgcttatcctctctctctctcctccacccaaccacagactcccctggcctgggacagtctctctctccccatcatcaatgtacttgcacagtcaggctctgtcatcagcactcagaggatgttcgacaaatattgtaggggtgtttgattgacaagtggagaccactcccctttatgtgtcctgctccttgactcagtggaaccacagaggccactgagaatggacccagaagatagttcctgctaaggcaggggcagtgatagaacaggagcagctcatggagaagcctctaaccatctgccccacaggagtgtctgtccaagacagctgatgttccagtctccatatcagtccaggatgcatgtggagggggaagtcttcatctccctctgatccccggctgctacttcctgaaccttcttagggctaaggggctggcaggcaggcgggcggtggggctgccacacgctcccactcctagcacatatcctgtcgctacctcttctggcaggatgttattgcagagcccagggctgttattgtcaggagaatggctttcctgagctcattaaaggacctctgtcctttctccaggaccgaccccccttttcctctgtccccaatcaacactgagagaaaggggccctgggacaactccaggtggagcatcacagctccccttcctcctgctacttctctacttggctggctgatgactatgatcacagatggtcatcattcctatttgaatgcctttgtgtgcctaggcattaatggccattgactaattcagccctcacagcactcatgtaagataaatgttattattaatctcatttatacagtagagcaagctggggctcagagaggttaaatgatgtgctctaactcacacagcaagaaggaaaaggaacagtaattccaacacagttcttcctgactccaacaccctttctcttgcaacacccacttgctgccatgagctagtttttgttctgtgtaagtggagaattgcagtgaacgagggggaaaaggccttcccaggagccagcatttccctacagggagtctagaggtcacaggatcttcctcacccttgagaaagagctagaactaagatgtaatagtgagatagcacccatcttgtgtttgctcaggccttaaccaaatcaaagcacttccacccctgaaggagagaagccattgactcttcatcattcatgggacaaggcacaagaagaatgaagataattaaaaactggaaataataagttgaatttctcttaagtgattttgtgttgatggagcagaaatgtagcagctaagagaccacagcttggttaccactagctgggtaatcctgatatatcacacctactagggacctcattttcctaatctgggaatcaggaataactacccccaccatacagcatttgttatatgaatccaatgactccatgtctatgaaagtgccttataaaatgaagcatacaacccctgcctcagatttgctgaaaagattaaacacattgatacataaagcacttagcacagtgcctggaattcaagtgctctataaatcaatggcagctgctactttgtcacaaagtaatcatttctattaatgcatttattgttctgctgaagttgtatttgcagtcctgcagcacagcagactctgggtaaggaggcagattgggcctacctggtcaacagcaagaaccctcctgacttccacactcaacacctagagccatgcgccatgaaagagcaaaaccagcacctccctaccacctaataaaacagagagacaggccaggcacagtggctcacatgtgtaatcccaacactttgggaggctgaggcagaaggatcacctgagcccaggagttcaagaccagcctgggcaatacagtgagatcctttctttacaaaaaataaaaaattagccaggcacatgcttatagtcccagctactcaagaggctgaggcaggagggtcacttgaggccaggaggtctaggctgcagtgagctatgatcacattactgcactccagcctgaaagacagaatgagaccctgtctcaaaaacaaaaacaaaaagaagagaaagagagacagacagataaacaaaagatgctcttagaacaaagatgtgatgggaagagcagagctgtctgcagtggcattctctttgttaccaccagagctaagtttgctactggacccttatctgatgcctaaagaaccaaaagccttttacctacataatctcaatcaatcactaccacatccctaggaggtagggactgtcactttccaaagaaggagatcaactttgagaagtttagtgacttgcccaagctcacttagtggtgtgatctgaacctcaaaatgactctaaagcctactttcttaaatattcaagccgggtccaaagaccagtccatcacttactggctgtggcttgggacaagcctttcttgaatttccattttccttgcctatgacataagtacacataaaactcaccttgtaggactgctgtggccattagggtcttgtggctacagccccaacgtaatgcctgaccaaactggtgcctggttcttctcatctttaggctacatcaaattttcccaaacctgcttgaccataagaattacttggcatgctgaataaaaacagattcctgaaacctgccccacacctgctgaattcgaatccttagaaaatagttcccaagagtctgtatttctttttagaagtctctcatgttcctaattttttttttttttttttttttttgatacggagtcttgctctgtcaccaggctggagtgcagtggtgcgatcctgcaccactgcaacctccacgtcccgggttcaagccattctcctgcctcagcctccccagtagctaggactacaggcacatgccaccacacctagttaatttttatatttttagtagagatgggttttcaccgtgttagccaggatggtctcgatctcttgaccttgcaatccacctgcctcagcctcccaaagtgctaggattacaggcgtaaaccaccacatccggccgttcataattcttataatgagataagcttgagactcagtagaccagtgctggctacacattagaatcacctgggggagcttttgaaaaatcccaatgacagacctcactcaagacccactgacttagagttttggaggtaagcccagccagacatcaggacttttccaaagtttccagatgataccaatgtgcagccgcaggggtatgctgaataaaaacagattgtgttatgccaatgctgttcacaagaattttgtgccatgatggaagtgctccatatctgtactgtcccgtgtggtaagtaccaggcacatgtggctatagagcccttgaaatgtggccagtgctgccgaagaactaaaattttaattttatttaactttaaataatataaattggaatttgcatagccacatgtggctattggctaccgtattggaccactgcagctctagacctaggagggtacactacccctctagataacaggcagggactgaagaatgaaaatgaggggctctcctcccttcataaaaggattgggaggcatgctaagacatgtccttggaaatcaaaatgtttttgtgggctctgagataacaattggtcctggtggtctgttccatttggtaaattcccacctggcccctttcttggctcaactgaagaaggaaaacacccatctccaagcttccagcaggagccttgaagttgcaggaaaagccaggggggcaggctgaggagactagcttctattttaaagacgttttcctctttggaataagctccatcctccaacggtgcagacagggtaaaggttgtgactgacattgtgttgctggacattaaacagcctccgaggtcacccggcagctggtgggtatgtcctgacggacggctcttaatcgggctcacccctgcccctgggaaccttgaggtcctgggggtggggcaaggaaagggaggccagctgttggtgatcaccatttgcttttcccttccccctccacattccctgcccctctatgtcacctcatcctgcaatgccctgtgatgatggatgtttgtgagttaaatggatggacaggcaggcagggagtgtgagtgcagggagcagaccacccgggcaacaacacaggaagctctgttgggtttcttctccctggtgtgagctcgttcctggatgacaagctgtgatttccaaccaaatgttttcttacagtcaccacacctattggttttctctcccctggcaatatcttcagtggggcttcagagagatcatcttacttttgctccattagggcgatgagacttcgagaccagggtacagggtcatggaagtaatacaaagctgcagaagaaggcctgggttggggcaaggcaagcttctgagcccagtcacttcacccttaatatgggatagcaacacccacttaacaaggtggttgtgaaaataaaaccagatgatgattaatagatgaatgtggctgtcaagtagcaggggtactcaagtacgattcacccttgaataacacagcctgcctctcccgcctccccttccatctcctccacctcttctgcctctgccatccctgagacagcaaaaccaacccctccttgcctccttctttctcctgggaatgtccactcttttggcttccctgagccacgttggaagaagaagaattgtcttgggccacacatgaaatacactaacactaacaatagctgatgagcttaaaagaaaaaaaatcacaaaaaagtctcataatgttttaagaaagtttatgaatttgtgttagacagcattcaaagttgtcctgggccacatgcagccgtgagccgtaagtttgacaagcttgctctactcaacgtgaagacaaccaggataaagactattatgataatctacttacacttaatgaagagtaaatatattttatttccttatggttttcttagtaacattttcttttctctaacttcctttattgtaagaatatggtataaaatacatataacatacaaactatgtgttaatatgtatgttatcaataaggcttaccatcaccagtagactattagtagttttcggggagtacaaagttatacacggatttttgactgtgcattgtttaagggtcaactgtagtttaaagtctgagcttccttaactctgttccctaggatttcttagccttcaccttgccctttttcctaaattttctcttaatatttctcaaccctaagctgtgacctttgacccctgtggatttgttaccctaagaaaatcccacaaacaccctaacaatccccaggcctgaatgaccacacacaatgggtatggattccccacctagagtcagacaaccctccagcaccattttctctacaaaccttcagttccactcaagaagcaactgatgtgtgttactggtttggtggctgaacctcatgcatggggtggaacagaaagaaggctataaagtcggtaagacacgtgccctgcattcaaagaactcctaccggtgacaataactcacaacaccaggtttcaaagcactgtcctgactactagctcttaaaacaacccaatgaagagaatgtggagattagtatgagaattcccatattccagatgaagacattgaagttcagagagttgaaatgttttgcttaagttcacaagtagtagatgtacaaccaagtctcaaagtcagattttttttacttcaaatgataatttgcatgcaaagcactcataataggaactgaattattgatgagcattcttcattattgtttatccaaattccatgccctttccaacagaagggaaagagtgtgcattttaaaaacaaacaggctgtgattaattacaaacacaggtgtttaaagaaaccagatattaatatacacttacaagccaaggaactatattcatcttttaaggaacttggtaatgaatgtgggttgggaaaacttttggagacatgatagttttaaaaagggtagtatttgtatggggagagagaagaatcaggggccttccagacagggggtactgaagttagcatgcacattttaccaatttatacaactcagctatgaggccaagcattgaagatgcagaaataaataagacaaggtcgctgccctggaggaaattacagtccaaagctgatgcttttctatgggactgtcagggaggatatccaggaaagtaggcaactcgagctcagatactgaaagggccttgaatggcagtccagggaattcggagtttatcctgccggcaaggggttgtgaagaggacttttcctccaacccctttgtcctggtcttggacaaagggccacacatgacatagcctcacagtccccctttagctacctcccccactaccccgccccaaagctcattattctgtaatcagttcggagaaccacaagacaaggcgataaatctggtccttagtgacactcctctttcccctcccccaagcctttgttcctccctttgtctggagatgaagcctcccacccacccaccggctggagcttccaggctccctcccttcccagcccctcctctctctcaagcgcccctgcaaatgggctttatggtttccacggaaaccagagaggcccaggagccagggggtcttttgcaaaaacactgtttgtgccaccatttggggtggcagagacagtgagaaagtgcaaaaacagcttacaaatgcctgcccatcatgctctcgatctggtttaagtggggaaagaggaactgagctcatttccacttcaaaggggctgtgttgggttaagtccaaactcagcctggcagccctaaaggaacacacatatgcacaggcgcacacacacacacagacacacacacacacacagacacacacacacacaaagacacacacacacagacacacacacacacaaagacacaaagacacacacacacagatacacacactcatatacatacccagtcacactcatataaacacaaacataaacacacacacactcctatacctacccgcacagccacactcaagcacaaacatatacacacacacacactttcacacatacacacaaatccacactcaccagccccgcctttcccagtacagctgggctcagtccactaagaaaacaaaccttccataagacagccctgcacttagaacaccctctgtacattcccaggatgcaaaaacccttccaatgaccacaccctcagaagcccccatcccccagccccttgtgcctcagtcccctggtcttaaaactgacagttcaaaacttgcttcttgcctctttaagcacctggtgacaggagagtcacagatgctgcagactggaagccccccacccctgtcggctttcccacgtgatcccagcagaagccattgatcctgctcaacaggacaaaggatggtgggcaagtgataaatgaggacaggagagaaacatttcatttcccactttgatgggttccagcctgaagatccagacccctaagagtgtattcaggttggaaaggcaccctccaagtaccagtagtattttcagggttctgtggatgctggacaagccatagcagtgcaggggaatgaaagtgtgcagcctctatctttgtaacaagtaacagcattccagattgtactcatcggccattacaaagcagtctggaatctggtgggcggttctggtgttccagcttaattttttagtaaataaaataacctagggattagttcttatccagcacagtctgcttgggagaccagatatttctgagcatcagggggtcttggtggtggggaaacacagaggtcaagccccttgagtcggtgctttatgtagggcccacccgaagtccttgcttagtttactctgtacctctactttgcattaattctttactcctcaaagggaaaataagagacccctcctcttcctctgcctagatctcacccctgaatgtggagtggaatttaaatctggtcctgtctctgcttaaatcatttatccaccacctgagtggcaacttggtgtagtagatttgggttccaacttttctgggtccctaacctgactctttcaattgcaatattgccttagcaaagttcatttaactctattttttttttttaaacagagtctcactctgtcaccaggctggagtgcagtggcatgatcttggctaactgtaacctccgcctcccaggttcaagtgattctcctgcatcagcctcccaagtagctggaattacaggtgcatgccaccacgcccagctaatttttgtattttcagtagagacggggtttcaccatgttgcccaggctggtctcgatctcttgacctcgttatctgcccgccttggcctcccaaagtgctgggattgcagacatgggccaccacacccagcctatgttgctgtaaatgacaagaattcattttttttatgagaatacatggacacacggaggggaacaacacacactgttgcctgtcagagggtggggggtgagaggagggagagcaccaggaagaataactagtggatgctgggcttaatacctgggtgatgggatgatctgcacagcaaaccaccaggcacacattcaccctatgtaacaaacctgcacatgtgcccctgaacttaataaaagttgaaaataaaaacataaaataaaaaatcaagatttcatttcttatagccaaatagtgttccattgtgtatatacaccatattttctttattcatccattaatagacactcaggttgatttcatatctttgctatggtgaaatgtgccacaataaacatgagagtaaaggtataacctgatacactgatttcctttctttttcttttttttttttttttgataaatacccagcagtgggattgcttggatcaaatggtaattctatttttagttttttgagaaatcaccatactgttttccatggtggctgtactaatttacatccccaccaacagtgtataagagttcccttttctccacatccttgccagcgtctttgactctaaattttatctgcaaaatagatagaataacataggaccctctcctcaagacagttgcagggattgcaagataacaatagtaaaatgccctgcaaaagtgtgttcaaagcacactacttctcttcagtgtgctggtagatgtgaaacaacattaatcatgttctctcttgccatgttgtttatctcaaggaggcaatagatcttatatgtgcacattaatgcctagtgtctctacaccagtccctcactacccatgaaaattaattttgaaccaatcaattttgagtgcctaatgtgtattctcagcacaaagatccaagcaaaactgtaaagtgcaaaaaagaaaaatttcattgttacttatgtacaaacaatttactgagcaatttgaccccacataccaagcactgcagacgaccagggggtgagtaaaatgcaatcactttcaaccaaacagtacaatattgtagttaagaaatcctcgggccctggatcccatccaccctcgttcaaatcccaactatcccttgctggctatgatttcatgtttggcaggttacgtcatccttctgaacatcagtttactcatctgtataatgggggtataatggggctgatcacaatagcccttgtgcctcccagaattaaatgaaattaaagcccatgaaatactccagctgaggaagtgtccagcttggaaattgttcaattgactttgctgctggtgctgctgttgttattattattattagcaatgccacttaagtctagaacaactcaatcacaaaataatacagtagagcagagaaggatagggagaacagtgaagagtccaggggaccctgggaaccccacttgccctttccaggtctttgttacataaagctaaatggagcggtgagacccaatgatccttcatcgctcactgacttcaaggagtactcaagaagtaagggtcacaaacaaacaacaaaaacaaaaactaccaacgaactcataaagcttacagttcagctggagtcctcccattcaccttggttccctgactgccaaatttttttctggaaagctccagttgctcctccctgtgccttcccctcctccccaccaccttcctcctcctggggaagcagaagttcctgagggccagagaggaggtcagagatctccaccggcctggccaagagccaccaggacagcttcctgccagcagccttcagtaaggagctgtccatctcagggaaacccaaataaagtcacctctccacccgagagcattttgccctttaaggaaagattgtatttggagcccagaacatatttatggcttcatgaagatttcatccaaccttttacggacagagtcagcatcaaaggggcctgcccaggcccatctgctaccactttcataggctgacctgggcctggatcctgaataaattatgaagtcactgaactttgtttctgataagccccttttcgtgctttcttcccttttctacctccaatcccacccgtcttccacaccatggccaagtacccttacaatctaaccccaatcagtctttccaaagatgaggctccaagtaattattccaaatgactatctaatatagcttaatacatttccaccctagaaaagtttaaagaagatacataataagcatttgtgtgtccagaattttacagacatctcattttattctcacaacagcctataagtaggtacaattgccacctctacttggctgacaactgaggctcagaggtgttttctaactcatccatagattcaattgctggtaagggaagagctgacagttcacctgaggtctctgtgagtccaaagccgtatgcatctcacgataccaaaatacaccaaaagacagaggaatttctggcattgccaggtgtctaaagggatctgcagggagagacagaagtttgagaaatctcccttaaggaactaaaatagatctaccatttgatccagcaatcccactaatgcatatctacccaaaggaaaagaagccattatacgaaaaagatacttgcacgtgaatgtttatagcagcacaatttgcaattgctaaaatacggacccagcccaaatgcccatcaaccaacaagtgaataaagaaaataaagaaattgtggcatatacatatatatacgatatatacatatatacaccatatatatatatacacaccatatatatataccatatatatacataccatatatatataccatatatatacacacaccatatatatatatatatacaccatatatataattgtggcatatatatatatatatatacacaccatggaatactactcagtcataaaaaggaaaaaaataatggcattcacggcaatcttgatggaattgcagactattattctaagtgaagtaactcaggaatggaaaaacaaacatcatatgttctcactcataagtgggagctaagctatgaggatgcaaagccataagaatgatacaatggactttggggactcggggaaagaatgggaagggggtgagggataaaagactacactttgggtacagtatacactgctctggtgatgggtgcaccaaaatctcagaaatcaccactaaaggactcattcatgtaaccaaacaccacctgttcccccaaaaacctattaaaataaaaaagaagtttcaggaatctctatcacactctgtaaggcagcttccaaagcttccttctcctggaaccagatctgggtctgtgcatgactgaggtgtcaggcaggacagcctcactctaggacacttctgatggcaacagttagccacgtttcagcatccaaaaaaagagaattcaccggacttcccagcaattttaggaaaggaaggcaagagatttcgagacactaggtaaatttttgacattccattagatagataggtggataggtagagagagagagagtatgaatacagattgagagttttcatgccacaagtttcatgtcaattaaaaataatccagtgggactcaggagcacggcatggtgaaacttgcacggattttacaattaggcaaacccagctctgaatcctggcatggccgtgtactgatcacatgaccttgagcaaattgcctaacatttcctgagcttcagttacttctgcaaaaatgagatgataatacttccttttacaaagttattcaaaggatttgggattgtttatgtaaaacacctaacagtgtgctgagagtggcagatgtccaccaaccagatacatccccaggctgaacggaggccaagaagcacttcacccattccatccttatctctcaaagtccagggcagttccaaataaaaggcagatattttcaccaaccaaagcagctgctctggctttcccggaacaggcctctgcctcagagcccttcattgaccacccatgtttccatggttgaacgtttacaggagaacattcagcctccccttgtggttcatggagcccttaaatccccctccagcctcacctcctgtcattcccctctcccactcacacacctccagccagacactcagtgcaccattcaccagacacagtattccgtggctttgatcacacagttcccatttcctgaagtgtcctcctagccctccacctaaagaactccctgtcatcattaggagcccaacctaaatccatcaagcggaattaaccgctaccttctctgtgatcccatcacactttacaatgaatggagaagaggaggggtgggggcaaggtgcacaacttgctaagtgcccactatgcgccactagcaactccacagactctaaccaccacatgagttaagccttattatccccactttgcagataggaaaactagttcacagagcccagcgtatgatcctgaggtcaaaaggctccaaggcccctttcccctacttactatactccatggacacatggacatggtcctgtctgtctcccacactagactgagttttctgtaaacagagttcatattttcatcatctctgaaacctcacggccccctcggctcaggctcaagcccagtccctgacacctagcaagccccactaacattcagcatgcatagggagctgctgggtcatctgtctatttattatagagactggatggaaagaattctggatcactgaggaagagcatttccaggttgaatgtctcagctttcttcatggacaccagttcgctggtgggaggctttaaccttgacaaccatccactttcgggggcttgaagaaagatttcctgtatatattttaactgcattcattttaaagggaactctaaatttttaatacaaaaagcccaagtgtactgtgatctctgagctggctatcacacttcgtaaagataggccatgggaccaggaaggaccgcagctcagccaggactgcgcaacttggggcagctttcccaaattctaggaaccaaggtccaaggaaaccaacaaccttgtggccaacctttgcacatctgtggcacacaaggtttttttaagcagactcactatgtttgaggatcttcccacaatctgagcaccactttccaagttagaagctagaattcactttcccagattcctttgccactaatgctagcattgacctgggctccaccattcaccaagtgtgaaaattcactttgaaagaaaaccacctgaggatccaggagctgcatgaagccgattttctgtctaaggagggaaagtagcagagaatcttcaaagtctcagagacagctgcagtgagagttctggagatccagtcccagacagaacttcacggtgcaagcagagaagtccctactagagacagataattctcagatgattccttcctgcagcccaatgctccagagattctgtgagctccctgagttcctttatcaaattcattttctgcataaatagctgagaggggtactgtttacatcttgtattgtaaagaggggtattgtttacatctaagatccccgaaggatgcaaacctcatgcctgccagcctaacaaaatcaaacctcatcggactgaatagacctcacagcaatatctgactccccacctaaactgggatctcatgtgcctggaacatgctgagtagaaaataaatatgtgctgaaatacaatgaagaaaagccacaagactccttgttcacaggttgaatcttggactctgagaccctgcaccgaagaaggaaaaggacagttcccatcccatatgctaaataagtctctgcagtgcagtgggctgtctttgtgactcacaagctggttctgaaaatcttctttcggaaggagtttccagaaatggttggaacaatggcagcatcactagaaagaagtctctcacctcccacagaagcaactttgagggacaacatatttttgaatttgcaattttggagtgtttgctaaaacatcacttccattatagggtgtcacagaaagcactgagtctcaggagagcagagtttagttccagttctgcccccaacctacagtgtgtctgaattatttcctccttctttgggcctcagtttccctgtctgtaaaatgagaaggttgatctttgctccccattgcctctttcaggctctttctccttcaaaaagctgattcctttgacacctatgccattcaaccagggtgtccaggcacagctgtacaggttatacaagtaggttgcatcctggccaaggaagcccacctgagaggtaagtagaatccagcccacatccccacaggcgtcacccttgggtgggcccgcttccactggagaaagactactttttcctaatttgtacaaaggcatcatatgagctagccaaggccccaatttcagctattttatacccttgccctcctccttactggggtcattctactcacctcttaatcatccttcctccttcactgaagactttaggtcctggttacaatcatgttcactgccctcttcctacaataattctcaaaacttcaatatctgtatgtgcaactcaacgagccacttggtcttgttctttgatgttttctaccccaccaacctttgtcctcactccactgcagccacccactcaacagccacaccatagaccatgacatggccagaaatctctatttccaaatccttctatttggccttcatttccagtaattctagctcatttgctctggtacccttcatccccacacttctggccccttatcttgaccactaacccattgatctcaccacacacccaccctccagcagccacttcctgcctttgtttccttccttacataaattagattccattgtccttctttataattactctcacaaacaatcccaacgccctggagcctctctcctctgtcacaatccctttgcaaaactccaacatgtgcttattaccttctctgtgcctttgctgcagcatctaaatgttgccagagaacttcacacaacctggctaagtgatttcactttaaattcatggacacaaatctcaagtggaaaattggagctggaagcttccctaggaatttttgttttttcgttgcccaagataacattttatccccctaaaactttctagacccctttccctaccccatgctcagcagatgatctcattttacaattcacttagaaatgggaggttacaaagccaggacttcctgatcttcccaccaccaaactctaccaacttatctgcttcttaatccatcctctcctacgtcctttctagtgcactgaataaaatgtcctctcttcctaggaaaggctgaccctcccacatgtgctcctgcctgcatcccttctgtctttcttgaaggcttttctcctccagtcatctcttctccacaaaagcatcagtctcttcatggctcattcttgccctcaccctcacttatagttttggtctttcttagattgttactagcacacaaacatgcccttgtatctttaattttttaaagcccttctctagctattgcccatttctctccttaccttcccagcaaaactgttattgttttggggtgtttgtttgtttgtttgtttgtttgtttgaaacagaaactggctgtgtttcccaggctggagtgtagtgacacaatcttggctcactgcaatttccacctcccaggtccaagctattctcctgcctcagcctcctgagtagctgggattacaggtgcacaccaccacacccacctaatttttgtatttttagtagagatggggtttcaccatgttggccaggctggtttcaaactcctgacctcaagtgatctgtctaccttggcctcccaaagtggcaaaactgttgaaaagagttgttttcacaagcattctccaatcctcctatccactcgtcagctctctgcagtctgtcttctactgtctgtcttctacaaacacaacctgtagtctatcttctatgaacacaattcactgtaacccctgacccatgtcaccaaagacctctgtgtcaccaaatccagtggatcgttttctggtttcctcttatctgacctctctgtaggattcttcacagttccatctctatgatgttcagatttttatcatcagtccagacctctcttccattacatatcaaactgccttctcaacatccgcacttgaatgtcttattaacatctcaggaatgatatggtgatgtagccaaaacctaagctattctttctccagccttccctatctcactaattggtatcaccctcttcccagttgctcaagtcagaaagtttggataattccttctttctcaccccctcatccacccactcactcaaacccaggcgatcagaagtcctattcattctgcatcaaaaatatagctcaaacctgcctctcttctcagttgctgtggataccaccctaatgtgacccaccaacatccctcatcttggcctctgaagtagcttcctaaccggtctctctgctttccctcctactccccttcaactcattccctatgcaacagcttgaggaacaattttaaatgcaaattagattgtatcatttttctgatgatttctcattgcatttaataaagtcacacttcttaccacagccttcaaggtcctacatgacctggcccctttttggctctctttgtctcaggccaccccctgcctggcttcctgggactagccttccagttgccctgacaaccttcccagaacctactgtgatctttcttgcttcacagtctctgcatttgctgttttctgcatctgggactctctgagcctccgttgcacagggcaggctcattcttattcttcagatgtcaacggaaaggtcagcttccaagatggccttccccaagcaccttttctgaagttgtacttaatattattttccacttcagatgttatttccttcatagtatgttccataactggccatttcataaaatggccagtttatgtatctgtgtattttctatctttccctacaaaaatgctaagctagacaaggtctatctcatcactataatcctagtggctagctgagtgagtggcacacagtaaatgttcaacaagtatttgatgaatgaatgagtcatcatgatttgtccccagtctaatccttcctcctttattccacattcctttgtatgtatacccaacacacaagctagtcaacttgctcctcagacctcctatgctcttccttaccctctttcacactgtctcctcaggctggatgcctcctaccacccccattccacattcaagtcaccaaaatcctagttatcctttcaggcccatcttcaactctaatgcctgagattccaccattagccattcttagaattatagaatgtcagcccttatagatggtggatcaaaattacatttatcatttcaattgcccagagtgctgcatggagagggattatgagttttggcaccacaatcataccagaaaggccatccataacaataggaaggttatgactgtcccaatctttttcaagctcctcatgatacagatgggaaaactgaggttgaggcaggtggcatgacttcccagggaacacactaagtaggagacggtgatgggaacagagtttccatctctacatcaccagtgtggaattatatcaggctaccttttctacatcccagtctgagaagctgaggctcacatagacttgaaaggtgattgagattgtgaatttttaatttaatatatatatatatgcaagatattatttttatctttctttttcaacaaagttctgaccagattaatttcatatgcaaacttctgtcaattttttctagttttgttttgatactaccaagataaaaatattttaaatacaagattaattaccaaccaccacattcctggaattgtgcactctcttcttccttgcacccttgcaaagatcctccaagacttctgtagtggaaaggagcttctcaagtcacttttctttgtaacaggagtggcaaacaggagaatgggaggaaattaaaacctatagattacttcttatgtgctggagactgtgagagttccttgcaaacattacctcaggtaaccttcaaaatagttctctgaggaaattgcttttaaagctattttattttgacagatgagaaaacagcctattaatgggtgttatgcccctagatctttgctcccctgaatgtagtctatgaactaatagcatccaccattgttggagggctttaagaaacaagatactaaggacaccctctagatctgttgaatccaaatctgtattttaacatctcctaatgattcttctgcacacacaagtttaggaagcaatggtctagataatacaaccaaataacagatgaattaggattctaattaggtctgtctgaggactaattcccagaatctacaacaaactcaaacaagctagcaagaaaaaaacaaacaatctcatcaaaaagtgggctaaggacatgaatagacaattctccaaagaagatatacaaatggccaacaaacatatgaaaaaaatgctcaacatcactaatgatgagggaaatgcaaatcaaaaccccaatgcaataccaccttactcctgcaagaatggccacaatcaaaaaataaaaaataatagctgttggcatggatgcagtgaaaagggaacacttctacactgctggtgggaatgtaaactagtacaaccactatggaaaacggtgtgtagattcctcaaagaactaaaagtagaactaccatttgatcctgatagcccactactgggtatctacccagaggaaaataagtcatcgtatgacaaggatacttgcacacacaagtttttagcagcacaattcacaattgcaaaaatgtggaaccaacccaaatgcccatcaatcaaggagtggataaagaaactgtgatatatatacatatatatgtatacacatatacatgtatatacgtatatacacgtatacgtgtatatatacatatacacatacacatatgtatatacacatatacacatacacatatgtatatacacatatacatatatacgtgggtgtatacatacacatatatatacatatatatataaaacatatatacatatatatacacacacaatggaacactactcagccataaaaaaagaatgaattaatggcatttgcagcaatctggatgggatcgcagactattattctaagtgaagtaactcaggaatggaaaaccaaacgatgtatgttctcacttataagtgggaaataaactattcggatacaaaggcataagaacgacacaacagactttgggaactcagagggaaagggtgggaagggggtgagggataaaagactacaaatcaagttcagtgtatactgcctgggtggtgggtgcaccaaaatctcacaaattaccactaaagaacttactcgtttaaccaaatatcacctgttccccaaaaacctatggaaataaataaataaattaggtctgcctgactgtacccttaacagaatggacacccaagacacaaacatcgtggttttaaaggagacaggttaagtcatcactggtataagttgtacacatcagcagcacatctgattacctgtctagtcctatgaccatcccttccaaagctgtctacaatgtagaaaggaggaaacctggtgatgtggaaagtaaacaggcctgaaaatcaagagaaccaaaccagccaaatcaaccactacttagcaatggcccagtgtcccagggccccattttctttaccagtgaagtgggactggagggacaattctgtgcagctcaattatgtggcattcctcacatcagcccactgagctgctcctgaaaccatcagagtagaaatcacttccttctccaacctgtaatgtctatctctagtcaattcctttggtaattgctgtgttgctttttccaaacataaaaatagcttattatctgcacagcaaataaccatcaccttgcaggggtcctgacagtgccccagtacagagcttgcaaccagctgaatcctaagaatctcaagggcttccaggtctcatcccggaagccaactaaccaccatattggtgtatctattttcctaagggcaagggctgcatccattatcttcattcctggcacgttgcagtgacagtcctcagactctgggtcactggctggccctgctaccaaagttttttttctgctaaagagaccagacccggtccacgtagagtaaagcctgggtccctctctctctttctatttttttgagactgagtctcactgtgtcgcacaagctggagtgcaatcgcacaatctcggttcactgcaatctctgcctccctgggtccctctcttatcatcagaggctgcttcatccctatcatacaggaagacctagtgaccttagggacatattccagacccagaagaaagactgccaaatcttcctcgatgcccttaaacatggcccagattttctcagggaacggaagagaagttttccttgagtatttttatacagggggaaatccaagatcttattttctattcaatcggtacatcccccaccctcaccacaaaagtccttcctccatgaacagtggatgtccagacatggattcccagaaactgtttgactcagtgtcggctccacttggctcatccataacttccccaggaagccaaccagctttgcaaaccatcatccatccaaagagacctgaagacctagtttccagtatgaacactgaactggttgccacaatgccataatttgagaaattctgctgagaagatcagaagagcccatgtaccatggtccagccagtccttttttctgtcactgggtccccagagggcagcccttaggagccagaacatttggtttaattgggtcaggggttaataatcttagtacttatcaatcccagtgtgtctcttgtagcaccagtcctaacttcatctttctgggctattttaaaagcaatcaacttgtttgctgtaagcaaaaaccagcttttctcaccttttctcctgcactttctctattcagttgttcgattatttaagcaaacatcctttaagcatctgccacattccaggcactgggaacaaaaaagatgcataaacgtagccctagtctagaggatctgcccagagagcagaagagatgagaaggaatgcagagcccagacagagtggccagatctgggcttttctagagacctgttccctgtgcgcaggggacaaggggctgggagtactagggtgctggccaagtggggtgcttcaagtaggtttggggaatccatttctgattatccactgtccatgtaagaagctgcctcaagtagcaagagatgtgccatataactaaataataaagtacagaacaatattcggggagatgctcactgagcacaaatagtcctttgtctcatgtcctcaggtttctagttatttaggcaagaaaatgcaccctaaggtggctttggaagtaggagaatgtcagacatttatacccctgtggaattgaacgtgggcctgtgcattgagccagcacactagccctgtcttgggacccctctgtggtgcagaccagaatatgggtgtttgtccccactgcatcctgtacttcctccgcaatatgcttactgcactttattattgattgttaaactccaaaagagcaggaatcacatgtatatcttgtattgcttcatctcatcaccaagcatgtgcctgatatatggacatagaaagatatttctacatgggacaaaatacctgagcaatgttcctccaaaccgtcaaggtcatcaaaaacaaagcctgtgaaactgtcacagccaagagaagtctgagacatgatgactaaatgtgatgtggtattctgcatgagatcctggaacagagaaagaacatgagctaaaaagtaaggaagtctgaacttcagttcatagtgaggtatcaatagggttggacttcagttcatagtggacttcagttcatagtgaggtatcaatagggttcattaattgtggcaagcatgtcctgctaatctaaaatgctaataataaaggaaactgggtgtggaatatatgaggactctctgtattctatcagcaacttttctgtaaatctaaaactcttctgaaacaaaaggtttattaacaaaacctcgaatgatcctcaagcacctttgtgccctcctacttcccagttggtggctcatcatggatctgactctccacttgccctgtgtgttctcaataagagacttcctctcctttctaagcctagagtcacagaacacgctcttgcctggcagtgaggaacctgagatctagtcccacttctgccaccatcccactatatgaccatgtgtcaatcatttccccctcccaaccataagcctcagtttacccctctgttcaatgagaaagtaagactagaaggtgcttctagtcggtccatgaaggcagggatggtgtgtctcattcacggcagcatctccagcttctaagacagtcccaggcacaagacagacactgaaaaacaaaaaaggattcaatgaataaataaatgagcattgctctgatactccaacactctaactagaaatggaaacttctagagtgagctcagcatctcagaacctcctttcttgtgtatttgatttcaagctccctgggtcacatctttgtgtctgtctccaacgtcgtgcatggagactttataagcatttgtgcaatgattgagaagagctggccctcccagatcaggccctgcaagtgttgaactccttggaatgcagacagtggtgtctgctgagacggtagggaggtgtgtcacatctaaagcttattgcgctgggggtcatcctggcttctgaagttggggaggatcttcccacggaatctatctgcctctttttaacaagctcagactgaaagggcctgggcctcccaacccctagttaggagaccctggcagataacttagaggaagatagctcaaacccagggagggaagaaactggaagtgacaaaaagaataaaaaagaaaagaaaaaaagaaaaaaaaaaagccctgcacaaccccaattgtctagagacctggattcctggattttagtcccagcccccagtattctcggcatccacgccatgagaactgggcaagattttctctgtgcctctaactggccaacctcccagagttggagtgggggtgagtaaaaacaaggatgagtgttcgttccagttcctcagaggggctgtgttctccgtgggcaattcctctgcccagaacattttccccactaaccacttacactccatgcttcagagcaggctccaggcaggcaccgtggctgtaaagtctcccctgacgccctgcctgactccttccattgtctccacgacaggccctcgaacacttcacacattcccttagaactcaccaccctgggctgtaatcgcccatttcactgtctgcctccaccagccgactgcgagcccactggaggccgctcaccatttggtcctgcgtccagcatagcgcttggcacatacgtgataaataaggacttgtggaacaaacagatgaatgaatgggtgaataaatgaaggcatgtgtgaaccagtgaattagtaagtgctgtcacaaccaagggaaggctaaaaccgtatcatgttttggatagcttacctcagatttcccaggagacttgctgaggttttttacagcatctgtaaaaataatgataatagtggctccaatagcctgttgttgccgtcgtaaattcccaaagggctttccctggtgtgtttcagaagagggagagcaggcactgattccctgccctcagtctccctccctgcctccccgtttcttcttcccacctatgttctcactctctgtctctctctgtctctatttctctctcctcactctgcctctctttcacggtctctgtctctgtctttgtctctgtttctttctcttcccactgtccctgtctctctctctctctctctctttcactctctctgcctgtttctctctcttccctctctctcaatctctctctcctctctcttctctccctctccttctttctgtctttttctttcactatttctgcctctgtttctctttcctctctctttctgtctctcttttctctttctctccctccatctctgtctcttacactatctttgcctctgtttctctcttctctctccctccctctgcctctgtctctatctttttcactatctctgctctgtttctctctcttctcaatctctctctttctccctgtctctcctctctctccctcctttgtctctatctttctctttcactgtctacctgattcactctctcttccctctccctctgtcttttctctctcaattcctctctccctgcttctatatctctctctttcactatctctgcctctgtttctctctccccatacttcatttctctctctctgtctctctctctctttctctctccccctctctctcttcccttgtctctatctttctcttcactatctcgcctctctctcttctttctctgtccctctgctttctcctctctgccctgtctctgtctttcgggtactctccatcatttccctccccatccccattcactctccactgtgtgccaggcatctcacacagaagttctcactttccttcccagacacctagtgttgcctcatttgtactttgggagaaacatccccttaccatggtcaagaatgtgggctttgggcttgggagccagacagattcagctggttccaagcccagctcttccacctgctagtctgtgatcttggtcaagaggtctaactaacctctctgagtctcagcttccttatgagcaaaatgaggtgaaaatgccccctcttaaaggatggctgcaaggattaaatgagataatgcttgggaaatgcttagcacagtggcagaactccataaatgcgagctctcttgttactatcagttctactatgattattatttttttgcattgtctgcaaacattattaatacccaggagatcattattgaagatgacaaatacctctggactgaatagccccaccaccacgccgaccccaggatcatttcagccccttcccccctcctccaaatagatgaccaccaaagcccagttggctgtggttctgaccctctggtcttctatgcccactttaatttgggcagaatccatctggatagagattccctaagcctggctttcccaaaaaattatctgagtttttagtaattgtaaatatctggggtgagtcaaggagagtctgactccataggtctggggtgatacccaggaactgggtattttcaaaagcatccagagcgactctgattggagaacagggattcaagcttctggtctaaactcagtggctactcaagtgatattaagtaaatcaatcaataggagatggtaaatgaataaaggcataaatgagtagatggattggtggatggatggatggatggatggatggatggatggatggatagatgagtaataggtctacctccatgggaagaaatgctggtatttttccccagacctatgctctggcctccaaaatatgtgtccttttgttctgtggcttttcagagcacaacacttagctgctcccctgtttcttttcttccttttcttctctgcacctaaatcccatcctttccctttcctgatgcctatttcctccatcctctctccctcctcacttcgccccctccttttagctcccacttctctttacggcttccccctcaccatgctgaaacatttgcaagctggaggttatcccaagacactttcatgagaagaggctttggacacaacagttgcacaaacagttttatttgatgaaccacagtgactaacaggatcagaagacagtgcagatattctgaagaaggcactgggggaggtaagggggtatcacagcaggcagcctcctctgcttctgtcccagttcacagatgagttccaggcaggaagtctctgcaggtcacccacggcggcctcagagggacaatttcttcccttctagaagcctcttccagtgttcactggatgctttgaggacagctctgggcagaggaggtgactctgtgaaagatgctatcttaagatggggagactaggctgtgaggagccccttcccctctcctcctccctctgcccccagagctggcgtcattccagggagggtcaagatgtccattcacatcaagctgggcttttcttatctccatcgctcatgtcttgtccttcactttcatagtcctccaagaacaaaacccggaatagacactcccagcctctagctcacaaaggccccactattaatttcatcctcacggtctctctggcagtgtagacatcatgctatctgcacacttgctccagtccctattcccagatccccaatccagcagaagccaagaggtccaatcctgagggtagggagagaaaaaacccaccccgtgccttttggcccatggcaagcaggaggcctccttaggaagagggccactgaccctggaacaagaaaggacgatgagggcaatgctcataaatttgaagccaggcggggaggcaccggaaaggggccaagggaggctctcaactagcctcttacttccttcatggagcccaagaaggagcaggaacccagctcagtaggacgcccccaagccttggcgccagctgctttggggagggatctccctacaggaagcctggtgcaaacattaacttcgctcctaaagcacggaccagccgttggagcccccaaatccagtagggggtggactcatttttattctacatttttcattgcctcagtgtgtccctgccccccaccaccccctgccaagggtaatgtgtgaacagcagagttgcagtggattggggggtgggaggaggtttgtccaaatgacccaggcacactgctgttcatgccaggctcatccatatagacatataagtacaacacacacagacaagaggcaggcatctccatcccaaacaacatgattcttggcaccaagtagcaccagtgcccaagaaagaaatcagtatgatggggtaagatggggagatgaggaaacccttcagctcacagttatgcaggaagtcagaaacaggaggactcggggagcgagccaagcagtgtctggactaaatgcagacacccctccatcctctctctctagttgcccttcccaactccccactcacataatgaaaccaaggagtacatccattcatcagccctgctgccttccaattctcatagaaaatagaggaaggatgaaagggtctgcggacaatttggagaaagcatatctagtcagtgtcatggtgccatgggggaactgcaggaatcacactagccccaatgcccaaaatgacagggaagtgatccaattttagggcaagctctggagctcccagatctaagggctggagcactaattatctgctgacacacagcccacccccggccccagcccattccctgcctcacactcagaaaacaggatagaaagcttaaacagggtcagtctctgcaagctttgggatccttaacccttagtagcccttcagaacaagaaagtagacaggggagaaggatggatggagggagggggacgagctgtaggaggggattggtgccagaaggcagggataaggagcggcagctgcagagagtggtgggggaaaaactacaaggaaagaagggaggaaagacgattgcagatctaagaaagcagcagagggaaacaagcttttactgcgtccagactcaggcttcaacattttaggatcaatatgactttcccccatttcataccaatggagaaactgaggctgagtgagattaaatggcttgtgcagggtcaggtctgtagagcccagtctctttcccctaaaccacaaacctctgagaaagaataaagcagtagaggaggacagggcggggcagaatcaggggacagttaaaggatgtgcataggacaaggagtagccacactacctgattttcagaaagcaagggcaaaggccggggagggccgagggcgccaggcttctctctgctcccagccctagtcaaccacaagggtctcctggctgtaggggatggatttctcgtgcatctgttctccacccttgcccatgttggggccgggcccctggctgtgccccgaagagtagctggctgacttctccccactgctgctgtgggaggtgcctgcgcacctgctctggccacagcagagcatggaggtgcaggcctggcggaagcgggggtcgaaaaaggcatagaggaaggggttgaggcagctgttgacgtagctgatgcaggtgcagtaggggaagatgttcatgaggaagaggtcaaagtcacagggccagtgcagcaggctgcccagcatgtacagcgtcttcaccaggtggtagggcatccagcacagggcaaaggtcaccaccagcaccacgatgatgctgagcagccggcgccgcttccgcaggccctcgatgcgttccttgcggaagtggccagcgatggtttgggcgatgaagaagtaacaggtcagcatgatggtgaagggcaccacaaagcccacggtggtggacgagaccccaaggcccacctcccaggcccactctgagctcacagtggccaccatggagtagtccatgtagcactgcaccttagtggtgttctccaagtccccggtggtgcgtaacaccatgacaggcatggccaggagggcggccagcacccaaagaactgccgtggccacggccccgctgacccgcagcctcagccgagcattggccactggcctcacgatggccaggtagcggtcgaagctgaggccggtgaggcagaagacgctggcgtacatgttgacgaagatgaggtagctgctgagcttgcagaagaaggtcccaaagggccagtcatagtcccggtacgtgtaggtagcccacaggggcagcgtcaccacgaaggtcaggtcagccaccgccaggctagcaatgaagatatcagctgagcgcctcttctcccggctgctccgaaacacggtccagagcaccagaccgttgcccgtggtgcccaggaggaagaccaacatgtagatggcagggatgagggcccccgaggatttccagtctgtgtactcacactcagactggttgtctgccccatagtagttgtcaaaatcaccaccttcctccatgctggggagtggagagaagacgggcagagggtcccagggacaccagctccgtggctctgtcacccactctgcaagcagcctgaatttctggcttgagcctcagagagtaagaagggctgaagatgcccagagtccttcccagcactcaggaggagctgcctctgggttctccagaccctggaggaagcctgtctcctgcagaatttcctccaccctctcttgccttacccccgtctcagttaagacacttccttgcaccctcctgcgtccctgtctcctccttgactcgtcactccctcctcccacctccagaccagcccctctcttgtgagtcaaaatctttagtgacagcccactttttttttctttttgtcactgagcaagtggaccaaattgacccctactgtgctgggctgaacattatctgtggttttgcaagtcggctttcctggccccgcctctcctttctggctgccacccaccttcaccccagcccctatccctccactttcctctggccaccacttcctgcctgccctttagggcactccctcctcagtgctctccgccctcctgttctcacccccttccatccaatctaaatgggacacattatgcagattgcaatccagcttgagctggagccagggctggtagggagcaaggagggtgtaagatttcgcaggatgggcagggcatctggcaggtcctcctggcaggcaggtctggccccctcggctcccttcctgcacccccagcccccacccagaccaggtgtcacaggagggtcttctcccactttgcctctcgcttcatctttctctaccccttggctttgaccctccctctgagaacttttgtggtttctagagaaatacccctacactagtgtagactgcagtttgcagtttgcggaaggcacaggatgctttagaaggttgagggcagtcacaggatggaagaaacccaggacaaaattatctctaaacaagtcattaaatctgtaggaaacaaaggcccaacaaggtcaatgttttgcccaaggtaataaagtgaattatgctacagttgattgtttgcttttcttcttctccaaacacgaacataatacagtacctggcatgtcgtaggtgaatggctgttttgtttgtttgtttgtttgttgagacagagtttcactcttgttgcccaggctggtctaggctcactgcaacttctgcctcctgggttcaagcgattctcccgcctcagcctccctagtagctgggattgcaggctaatttttttgtatttttagtagagatggggattgaccaggctagtctcaaactcctgacctcagatgatccacccacctcggcctcccaaagtgctgaaattacaggcatgagccaccacgcccagccaggtgaatggctgttgagtgagtaagtggatggatgatgtaggaggtgagtgaaaaggggaatccatagatggaagaacaagtgtaagaattactacgtggtgagaacatggatacatttcagttctcaggtttttactatcctccatggaccaagaggtttctgtaagaccacagggggtcaggaagttgagctagaggaggggggtctctagagaaagactcagtcactggaggatactgtggggaaagttccgtagacgctcactttgccccagctcatcacctctccaggacctctaggaaggaaggaagatgcacgacgtaagattttttttaaaaaatacgcaccctttccacaaccttgaagagttggggaagccacctgacgttgggtaatagtgagatggaacaaaagcaggagctgagccttagccacttccaaggcaaaagacttacaaaagctggaattggccaggcaaggtggctcatgcctataatcccagcactttgggaggccaaggcaggaagactgcttgagcccaggagttcaagactagcctgaataacacagccaagaccctgtctcacgatatatatttgtcttaagttactacaaaagctggaatccattttctggagggagagtggatcagaaaatcatatgcagagatcaaggaactcaaactcaaatacctgcaaaaaaaaaaaaaaaaaacaggccaagagtgtagtttgagacaacagagaatggtaaagactgtggcaaactggagggtgcccattccatcatagggaggcctcttcagctctagtctctggttgccacgcagaaagctggaccccagcatgactgtgtcttaggatttttccaagataatttcaaattatggatttttgtaagaaattttcatatttgtaaattttggaaatcaattttcatttcaaaaaaaagcacagaccatgataagtaaaacatgtctttggcccccgagtttgtgttctttaaacaggagcttgccaagaatagaaggcaggagaagaaccaagaggatggggcaggaggaggaaacaaaaaatgcaaagatagaaaaagccttagagtgcagtggggagatacaggagagaaggctgcagagagagattagaaagatcctgtgaccttcacctaaatgcaatgtggagtaatgaatatgttggtgtttctcatgaggactacatgttgcccacttattgtgtgccaggcagtcttctaaacattttatacattctacatatatttattagtatctcttttaatcatcaaaatgacattaggaggcaggtgctctcattagcctcaatttacagatgaggaaactgaggcacacagaggtgaagcagtttagccaagatcacccagcagtaattgtcagagccagaatttgaaccaaagctgtctcgagcccatggactaaaccactatgctcccaaagaaaaacgcccaatcctttttaaactgaggggtgtgtcatctgttaagcattagagaaatgtagggcgatgccaggatacctgtaatttaccttaaaataagctctaccgggtgagattttaaatagattgactatttagagacacacctcacattgattagacagggtataagaatcctgccaggcaataatctgtacacttagggttagcaccatagacttgatccagaaaccaccctcgctctagccattgccaaagagaattcctggttgcctggtgccagctagaaagctaacaggactctgcccctggaatcttggcattctgtacagtgggggctgggacacccaataggatccagagctggggctccatccatctcacagtacacagggtaggaattgcttggggcctcttgctcctgcatttttggctaatagaagttctgcgcagatggcagaatggttttcattatatcctgacattcattcatcatatcatgattcattcatcatatcctgatataatagtttcattatatcctgacattatatcctgggtggaaatagctaacctttggctacctcagacttcttcagtggtaagctatctttacctatcaaaggggaccttggagttgtgcagggaccagcttggctcaggtcaggtcataatagatggcccttttacgagtaaacattactgcttgggacattccaggataaagaccaagtttgagacgcctagtgatcccagttctcccatgattctcccagaaagatgcagatgtctgtgccctttgcagctagaaatacagagagaagggagctagcaagcttgaagcgtccctgaaatcaggtccctgtctccctcccctcagctgttcccactggaatgctgagccagacaggagcctggagacccctgtgggaaaggatatggatccatcgctttcatctgccgacctccaggatgtctgccctagagggagtaaagagcagttaatctcactagagttaccagactcattgagaggggagggaattgggggccagggctgggaagaggccaccgtttgggaccagagagggtagagtgctgataagatccggcctccaaccaggagccagctgtagccagatggcctgagtgccccctgcaatgacagcctgaagtgagcagaattagccagctcactccttatcctgcctgatctgatctgtccctgttccgcattgcaccattccacacagaagaaagactggaaaataggaccagtagctgaagatgaaacttgtgtgtcccggggctcagaagtatatgggtcctgggcctcacagactagacatatacaaggcctgggacagatatcttcttttcatttctgccccccaccctacttggcacctggtaaaagtctgttgaattaaagcaatagaaacacactcaggagaagggtgtaagacttggatcttcaccccagagctgctctactgcattaacaaggcaacttttgctaaagtgatccaggaaattctactctgagctatctggcaaaccaatagctaaggcaagcctgttggccagggtgcccagagctggcctgccctggagaggcctgcacaggtttcctggggaacttccctctctctcctgggtgatttcttcagggcattgtgatgagaccgagatgaatgacacctggcagaaaagcactccagacctgctagtgccctttgaggagagcccagacttctgggagacctctggagtcagtggttcaatattcctcacccaccccaccaccaggtttgtgcctggcctaaaaagcagagcctgacttggcctgtgcccatggacatgctgactatgtgtgctttgtgaatgcaagttttctgtgtgccacgaggtcacttggatgtgaagtctgtaggtgcttgtgagcaggctgaacttgtgtgtgcagggggatatgagtgtgcactgggtgcatgcattcatggatgtagaatagtgtgcttgcttgtgcatagtgaggtaggatgtgcattgggtgcatgcattcatgcatgtagaacagtgtgcttgcttgtgcatagtgggctatgatgtgcattgggttcatgcattaacgtgtatagaagagtatgcatgtttgtgtgttgtaggcatgagcgtgcactgaatgcacacattcacccaaggaatgtacatcaggtgcgtagaagagtgtgcttgcttgtgtgtagtgggatcagagatctgaataaacacactgcttgcagtacagggacgggtgcttaattggatctctctaagagtttgggatgaagggggaaagctgaacccctctgctgggacctcacccagaccctacccaaagctgtgggctggggacatcaaagtggaagtactgcctttctgtgctcttcctgacttctcacctcctccctcctagctctctgctccagccccctcacttcacaggtctgctttgaactccacagcttgggaactcacccagggcaacagggcctttttctctccttcccgagaggcagccacagctgtgtccctaacagctggtggtgttatctgagggaaaggggaagggagggcagggacacaggagggatataacttgactgcccagcccacactgaggaatttcacctctcctttctctagcacacaacacccccaacccccaccctaggcacgcgcgcgtgcgcgcgcgcacgcgcacacacacacacacacacacacacacacacacacgcccgactttagcagcttatctgaccaaaaacaaatggtcagccgctttctgataattaaaacagaaaccccctcagcaactctctccacccccaaccccaccatttggtgctatttcaaactctaaggccatactgcaaactcccaaaactccctggtctctggggacagcaagaaaactcacccactggcctggttctagctttctgtgattctgggaattacggctatggcttccaaatatggttggagaagtgggtttcttgccaaaaagatcaaaagtgggagacaaactgaccctcttgttctaatcccatcctaattcattcactcagaaaatattgagcaactactggccgggcacggtggctcacgcctgtaatcccagcactttgggaggcctaggtgggtggatcacctgaggtacggagttcgagaccagcttggccaacatggggaaactccgtctctactaaaaatacaaatatcagccggacatggtcgcgggcgcctgtaatcccagctacctgggaggctgaataaggagaaccactggaacctaggaggcagacgttgtagtgagccgagatcgcgccactgcactcctgcctgggcaacaagagcgagactccatctcaaaaaaaaaaaaaatttgagcaactactatgtgccaggctctatttgaggcacttgccatacagtgtgagcaattcaaactatgccctcggtttcctgcactttcattctagaggtggaagagagacaaaacgatgaaagttagataaatgcagtgaactcaggtaatgataagcactacaaggaaagataaactggagagtgatgcagcgagtaaatggaaactggcattttcagatagtatagatcggggaggtttcttaggagtcactgatatcttacctgaactccaataactagcttgctttgtaccctgggcaaggcttttgcccctctctgtggggcagtttccctatctgtacatgaggacattgggccagccgggtttagctttctgtgattctgggaattatggctatggcttccagatgtgattggagaagtgggtttcttgccaaaaagatcaaaagtgggagacaaactgaccctcttgttgatgaatggcaagatcacatgagaatccattctccccactacagcaccaagcccactttgacgggctatttgttagactgtgagaatcaggggtctggaagcctttccttatctcccacccacctcctgtcacccttttacctaccattacccacccatccccccattacttggcacagcgccccatacatagtgccctctaatatatgcactggaattgtaggacccccatttctataacctctggagtaatcagaactttcttaggggctgggcctggtggctcacccctgtaacccagcactttgggaggccgaggcaggtggatcacctgagatcaggagttcgataccagcctggctaacatggtgaaactctgtctctactaaaaatacaaaaaattagctgggcgtggtggtgggcacctgtaatcccagctactcaggaggctaagacaggagaatttgctacgagccaagatcacagcattgcactccagcctgggcaacaagagtaaaactctgtctcaaaaaaaaaagaaaagaaaagaaaagaaagaaagaactttcttaggacaatctggatggggcactgggttcccatgacttagaacaggaaagatgcctaaaattttattagcccattgattcccacgctgacatcagagacccttgatgatagaaatggggagagaggcagtagcttgggccagcatcagcatttcatgaaatcttgtggaaatggcacattcttctcaccctatcataagacaaccctctttaaatgtcaagcaccagcaccgcgagcttagactttaataaattagtgtggggactctggttactccatgctgctcatcatgcctttgatgaataaaaactgagcagatgaattcattacgatttcataaacaaggtgggttagatcaacagactccttccttaaaaaggattcactgtggggagaacaggaaagcaagcgagacagtctttgatgccgaaaggttgtggctgcgatctgctccacacacttgaccttacccatggggaaagcaagggagggagagccaaagggttttgtccaggtgtccctatgccaccctgtcttctcctcccacccccaaaggttaggtgcagaggaagtctttgtttggctgagaggagggacttgggcaggaggccgtgaagacaggaagtacagcagaggcaggaaggaggtcaggaagaggctgacaggaccaggccggctcacacccagccctgccagccttcaggagagcaacatggtgtttctaaaaatatttacagtgagtctacattaatgagatatatatcagcactgacaatgggaggatgctgggccaggccctccagggtagaagaccaacgcaattagcccagaggctgcacaaaggcaaggccagccttgtgggaaggccttgtgggaagtcttggggaaggccagccttgcacagctaaaggaagaaatcaagccttccaccctggggcccaaacaggcaatggagaccacagtgccccagaaacctcccccggggaacagaaaacatcccagattcctggacctgcaacgagcatcttcaacctgcatccctgaccccaattcagtgcagaaatctcctctaccacacccactctgaggaccatccaggggtctcttgaatacccctgttgaccagacactcaatcctgcctccagcagcccattcccattccagtcttggcatccctacccattcaaccttcaggagaggagagtggttcagaacatagaggctggagtcaggtagccacttacaagtcactttgccatggagcaccaaggagtgtgatcatttcaagtcttggttttctcatctgtaaaatgggagtcattagaatatctactgccccagggctggtataaggtttcagcaaggtaaaggagacagcatgcaaagaatgccatgtggacagaaagacacaggctaaggtgcaaagatgcaaaaggaagaatttaaggctgggtgtggtggctcacgcctgtaatcccagcatttgggaggccaaggcgggcagatcacaaggtcaggagttcaagactagcctggccaagatggtgaaaccccacctctactaaaaatacaaaaattagatgggtgtggtggcgggcgcctgtaatcccagctactttggaggctgaggcagagaattgcttaaacctgggaggcagaggttgcagtgagccgagatcgcaccacttcaaccagcctgtgcaacaaagtgagactccatctcaaaaaaaaaagaagaagaaaagaaaagaaagaaagaatttaaaatgtcctaggactaaagtgacataaggctaagaaaattagcaaatgccattttgctgctgaagagctaacactgcactgtgcagtgggcgagccacttgccatgtgtggctatggagggctggaaatgtggccaggcagatggtgggacactgcaggtgtagaacacacactgggtttcaaagacttagtatgaaaaaagaacgtaatatatctcattagtaatttttttctattgattgcatgtcaaaatgataacatttggatatattaaagtaagtacattgttaaaatgaagtctacttcttttgtttcttttacttttttttttttttttttttttttttgtgagacagagtcttgctctgtcacccaggctggagtgcagtggcgggatttcagctcattgcaacctccgtctcccaggttcaagtgattctcccgcctcagcctcccgagtagctaagattacaggtatgtgccatcatgcctggaaaatttttgtatttttggtagagacagggttttgccatattgaccaggctggtctcaaaatcctgacatcaagtgatatgcccacatcggcctcccaaagtgctgggattacaggagtgagcctccatgcccagcctcgtttacttttttaatgtgtctactaggaaaattaaaatcacatatgtaacttctgttatagtcctattggatagtgctgagctgaaggatcatatctaagccttaggatgctcactctggaggcatctttgaggatgggttagaatgggttaggctcgttaggaaacaggtgcagtttttcagaccaaaggcccatattaaggagaagactccaattccaattttccaactacaggtagtttgtcaccaacaaacaaaaaataataataataatagtaatggctcttacttattaagtgcttactccatgtcagcaggcactgttctagtgaaggaccatgattttaaaaagtgaactctgaaagcaaaaatgcctggttttaaatcctggttctgctagtatacgaagcctcagtttcatcaaccttaaaataggggtaaccgtatatagcattgtcagaactacgagatactaggcttagataccagacttaggtagtcatctcttactattactcccatagtaacaggtagtgtattgcatcctaataggtaaagtttattaccaggcactgtataagcacactcaaatgctttttcccaatgtaagttttaatgccatcttttacattatgatccccatcttacagattggtaaactgaggttcacagagttacttcactgtctcaggaccatgtggcttctgagtaagacagagaggatttgaacccttggctttgactctagaagcctgagagctctacagccaggatgtgttttcccagcacaaggccatgcttctgcattcagatatgacctgctgttgcttcattggagggagggaggagatttctcagcagagaaatctcatagatggatcccgctctctccgcctgtctgaaggatgatgagattgagtggagaaaaattcacgtaggctcacagctatgatgataaaaagataatttgtaaaaccagcatgtttgtaattactcattgacccatagacataaagatcaagctatgaataactccacaatagcagcaataacaatactagtgaataatactagtaactatttacaatagcaaagacttggaaccaacccaaatgcccatcaatgacagactggataaagaaaatgtggcacatacacaccatggaatactatgcagccataaaaaagaatgagttcatgtcctttgcagggacatgaatgaagctggaaaccatcattctcagcaaactaacacaggaaaagaaaaccaaacacagcatgttctcactcataagtgggagttgaacaatgaggacacatggacagagggaggggaacatcacacactggggcctggtggggggtggagggcaaggggtgggagagcattaggacaaatacctaatgtatgcagagcttaaaaccttgatgatgggttgataggtgcagcaaaccaccatgtcacatgtatacctatgtaacaaaccggcatgttcttcacatgtatcccagaacttaaaacaaaattattaataataataataatggctcttatttattaagtgcttactccatgtcagcaggcactgttctagtgaaggaccatgatttaaaaagtaaaccctggaagcaaaaatagctggtttcaaatactagttctgctagcatatgaagcatctgtttcatcaactataaaataggagtaaccgtatacagcattgtcagaattctgagataataggcttagataccagacttaggtccatgcctggcacagggcgaatgctcagttaatattttctgttacaatcatctcatttaatccccacaataacccaatgggtagagaggatcaatagtgataaatgggaatgataatattcccagtttacagatgcagaaactgagatgcgaaacaataagaaacttgtctaagcccctccatttctcagtattggagatgggattcagacctaggagggctggtttgaggatctacagtcttagctaccatggtagacagttcttaatcttaaacagtttgtaaagtcatatttttttcttattcttcttctttaatgatactttccatcttagttccttgaagtgtggaacgtgcctgccctgcctgcccgaggctttgacagaagccagtgggatagtcaacagctttgtgtgagcacagagagaggtagctgccccagatgctgcggggctctgctgatgaatgacccatcagtcccacgggtcccacaggtatccagcggcaatctacccttgccaagaaggcacaacactagtgctacagcatctcccactcttaggactgctcgggccctaaggatggttggggaggtggaaagaagaactggacatgggtgagccagctcaagcacagagctgcctcctgagcctggctgtttggctgctgtgcctctgatggctcggtgatgctgacctctggtggagatgagctgctactcagctttgacctccctctgagcaccaggagtcccagcactggcttccagccccgccaccgggatgcagacccagcagcctgagaccccagagggtgcacctaagcccaacctctctccttattccagggccctgtggtatctggaattgtctaccctggaaccttatggctcaggggctagaagaacccttagcaaaaggaacttacccagctgatgattctgacgctgaacaggcgctagaacccagcacaggtcccagtctcttggagaattaaggcctcggcctctccacctagttcagtgttttgagctacacattctcttctgacatgtggttacaccgcactgtaataatacatgttatgacactgatatagaaaaagaaatgttcttgtctaattattttctgtttgggtggtgcagaagtaaaaccaagagacgttgaatcatcctcagtattattcatcactcttcctcaagcatgtcactctgtaaagacaacatatgaaagggaaaatgttaccctgttttattaaggaaaattatggactagaaaaagactgcttttttttttttttttttttttaataagctgacattgatagagaaaagttgagccaaatctctggaggcaggaatctcattgctgagcacccagcgccaccttgtggcttctcgcaggacagcgattccagcttccactcatttctctacccaccactaaaaacatttattgagcatttcttatgttccctttcacctgtttctctgctgagcctcttatctgaatcttttatctcaatgaactttgcactgaaatctctttcgatacactcattaattttagcatgggaaaaagggtggctacacagttgagttggcccacataagaaggtgtctatggagatattctttatccatgagccctcccagttattccaatgtcagccctgggaccctgaagatcactcacagatttaattagacaaatctcttaaaatatgtgtgtatattttcctttccctctttctctttctggaaatacgttttcctgtttctcaggcagcagccactctgttaagacaggggaacttcagcacagacaggttgcgggggacacagctaattagtgactgagccagagctggggcttcagggctttgtgactcactcaaaaaatgtccactaagtccttcctctgtagggggccctatgctaagctcaatggtaaatcaaaaccccacccaggattctgccctcgaaagatagagccttgtcagggacagaggcatctactaaagaatgatgtaaaaaagtaaagctgtaactgtgcaaagtattacaaggaaagctatgtccacagacaggatttttagagtctgacttttagagacttcaaaggactgaaaatatcatggccccctccataatctattttaaatgaaaacaaacaatttaaagataggtgtaaagaaatccccaaaagccttatttttcccataatgacatctttgaaagtattttatgctaaaaggaatcaaatagcatgttatgaaaaattgggagtgaccctgtttctctaatgcctgaaagagatgtttagccagcctgttggggaatccaagagtggatacatatataataggagtttgacctagtcaggaaaatttggaaagactttcctaagaaaatggtaacagtactgagagctacaggataagaagcagagaggtgaaaaccatgacgaacaattacagcaagcactgggcagatcgtgtacttcctgcagaagctgggagcttgagacagcagaggctgaaagaaggtgggagaggctggaacagaggacaatgaggactgtgttgtaagagaagacaaggagccagaccacactgggctttgtgggacaccttgaggagttttgcatttatcctccaagcaatggaaagctgtcgaagggttttaattggcagggcatggtgacatgatccagttggtgttacagaaaagccacactggctgtatgccgaaagtggattggcaagaggacaagcaccttagcctactgtcctagtttgttttgtgttgctataacagaatggcatggactgggtattttatatcaatagaaatgtattggttcatggttctggagactgggaagtccagtgtcaaagtggtagcatctggccagggactccttgctgtgtcatcccatagcagaaggtggaagggcaagagaacacaagagagcaagagaaagagcaagaggacaagagtccaataaggagacattaatccccaggagagatgataggagcctggattaagagtgtggtagataaaatggagagaagtaagtacacttgagcaatatgttggattgggtatgggtgtaaggaaaagggagcttttgggacgtctcaggtttctcacttgctggaactaagtgaacatggtgtggctgaactggtgagcaagccccagtatctactttctcctttttcctttagtgattgcaccggagtcttcttacatgccaacttccgatcttctcaggctcacttaccttttttatcagctctgggagggttctgatgaacccaaagcaggcacagtctgatagctccctcccctctggtccctgctatgcctttctcacctcccacatgggcttttgctgatttcactggacttggcacggtgcccatgttttgcatggaaactcagaaaagcaaaagagtcggcactccactgaaaaatgttttgaccaatgagaagagctactggagatattcttcctgcattctctcccagaggtctggtcctcaggaacagttgatacaacttcttggtgtcttgtttctatagattggtcaattaacatgtaccctcatattagttgtctctcttttcttcttcactctccttgttagttactccgatttgtaaactttcagctccagactcgctttccaggaaacacagtggtatggtttaaatgtgtcccgcaaaatttatgtgttggaaacttaatgcccgatgctacagtgttgagaagtgggaactttaagaagtgattaggtcattagggctcatgaattgattaatggatggattaataccgttggatagaccaatgctaataaatggattaattccatgacctcaaagtgggttagtcatcatggcagtgggtttccaataaaagagtaagttcagcccccttcgtttctcttgctctcttgtgttctcttgcccttccaccttctgccatgggatgacacagtaagatgtccctggccagttgctaccactttgccactggacttcccagtctccatagccatgagccaataaatttctattcatgtaaaatacccagtccatgccattctgttataacaacacaaaacaaactaggacactagactaaggtagtaacataatatccaagttttagctgggaacacactacccagctatagactagatgttccacctccccttacaactacctgaagccttatcaaagtgatatgaacaaagtaatgtgtgcttcttctgggtcacattctttaaaaaaggaaattgcttactctttatatcctttgtttctcctttcttatatggtggaacatggatgtgggatagcaagccagctttgaccatgcagatgaagacaacatccaaagggaagcagggcaacagagcaacaagacactgtgtgcaggagagctgagccacctgtctacctctggcttattaatagaaagaaaaatgactatttttaaagccctgtctctctgcctcatatcttaacttgtaccataactcacacttagggaacagtggaacaagaccaggcttgaatgcagtttggggcatgttgcatttgagaaggttttgagaaatgtaagtgaaatatcaattgatcagttaggcacaagtccagatatcagaggagaggtctggactgaaaatacaagtttgtgaatagtctccctttcacgtgtggttggtaattaaagtctctgtgtgtgtgagatggtctaaggagaaggaagagagtgagaagagaaggatgcctgtggtaggcagcttctaaggtgaccccaattatctgcacctcctggtatttttgtccatttgcaatcctctccactggaatgtgggctgaacctagcaactcatttcaaaaatatataatatggcaaaagtaatgggatgtcacatccaagactagatgacaaaaacactggcttccatcttgaatgccccctctcagtcactttttttgagagaagtcaacttcatattgttagctgccctggggacagatctatgtagcaagggcctgaggaaggcttccacccaacagccagtgagggattgaggcccttagtccaacatcttgtaaagaaaggaattctgccaactacctgtgaaagagcttggaagcagattctacaatccctctcaagctttcagatgactgagccccagctaacaccttgcaacaccttgactgcagctgtatgagactctaagtcggaaacatctagctaagccacagccaggctcctgacccagagaaactgcaagataaaaagtgcctgttgttctaagctactaagtttgaggaggatgtgcttctcggcaattaataactaagacagagcccatgcaagagcccggaggaccctgtcgtttagtagctggtagaagagaatgagcctagaaactttatacatcagggcaatccaggtgagtgccaaatgtctttatcaattttgtgctgctataaaggaatgcctgaggctgaatactttataaagaaaaaggggttatttgactcatcatttaatggctggaatgtttaagatttggcctctgcatctggtgagggtctcaggcttcttccactcatggcagaaggtgaaggggagccagcttgtgcagagatcacttggcaacagaggaagcaaaagagaaaggggtgggataccaggctctttttagcaaccagctcttacgggaattaattgactgagaagccactcgcctctgagaaagggcattaatgtattcatgagaaatcttccctcatgaaaaaaacacatccattgggccccaactccaacactggagatcaaacttcaacatgagtgtcggaggggacaaacatccaaatcatagcaccaagtcatagaatccaagagaataggactggtcaataaggagggaatagtcaagagtgttgagatgctgctgacagatcaaataagatgagagaagaatacttgatggacattgagatgcagaagtcactggtgaccttaggacagaagccaatatctcaggactgatagccctggaaaacccaaccatttaaagtctctagaaaatatcctaagggtatatagcaagtgaaagatttactcaaaaactaaagttcaataaaacagcaagagtctgtggaacttgagctatgacatgtgtgctcactctctcactcactctctctctttctccctttctttttcccctagtcctacattctcagtccagacagacaatagaggcctcagtctgaatggatgtggtcaaaaaatgtgactccttcttccctaagttcccagacaagggacataatatttcactgggaaagacagaccaccagcatttctcatttccccaaactccaaaatgcagaggataaactcctgggaattgtgggcaaactatcaggagctcactttctccagctagttctcattcatggagcagagattcaccccaggcacagcagactgagaatacgggggtcttgattacctttagttttaaggcagaggttccacactggaggaggcaaactgagaagaggaagagttaaccattatcactcagtgccctgctctagtatctgggtatcactctaagagaagtgggtcattatccctgttcccagatccagagaaaaggttcaaagactttggccagtaggagaggcagttaataagaacaaagagctccaaagctctctcaagatgaactgactttatttgaaacagagtgtagggaagttcaagcctaagggtgctctaaataacagtggagattttggtagtaagtaataataaatggaggccagtagctccattagggcagcaagataaaccaaagtccagatcatttaccagagataattatggtaagagtgaactaagaagaggcttcctggagtcaggtaaaatctattgttgagttccttcaaagtcttgcctttaaaactacctccgcaaaagtcctgcatttaatttgatcaaactgcaaagcaatttatgttccagtgcattgtttaaaacaatagagaaatcagatgacaattagtgaagggtaaaatctcaatgttatactagcagtcagacagtttaacaaagagatctaagaaagagacagtcaaagacagtccagctgagactagtcatgcaggatgactgtatgcatgcccgtggctaaactctctgaggagtgacattagaggcttcatattaaaggggaaataggcttcactaaaatagttcagccaagtcactagacaaataaacacacaaagaacaacaagaacaaaccatggagggaagacagactcactctccagcattcctaaaatatatgacctaaaatgtctgggtttcaacaatgaaaaaatgcaaagacatgcaaagaaatggtaaaatgtgccccatacctgggggggtagtagacaacagaaaatgcctgtgagatggcacagatatagattttaaagatgtcaaagcggccatgataaatgctttcaaagaactaaagggaattatatttaaagaagtaaatgaaggtatgatgacaatgtcttatcaaaatgagaataaaaataaagagacagtgattattttaaaaagaacagaatggaaatcctgaagttaaaaatgtatagttgatatgaaaaactcactgaagtaactcaacaatagattttacctgacagaagaaaaaaatcagtgagcttgaagatgggtcaataatgattatgcaatacaaaggacaaagataggaaaaaaaatgagaaaataaacaaaatagaacacagaaatgtgagataccattaacagcaaaatatgcataatgagattgctagaaagaaatgacaaaaagaaatgagcagaaaaaatactcaaaaaatggtggaaaagttcttatttatttatttatttatttattttcaagaaagccttgaacttctgggttcaagtgatcttcccctctcagcctcccaggtagatgtgcaccatgatgcccaactaatttttttaatttttttcgagatgaagtctcactatgttgcccaggctggtcttgaacgtgtgacctcaagctatcctcctgtttcagcctcctgagtcactggaattacagacatgagccaccacacccagcaaaatgtttttaaatttaatgaaaaaataccatctaagaagctcaattaactccaagtaagataaatacacagcaatccacatccaaacacatcatggtaaaaatgctgaaagccagagataaagataaaatttgaaagcagcaaaagaaaaatgacttgtcagctagaagacaaccctaataacattaacagtcaactattagacacaataggtaccagaatgcagtaggattacatacaaagtgctaaaagaaaaaaaaatggtcaactaagaatcctatatccagcaaaaacagtaaaaaaagactaacaagccaacttttaaatgggcaaaggatttgaaaagacttttatccaaagaagatatacaaatagccaataagcatgtgaaaagaggctcaacatccttagctatcaggaaaatgcaaagcaaaaccacagtgagatactatttcatacccagtgggatgactataatcagaactacagacaataagaagtgttgatgaggatggagaaaaacgaaccctcataacactgctcgtggaaacatacaatggtgcagttgctttggaaaacaatctggcagttcccccaaatgagttaccatatgacccagcaatttgtctcctaggtatatacccaactcctattatctggatgtttatgtcccctcaaaattcatatgttgaaatcctaacccctaagatgatagtattaggaggtggggcctttgggtggtgattagatcatgaggtaggagctcttgttaatgggattagtactcttacaaaataagccccaaagagctgccttgccccttccactgtaaaaggacatagcaagtgacaccatctgtgaggaacaagccctcagcacacaccaaatctgccagttcccttgatcctggactttcaagcccccagaactgtggcaaataaatgttttctgcttataagccacctagcctatagtatttttgttatagcagcccaacagactatgacaccaagaaaaatgaaaacatgtgctcagacaaaaatttgtacattcttgttcatagaagcattattcatagtagccaaaagggtgaaacaactcaaaggcctaacaacaaaaatgtggtacctccacatagtggaatattatttggcagtaacaaggaatgaagtacaaatacctgctacaacatggatgatccttgaaaacattgtgctaggtgaaattagccagtcacaaagaaccacatattgtatgattcaatttataggaaatgtccaaaatagacaaatctatttttaaaaagtatattagtggttgcctagggctgggcaggaagaaggggaggtgaaaagggcagtgactgctaatgggtatggggttatttttgcagagtgataaaaatgctccaaaacttattatagtgatgagtgcatacctctgagtattctttaaaaaaattgaattgtacactttacactcccaccaacagtgtataagcattcccttttctcagcaatcttcccagcatctgctgtttttttactttttaattctgactggcatgagatgatatctcattgtggttttgatttgcatttctttaatgactggtgatgctgagcattttttcagccattgtgaaaagcagtgtgatgacttctcaaaaaacttaaaacagaattattgggtatgtacccaagggaacataaatcattctatcataaagacacgtatattcattgcagctactcacaatagcaaagacatggaataaacccaaatgcctttcaacagtagtctggaaaaagaaaatgtggtacatctataccatggaatactatacaaccataaataagaacgagatcatgtcctttgcagcaacatggatggagctggaagtcactaccctaagcaaactaccacaggaacagaaaatcaaataccacatgttctcactggtaagtgggagccaaacaaggagaacacatggatactaggaggggaatgacagacactgggtgggaggagggagaggatcagaaaaaacacctacctagtactatgcttattagccaggtaatgaaattatctgtacgccaaacccccatgacatacagtttatctatataacaaacctgcgcctgtatccctgagcctaaaataaaagttaaaaaataggccgggcatggtggctcacgcctgtaatcccagcactttgggaggccaaggtgggtggatcacttgaggtcaggagtttgagaccagcctggccaacatggtgaaactccatctctactaaaaatacaaaaattagccgggcatggtggcacccacctatagtcccagctactcgggaggctgaggcagaattgcttgaactcacaggcagaggttgcagtgagtcgagatcatgccactgcactccagcatgggtgacagggcgtgactccgtctcaaaaaaaaaaacaaaaaaagttaaaaaaaatgaataaaaataaatacaatttttaaaaagaattgtacactttaaataggtgaattgcatggtagatgagttatatcttaatatagctgttatcattttaaaatgaaaagaatcgagttaacaattaggtgaaagcaaaaagagataatgatagctacccaggggaggaattcaaggacacgtggaagggctagccttaggcaggaggacggaaacctcctacccagggcactgggatgaatgcaggtgttataggtttgaagatttggatttgagagtatgactgttctcatctgatggctgaacaggtagacagagggtagcagccttgaagtttgaagagaatgaagaagaaatgaaatagttgtggaggtgagatggtggtcgctgggagtcctgcagtactgatgttgcctcacactcctgcccactggcgccctcttcccacttctccctttggccctccatgcaattcataaagggcttctcaaacaaaagtgctcattgggggtcggagtgagggaaatggttgccaggacaatgagaaattgataaccttattcagatctggagaacaatgacagagagaaagtgcaaaagaggaggaagtcaaaatggaaggggagaggagagggaacctccagctccaccctggacttttctaaatctttcagcagggccatctcaggagcacgcaccatgtgccattgcacagaactccatggcggaagtggctccctgtggtttcacgttctgctttcactgccttgaaattcttaacaagttgagttttttgttttgttttgttttgttttgttttgacagtgtcttgctctgttgcccaggctggagtgcagtggtgccatctctgctcactgcagccttgacctctcaggttcaagcaatcctcctgcctcatccccccaagtagctgggactacaggcacatgccaccacacccggctaatttttttgtatcgagatggggtttcaccatgttgcccaggctggtcttgatctcctgagctcaaacaatctgcccacctcggcctcccaaagtgctaggattacaagcatgagccaccatgcccagcctaaaattcttaacaagttttaaataaggaagtgcacatttttatttttcacaggcaatgtaacccatcctgccctataaagaacacttgtctatccagaacatggacaagaatgtcttctctacagctaggaaaaaaattacaaagctcagacctggactgacccaagaataaaaccccagatcatttgtcttccatagcctagttactcttactttccacaaaattccagtgaggaactatggtgtttaaaggtattcgttgccactccctgaggaggggggattatgcttcctcaccccactgacatctggcttggtcgtatgatttgctttagccaagaaaatgcctcacttctaagcagaagcagaccctgcgcatgtttaccgtgttttattttccttctgttacaaaaccagtaatggatagctgggcacgatggctcacacctgcaatcccagcactttaggaggccaaagctgggattgcttgagcccaggaattcaagaccagcttaggcaacatagtgagaccctgcctttacaaaaaaatacaaaattatctaggcatggtggtgcgcacctgtagccccagctagttgggagactgagctgggaggataacttgagcctggaggtcaaggttgcagtgccatgatggtgtcactgctctccagcctggacaacagagcaagactttgtctcaaaacaaaacaaaacccagtaatgccccacatataggctaccgatcagccaagatgtcatggggtagtgctacagctgacctgggtggagatgcagagggagcaagagatacatttggttgctttaagccgctggaattcaagagttatttgtgattgcaacataatctaacttatcccaacttatacagaatggcctcattttccaggatcacatcaacttaaaatcagcaaattttgagatcctagatggcccttctctaacctctagaatctgactttacacagaatatatatttgttaaattcatagatgggaactcctattccctctgagtcagatccagactccttcatctgcactcacaaccctccatggccagtcatttcagccttgcctcccactaccccacaaacgaccaatttgctcactcacctgcacatgcttttcatttgttcattcattcattcatgtactcttttatcccatatgtactggtgcctgctctgtgacaggaagtgtgtgtgatgcagaattgtttacacagacatggtctccctgccctggtggtttagttcccagaccttggtcttgcagtttctctctgccaagagcattgttctgacttttctctgcctgtctaagctgctagccctccttctacacacaactcaaaccgtgcctccttccactaactcatcaccccacttactctggcgcaccctctccttcctctcaactcctacaccaatcattttcttattgattacgtggcaattagttacatcctgcctgatagtctctctcctattgtgttttgaacgactattgaaatctcattattaaaatatgtataccttttctcatgttcttaaataggtccatagaagcagcacatcccctaatgcaggctgaactaattaattaaaaccaagtgtagtacctttatttacattagaagacttgtctacattcaaccaactttgacctagaaaaatggcactttcccaataatcatatgaaaaaaagctcaacatcactgatcattacagaaatgcaaatcaaaaccacaatgagataccatctcacaccagtcagaatggtgataattaaaaagtcaaaaaataacagatgctggcaaggttgcggagaaaaggaacacttttatgctgttggtgggagtgtaaattagttcagccattgtggaagacagtgaggtgattcctcaaagacctaaagatacacatgccattctgaagaataggacctggagccaggaaacctgagagctgcctagaactaaatcagagacccattcagctatgacagaaaatactctcttcatttacatagggcatacacccagtaaatgactttgtaactttatgcttttcatttacataaggtgtacacatgactttgtaacttcacttcatcctcttggttttttttttgtttggttttgtttttgagatgaagtcttgctctttcgtccggctggagtgcggtggcatgatctctgctcattgcaacctccacctcccgggttcaagcgattctcctgcctcagcctcccaagtagctgggactacaggtgcatgccaccacgcccagctaatttttgtattttttagtagagacggggtttcaccatgttggccaggatggtctcgatctcttgaccttgtgatccacccgcctctgtctcccaaagtgctgggattacaggcgtgagccaccatgcccagcctacttcatccttgttatttacatagggcatacaccaagtaaccaatgggaaatctctacagggtattgaaaccccagaaaattttgtaaccaggacccttgagccacttgctcagggcctgctcccaccctatggagtgtgctttcatttgcaaaaaatctctgcctttgttatttcattctttccttgctttgtttgtgcattttgtccaattctttgttcaaaatgccaagaatctggacaccatccaccggtaacatatttttgccagcaagccaggaggtaagcccaaagtttgggatttatttttctcctttctcttttcctttctactccatacaggggaatctcttttttttctctctctctctctctttttctttccaactcaggaaccttggtgggcagttcctaaacacagaggtaactgcaggtttctggccatggccactctctggtgaaactaaggagtttccgtatggaggctcctgactgccgtcacctggttcaagtaagagacctgggtctttttcccttttttcctttttctttttcggtctttcagtggtcacttcctagcagctccttggcaattgagggcaactggccagggccaccctcgggtgttgcctgaaggccaaggagtgaatggggatagttgccctgcctggagagggaaggactcttttctatcttttttggttgtggtccctgattcctacatgtggctcagctcattcaggcaaattcacatacgtttcaggacagttaaaccttcttttcttatgctaaattcttcccttcccctactcaactggctagggacaaaagaaacccacccagcctccagttcctaacatgaaagttcatggaacgggaagcatgggaaagtgtggccatatcaaattataaggacgccggaagtcgaggtcttcatccagggacaaaagcaaagttcatagtaggccatcgcctctggagggaaaatatgcaaagcggcaccagtgcccacctaaagtcagagatgtctgaaactctaagattagacaccaaaggggggaccccccaggggatcccctggacctcaagctctccaaaggggatgccctcggcagaagttctgaggcctagtactaagccctccttagaattttctctcacagctgcaataccgtttggccccaatattgtttggaatctggagtttgctgttgaatgggaaagtaggttggagttgtaatagctaggcttttgtgctgctgttctaagcaggatcaggcctggttattacgtgatgttctcctgtggtgctgtctgaccccagtgttctttggagtctggggaggtttggcctttaaaaatcaaacagccatggaaactgctgtacccaaaattttggttcacacccttcattggattacctattggggcaaacaaagtaaaactggtgagtttgtattgctatctcatggctagagttccaaaataaaagctattggatcttcatttatgtgtgttgtacatatgtctagatgtgcttatttgtatgcacacttattgttgtatattgtgtctactaaattggcttataagtaaaagagcacttataaattaagtctaagcaattttcgagtgcacatgatttaagtataactttactaaacaagctggttttaaaattattggtaaaataaaaatagaaatcccttcagaattgtcaacacatggtcaggtgcagtggctcatgcctgtaatcccagcactttgggaggccgaggcaggcagatcacctgaggtcaggagttcaagcctgaccaacatggagaaaccccatctctactaaaaatacaaaattagccaagtgtggtggcacatgcctgtaatcccaactacgtgggaggctgaggcaggagaatcgcttaaacttgcggggtggaggttgcagtgagccaagatcgcaccattgcactccagcctaggcaacaagagtgaaacttcatctcaaaaaaagaattatcagcatacatttctgtctggattttatatttgtctctgctagatattttgaggtgtcaggctttggcatagaaggttataaagctataagccaaaacaaaatgatcttggtttgcatgcctttttctgacaaatgagagtaatttaatgttggtagctaaatcttctgagttattggcaaaaatacatatgtatttaactttgaggcttttacttaggtttaggtgagcacctgatgttccctggctattcaaaacagtgatactgtctaatatctcagtttacagaagtaatctggataaactattaaaaatgaaagaattgagtacagtaaataagataaatattttaggtaaatttttgtgtaaattaaaatcttaaaattattgttgatgctcattgaatatctgggttatttccaattaagaaggggttgcgatatggggaaatatgtttctaaaattggggggaaagtttagataataaaatattccttaaaacctcatagagaataggagacatttgactaattaacattttcatagttaaagctcttaatcttgattaaagaaaaataagaaatattgtaaagaaatgtattggctgtttggcaattctttttttaaatataattaagaatggagctggatttagtgtggagacaaatttcacatacctgcttgcttcacactattttactgtttttcatggatagtgctggcactggaatacttactggtcatgtgcctagagtaaatttcttgattgcacaggatgtatgttgatattagtggacttaaggatattaaattgtgtatcaggaataaaatattcattatgtgggtttttagggcatctgggtaacaatgtagcctccagggtagattgagtaggaaaaatttatggttgattttctgtttatttgtttttgcttctaattttcttttgtttgctatttattctcctctgggctttgcttatgtgtggagatatataaagccatgatgctttctttttgtttttttttgtttttgagacagagtttcactcttgttgcccaggctggagtgcaatagtgcaatctcgactcactgcaacctctgcctcccaggttcaagtgattctcctgcctcagcctccctagtagctgggattacaggcatttgccaccatgcccagctaattttttgtacttttagtagagacatggtttcactatgttgcccaggctggtctcgaactcctgacctcaggtaatccacccacctcagcatcccaaagtgctgggattacaggcatgagctactgtgcccagctagccatgacgcttttttattttctagcagaaggattttatttggttctgtgaatagttactttgtttcctatgcatttctagcaagtaatcatttatttcatttatctggaattcctagactacctttgtccagcctgcaagaactgatggagcacaacagctttttcttaaaccttaaactaactttttggatattaggcttcctgatactttaagtgcattgagtatacattcatcaatagcattttagtcatatttctctctctgcctaatttctccaaaatctgtaaattatttgtgaatattcttaattcatggcaatgcatttgtttgcatacagttgagcagggttgctagggccactcagggaaagagaaccctgaacctgacatgacaccaataaggtaagaatttcttaccagtcagacttctgccccatctctctgtgcaaactggttgaatgaatggtaaaagattataagaagaaataggaatgtaaattttgtaaacctttaacatctttaatagacttcccaaaatcaaacttcagctttaaaattgtcatttctgacatctaactttgggatgctacagagggcccctgaagcatctgaaagagaggtaaacaggattatctgacatgtttagttacatgggaagcactgtcaaaataaaaaaaaaatgtttaatctttttcaggttatattttagtgaatattaatatatgttacaaaattgtatgggatttctaaaattctaaatatgtctgagtatataatatcagttataatgtttattatgttaagttactgtagaccacagaaataatcaaatttccttgtataaaactactaacccaaaactaaatgaataccaagaaaatacatttccagatttgcatgctaaatcagctgatactgaaattgtttagatatacaatttgaatgaactccatgggctaagtcaaattacctatgataacccatgagttatcagtgctatgcatctaaactggagaaacaaatggtattcaggaggacgtaagttcaacgttaagcatggactcatggagaaccagaatggctgcctgtccttcctgagtcctaaaagctcttgttattaaaggttctgcattccatggcctgagaaggactctgtacttctatatttgagaacttgtgagcaaactgtaacctaacttaggaatatgcacatgtaacaatagctgaatcttggccaatcccagcaacccaaaatcaggccaggagataaggtacttgttaacacatggaaggagagatcacctgctctggataatagtggccatttttatattgcccccttccaaacagatggaattacttcctctgtagtaattaagcagaatgtttcaattcatctctataataaacattcttgacagcatcggtatccacccccgatattcccattcgatcttttaaccaaattcacatcctcttgcctagagaccatcaagcttcagatgatcatgtgacaaggtttccagcaagttccaagtgaaggcactacatcctgccatcaagaatctaccctgtctccactagacagagtagggcaagagttctatgattcccaataggtagggactacaccccaagccaacacgaagcagttacagaagaaagaccatcagtccctctgcctcccataaagatttatggggatcacatctttcaggtgggaaatgaggtaatagaatagggcctggaggcagggaacctaagaacttcctagaactaaatcaaatggaaacacttgagctatgacaagaaatatcgtcttcatttacatagggcatacacccagtaaatgactttgtaacttaacttcatcctcttcatttacatagggcatacaccaagtaatcaatgcgaaacctctagaaggtattgaaaccccagaaaattctgtaactggggcccttgagccacttgcttgggcctgctcccaccccatggagtgtgttttcatttgcagtaaatctctgcttttgttgcttcattctttccttgccttgtttttgtgtttagtccaattctttgttcaaaacaccaagaacctgaacacccttccaccaataacaattcaacccagcaatccaattactgggtatatactcaaagaaatataaatggttctattacaaagagacatgcatgtgtatgttcattgcagcactattcacaatagcaaagacatggaatcaacctacatgcccatcaatgatagactggataaagaaaatgtggtacatatacatcgtggaatactatgcagccataaaaaaagaatgagataatgttctttgcagcaacatggatggagctggcagctattatccttagcaaactaatgcaggaacagaaaaccaaataccgtatgttctcgcttgtaaatgggaactagatgatgagaacacatggacacatagaggggaacaatgcacactgtgacctttcagaggatggagggtaagaagagggagaggactaggaaaaataactaatggatactaggcttaatacctgggtgatgaaataatctgtacaacaacccccatgacacaagtttgcctgtgtcacaaacctgcacatcctgcacacgttcccctgaacgtaaaataaatgttaaaaaataaataaataaaatggtggggaaaaaaaggtaatttcctatggttcaacctaataattctactacaaaaagtaatctgttagctttggagtcctctcccttgctaggggtttctagggaagaacacacacgacctgtcctcacaggggtcccacccacaggccccaacacttgctaaaatctgtgtcccttagagaaagcgttggcaggcattggggaaataactgtttctggggcatcagcccctttaaacctgctgaccaagtcctccgctctcattctgcagtaggaaagtttacaaagatacagcagaaagaacaaagaaaaccaaaaaaggagaaggtactaattgttgagcatttactatgtgccaggcccttagactaagtgttgcatatatatatgcctattgatcctcgaagacaatcttgaaaagtgagcacttcccattttccagagaaggaaacaggcttacagacatgacgtgtcgtgctcacggtcactgagcagtgaaagccagtgtccccttatctgtgacattcacacagctggttatttgggagtatttgatggttttgtgagtatttgatggttttgtgaagcctgagctgactcctcttgtacccatctgcttgactggtacaatcatctccctcacacagatgcagaggtgagccccgaagacattgagccttgcctctcccatcacacgacaaatgaatggcaggaggattgcttcaacagggcatgccagaatcatcccactcagcctggtagcttcattggcacggttgagctggcaacaggacactcatgcagggcaactgaataatcagaagcagtgaagcctgaactctaaccactatcattttaaagattattatgtgcagttatttcagctaattaaaccttgaatgcctgccagctcagcttccagctcagagaggaaatgcaggcatgcatggcctcagcttcccttccagcctctgtctgcctttcagggggagcagagctcagggcctgggaatggagggagcaaggagagcagcagagcaagggaggggccctcaaccactgtggccacttccccatgggaacgcagaagttctgatccttgccacgggaaaaatcccctgacttttgtagcctctcaggtatgggaagttacagcctgttctttactggttcatcaaggaagagtcgggtctggcttgaagcctggaatcaatgccaagagcaaagttgaggtcccagctttgtggctggtagctgtgaggacctggacaatcttctgttttctcacctgcatgctctctgaaatttagcttttgcacaaaaatgtgtagacagggtacctagtgctgtgcctatccaatacagtagctacaagccacatggagcagtttaaatttcaatttcaatcaattaaaattaaattagattaaaaattccatttctctgtcacactaggcaatttcaagtgctcagcaagcctactgtattggacagggcagctacagagcatctccatcatcacaggatgttccaagggacagcactggccctgtgattgagggtgtgaaccctgattcgactcaaggctgcaatcccagctccaccgcttactggtgaccctgggcaagttactgaacctcctcatcctcagtttcctcatctataaaatgggaaataacaggatccagcccagagtttttgtaaagattaaatgagaaatgtgtgtaagatacttagaagaatgccaataatggtaagcacactataaatattagctcttagtaacgtattttgaggagtatgtataaattcattctattacagggctttacagttctaggaagcagtttcaaaatcactatctcatttgaatttcacaagggttccatggggcaggcggtggcacaaggagtcggctgaacccttttattaatacaagaaggcaaactaaaatccataggttacctgactgcctgaaagggtggcagaactaagaactaggacttgaactaagggaataattcaagaactaacatctgtagactcttttacacttcatgtcctatttcagatttagcataatctaaggaggccaggaatgtgcctcccatgttaagagtgaggttctgcgatgggagtaatcccaacgctcactagcccttacatagcacagcccttggtgctcttgcacttggcctccctccctccccatgttccttgtccattgctaccttgggcttccttacagcatgggggacccagggtaagtagagttttacatggtggctggcttccaagaaagaggtggtgaagctgctagtcctctttggtcttaactgtatgccaggcactgtcctgtaatctccacaactcagtagggtaggtacacatatgattcccattttagtgatgatgaaggtaaagcacacagagggtaagcaactttcccaaactcagccaggaggtggtggagtcaggacatcagagcccaggaagtctgattccagcctggcttcagagatgtcaccattcacctgcccacatcccaggacaggtgcaggaagcaggaggacagagctctttgttcccagggcctatttctctccttctctcacctgtgtccaaggacaggagagtacagcccctctcccagcctcaggggattgctgggggacacaggtgcctttctgtgcctcttgctatcaccctgtattcctttgttagtgctgccataaaaaagcaacacagaacagagggcttaaacaacgtaaatttattttctcacagttctggaggccagaagtcccagatcaaggtgtcagcaaagttggttctttctgagggccatgagggaaagatctgtcccaggccccttttgttggcttgtagatggcctcagtcttcccatgtcttcacatcctcttccctctgtacaggtctatatccaaatctcatcttaggaggaccccaatagtattggatcagggcccaccctaatgatatcattttaattcaatgacctctttaaagactctatctctaaatacagtctcagtgtgagatactgggagttagaacctcaatatgtgaattgggggacatagggaacagttcatcccataacaccctctgtctgccttcatccttcttgctcaagtcaattcttggccacctggagatgtcagtgggagcaaggttctgggggcagaactgtgacgagctgatgagtggaaaggagtgttgatgaaagtggaaactctggagaccaaatgacagcctaaaccactgtccactgaaatgatggggatgggcctgtggggaagggaagcattattaatcattgcaattagcctgagcaacatagcaagatcctgtctctacaaaaataaaaataaaaattgccgtgtgtggtggcgtgtgcctgtagtcctagctactcagaaggcggaggcaggaggataccttgagcccaagaatttgagtttacagagagctatgattgtgccactgcactccagcctgggcaacagagctagaccctgtctctaaagattttttttttaattcactgaaatttactgactaccttctcattgcatccccataacaggcctgtgataacaaattggtgttattatcccatttgtagttaagaaaaatgatactcagagtgatttttaaaacatctgggttaaggaagctatggagctaaggatcatgggtcctaacttctcaggataagagccagatcaggctgggcacagtgactcatgcctgtaatcccagcactttgggaggcaaaggtggatagatcacttgaggtcaggagtttgagaccagcctagccaacatggtgaaaccctgtctctgctaaaaatacaaaaaattagctgggcgtggtggtgcacacctgtaatcccagctactaaggagtctgaggcaggagaattgcttgaacccaagagtcagaggttgcagtgagcccagatagtgccactgcactccagcctggataacagagcaaggctccatctcaaaaaaaaaaaaagccagataattcattctatttgtttggtgattatgtaaaattaataacgaagctttactctgagtatgaaaataaacaaaaactattgctatacccaaaaaagtctctgacacacacacacacacacatacacacacacacacaaaagggtatttcattcatgctgaacctatgcaaagtgaaatgaagtaactcatcaaaagtcaccacctagcatatggcagagctggtgctgattcatccatgttttttcaacattagcatgcagaaggggtaactggaaaaagatcccactaccccagagatcttagagcagagttctggaatgagtagcctcagcagtcacactgcttggtgggtgtagagatggagtttgaaaggttctgggtgtggtcagcagtatatacaaatggtgccatagattccttccctctctgctcttgctcacaactcctcacatcaagaagttaggtctttttttcctccccttcaacttggggtggccttttgatttgctctagtgactgaaatatgacagaagtgacactgtgaccattctgaacccagaccaaagaggactagcagctctgctacctctttcttgaaagctagccaccatgtaaaagtctagttaccttgggtccaccatgctgcaaggaagcccaaggtagctatggacagggacatacggagagagagggaagccaagtggaagagtaccaaggtaccagatacatgcatgaagccttcttaggctttccatcccaggcccagcttccagataaatgcaactgagtgagtgactagagcagaagaaccacccagctgaaccctgcccaaattcctgacccaaagaataatgagaaaccataagtcattgttgttttaagccattaagtctagggatagcttattttgcatcaagagttagttgaaatactattcggagggagaggagctatggaggtatgttagaaaaaacatacgtcatcaacaggcagggagtagggagggcaggtcaagacaagaaccagcaagagaatatcatgagccttagctgggcaaacaaggtattctacagtctggccccatgacgatccctggcacttcaagcacacatagctatggggtgatctctaagcctcacacagctttgcctttgcacatgccatttcttccttctggtgtgccatttgctcacttatccttccagatccagtatcaaagacagaccaaaaacccataagaaggcaatgggttttaaggcccttagagtctgactcttgtctatttctccagcttcatctcttaccctctcctctctctgtgctccaaacacatgggtcttattataatagttatttgcaccttttgctccttcccttggaccttttgctccttccctctacagaacctttggaggtgctatttcctctgctaaggatgcttccacacatccacactcattccacttggcctagttaattcctgcttctccttcaactatcagctgaagaatttcatcttcagggaagcctcccatagatcccaaagtcaagtctggttcccttgttatttattctcatagcaccatattcctgccctgcccccagggttattctgagaaatctgatgctcctgttctcatagactgtcagttcctttaggtcagggaccatatctgtctgtgatcactactatatcaccagtgcctagaacagtttgtggcacatagtagatgctcagtaaatacctattgaatgaatgaataaatattatctcctctaaatagctttaattgacactcctctccctctgagattcaggctttctttctcttcttagttcccaggacacttagtttttatctcttatcatcttatgctgaactgttctggttatgtttgtcaccccttctaaactgtgactgtgataaggcccaggtcctgtttgtttttctatcccaggagcacagctccgaaactggtgcagactaatttgtccattgtaaaatgtggaagcttggaaagatagaatgtggatatgcatgctagtggtgagattccccatctcatcatattcagaaagccaaaggtgttctttagaaaaccatgccagtgtaccagtcagaaatctcaaataactgaaaaagtcagctgccttcaggtatggtgggatccagggtctcacatgcaggatgtggcctctgtctttctctcaacactatgctttgctgtgttagcctcaatctcaggcaggctttatctatacttactaaattccaaaagctctaggtcatatccttctaactatgagtctattaagagagattctattccccaatagttcaaataaaagttcaccaatagatgccaaaattattatagtaaaaacgaaaaaaaaaaaaaaggcttaccaatagaatctcatgggaccagtttggtcacatgcccacccctgggccaatcactgtggccttcaagaatcaacactgtaatttatcaaacatacatcacttgcccattcctggagctggagatagaatcagtctcactgaacctatgggtttaaagtgggctcagcaaccaggagacagggcaatggaaacagagcacataaagtccaccacattcactctgcccagactttacagctctgttcactggaggtccaaacacaccgaagagtctgcaagggatgttgctgcagaggccatgacaggctgcgcatggtgacatttcccactcactgtggctctaagatttcacttgagcggagcatagcctggctttccatcatcctgctctccttccaccagcccagaagcctggtggagtgaaagccagagactccatgactaaggtgccctgggttggagattcccctcacccactcctcctccagagagaacacacaccaagaaaaagcacttagccttgatgctggtttcctgtcactgcccttcctattgagacgtcattggccttggctcccctcctgacttttctatttttttctacctcctgttaaagaggcaacaggtccgtaaaagatttatcccataacctagtgtttcaggctctatctccctgttggttagactccctgacagattaataaattaacaatttattaattgttttgtttgttggatggatggatggatagatggatggatatacggacagatggacagatggacggatgtatggatggatggatggatagatgaatggatagacagatggaggagtgcaatgaaaggtggatgaattggttctgagtgccctcagaacactctatctcctcttctcatatttaccaccctaggctccagattactgtaaatatagaaagccctttttcttgtcctgaaatcagtttcccagccctctgttattataacaatgactgacatttaacatggttaattcttatccaggcacttaccctgagctaagtactcattcaatctccacaattccattgtctggatgctgttattgtctacagtttacagtgaaggaaaccaaggctcagagacaggaacttgtccaaggcacatagccagtcaatggcagagccacaactcaaagtgtgtgaccttagccactatcttctagggccctatgaacacgaacctcagccctgtctacaccaattttctccttctcccttgaacatgtcatatctttattcatggaataaggtcttcctcctcacctctttgtagactcatatcaaggaatacattcatcaaagtaacactaagttacactctgaacaaataaaccctcaaatttcagtagcttatcacaaccaagatttatgtcctactcatgttatggactaatgtggggcaagtggccctcttccatctgatagcaggcctctaacaggggaagggagaagtggaagaggagcactggctctttgctgcctcagcctaaagggaacacatgtcactcctgctcacatttcactcacccaactaattagtgtgtggacatttggcgaacatatcccatctctgccacaaagaaccatacactcctgccaggggtcatgcagaagataaaggctcagccagcccagtcagtacaaattcggagggtgggggaggaaaaaagagccaagattcaagctggtggctggggagtggagagagatggataggattccagaattaaccaggtacaaagcacccaacacttgggtggtaccccatgtctattgtcttgaatattgctcacaagtaagccaaccctaagcttcttggctaagactcagaactcttgttatctaagtttatggctgttaattacaggatttgacctgaattaactgaacaatgcagtaagaataacaatccctgacttttcaaattttagattttccaaatcatacagcaatcatctcatttgaccctcacaataaccctatgacgtgggtatgctatctctacctctattgtgcaataaggaaatttagagaaggatgatgtaattgaccaaagtcgctcaaaacctgtgcgtgctgcaatacatctcagtgccagttttctaacacatgctaggggctctttcttctacagttcagctgtgtctcatgcaaatttctatggaaatcgacaatgtgaaaatgaacatccataggatttgaattcaaaagcttgggtgctctgtgcaccaagagggagttgtttaagcatacaggtgttgaagtcagatggcctgggttttaatcccagttctgccacctgctagctgggtgatagctggcaagttaatctacctgcgctcactttccttatttggaaaataacgatccactgtacctaacccataagattatggtgaggattaatgagacaaggaatataaaattcttagcacaatgcctggctcaacattagaacttaataaacactgaatgatgctgccaagatctttctgggagacaaaaagatgccactattccttccttcctccctcagggcatgatcgtgtctcactacatgacactcttggcttcagctggagaacaaacggaccacctaacccggcttcacgtgcaactgccagcgagaagagaatgttcttttctttgtctgagattcggcactggtccctgccgccaatcccaggtcttcgtctcagatttgctgccacccagaggccatttcatgccatgccaggatgactctccaaagtattccttgggacagtggttttcaaaagaaagtgggacgtgggggcagggaaggaaaaggaaaacatatccaactcttggaggcaggcgagactgtgggggttgaaacaatcccgcaggggatcctggcgtgccctccactctattcacttgaggttgtctgatttcggtgaagccaaaaagagagaaaaggggaaagggagtcaaaaggatcctgtctgtcttcaccagcactaggaatacatgtcagctggtgctttcaaggggtgtcagctccacctcgtggaatgagagccagtgatttgtttctgatgcaattcttcagccagtaaacacaggaacaaggaagttatgacatgacctttctaaagatccttaaaaataccccagccaaagggactagtatggagaatatgtaaagaatccttgcaacctggcagagcactgtggctcatgcctgtaatctcagtactttgggaggacaaggtaggaggatcacttgagcccaggaattcaagacccgcctgggcaacatagtgagacctcatctctacaaaaaaaaaaaaagaacttttacaacctaatcatcaaaagacaaatcagcaaaaggttttgtattagtccattctcacattgctataaagaaatacctgagacaggataatttataaagaaaagaggcttaattggctcatggttctcccggctacacaggaagcatagcagcttctggggaagcctcagggagctttcaatgatggcagaaggcaaagggggagcaaacaactcatatggccagagcaggaggaagagagagagtggggaggtgctacacacatttaaacaagatctcgcgataactcactcactatcaggagaacagcaccgagggagtggtgctaacccattcatgagaatttggccccgtgattcaattacctcccaccaggccccacctccaacactggggattacaattctatatgacgtttggtgggagcacagatccaaaccatatcaggtccgaatagacattttttaaaaaccaataagcacatggaaaggtgatcaacatgatcagccatcagggacatgcaaagcaaaatgataaagacgtgacctcatacgcactaggtgagctaaaatcaaaaagacaggtgttggtgaggatgtgaagaaaccagaaccctcacacacttccagggggtggtggggagaatggaaaatggatgcaacttcttgaaaaacagcctggaggcttcccaaaagttatagagttacagtatgactcaacaattccactcctagttacatatatccaagagatgaaaacatatatccacacaaaaacatgcacaccagttcccataaaagcatcattcgtaacagccaagaagtgaaaacaacccaaatatgcaccaaataagatgtctatagtcggcagggcaaggtggctcacgcctgtaattccagcactttgagaggcggaggcgggctggtcatttgaggtcaggagttcaagaccagcctggccaacatggtgaaactcatctctactaaaaatacaaaaaaattaccaggcatggtggcacaagcctgtaatcccagctatcaggaggctgaggcaggagaatcaattgaacctgggaggcagatgttgcagtgagctgagattgcaccactgcactccagcctgggcaacagaaggagactctgtctcaaaaaaaaaaaaaaaaaaaaaaaaagatgtctatagtcatacaatggaatgttatttggcaataaaaaggaatgaagaaccaacacatgctataacatggaggaaccttgaaaacattatgctaagtgaaagaagcagtcacaaagaaccacatatttcataattctgcttatatgaaatgaaatgaaatgtccagaataggcaagtctatagagacagaaagtagatttgcagttccctagggctggtgcaggtgtgaggctgggggtgggggcagacaattatggctaaggggtacaaggtgtctttatgggctgataaaaatgttcttaagttgcttgtgatgatggttgcgcaactctgaatatactaaatgccattggattgtacactttaaatgggtgacttatatggcaggtaaattatttatcaataaatctgttaaaaaagtaaaataaaccaagctcttctttgtacatctgcttgaggcaagggctgtttccaagcccctgtattcccaacagtatgaatcacagaaggaaaacaaaatcaggacctctggaggctggaggcaggagagttgtgttgtactgcgaccctcagtgcccagagggctcttcagtcctggcagcagctcattgctagtggccagcatggccaggcccattggagctggaggatacactgtaaccggctgacaccatgtacaaaggagagtaacacagctctactaccaccaggtatggctgtaaggccgggtgtggcataaactctgccaggtcacacatgggctccagacccacagacacctgctctaaagacagagacagaacagaagcaacatgagctttggcaacagacagacccaaattggcaccacgtcccttctatctatcagttctatgacctcaggcctatactcatcttttaaaaaattcaataataatgcctatcatgtaagctgccctcactcttcccttccccagtttgttttcttttgttgggctaacatttattgaaggcttactaggtactaagcaaatgctgtacaggcattaatatctctgttagtcttcactgcaatcctggacaaatccagaatgatttcttattatgccatttttagatatgaaggaacagaggttcaaagaagtcagacaacttctccattgtcacaaagctaacaagtggcaaagccactatgcccaagtcatcattttaagtatcatactatactctatccatatagcaagagtgacatttcataagtagataaagagcttgacacagagttgggccctcagaaaagagtcctgagagccaggtgtatcagtctgtccccacactgctataaagaaatacccgagactgggtaatttataaaggaaagaggtttaattgactcacaattctgcatgactggggagacctcaggaaacttacaatcgtggtagaaggtgaagggaaagcaaggaccttcttcacatgcaggcaggagacagtgtgagcaaagggggaagaaccccttataaaactatcacatctcgtgagaactcactatcactagaatagtatgggtgaaaccacccccgtgatccaatcacctcccaccaggtctctccctagacacatggggattacaattcaagatgagatttgggtggaggcacagccaaaccatatcaccagggtaccaaaatcattcccccgagtggtgtccctatccagaggccacagacaggacacctgccatgtgaaggtgacatggcttgaatgtttgttccctccaaatctcatgttgaaatgtgacctccattctgcagttatacaagcagtaagaataaaacaaaatgaaacaaacaaaaaagaaatgtgacctccaatattggagatagagcctaacaggaggcattgcaacatggggatgaatccctcatgaatggcttagtgccatccccttggtgatgagtgaattctctcttggttagttcatgtgagatcttatcagggaacgtgccctgatagtcacgtaggttcttttctattttgcctaagcgtcagccggtttgagaaataaagggacagagtacaaaagagagaaattttaaagctgggtgtccgggggagacatcacatgtcagtaggttccatgatgccccacaagccgcaaaaccagcaagtttttattagggactttcaaaaggggagggagtgtacgaatagggtgtgggtcacaaagatcacgtacttcacaaggtaatagaatatcacaaggcaaatggaggcagggcgagatcacaggaccacaggatggggcgaaattaaaattgctaatgaagtttcgggcaccattgtcattgataacatcttatcaggagacagggtttgagagcaaccggtctgatcaaaatttattaggtgggaatttcctcttcctaataagcctgggagcgctatgggagactggggtttatttcatccctacagtcttgaccatagaagatggccaaacccaaggggtccatttcagagacccagcctcaggcacatattctctttcccagggatgttccttgctgagaaaaagaattcagcaatatttctcccatttgcttttgaaagaagagaaatatggctctgttctgcccggctcaccagcaatcagagtttaaggttatctctcttgttccctaaacattgctgttatcttgttcttttttcaaggtgcccagatttcatattgtttaaacacacatgctctacaacttgtgcagttaacacaattatcacagggtcctgaggcgacatatatcctcctcggcttacgagatgacaggattaagagactaaagtaaagacaggcataggaaatcacaagggtattgattggggaagtgataagtgtccatgaaatcttcacaatttagagactgcagtaaagacaggcataagaaattataaaagtattaatttggggaactaataaatgtccatgaaatcttcacaatccacgttcttctgccagtccctccatttggggtccctgacttcctgcaacaagatctggttgtttaagagtctgagacctccctcttctctcttacactctcaccatgtgacacacctgcgccccctttgccttcctccatgattgtaagcttcctgaggccctcaccaggagcagagccagtgccatgcttcctgtacagcctgcagaactgtgagccaactaaacctcttgtctttataaattacccagtctcaaggaatacttattctttgatagcaacgcaaaaacagactaatacaaaaaaattggtaccaaggagtggggcattgctataaagatacctgaagatgtggaagtagattttgaactgggtaacagttagaggttagaagagtttggagagttcagaagaagacaggaagatgagggaaagtttgaaacttcttagaaactggttaaagggttgtgaccagaatgctgatagaaatatggacaatgaagtccaggctgatgaggtctcagataaaaatgagcaacttaatggagaatgcaggtcaacaaaggtcacccatgttacaccttagcaaagagcttggctgctttgtgttcaggccctagggatctgtgggagtttgaacttcagagtgatgacttagggtatctggcagaagaaatttctaagcagcaaagcattcaagatgtggtatgtctgcttctaataacctacaatcaaatacagagacaaagaaatgacttatgtttggaacttgtatttaaaagggaagcagagcataaaaatttggaaaatttgcagcctgcccatgtggtaaagaaagaaaaagcattttcagaggaggaatacaagcaggccgcaaagcaaccacttgctagagagattagtgtgactaaaaaaggatccaagtgctaataagcaagacaatgggaaaaagggccttgaaggcattttagaaatcttctaggtagcccctcccatcacaggcccagaaacctaggaggaaagaacagtttcaggagccactcccagggcacctgctgccctgcacagcctcaggacactgtttcctgattcaggcctactctggctacggccttggctcaaaggggcccaggtacagctcaatctgccactctagagggctcatgctgtaagccttagtggcttccacataatgttaagcctgtaggtgtgcagaatgcaagagtgaaggaggcttggtagctccacctatatttcagagaatgcatggaaaggcttaggagcccaggcagaagcctcctgcaggggtggaccccaccacagagatgttctactagggcagtgcaaagggaaagtgcggggttggagcctccacacgaagtccccactagagcactacccagtggagctgtaagaaaggggccaccactcttcagaccccacaatggtacagccaacagcagcttgcatcctgagcctggaaaagcctcaggcactcaactccagtccctgagagtggccatgggggttgtaccctgcaaaaccacaagggcagactttccaaaggccttagagtcccacccttccataagtgtgctctggatgtgggacatggtatcaaaggagactattttggagctttaagatttaataactgatctgctgggtttcagatttgtatggggcctactgcccctttcttctggctgatttttcccttttggaatgagaatatttacccaatgctggtaccaccattgtatcttgaaagtaaataacttcttttgattttacaggctgataggtagaaggagtgagtctcagataaaacttaaaactttggacttgatgctggaacgagttaacactttggaagactgttgcgaaggaatgattgtatcttgcaatgtgagaagaacatgagatttagagggccaggggcggaatgatatggtttggatgtttgtcccttccaaatctcatgttgaaatgtgacctccaatgttggagatggtgcctggtgggaggcaatttgattatgggggcgaattcctcatgaatgactcagcattatccccttggtaatgagtgagttctctctccaccagttcatgcgagatctgattttttttttttttttttttgagacagagtcttgctctgtcacccagactggagtgcaatggtgcaatctcagctcactgcaacctccgcctaccagattcaagcaattctctgcctcagcctcccgagtagctgggattacaggtgcccaccatcacacccagctaatttttgtatttttagtacagacaggatttcaccatcttggccaggctggtcttgaactcctgacctcatgatccacgcacctcggcctcccaaagtgctgggattacaggggtgagccaccatgccctgccagatctgattgtttattgtttaaaagagtctacgacttccctcttctctcgttcttgctcttgcttttgctctgtgatgcacctgctccccctttgctttcctccatgattgtaagcttcctgaggccctcaccaggagcagatgccagtgccatacttcctctgcagcctacagaaccatgagccaattaaacctcttttcttcataaattacctagcttcaggtatttctttttagtgatgaaggaacagactatatggaagggaaggataatgcctttggtaggcacatctcttgaggactgtgtgcttgggactgtgttaagaaatttacttgcacatgccagaaactatctgtcctgcccaaatattccatgtgcccacctcttcaggagactctgatggaaaggcaacagtctacaggaaaaatcacacccttactagaagatgccttttgctttagagggaaatggggcagacaatttacaactggcctaaagtgaataagagaagctctgaagacaggaccagaggatgaggccaaggcagggctgtacatcagccagtagacttactgtggaacagccacccagtgagaaggctaaacttacccttcccaagtccagccacttctcaccctgtgttccagttactactgtgacataacaaattaccccacaatttagcatcttaaaataaccatttttattatgcacctttattctgtgggtcaggaacttgggcagggcatggggtgaagaagatgaccagagggatgacttgtatctgttccataatctggggcctcaaccagaaagacccgaaggcaaggaatgatttgatggctagggttgtagttatctggggccattttgctcatatgatgggagactcaaaggctgggattaacaacagagcacctacatgtggtctctccctgcagcttggcttcttcaatgcatgatggccttagagtactgagacttcctacttagtggcccagtgctccaagcaccagtattccagctactaaggcagaggaatgtcaaggaattaaggaatttggaggccatgttttaaagccttcacaccctgccttccaggaaggagccaagctcaagggcatacgcaaaagagactgcagaggatcttcccctatccggggacctatttcctgcagaatccaatggcagaatctcccaggcataatgataagcaaaagaagacaggtgcaaaagagtgctaactgtgtgattccatttatgcagcattcaaggaaagacaaaactaagttcagaatagcagttacctgcaggtggggtgcaaaggggcacaagggagggagctttctaggcttctggcatgttctttatattaacacatttgttacacaagtgtattcatatgtaaaaaattaatcaactcctacacttaaaagtcatgcactggtgacactatggattttctacttcaataaaaattttgtaaaggaattatcctcaagaggagaggagagggggaacaaaagaattatcctatccagtcagagcacctcactctccatcccttcccctagccagatttacatgccctagaaaagcagactcccaggcctccccgctctggctagtcaatggcttgtggatgcagcattttaggggaggtgtttccagcccctcctctgcccttctgcccagacttccttctccgttttagaatccaaaagtgctggaaggtgccaggactatcatggtttcatttcatacccttattttatggaggaggaaagcaggaaggggagtggtggagggactggaatgtgcagccagggaggcaggtaaggcccggactcctctgacggccaaggcagaactacagtcttagggtctttctcttcctctcagtgaggcagagccctccattgcctgtaagactgcttagcatgggctctgtgcatccatgtctatccaaatgggagtttctggaaggcaggaaggtttctcacttgataagattccgggacccacctagctgggtggggattttccacagggcctaggcagagggataatgctagccactccttggggcatggctctgatcacagaccctgggggctgtgggcctgagagatttcacccctcccagaattaccagaaacggggtgggaagctgagatgcttttgctttccaatgaaactcccaaggctcaaaccagaagccagcatcttaagtcctaaaatgtgaagccccaacaatcaagccttttcctgatctggctttgcccatccccctaccaccccctgtcttttgcagtcagcttctattatggactgaatggtgttcccccaaaattcatatgttgaagtcctgaccccccagtgcctcagaatgtgactatatttggagactgggtcattaaagaggttaagttaaaatgagtcccttagaatggtccctaatccagtctgactggtgtccttataagaagaggaaatttggacacacggacacaccagggatgttcagaggaggcagggcaagaagtcagccatctgcaagccaaggaaagagacctcagaggaaaccagacctgctgataccttgctcttggacgtccagcctccagaaccataagaaaataaatttctgttgtttgtgtctgtcgtattttgttacagcagccctagcaaactatataactcccacttcctaagtgtcacacatttttacacctgcctggggtgcctttccctccctcctcccttgaccctctgaaaaacaccaccttccagtccttccccgccaattgctccatgacatgtctcttcaagccctgagtcagtgaaccgtgctacatggtcttgcattgtacacttgtaaccattatcatccctatgacaccatattttagtttatcttcttgacagaccttgaccctggagacacaggctattccttactcatctccgcatccccagcctgaataacaaatggtgtgataaaatgtctgctggattgaactgaactggagaatttgctgagtgcttcagcatgccctggcagggtctctgcccctcagaacccacacttccccttcactgtatccaccttgcaccctgcacaaatgccaaaaaggctttaaggtcaaaagaattagcactgtcttagaaattgtatagaggtggtccagatggtccccaacttacagtggttcagcgtacaattttctgactttaagatggtgtaaaagcaatacacattcattagaaaccattcttcaaattttgaattttgatctttgcccaggctagcaataagtggaaatatgctctcctgtgatgctgggcagcaggaatgagctgcagctcccactgagccacactatcacgagagcaaacaactggtactcgacagtatactgtgttgccagatgattttgcccaactgtaggctaatgccagtgtctgagcatgtttaaagtaggctaggctaagctatgatgttcagtaagttaggtgtagtaaatgcattttcaacttacaatggatttattgggcactaaccccacatcataagtcaagaagcaactgtattataaaactaagactgggggagggcaatgtttaacctggagacatggattactcatgataacaatgtaggatgccagggactgaacataacaaactcaaggggaggggaggtcccttctcagctatccagcaccccaaagcttgaatcaatagctccttcccacgctgggtgaaatcagccctgagctgtctgtgaatcagaggaagtgtttgtgtgtgtgtgtgtgtgtgttggagagggtccgggggaaggatgtatatgtgcatcctgaggtggaaaaatccctaaactcacttgtgtggtgaagcagggctggaggcttctagagccctagggagggcgcagcctttgactttcggacagacctggttagaagcctcactgctttgctgccagctgataaaggagccagatgaaagggcccagcatagctcctggcctttggagcatgccctttatccatagaatgctactcttctcctgatgtccatcttcccctagcgctgagcccaaagcacagaaggcatcctgtttggagccaggctggctggggttaacaagagaaaggcagctgtttcccgaaaacaaagggctgggtcaataaatctgccgcagcagccgtggatcagtgagggcaaaggctcccgcggggagcagccagccagtttctctgaaacgtctagaacagagccatccaggaaagcaaggctgaggcttgaaaggcccttaggtaggcctgtcctggggtcaatatcctcagagcacagggtccctctcctcacccccagcaccttccaggatcagactcagagtctcacagaatcacagagctggaaaggaccccaagcattctccaatgcagttttttatctggggaacatgttaagaattcatccatctatgaacttggataggtcagggggaaaaggttcactaatctataactgaagtttagcatttatttctatcatgaacctaagcaacaaactacagtagaatttggagaacctaagactttgtcaccaagagacatgacaggcatttttatagctcagtacaggcattccagaattctcaaaacattgttcattaatactacctcaaaattgtagtaatgatcagggccacctctagatctcgcttaatgcattaataaataagcattgttactgctatatcacaaatttgtttttttattttgacaactgtattttggtatcgttagttaactttgttattccatgtattttatattaccagccttcaaaagtgtccatgacacagaaaggattaagaattcctactgggctttaaccttcactatagatggagaaaccaaggtccaagacaggcagtgcttcttctggatggatggatggatggatggacgtatggatggatggatggatgaagggatggatggatagtgagtgcacagatggtgaatggattaatgaatagaaatgcaaatgaaaaaatagcacagtaatagcaataataaccctcatggactgcttaccctgagccaggcattattttaaactattcaattctcacaacagccctatgagacagataatattattttctccgttgaacagataagaaaactgagatataaagttatgaagtgacttagacaaggctacagagccagtaagaggaagagttatgtatgactcaaacccccaagcctgcactctgaacctctcttctctatgactgctctgaagcagtctggtgaagcttggtttagtgctgagttctgggatcataaagcaaggactgaattccaattctggccacttgcaagatggtgactctgggcaactgatttcactactgcaaatcacagtttttcatccataacatgaagacagtagtatcacctccatcacggggtcatatgtagattaataagtcagggaaagcacgtagcacagtgcccagtacatagtattgctagataattttgtttttaatgaattacaagaccaaggcatagacccatttagaatggtgatatgtgtctctgtgctaaaattgccaaaaataagaaaattaaagtaacgaggctagaaaacccagatcccagctcctggcaaaattgaagggtgtcaagaattaacaagttgagactgggcgcgtggtggctcatggctgtaatcccagcactttgggaggccgaggtgggcagatcacttgaggtcaagagttcgagacaggactggccaacatgatgaaaccctgtctctactaaaaatacaaaaattgaccgggcatggtggcgggtgcctgtaatcccagctacgagggaggctgaggcatgagaatcacacctgggagatggaggttgcagcgagccaagattgcgccactgtactccagcctgggcgatagagagagtctatatctcaataaaaaaaaaaagaattaagttgactttcaaactgcaagagtcgaaaaaaaaaaaagagagaaaacaagtgcctagtgaaattttgctttcaagatggtgtagacaaagggatggatttagattgaagtaaatatatgaaaattctagtaattagcgattccttctgaagagtgtggtatggaaagggaaggaaagaataaccttgcagtggagaaattcaacaagcgctactccagccagctggccaaggttaaagttaccggtgataaatcacattgatatgatagacgtatcatagatagatggcactttacctctaacttccaaaaattcatgtacccagtctaatcatgagaaaagtatcagacggactcaaatggaaggacatgctacaaaatacctgaccaaaacgtctcaaaaactatcaagttcttcaaaaaacaagcctgagaaactgtcccaatctaaaggaatctaaggagataggatgactaaatgtattgtagtatcctgaataggatcctggagcagaaataagaaatgaggtagaaagtaaggaaatgtgaataaagtatggactgtggtcaacaatactgtatcaatattgcttcattcgttgttgacaaatgtgccatactaatgtaagcgattaacattaggggaaactaggggtgaagtacaggagaactctctgtaccatcatttcagcttttctgtaaatctaaatctattctaaaaaagagtattggctttcaaaatgctagtagttgatctggtggtgagattacagacaatttcttttcttagttttatttgtctgtatatttttaatttttcttctttttgagacagggtctgtctgtgttgccaaggctggagtgcagtggctattcacaggctcgatcatagcatacaacagcctccaactcctgggctcaagcaagtaaactgagtctgtagctgggtctgcatttttcaaatggtctatgaggaatacttactgcttttgacatcagaaaaaaaattcaatgaacattactttttaaaaaaatgaatagatgttaggctctttggagacagtactatatacataaaagtataactagaatcgtccttggacagcaactaaaacccattttatcggccgggcgtggtggctcacacctgtaatcccagcactttgggaggccaaggcgggcagatcacttgaggtcagaagttcaagaccagcctgaccaacatagtgaaaccccatctctactaaaaatacaaaaattagctgggcatggtggtgggtgcctgtaatcccagctactcaggaggccgaggcaggagaatctcgtgaacccaggaggcgaaggttgcagtgagccaagatcgtgccattgcactccagcctgggcaaaaggagtgaaaccatgtctcaaaaaaaaaggccaggcgcagtggctcatgcctgtaatcccagcattctgggaggctgaggtgggaggatcacaaggtcaggagattgagaccatcctggctaacgcagtaaaaccccatctctactaaaaatacaaaaaattacaattagctaggcgtggtggtgggcacctgtagtcccagctactcgggaggctgaggcaggagaatggtgtgaacctgggaggcggagcttgcagtgagcagagagcacgccactgcactccagcctgcgcaacagagccagactccgtcttaaaacaaaacaaacaaaaaacaaaaaaacaccttatcgctctgcacccagggcctggcactctccccgggggagggcggtgtgcttctgaacctgccagcattttttctatctatgatacacttgctgacagaggtcaaagggctatcctgggtaagcccacactgctggctcaagaggccccaggcaaatcagccccaggaaaatctcgtccatcagcttctaggccaagcctcagcctgctctgtgtcatcagtctgggaggcaggaagactgcaaagggttctcagttcaccatacgaacaaaagacaagacgagactcgccaggaatgtgtggtttgtcccaggcactttgtcctgcatccagacctcaagcagtcagataaagctgattctttatttttgcacttcttttaaagctcaggctcagagagagagtccccagctcacaagagtcagaagcaggatataaactctgatctactcactccagagctgcccgcaagaaggacacctgtctctacccatctcgggaatgtgccatgaccacagcagatggctggactacgtttaggagacaggatgctggagaaaggaagtgtcaagcagtgggcagcacccttcagacctcccctgacaccaaccaccaccaccccaatactgagggctccatggacaaaggccagctcgttcttgggcccaaacaccagttgcagctcctggatcctgggaaacgtgggagagcaccagtgactgctccctgggctccactgctgttccattcccaagggcatcctccatcatcctggtgtccaattcctaggcagacaggccctccccttgctgaggacctccaaagcaggggccaggagagagacggtgctgagacatttctcactgtaattgcattgggaccagatcctttctctctctgagagttggcaacaagaaccccctgggaggaacaacaactcagctttctaacaggtagccttcttttttttttttttttttttttgagatggagtctcagtctgtcgcccaacctggcatgatcttggctcactgcaacctctgcctcaagcaattctctcacctcagcctctggagtagctaggattacaggcgcacgtcaccatgcctagctaatttttgtatttttagtagagatggggtttcatcatgttggccaggctggtctcgaactcctgacctcaactgatccacccgccttggcctcccaaagtgcgggtagtacaggtgtgagccaccgtgcccggccacaggtagcctctctaaaaggcctttttttttttttttttaatacaaacactgagatgttggcttcaaagtggtcccctgaaaggctgtccagttattctagtgacttctccagagctcattcaactcttcagaaattgccttcagggcaacctttgaccaaaattggtcaacctgcttgattgtccaaagtattccccaatacgggtagccaaagaactttcagctatttccaaaatccaaatccaggttcaaaagaccaagacctgcccctctgaggctgctcccgagaaccctgggcatagtctgagaaagggctgcatggcagacatatcattactttctcagttgtgttattggtcagacacctgcctttagggtcaaagcttgtgtgtgttggcttcctctctccctgactcccaacaaaggctctgaaccattccccagatgttggcagagtgtgagacaccctcctctgacttccctagaagcatggccccctgcctcttctccctcccagcacagagcccctgtgctgggatcagcccaggctccatttgttcctctaatgagtcttaagtatttggcttgggctcctgccagtcctactcctacagagaggagggagtgggaagaatgggacaaagctcatgccagctcagttctgctgggccctgggcagactggcacaggggctgcctggcccagacccacccaggggtgcagccaggaacccatttgaggcccacagcctttctttaccctccacttcaggggcttcccacagtgcagattcctgggcatacttcctgggttggaatcctggggtgaggctcaggaatgtgcatttttaataagcacctcaggagattgtgatgcataatgaagattaaaaaccagtgttccagaaagtcaatgacagggattcaggctggctggtgaaattgtgtatatgtgcatttttctagagtccacagttgtcaaattctcacattaggtcaatagtccccataaccctttaaaagttttttaaaaacaagcccgatgtggtagctcacagcctataatcccagcattttgggaggtagagacagaaggatagcttgaggccagaagttcaagaccagcctgggcaacatagcaagaccccatctctacagataatttaaaaaaataataacttagctgggcatggtgacacatacctgtagtcccagctactcaagaggctgaggctcaaggatcacttgaacccaggagcttgaggctgctgcagtgagccatgatcatgctacagcactccagcctgggtgacagtgcaagaggccatctctaaaaaaaattaaaaattaaaacaaaaggctaagaagcactagtaaatgagatgattcctaagttcagttgtagctctgacctgccacaacttggtgaccctgttcaggcccccatcagggcaagcccccaggatgctctccttttaccaaatctcagagaaagacaaggctggccttgaaaaaggctggcaagtctgggggaaaccaggatgacatagaactaggctgagggacaataggatggcaatggccatgggcttgggtgagaaatgacacaggttaggggaaattctagaatacctgggctgaaaggacccttagatatcaatgaaggcaaacctctggcaagaggggtaaatttttaccaatgtcaggtcctctctctctggccacatagctaaacctcatttctcactcccctgcagttggcggaggtaatgagttgaattctgcccagtggaatgtggatctctgctatggtctggatgtttgtgtctccccaaaatcctagcccctaaggtgataggtttaggaagtggagccttttgggaggtaattaggtcatgaaggtggaaccctcatgaatgggattagtgtccttacaaaagagaccccagagagctcccttgccctttccaccatatgacagtgagaaggcactatctatgaacaggatgggtgatatggtttggctctgtgtccccacccaaatctcatcttgaattgtactcccataattcccacatgttgtgaaagggactcagtgggaagtaattgcatcatgggggcagtttctcccatacagttctcgtggtagtgaataagtctcacgagatctgatggtttgataaggagaaacccgctttgcttgattctcattctcttctcttgtctgcaccatgtgagacatgcctttcaccttctgccatgattgtgaaatctccccagccacatggaactataagtccaacaaacctttcttttgtaaattgctcagtctcaggtatgtctttatcagcagtgtgagaacagactaatataatgggttcatatgaaaaggccctcaccagacatcaaatctgccagtgccttgattttggacctccaagcctccagaactatgggaaataaatgtttgttgtttgtaagccacccagtttaaggtagtttgttagagcagcccaaatggactaacaacagaaaatgtgtatcaagaagtagggtgcggctataacaaatatctaaaatgtggaagaggctttggaattgggtaatgagtagaagctggaagaattttgagatgaatgctagaaaaaaaaatctacattgccataaacagactattaaaggtgattctggtaaggtctcagaaaaggggtgctatagagagagcctcaatcttcttagagatcacccaagtggtcatcagaatgttggtagaaatacagatggtaagggctgttctgatggggtcttagatgaaaatgaggaacatcttattgaaaactggaggagagatgacccttgttaaaaagtggcaaagacatttggcttttgttcatgtcctactgttttgtggaaggtagaacttgtgagcaatgaaataggatatttggctgaagaaatttccaagcaaagtaatgaaggtgcaacttggctcttcttaaatgcttatagtaaaatgcaagaagacaaactctaaagatggaatttttcatcaaaagagaagcagaacttaaagattaggaaacgtctcagcctatttatattgtaaaaaatgagaaagtgtgctcaggcatttgctacagagactagcatgaatcagacacgcactattcttcaagacaatagaaaaatggccccaaaggcatttcagagattataggggctgccccttccatcacaggcctagagtgccagggcctaaggaacagaatgatttcaaaagaggagccacaggccctcagtgctcactgtccagcatcacctcaaggctctgctccctgcatttcagtgtagtcctcctcaccatcccaagttcagctccagtgcccaccctcctcccacccctaaaggacataggtggtaaattttggtggcatccgcatgatgccatctcagccagtacacagagtatatgtactgtggggacatggttaccttcacctagatttcaaagatgctccagagagtgtaaggggccagccaaagaaccaccgcacgggcagggccactgtagaaagccccaacaagggcaatgcccaggggagccacggaagtagggctaccttacaacccagaccagtggggccaccagtgtgtgatcccagcccaagatagctacaggtgtacaacctaggcacagagcctccacagaaaatgggaccacaagacagaactgccatggaggcaggaccaccaccccagtgggtccagaagacaggacttctctacccctgtggacttaag

SEQ ID NO: 2 - DNA, 52030 bp, Homo sapiens
ggaatagttcctggagaaagatctaacatctgcgcaggtctatcctgttctgtggctgtgtttactaaagtgagttctgcgcttacattcacacactgcaaactgcacgtacgatgtaagtgtacttgtcatgtagagttttaccaaaactatattagattatactctgaagcatgttttgcatctgacaggtcgagctcattcattcagtattaagtccctcatgggccaggctctagcctgtgtcctggtgataaggaggtgaatgtgacagaattcctattttcaataaactcactgtctggagaggaaggtgatcatcaaaataaattttagcacgatgtgtaatgattaatgcacagaggatctttggaatctccaggaagaaggcctgacaacctctggatgaaggatgggatggaagataaagtggacttagagagatgtatcgtgtggctctgtctttatccctggaaaggttagcctaagcatggggtaggtgctggtttcttttgctgagaaatgtctcagagaacctcagagtagcaggaggcctttgtgatagattggccagcagtttttatttccctgacctttcagcatcaaaaagtgccagttggaccctgggttttgccttggaattagtaaatagatagttcaacagcaatggttgaaaagcctcttggagtctgtcccatgagagtaaggatgttaattctgaagcctgggccattttccatcctgggaattatactctgccccttcctaaattcccattgtggtttcaaatgaagaggccattgttcactttccttcctaaaagaagcaatatatttaacttctggctctttgcctgactgatggcgagttgatctctctgaagccaacacccgtggcttcctatcaagagaaacactctgcctccatctgatttccttcttgggttgaagttatttagatcctacactggctcagtaggaggcttgcctgccgttatgtcctactaggtggtgacagggcccttgattccctccctgtgtttaatagaaataaagacagagtcttagcgctttagggcagctagttgttctcccaaagacctattataggaatttaaaagcaaatgtctgatgtgaaaatctatgctattttagggactttgaagcacaacacaaaagagtatcactttgttgaatagtttgggtccaggcaggtgtcctagagcagaagttttcatttatttttaagcaacaaaattctttctttcccaaatgaaatctcctacagaatccaaatactgaagggtttctcaaccccagcatgcttggtattttgggccagataattcttgttgtagaagctgttgtgtgtgttggaagatgcttagcagcattcctggcttctaccctccagatgacataagtggcacccacattctctagttgtgacaacaaaaatgtctccagacattgcaaaatgtccactggggagcaagatcacctccagttgagaatcaatgcaatataatctagaaaacagacttttaattaagctcctcctttagaactctgcccagggtccctgactgttagtgtttgagacctggtggggcgttctgggctacacagatgcactatcctagagggttaagttgtcattttaaaaacctgggacatagagaacttgttataaacaaagcaaaagagctaaaattagacaaatccacaagtactttgaacccacatgtacactaaatatttctgtctatgtattatgtatacattgtgtacttggatgtgtagcataagggaagagtcgaagggtggtgtctgccttactccttatgtgttgaggtttggctaccacaatttgccctgttttgccagagatgccagttgaaggcctgtgactgacagcccagtgatagagtcaccctcactgcacaaaatactcagcttctgtgggattcagtggccctggccctgctcatgggcattccgtggtgagtgggccagcacctgcagcctctttcaaccagactggttggttggagcttggaccatggttattccaaggtggatttttatccttctgccctttgtactaaatgggtcatgtgtgcctctgtacgtgtgtgtgtgcgtgtatgtgtgtgactacacgtgtctgtgtctgctcttctcctttcagaagcccagagccactctgggcctggccactctgagcattggttgtgtgggggatgctgggagcttggcacatgctactcctcctttttcctcccttagaatgatgaaagtgttgttggagcctgtcccacagacagaaactgtacctcatttatgtgggcatcagtataaacaagaacaataccactatgtgagtcaccctcacctgcggggagacgcataggctcagggagggaatttgctgccagagggtctcagcttcagtctgtctccccggcagactcatcctaacttcattccacagctcaagtgcagtgtgaatcagcaggcagcctgtgcctagctaaaggactctctctgtgttttgtttgtttggttgcttgtgaagatctctgggcacatacagcagtgttttattttcccaaaagatcgcatgaccactacagttagccttgggaacagattgagtaagcacaattcatttgatctatctgcagatctccagcccttctaagaaactcacttccaaaaatgtttcaggagtcctgtagaagtttgcttagagttccttaaagaggctaagcgtcagttgcaatatctctctatcaaccccatttcccccagtgccactgccatgggctggaggagtgggtaacttgcttaggaacttgggtaagggacctgaagctccccacccactctggccctagataggctaaagatttgaaattagtttgatttatgtaaatctctggtgtgaattattgctatcagataaatacacagaccattgaactgaaaacagagcctcaacacaggttgccattgctaatgttagtttatagtagccattatctagtagtgcctttatcagtgaccccaggtgtataattggtttgttgtgaaagaggagtggagttcccaggatgctttttgtcccccatggttcccttataagtgactgtatttcctgagctctctgcacttttgtcaccttttgtcatcaatatatacaatcagtttcaggcaatattgatagggatgtgaaaactaaacactataagattgaatggacttaatagctaggagttgcagaaaatattcctacatgtatacataacttttataatgtacaaattgttcttatgtgcatactcttgcataatcatcacaagtcagccttgtgacttaggctctctcttggccttatcttgagagaatttatgaaatctgataatattgcagccccgtaggaaaacttttcagtgccttgctatgatctctgaaggcctatactcctccttcccttcctaggctggtattgacctgctttttaaatctcatctcctaccagaccttcatattcattgtagttctagcagtagtgtactacttgtaggtacctgaatgtggcaagctcttttctacttcttactaatttatggaaacaccaatctacccattcattctttcactgctacttgtatgctaggcattggtgatatcccctaaccccctggtccttggtctaagcaggcataggtggtgctgtgtggtaagagctttggtcttggtgctaggggagctgtaaggagggcaccaagccttgacttgaagggattagggaggcttctccagtaaggcatctccaaagtaagtcattttagtgtgaggtggatgggagagttaggagaggagaggaagaaagagattagggcaggtgtggaccccaggctagacagggatggatgagtgataaggtcttgatagtgggtagtgccagatgcaggtgtatagttttgaggactttgccctaaggcctgaaggcagccattcaatgtttttgttttttgtttttttgagacagggtcttactctgtcacccaggctggagtacagtggcgcaacaatggatcactgcaacctcgaactcctgggctcaagtgatcttcccacctcagcctcatgagtagctggggctataggcatgtactaccatgcccagctaaatattttgtttttctgtagagacagggtctctctatgttgcccaggctggcctcaaactcctggcctcaagcgatcctcccctcctgccatggccttacaaagtgctgggattataggcgtgagccacagtgcctggccaattaaatgctttttaagtaggagcatagcagggtgcagattgctgcaaagcacctttctagaatatatcctgttcccctcattctccctctagtactttctagtatataccccgttcccctcatcctccctctagtactctgctgcttaggctttaggttcaattcaggcatcactgtctctgtatgtgctccagattccccgggcaccatcgggggtccctctgccccacagcaccctggacatgcatactggagataaagcagcaattgcttttgtattttaaccgtctagttgtgtgtccttcctcctagaccactggttttcaggatttaaaagtgtagattcttgttgcccaccccagatattcttacttactgtgggatagagtctaggcatccccattttttttaaaagaagcagaaacattgcagtaggctaatcttattgagggcctggtttatacattatgtgtgtgcacacacgtatgcacacacccccacatacttgatctcattcatgcttatatctgtatttcttttatttttattttctttgatcctgtcattcaaatatccatatttcttaagaatgtccggtataaatttgctatgctacaaatgtttgattgaatgaatgaatgaagttgcccaaatttacgtgtgaagatgtgttggagcctaagttaaaattcataggtgattccgaggcaaggccctttcacagcatcactcttacaccacagttgctacactgagatggcctttgaaaagcaggcagtcccactgaaccacagtatcctagagactaagaaacggggacaagagatctttaacttgcattttataattttcagtggcacatcaataatctgcagggatacactaataagatgggtattggacactcattacgacagaccctggaggatccaaaactgtaaaatctagctctgatctcctacaaatttttggtctacttgagggtaaaaggcagaaatacacagacagttgaatgactgtgccagatcgtgacagttacctggagtaccgtgcacagaagggcaatagaatgatgcagtgactgagtaagaaagagcatgccattagctggagtggtctgcagatacctttggatgaggaagcacttttatgaggtttggctttagaaaagtgagaagccattccaagtgggaggagtgacatgagtggaatcctggcaggagatgggctatgaatggtgagggcatcagtttgacaggaacataggtttgtttgtgtaggggagtgatggttgacacctaaagggagtgctggttcaggattgggagggccttaaatgataggtggggctataatacagttggggatgtgtggcacattcagcaatgtaaaagtctgttgtcctcgaccccagatccattcaccttattaatgaaggtcatgaatttttaaatataataactgatatggataggttttgtgtcctcacccaaatctcatcttgaatcataatccccaggtgttgagggagagacctggtgggaggtgattggatcatgggagcagtttgctctttgctgttcttgtgatagtgagggaattctcacgagaactgatggttttataaggggctcttcccccttcacttctcccacatactctctttctcacctgctgtcatgtaagacatatctgcttccccttatgccatgattataagtttcctgaggcctccctagccatgtggaactatgagtcaattaaacctcttttctttataaattacccagtttcagtatgtctttatagcagtgtgagaatggactaatatagtaaattggtactgatagagtggggtactgctataaagatacccaaaaatatggaagcaactttggagctggataacaggcagaggttggaacagtttggagggctcagaagaatataggaagatgtggaaaagtttggaacttcctagagacttattgaatggctttgaccaaaatgctgatagcgacatggacaatggagtccagggagaagtagcttcagatggagatgaggaacttcttgggaattggagtaaaggtgactcttgctatgctttagcaaagagactgacagcattttgcccacaccctagagatctgtggaactttgaacttgagagagatgatctgaaattggaacttatttaaaagggaagcagagtatagaagtttggaaaatttgcagtctgacaatgcagtagaaaagaaaaacccattttctggggagaaattaaagctggctgcagaaatttgcgtaagtaacaaggagccaaatgttaatcaccaagacaatggggaaaatgttcctagggcatgtgaaggacttttgcagcagcccctcccattacaggccccgaggcctagaagggaaaagtggtttcatggactgggcccaggaccctgctgctctgtgcagcctaggacttagtaccctgcttcccagccactccagtcatggctaaaatgggcaaatttttaaaaagtcagctcaggctgtttgcttcagagggtgcaagccccaatccttggtggcttccatgtggtgttgggcctgcaggtatacaaaagtcaagaattgaggtttgggagcctcagctgagatttcagaggatgtatggaaatacctggatgtccagtcagaagtctgctgcaggggcaaagccctcatagagaacctctgctagggcagtgcaaagggaaagtgaggggttggagcccccacacagagaccccaccgtggcactgcttagttcagctgtgagaaaagggccaccatcctccagaccccagcagatggtaaatccatctgcttgcactgggcacctgaaaaagccacaagcactcaacaccagcctatgaaagcagccaggagggggatataccctgcagagccacagtggtagagctgcccaaggctatgggagcccaccccttgtatccacatgacctagatgtgagacatggagtcaaaggagatcattttggagctttaagatttaataactgccccactggatttttgacttgcatcaacctgtagcccctttgttttggccaatttatcccatttggaatgggtgttcttatccagtgcctgtaccaccattgtatcttggaaggaactaacttgattttgattttacaggcttataggcagaagggacttgccttgtctcagatgagactttggactgtggacttttgagttaatgctgaaatgagttaagactttggtgaactgttgggaagccatgattggttttgaaatgtgaaaagatatgagatttgggaagggccagggctggaatgatgtggttaggctttgtgtccccacctaaacctcatcttgaattctaatccccaagtattgactagagacctggtgggaggtgattagatcatggggggtggtttcgtccatgctgttcttgtgatattgagtgagttctcatgagatctgttggttttataaggggctcttctcccttcacttctcccacacactctctgtctcacctgctgccatgtaagacatgcctgcttccccttctgccatgattgtaagtttcttgaggcctttccagccatgtgtaactgtgagtcaattaaacttcttttctttataaattacccagtctcaggtatgtctttacagcagtgtgagaacagactaatacaataaccatgttctcttctttctggagatctccagccacatttgacttccaggtctgtgggaagcagtccaaggtgcaacctggaacactttcatttggcttcttcatgtagaggaacatttacatgtcgctgcttgttccttctacccatggcctacacacactgccaagctgtcaccttccctgttgtgcaatgtgtttgccatcaccttcaatgaaaacaatttttttaatgtcagaattttcttagttattaatcccaaacacagacaccagaaacatgatccagaataaagttttcagttcacactgagcttgtcattgctgaagtaagactcttagctctttcacaagtatggacaaaattcagtaggaatgttttcatcacaagtcttacttcttaataatgctttgatctagggtaatttctctttagtcttggatggtaattttgcctgaggaaagtccaagaggtcttatgtgaagatttctgttttgtttagagtttgccacaaatactggaaggagaaagttttctgtaatttatacagctttacattagaaggctatagcttattttaaaatggtttctgttcaaaaaattttcacctacaattatagtataaagtgccttgtccttgtattcacagaggaattttctcatacaactacaagaaaatgtggaaagtttcgtctgagaaaattcaactttttcatctctcataatttactgcttcaggaatgcaatttaccaaagtggattagaaactgtttgtaatggggaaatacgtgcactctttgaaggcagttgtcgacaaagagtcaaactctaaaatatttgaagaggtttattctgagccaaatatgagtgaccacagcccaaggcacagtctcaagagatcctgagaacatgtgcccagggtggttgggttacagcttgattttatacatttcagagggacataagacatcaatcagtacatgtgaggtatacattggtttggtctggaaaggcaggacaacttgaagcaaggggtggggagttgtatgggggagggtgcttataggttacaggtggattcaaagatctgcttattggcagttagttgaaaggataaattagttattatctaaggacttagaatcaatagaaaggagtgtctgggttaagctaaggggttgtggaggctgagattcttattatgtagatgaagtctcataggtgtcagcccttagagacaatagatggcaaatgtttcctatatagacctaggaaaagtgctagactcaacagttaatctctttaggattgggaggacctggaagaggaaagatctagttatgttaaagagattctttacggatgcaaattttcctccacaaaaagatgctttgcagggtcatttcaaaatatggcagagaaacatattttgggataaaatattttgattttcttctttggttgttttgtttgtttgtttttgagacagggtctcactctgtctcccaggctggagtgcagtggcgtgacaatggctcattgcagcttcaaccttctgggcttaagtaatccttctgcctcagctgcccaagtagctgggactacaggcatgcaccaccatgcccagctaatttttgtatttttgtagagacagggtttcatcatgttacccaggccggtctcaaactcctgggctcaagtgatccacctgcctcagcctcctggagtgctgcaattacaggcatgagccacttcacccagctgttacctttatctatcatgtgatgctagctgagtgtggtggctcacacctataatcctagcactttgggagactgaggcaagtggatcgtttaagaccaggaatttgagaccaacctgggcaacatggtgaaacctcatctccacaaatatatatatatagatagatagatagatagatagatagatagatacacacacacacacacacacatatatatatacacacatatatatatatatatatatgtatctcacacgatgctataccagagtcaggttgagttggtatttttgtagagacagggtttcaccatgttacccagactggtctcaaactcctgggatcaagcagtctacctgcctcaccctccgaaagtgctgggattataggtgctctgcactctagcctttgtaacaataagggtctattctgtcggttttaggtctctattttagtgttaatgctggtcagttgagtctaaactccaaaagggagaggatataatgaggcatgtctgactccctctttgcgtcatggctttcactagtttttcaggggttttttttaatccccttggttgagaaggggtccattcagtcagtagggggagcttaaaattttatttttggtttacaaagtgaagagctttcctcttttaagtcctcaccatataaccagtctctaccagatgctgagaatagcttaaactttcttactgtcttatttgagcctggggtggtttgttgtaggaccttatcaacatagattttttgataattgatctactttacttcgatgtctgagtaaagctttaacctggcccttaaacaccaaaaatgctttagtgggagctcttgttttgggaaaatgaaaaatttctgtgtctatcaatcacggaaagtattccccactggtttgattctgaaattcaatcattgcctatgaaagttaaaaacatttttttttccctgtagctagcttttactctgtctctttaaaatacctttttttggaggagaggtggagcaagatatgtgtaactatagttttttctttttttgcttagatggtttaaagaattctgcatgtcatagttgacattccatcattatcctgtgttccacagtcaaggccaaaggttaaaaataagttcttaaaggaaacaaaattaaagcaagagggaattacctctgaattgttttagggactcctagcattttgcatgagtttctgttggtctggagaatagaaggaagcatgaaaggtttcattccttgcaaatcaagtgaaactggctcctaccctcttcttcaatacaaacatgaaacagaaaaagtagactggaagaccagaaagggcacagatttttataccattttcatcaggattactatgttcatgttatggaaccaatttgtaagttttgggtaacagcttaaatagaaacctatagaactgaggcaaccttctgtttctcaaagacagatggtgaagcaacatttataaatgctttacagttagagagcatatgctctcgtccactgttttttgtctttgtgatcaaagaaggatttgaggaatttcttttaaaaatcagtctgtacaataactgagtacattactttcagataatggagaatagtcacacccattgttttcagggcctcagggaatcctagaatgtgtgtcttgtggactgtgtcatagttggctagttacagtggcagttactaaaatcaaatctgtttcaattaataaagaacttgacaatgaaactaacaagacccattctaaagacgccaggaaaataaaataaaaacccatatgtactaggaattgtaaatagagaggaagcagaaacaatgtaatttgaattatcttattaaaattttaaaatattaaaatttaactagtttctcttattcagctagagacggtataaaaacttggcagaagtgtgatttttgtctatctgctggccagaataatactatatagatgggattttgtaggttttagtctctctaccccgctgtcttccaatatgtatgcagaaaaagtaaacatgatccctaacctttctgaaaccttctttaccttcagtctacacatacatagtcttgggcaaagagttatcaggtaaattccatggaatcaagagtagaggcatatgttgcttttcttaataggaaaaacagtgactgtgtccccctaaataaagtcatgattcaatgaaaagacaaacagcccaattttaaaaaaatgagcaaaagatttgaacagacattttctcaaaagaaatatgaatgggcaatacacacatagaaacatgctcagcaattttaggctttagggatatgcaaattaagaccacaatgagataccactagaaaggctaaaatgaaaaagaccaacaacactaaatgttggtgagactgtggagcactagaacttttcatatttttagtgggagttcaaaatggtacaaccgctttgaaaaatggtctggcagtttcttacagaactaaacagaccactaccttgttacccagcagttcttaggtatttacccaagacaaatgaagcatatctctgtcaatagaaaagcagatgccagccatggtatgattttgaacagccagtggaataaaagaatagggactatatctgaccttgctgttagagtagttaggcagatatgagcggggcaggagaggcccccctcccccacaggaatgtcaggcaaccatcaggtgatggtcaggtggttgttaaactgtctcactaacatactagttggtcacagcttgcaccagggaaagcagactcccagtagatagaaaacaccttaagctcttaatcagcagcttcctttttttttttttgagatggagtcttgctctgttgcccaggctagaatgcggtgtcgtgatctcagctcactgcaatcgctgcctcccgggttcaagcgattctcctgcctcagcctcccgagtagctgggactacaggcacgtgccaccacgcctggctaattttttatatttttagtagagatggggtttcgccatgttggccaggatggtctggatctcctgacctcgcgatccgcccgcctcggcctcccaaaatgctgggattacaggcgtgagccgcggcacccggccaatcagtagcttcctcataggatctcaggcgttggatgagtgggctcaaacttgcatgctaagagacaaaatggtggagtttagctggtgtatgaccttcctctaggaacactcaattggtaagggaaaaatgcctcaaatgaacatgtgcacagcttcagtaaacacactgtacatgcggcccttcccaagtgctggcaggccactgcacatgcggacagcccaccccaaggaaaaaccaagggaggagagacacaaacctcagcaccatgccattgtgtaaaaatcccaagtcaagcgtcggacagggtcctcggatctctcaaattgcccacttggccctcttccacgtgtactttgcttcctttcattcctgttctcaaactttttaatatactttaactcctgctctaaaacttgccttggtctcattctaccttatcctctctggccaaattctttcctccaaggaagcaagaatcgagttgctgcagacccatatggattcgctgctgctaatgttgccatttggatttttttttctttttagagtgttctagaatgcatgcttagagaagggaatgtaatcctaggacaccactcagctctgcaacagtatagctctgcagtaatgagtaaatgctgtttgttggtttactttaaagctctgctactgaatatatacaaatatactgttatattttcctggtaaaaccttttattatacaatcacttctttttatttctagcaattctttcttttacagtgtattttatctgacgttaatgtggctgcattgcatgtcttttggctagtatttgccctgctttgcatcttctttaattcatttactttctactttttctacctatgtcttaggtttgtgctttataaacaacaggtggttagatttcctatttctagaagtttattgagattctccctctaccactgtctctaccaattccattttttcccacctctattggtttggaagatagaatctctctctctttttttttttttttaagtgattatacttggaattttaacatgtctgtgtaactaagtctaaagttagctaccactttaccctcctactgagcaatctaaggatgttagaatgtttttaactccagtcactttcctcctggcttatgtgcatttccttgatatttttgatatctttttatttttcttttgaagctccattaactagatgttattagttattccatccttctttgtttctatttacttacatgtttataattttttaacataccactttcttgtatagctcaaaccttccctcgaacatttccctccttcttcaagtagattctcttcaagtacattctttatttagcgaaggtatttggtagtgaacaatctcagttttctgaaatatctttattttgccctcattctggaaagctagtttcttactttatttttttaatagacagagggtcttgctttgttgcccaggctggtcttaaacttctggcttcaagcagtcctcctgccttggcctctcaaagtgctgggattacaggcacgagccactgcgcccagcagtaacgctagtttcactgggatcatggactcttaagagctagaaggaacttcactgattatttgactcagccttttccatttccagaaaagcctgccaccaggcacagaaagaatacatgacttccagagtccctcgctatattgtccagactggttttgtactcctggcctcaagtgatcctcccacttcagcctcccaaagtcctgagataataggcgtgagccaccgtgatcccctttacctttttctttttttttttcctttatttcttcttaataaaaaaaaagtgggggggatacatgtgcagagcgtacaggtttgttacataagtatacgtgtaccatggtggtttgctgcacctattgacctgtcctctaagttccctcccctcaccccccaacctcttcaaaaaaaaaaaaaaaaaactatcaaaaaatggtggccgaggttcaaagctctcaccttggttttgtgataaaggtggttaatctttggtgttggatttcaaattagtctgtttacagtgagcaacagtacagcagttttaaaaatcatgttgaatcagaaacatgaagaaggcagcagagctttctctccaccatgcacttggagaaggaaatgactcggcagcatactcctcatggctaatatcctttaagatttttccaaaatcatgaaaccagaacttgagagcttaaaagggactttagaagtctaccagtggataagagagaaaaaaaaatcaaaagggaaatggaataagccaaactaaagggcaggattcttttagttctcagcaggaaacatatgtttatctggaaaagattttggttggacaggttgtttgtgtaagaatcttttttgtgaggaaggaagatcaacctgggcttgaaggctggggtgggggtagagagaacagtccctgagcttcaagtaaatgagaaaaatacacagattttaaacagaggagccctgctttcacggggatcatatgatcgtgctatcagcttacttttaagttgtgataatgtatacatactttatgaaatatgattaaatggtaactttagtattagtggctacaagaacagaaacagcaacttttaaaaagatatgaaaagatgttttgggtgaaagtagttggctgcgcaacacatagagtataacatacatcattaatttaagactgtagatgtacagtatatatttggcatgtacaccaggattttcagtgattattgatgtaataagatttatgatttttaaaaagaaactcttcatgatagttttctgtaccacttacattttccatagtgatcatcatattgtctttataaacaatgacaacaataaatcaaaaagagagatcttacatggtgcctcttctctgacagctgagaccacatcatagctacgttgttcccttacattgcatttacattacatttaaaatgttatcaggaggacagaggctcatgaatgagacatgttctctccagttaaatttagtaaagaagaatcagagaaaaatctccagataactggaaaatgtttacattttcaagtatataaaaccaggatacattatcttgtttaagcttggagttgacaataataccattttgagatgtttttcagatagaatgacagtgagagtcatagtggggttgcacctccacatgatgcaacccaaatgtgatggaacagaaacatttttcttcatcatcctgaaaccgagcagtgatgatccgctgaggaatgtttaattttcatgtattaaaaaatgaagaatgtctgacctactgttaatgagaaaaaaaaacaccaaaaaggaagaaagaagaagtggcagctaaaagcctttttatggatgacgtcaatagaggaaaatgaaatattcatagaatcacagcaagagaataggaattctgaacatttttgtagaaggaaactgtaggattcttgcaattgatagtaggacaggagggaatttccatccttatgtgtgcagaaaggagtagacacagtgaaatttgaagactattgtaaacattttctacttcacttcaccttgaagaagtctacagaaagcatatatggcatagctgggcagagtggctcatgcctgtaatctagcactttgcgagaccaaagagagaggatcacctgagcccaggagtttgagaccagccagggcaacacagtgagaccccaactctactttttaaaagattttttaaaagcatatatggcagttaaatttcagtgagaaatcaggaccagggtaaatgtaaatacattactatagcagataaataaatatggatgccccacccatatttcctttcatcccacaacagaaagctggaaatgctgaaaaattaatacctcccacccctgaagcagccatcagccagtgaggaatgggagccagaggataaataacccaacttcctcacccgctgagggtgttcaaccaggtccttgaaattcccgacaggaatcgagcctcagttgcccaccctggaaacctgctcagtaatacgcttatcgttggtttccttccctttcctgtctcacttctgtaatcccctggtgcatctgggggttccttcccgaataaactacttgcaacttaagtccctatcttgaactctgctcctgggggaatccaaacaagaacaggtggagacagagtagaacaaggataagcaaagcaagagaggagatctgaagacgtctctaaggttgtttgagtgactccaaggatagtggtacaattagctaggagaaagagagggtggaggaggccgagaagagggttcagcccaacaaatgccaagttttaggtagcttggggataccttgaagagatgctgacaagctacttagaaataagtttattagctagaagccggagctggccaggcacagtggtatgcacctgtagtcccagctactctggaggctcaggtgggaggatggcttgagtccagaagctgtttgaggtcagcctgggcaatgtaatgagacctcatctcaatatcaacaaaccttatctcaatataaatgaatgaatgaatgaatgaatgaatgaatgaatgaaaccagagctatagctgtatatttgggaatcctcagtgtacaggcagttgatgaatgaacatccagaaagactagaagcatggtatgcagggaaccttggagcatacacatatttaaaaattgggtggaggagaaaagccagtgaaggagactaagaaagacgattatagagagaagaaccagagctgttattggagccaaggcctctttcttcaaggactgagaaggggagagtttgtactttggagtttgcacagatcacagacatgaaaaaggaaagatgatgatgagtggaaaatgggaggtcaggactctcctcccatcctgatgtggcaagaggcttttctcatggttctggatggaactttggccctgttgcagatgctcacatgcaacgttctgttctaatcactgttctgctggcagaggaggctggagtgatgagtgagcatgggctcagcttgcgtggatgctatgagccgtaggtcctactcaagagagtcagtctagtaacaaattattttcctctccttcttttgccatcccatttagtttgcaggatggttggtgtttgtgtgggctgagattcctcagagggaaccaaagccctgagaaacatgtgtagcagctgttgatttgggtctccacagtgagatccgggtctaatacagtgagtccaaccactttatctgagtttggaaataactatgagtacatagttaagctgttttcaaaaaacacagaggattctgtactgctagtgccattgagggggcttttgctgccatttattttcaacttatttttttgaggtggagtctcactctgtcgcccaggttggagtataatggcgtgatctcagctcacagcaacctctgcctcccaggttcaagcaatcctcctgcctcagcctcctgagtagctgggattacaggtgcccgccaccaggcctggctaatttttgtatttttaatagagatggggtttcaccatgttggccagacggtcttgaactcctgacctcaggtcatctgcccgcctcagccttccaaagtgctgggattacaggcatgagccgccacacccggccttgctgccatttttaaataccaaaaccttttgttgcagtctttgtcacaggtgctggcacaaaattcctggtaccttctccctgctctaattcaccaagttctccttcctcttttccaaagactactaacatggaagtcttccttaattagttacaggactatatctgcaactcctgtgatttattcgctcagtgcccgtgtcactgtcacccactggtggggtgtgcttgttgtaaatacccatttaaatgtttacattttcaagtatataaaaccaggattgtaaaataccgttgtatttccagttgtaaatagccgtttagttaagttaggcatttgtttatcacgcgtcctaatctctgtttgtgtcgttactgtctcagagatcatcatcatctgtgtttacatttttatgcatcattctttatgcaagtagttcttattcagtgtttttgatgatgttaagcaaaattctgttgctaccttacctcattaccttcccgggatatattttgcagatataaaccaataatattttctggttctttcttgactagtagcttttggttaactctaaggtttttctgtttgtttttctcacagttgttttctcccacgtgtacaaatgataaaacaaattatctcaggaagaagaaaaaagacatttgatagtatatccagtagttcaacaatagtcaacggagtctggtgtcctcacttctgtcattggctgcttgacctcacttaaccttcctgttagtttctgtttcctcactagaaaatttattactttctagggataccaggagacctggtgttaataaagggcacagtgcagggtgtgtggaagattttagtgtcagttctagcacaagctagctaaatgtgtctcggggtacattaacttctctaggccccaatttcctaggtattcattcttcttctagacaatagacagttgaattaaaaaatcctttctatcgaccaggtgcaatggctcacacctgtaatcccagcagtttgggaggccgaggcaggaggatcacttgagcccgtgagtttgagaccagcctagcaacacagcaagaccctgtctcttaaaaaaaaaaatcctttctattcctagaactctagggtagtgtgaagtaagggttttcacagttacactaggtaagttactacctgtcgggtacttctctaattatgctgtgcttattaacttatcaagtcctcaaatggccctatgaggaacatatcctttatgttctatagatgaggaaactgaagcattggatgttaaacaacttgcccagggccaccgagcttataagggaagaagccgtctgcacaccccaggctccagattccacagcctcaaccaccaaactctcttgtctgtcgtcatatagaaggctgttctgttttctcagtgtagcccccttaaagataagccacagtaaaccacagtgcttttgatattaggcaaattaaacttgagttcaatcgtatgttacactgaatcaaagttatattcgctgtgcattttacgccagtaattctggaaactaaaaagtgccatgccagaaattttctctgatttaatttgcttacaatcattttaaaaatgaggagctgtgatccacgcttttgccccaggttggaatattttagaagtactttcaaatatatatttctgctgctgggatataaaaccataaattcgagcagatagaacaaaagaccaaagaggatgatgtatttactgtttttgagttaaagttacaacagtttacagaatcacagcaaccgagaataggagttctcagagacatactcctcatccacagaagcctcacatgaccaacacaagtttcagtccagtgcaactttttgcaaatgaaaacttaccctggggttgtttggtgactcctagtcacatgtgctttctaaagttctcaaaacaagcatggggtgacagtccagttttatgaaatataattttagttcagaagagatgggattttagaatagttaatcttcattatatttgacataattactctgcaccctgtggatggggtggcttttcaaaacccacttctggtgaggcagaccagggctccaggccagcacccctctggagattggaatgtgaatttccgtgctgggacacagagccagcttcctatttatttagagccattgggcacctaaggagccaaacagaagttgtgatgcaagttttggctgcagagcatgttagggttgcctgacgtgtctaactaaaacatgatctgagagatttctcaaagccctggttgatttaagcaccatagtattcaatagggtttttttttagtccttttaactcagtcctaagtaaacaggaggcggtgcaaacaatgaggaagttccctctgcattccgatggtgaagtggggttggagtctgtaggcaagtgaagagcgtgtgggagagctgtttccttttgctcatggcagcagtctagaacatggtgacagtgaaagtcattggaagccatgggggcccattttctttatgtgaccaaatgtcttgcaaagtcccaggtgtgtgtgtaggtgcatgagtgtgtatgtttgttttctaaacgaagcacacgtgcaaaataatgtaaaagggcagagtgaagtttgtccatgtccctgtatgaaagtgcatgggaagtgcctggggcagaagtgaaagagacacggccccaggtcgtgagagaagacattgaggtgcactcccggcctgctgtgggaacagtgggttagcatgtcaccggtgcactcccaggctgctgtggcaacagtgggttagcatgtcacccggctttaggatttgggggagtttgctcaggctctgaggcaggtaggttaaggctaatttaactcttaatggttggaaggaaagagggaagggccgtttaggaaggtgaaaaggcaggagtgaaggtggcaagaaatgattggcatgcagttttttcctaaatcctcctctggttataaggaatcaactggaaaattgcagtggcaatagctgttaagaaaaaaaataggattcagttgatgaaatattaaacatgcaagtgggctggtcttttaaccaatctttgtagtattaagggattaaaaagttaattcttggtaattagatccccagggagaacttaatttgcatctatacatgataagtgtttactattaataattcaagacaagttttgctttggccggtattgagctgggtctcaggctggtgataacagaggctggatagagagtgataccattagcctaattttgtcatctgcaagcaaaggaatgttaaaatctgttcacttcgtggtttctaaatgacttctagaagtatcatgagtggaagagggcagtgtagtgtagtaacttttgttcttccgcagtccttagaacctgtcaggattattggttatgacactgttcccacagggtggacaacaggggcatttttatcttcttgagacatatgggaaacttaagaaggaaagtaactaggctgtttgccaaagattacacctacctccaaacaaaaactgaggcaggtataattttaactgtgctggaatctctgaagatgttggtgtcttggccaaattcatttggactctttctgcatttcactctttgagatctattgtgtagactctaatttctttgacaggtattgtcacattggtctccacaaaaattattattgaaagccaggcatggtggctcacgcctgtaatcccagcactttgggaggctgaggcgggtggatcacaaggtcaagagatcaagaccatcctggccaacatggtgaaaccctgtctctactaaaaatacgaaaattagctgggcttggtggtgtgcacctgtagtcccagctgcttgggaggctgaggcaggagaatagcttgaacctgggaggtggaggttgcaatgagctgagaacacgccactgcactccagcctggtgacagagttaaactctatccaaaaataaaaataaaaaataaaaaattattattgaaataactacattttttttcttttttttttggagagaaagtcttgctctgttgcccagctgaagtgcagaggtgcaatcatggctcattatagcctcaacctcctgggctcaagtgatcctccctagtagctgagaccacaggtgcacacttggctttttttaaaattttttttggtagagaaggagtttcattatgttgccagactggtctccaattcctaggctcaagtgatctcccacctcagcctcccaaatttctagaattaaagatgtgagccactgcacttgccctaaataactacatttttttgaatgctgcgatgtgcctagctttgtgctaagctctttgaatgtgttacctgattcagtccttgggaaaaccctaggaagaaggggccacctgatttctagatgggaaccctgaggcttagcaagaaaaagagagccggtgtggtagggtcaggactggagcccggtttatactcctactgacaggatggggatgcctgtttcacctcacccttgttaaaaaggacactttagcaatccttttaagtatttgccagcttaagaggtaaaaatgtgatttagcttctggttcttatgcgtccgtcttgggtagcattggacatcttggcatggttattggctgtttgcacttacagttttaggaattgcctcttcacatcctctgccccatttttactggattgttcatacttttaaaaattgatttctgtgtattataaatagtaaccctttgtcatgtgttacaagtcctcttctccctcagtttctttctttcaattttctgctttgtatttttttttttttttttgccaaatagaactttaaatttcttaaattgcaaatctgtgaactttggatgcctttagagtttattatccctttaaaaaaatttttttaagagatggggcctcagtatattgtccaggtggtcttgaactcctggactcaagcagtcctcctgcctcagcctcccaaagtgttggtattacagacatgagctactgtacccggccaagttttattatccttatcttatcctaggagtataatatttttctgtgttttcttattgttctataatttcctttgtaacaggtatctgagattatctttgaaatcttctgtctcttccagggtttaaaattttttagcaaattattatttcatggtttcttaaatttcctttgtttcaatgattaaagcattattgaggaaataagctatattttattaatcacaagtctctgcatacactagaaaaattctagtataattatttggaagacataatgatgtagttttcagagtcctcagattagaagccactgttttaaaagcgtgaggttagaaaaggaattttgcacctattccaggttaatttctacctattccaggttaacagaggtgatcctgtaccaatgggttttggacagagctctcatagtctgtgctgtgggcgattccctcatttagtttgcgaggataccagaattaaaggaaaataatgagaaatgcttatagctcatagatagacgcgaggtcacagacagccagactggagctaggagaccatgctgatgtcacatactgccctatcagaaccctgggaaacccttggtctgtgctacaagagattggcccatgatagccagttataaccaggcagtgggcatctcaggggtttgcataggtctaaccttaaaaagccacatgactgggtgcggtgacacacacttgtaatcccagcactttgggaggccaagacacaaggatcacctgaacctgggagttggagaccagctctggcatcacagtgagacctccatctctattaaaaaaaaattatttttaattagctgggtatggtggtgcatgcctgtagtcccagcaccttgggttgctgaggtaggagcatcacttgggcctgagagttcaaggctgcagtaagccatgattgagccactgcacttcagcttgggtgacagagtgaggtcctgtctcaaagtaaagagtcctgttaggaagccataatgattttaggggggatggtttagatcttgaagaaagaaaaaataatttgaggagtgagcacttgaactatcaaactttctcatctattaggtgcgttaggttccttacatcttattctttttttccttccttttaatatatagctactctccagagagaccggatcttcaaacattttaccaggaagcgccaaagggctatgcgaaggcgagtccaccagatcaatggacacaagttcatggccacgtatctgaggcagcccacctactgctctcactgcagggagtttatctggtaaggggttcccttacttctgtcctcctcagagcttccatctaataggcttcctctggttatgtatgacttcagcagaatgcatgattatttaagaattctttcaggatctgatttttgcctcttactctagtatgttgacccagtaatttaaatctccattgcaaaaacatctaaataatttacagatccaagttttagataccatttctttttaaaatcatcttggtaaagatgctaatgaatcctaaactggtcaacaagcataaatcagagaaaaaggctaataagtcaataaacaatcagtaaattggataatcagcccctaaggaccaaagaataatcaacagtattgctttaagaaggcatgaatcgcataatgacttatggaaattaatggttgcgagaaaactaaacacaaagtagtcttcggcctcttggtatgtaacagaagataatagccttcagtgatcatatactgtaatctaatccctaggaattagtaggaactaagataattcaacattgagaaattctagagatttggggcaatatcttactagacatacaactgaagcttaagtctcagtctcagtggcaaatgtaaaataaatttaagcagtagttatctttagtgataaattaagcatacttatttggaaagggaagaaagaaagatgccctcacacacccatctcagaaggagaaaacacagtttggtagcgtgaaaggagatctatatttttgttgtcaactttaattattttgtttagtgaaggcagaagcccttggatcagagcagtttctgaatgttgatcaattactttgtattttgttttctgtgattattttggattttatatcttcaacagtcagaacctagggctagttagtatttctgacatttttaatcaagaaacaacgattttgagatgctaaaccagctgtagatatgaagccaacaaatgtgtcataacatttattgtatttagggaaaaaattgtattacttttttttgggaaaaaattagcagccctggcgtctttgagttcacgtttcctctttgcatgaggctgcactcgacttagccacagccccacctctctgtggtttgctcacttgtgacctaagggtgcttgcctcttgcagtgtatttcatgtcacacttttctgtttcttttacatgacctgccaggcccctgtaggcattagtggtgatggcccctgacctgaactgacattcataatggaaacttatttgatcatttcactgacttttttccaccagagaaacattaactgtacccaaccagcctttaacgctccccccctccacacccatacactgtcaccctcatactctctctctcacacacacttaaaatattggtgtatatattaatatatatggctaagagtatggactctgacatccagtctcatcaccaactgggtgtgtgacttagtcaagttgcgtagcctgtcaatgtctcttcatttgcaaaatagacacaacaatcttattttacttgtataataaactttcatatagtacttaaagggccaggcactattgtgagcattttatattacaacttttaatcttcattagagccctgtgcagtaggaacggttatcattagtcccttttatggatgaggaaactgtggctcaaagcatcaggtcatctggcttcagagtctgtgctcttaaccatgttattttgtgtgctgcctcccgtggctttttgtgaggattaaatgaaatggtatatatcagctgcttggttcagtgcctggcacatcctatgggcttagttactgtggtctttgttaatgagaatgagaatactcctaatgttaccatcattatcatcacccatagaatatatttgggagaggcaaagatggaagtggtgggcctgaagggcatattgtggcagcaactctgcccacctgtggactccaggggtgatatggacacagggacaagggcaggtcccctgggacaggcagctgcggtagactcatctgtgcacttccacagtgtatctgtgtccgtttacttcagcatctgaacatctttatgtgagagagaataaggtgggaactcttcaggccaaggcttaatcacacgtacagatgaggatttattgccactgaggacttaggaaattgccatggaggtagatggaatgggaatggtttttacccacccatttgtatcatacagcacattctaatccagcacatggttttttctggtgtctcatagtaacaaacaatggctcagaaagcacatgatctttgcttcttccctgaaattgtgtttttcataaaggaggagaggatgaaatttgttttccttaaggactataagagggttattgctgaaatacttgactgatggtagttttcttctcttcaacaggggagtgtttgggaaacagggttatcagtgccaaggtaaggaaacatttttaaaaccatgtttcattttgttcctatgttaaaagaaatgattataccaagagaaaacagggtatcttcccttaatattgtgataaataactctctgtaggtcaaaggaaacccctgtgtagatacaactctttagtttggtgagatcttggccatattttagacaatgtaaaggggacgtttttatttctaaagccaggatacctaataaattttcattgtttaaagtcttgtctctgcagtaacatgagagtactaaaaggtaatgtgattttgtgagcatactcttaaattttgaagataaacttttgttttttacaatcctattgataatagatagggatgaaaagtagcacactgctgttttctctttttccccgtccagggaacataagctattatccaagttgttttttgaaatgttatgatttcaaaaaaaaaaaaaaaaagtgagaaatgttcaaagttaagctgtcagtgatcttgtaagttactacatgttatatttagttttcacaatctaagatgaaaccaatttactagtgtttctagcataattggaaagaaatacaatggcaggaatggaagaagagccctcagtcaggtggtttagccaggcccatcaggcttgcctgtcatttgggttcccccttaacctttgacatgagtatcatcacatagtttatacacatggtacctgtttcactatgtggatgattgataaaatagcaccacatatcctagagcagtagcctatgtgtggataagaaaatctcaccttctcttctggaagatgtgaggtaaaacattacttcccttggatggactgaataagacatactcttggatctaattttgactttactatgtatgaacaattccattgaactttcaaagttaggaaaaaacaaatatataatcaatatgacagcaataattgacctgtatgctgttaatctcagtcccaagtgagaagacagttcacaagccagaatcgctgtcactttgtggcatgggcctgtgagcacaggaatatgccctgcagtccatggtagcccatcttatgcacaccaagataagttgagaagctctggataaacaagattatggttttgagtttaaaacatgggatttaagatacatctctattgttattttatttctttttctctttgtcctatagtgcctggatatttgatggcctgattttcagtgggtcacctgctatgtaaagagcagggaagactattagttaggagacatgcagacaccactttggtgatgtctccctaaggcatataaaaccaaatgcctggttggagagaggtatgccattctaacactgatcaaaagcaaactggcataggtatttcagacagagcagacttcagaaaaaggaaaattatcaggaataaagaggggacaacaataaaggggtcagttttccaagaagacttaacatttttatttgttttgttttatttaaagaccagtgtagtagtacataacaatttttaacatgtatgtatgtacctagcaacaggaatcaaaatacatgggacaaactgatagaatcacaaggaaaaatgaacaaatccacttctatagttggagacttcaatatctcagtaattgatagatccagcaggcagggaatcagtagggatataggtgatctaaacagcattatcacaactaggtgaaattgacgtttatagaatacttcatccaacaacagcagttttcacattcttctcgggctcacaggggacatttaccaacatagaccacattctgggccataaaacataccttaccaaatttaaaagaagagaaaccataaacggtatcttcttagaccacaatggaattaaactagaaatcaattactgtaagatagctggaaaatccccaaatattggaaggttaaatgtcacattctaagtaacacatgggtcaaagaagaagactcaagagaacataaaaaatatttagagtcaagtgaaaatgaaaatataacttataaaaatatgtgtgatccagcaaaagcagtgctcagatgggaatgtatatatcattaaatgcatacagtaggaaagtagaaagatctaaaaccaagattcgaaatttccaccttaggaaactagagaaggaagataaatttaagcctcaagcgagcataagaaaagaaataatacaattagaacagaaatcagtgaagttgaaaataggaaaacaacagagaaaatccatgaaatcaaatgctgattctttgaaaggattaattgataagcctttaccaagctaaccaggaaaaaaccaaatacctcaaaaaataaagtatttctgtgtgaaggatggttttgagatcccctcatataaaaagaccagttctctgggcactttcaatttggttctcctgagaattgaatctgatgaaagctagatttgttttaaggtatgatgctatttactgacatgtcacctctctttatgtagttaatcctaaaaccttccacatgtcttgaaagatcttaagatgatatacatttttatactgcttcattgcatgtgtttatcctttgatgacaagctcaactatctgcagtgtatcacttatctagatgagaatgaaaaaaatgttattccgaaagttctaaatatttcaacagttgattaagccattcagcaaacatttattgaatgcctcctctgttcaaggcatcatgcaaggaattctgagggcattacaaggctgggtggaaggtttgaaatgaaggggcaggctaagggttgtgggagatcagaggagggcaggtggtttcagtctagaagtggggatggggtagagtattttcatggaagagacattttctgagttacatcttgtcctaaacttaagatttggttagttgataggcaaaaaggatatcccagggagagaaaagggcatcgatagtttgttggaagggtccaggaagagcaacccatgtagtttttactggaacaagtagatcccatagaagaaaggtgacaggtagcttggagcagcctctggagggcctggaacctgggtgggaaatttgaactttgttgatcggcagtgaattaccatggaatgttttcgattatgggggcaggactagagtgttagaagagtcctctggtggtgggtgcaggatggaataaagtcaggaaggaagaaataggcccaggacttgataagggatagtttgagcctgagcaaaggtctgtgttaggatggtggcgaaggcaataggaaggatgggcgcaccagctgctgtcaagggggccagacgagtccagtcttgttggcacggtgcagccctggttgactctgctgtaactggctggactgtgagcttctgataaatgtataattgctggatttaatttcctagtgtgcacctgtgtcgtccataaacgctgccatcatctaattgttacagcctgtacttgccaaaacaatattaacaaagtggattcaaaggtaagaggatagcagtttgctgattaaatgtgcgtgtgtattgtgtatgctgtgtaccgccgtctctctccctccctccctcctttcctccccctcttttccttctccccgtccccaaacctctgctttcctccccctgccacctcccttccacattctttgaagccaagggttgtattagaatagaaatgtctctctccctttttttaaattatggcatttatatttctgtatttatgtggcatcatccctcctttgtgaggaccccattagtggttggctgggtataagctatatggattggttctacttatttggagagacttttggcagagtgaaaggagccctgaacttggcatctgacagcctggatttgaatgctgaatccagcctctgttagccatcttctctatgagctgcggcaaggcctacaatctctcttttcctcagtcttaaaacaggatcaatgtttccctcacacagtttttgtaagttaaacaacatagaattgatgtgatacatatgttaataactgtaaattatgtttcctaagtaaaaagattgctggctttctccagcctcttaagaaatttcagggaagatgtctctctctctctctctctctctctctctctctctctctctctctgtgtgtgtgtgtgtgtgtgtgtatgtgtataagcttcaacatcattcttgccactcatattgcattcatctgcaataggcatacattttaaattctagaggtaaaagcagatggagtgttgctgacatttagcagtagatttgcagaggcattggaagaatgactctggaacacaatggttctttatagtacacatataggattgctaaagagtatacctagaggtataaaccacagcttttccaagaaagaggccataaaagtttcaagaggagtaatgattgccctactccttgtcatgcatggatctgttgttatagcatcaaattgtggtatggactgcagagtacatgcctagtggaattatttgctggaaactgagatttaactcagattgtcagattgcaaatgccaggcaaagaaaggctggaagagctgaataagtagttactttaatgtcactttggaataaattccttctgtggttccatggcctctgagtctcttctttttatttttcttcccgtgacacaatgtctggaagaacttacagtgatagcaagtatatatagtgtataaaagacccatttaatcctgaagttaaaaggttctgggaaatagatgggggtgttaggggattggcaaggagagaaatatggcctgaatatgaaagatgaagtgaaaatcttaatttttatttctcaaaatggcaaatgatctaatgtgtttcccacatttctgtcttgtcctgttcattagaaaatgtgtttagtctggttcgtaagacctgagtaattaatcagggctgagtacagtaaaataatatacctagctcaggtgtcatagtgacactttgcatttgatgggctcctattacagtttttacaaaggacattggtatgacattttagctcttgtccctttatttccttttttacagattgcagaacagaggttcgggatcaacatcccacacaagttcagcatccacaactacaaagtgccaacattctgcgatcactgtggctcactgctctggggaataatgcgacaaggacttcagtgtaaaagtgagatgctgaggtgctgggtacccacctcttcatgggaacactatgccctaagctttcagaattctgtggactcagaatctgtcctagaactgatggttttagatatgttgtaaatcaacatcttagtcattttaaacttacatgtttcttgtcaggcatgtaaagagaacttcatctgatgccaacattttctaatcataggacattaagtcaaatggtatattcagtgatctagctcgctagctgacaaatacagcctagacactgtattttaataggtgtttggaagagaaaaagatgggatcttagtcagaaatggaagatggttgagtcaggtgagacactaaaaagaaaaaaataaataaaaatttcaaagaaatggaagttaggaggaggtgaggaggtgagatggccaaatgagaaggttgggttaagagtaagaaatggcagaatagatctgatgatctcaccagaaatcagcaggaatggagttgtcacagcataaaaagagctgccctgggtcttgtgaaagagattggagacaggggtagaatgaaggaagataaagccttgattgtaagcttgcatctcaccagctgtgcccagtggagtgagagtttggagctttggccctagcaaaggctctgagtgggtgagagctgatccctgtcctgttaggagttagggagctgttggccttgtgccatcttgtatggaaatgtaatgatctgactgccgtggggtgcacctgaagcctttcttatcagacacaccccaagtcgccagttgaggagaacaatggtgaggtagaattggcatgagtatagccagacaacttccattgactgcttggacttacagagcctgcttctgtcaacaaaaaggggccagtcacttgtctttaagaagggcacattcttctatctctgtagaacactaataatttttttctcataaaaccaaaacagtagccagctcatggataacttgtagggcgttggtccaggtgcagcttgatcttagcgtgacggtctcacctgggtatgggttcccagtgggaaatccctttctgccttgcattgttgctgatgcgcagagccatgcatggggctcatctgcattgtcctcttccctagtggcctgagggtagcacacctgcagcaatccggctcttgtgatttttgcctttatggtcacttggtggttttgaaaggcttctgtaggctcgtgtgtcttacacctgtcttcctgagaggggtgggtttcccatgaagcttaagtctcagggctcctctctctcaagggcaccttccaaagccctctacttgatttggtgttctttttcttaaagaaggcctcctaaattatgtaaattcaggccccacaaaacctggatctgtgtgtgcttccaagatggactgactccataaaacaggaatgtgttgcttgtcgcttcagtttcacctcccatcagcagggcccatgtcaaaaaactgtctcagaatttattccagctgataacgcctcagatccttcattgtccccaggcagcttccggtgcctcaggagcttagctttctttgctcgcctcttcaactgccatttgtactctgaccttcagaaagttttaaaagttgcctttccccactatgaagcagttagaaccacaggctgcaaatgctgagaaccacaggctcacaggatgcccatctctgtgctgtgaggcctgcccaaccacaacaatactcagaaagaaagctgcagatagaggtttttcttgtagaaacaccatttccatcctgtttgaacagtgatctacatgtggcatttaaactgctaccttttatttcactgttgagagacagaaaaaaatacagttgacatgattctttgttatttaattgcctgtgaattgccgctacgataaaggttttaatcttagatcatactaaggcagcaatgcctgttacatgtgatttagatgaatgggtttcatcaaatcatggtggaaaaaatgtatttgaaatatattattctttagtatgcttgcctttctgcttctgcagaaaacagaaatagataaaatgtcaggaactattactagtgtgagcttaattttatgagaaaatgatactgttcagccggtataattcctctgttataatcttttaggctattaaagttctctgttgcagcacatgttgaattggttcctttctgaacatggattctgttttccttttccctctagtatgtaaaatgaatgtgcatattcgatgtcaagcgaacgtggcccctaactgtggggtaaatgcggtggaacttgccaagaccctggcagggatgggtctccaacccggaaatatttctccaacctcggtgagactttgctttttttccatgtctcgtaatttagaagttgctcagatgtgatgagcccaaatgagagcatgtggcctcatcaaagttcctaatcttagcaaatacaataaacagacttattgcctttctttctttctttctctttctttctttcttttttcttttctttctttctctttctttctttcttcctttctttctttctctctttctcccttttttttctctctctctcctctcctttcctctcctttcctttttccttccttcctcccttccccttccccttccccttccctgtcttcattcctttttgagatggggtcttcctctgtcacccaggctggagtgcagtggcacaattatggttctctgcagactcgactgcctaggctcaagtgatcctcctacctcagtctcccaagtaggtgggactgcaggtgtgcaccactatgtcagggtaattttttatttttttttttgtagagatggagtctcactttgttgcgcaggctggtctcaaactcctgggctcaagcgatcctctcgctttggcctcccaaagtgttgggattgcacaggtgtgagccactgtgtctggctgaaatagcatttttaatagcaatttgtttcacggataagattgtatatacagttatatgacaagaaataatgagcatgagcaacaagtaataaacagaaagcaaaagaaggacttttttcctttctttccttttttttttcttttttcttttttctatttgttttttgagacagagtctcgctttgtcacccagcctggagtgcagtgatattcgctcactgcaacctccacctcccaggttcaagcaattctcctgcctcagcctcccgagcagctggaattacaggtgcccactaccgcgcctggctgatttttgtatttttagtaagacggagtttctccatgttggccaggctggtctcaaacttctgatctcaagtgatctgcccaccttggcttcccaaagtgctggaattacaggcatgagctgccacgcccagcctttcttttctgttttaaaatcagcttcctgtcactgtggtctggtgaaagcctaaaacccaggatcctgggttatgcttttgtctggggaccaggcaggacaggtgaccagtaactatgggcgtcagcgtcctcacctctaaagcggttcaacttgaagatctcagaggctctgcacagccctggagtccagcagttctgaagttttggtttatggattgtgaatttatcattcacattcagcaggagacaggagtctctccctctggactctcaggagtgagtggagttgtgtcagttgcagtgcacatgtttttgtcagcactctccctgaatgacctccacctgctgtcatccagttgagccttggctgcttcccaagcccatgtcttactgttgccttcctggtcagcgtctagagatgggaaatagccatgtaagatctgaacggtgtagcttggtgtccagttcaggaaaattatcgactcaactaattaatctctgggtgctgaggaagggaagtgcaattactgtgagattcttgataatcctgaaaatgtcagcaccaaccacctcatttcctttttactgaaggactcctgcctagtagatcagggactttcattgacaatttttttttttttttgagacggagtctggctctgtcgcccaggctggagtgcagtggcacaatctcagctcactgcaagctccgcctccctggttcacgccattctcctgcctcagccttccgagtagctgggactataggcacccgccaccacgcccagctaattttttgtattttttttttttttagtagagatggggggtttcaacgtgttacccaggatggtctcgatctcctgaccttgtcatcctcccgtctcggcctcccaacgtgctgggattacaggcttgagccaccgcgcctggcctattgacaatttattgaattgccactacgataaatgttttaatccttatattagatcatactaaatcagcaatgactgttacatgtgatttagatgaatgggtttcatcaaatcatggtgaaaaaatgtatttgaaacctattatgtaaaaaaatgtatttgaaatccggtctgtctgtcttgtgggtaggcgggcaggatgccaatattgtgcggggattgctaatgcatgggacagttatccctgaagagcagagattaaagatatcagcctgaactagggcttctgagatacaatgggaataagataagaataggaataaagatcatgcagcacgatctttaggaatggacagtgcacttgtcatttgaatcaagtatccttaatccatgacatccggtgcctggctggatgttagacttttgatctgttgtctagtggtagcttatcttataaatgtctcaaggtgggccagctgcaaaagtcaccttgcctttaggtagcaacatcctcaggtactcagtaggatggaactggaaagccaactgaggcaggcacacttaccttccattcattgtgttgaaactagaactctcatcatgctggtgagagtgtagattggtcagtcttattgtagacggtaagttggctgtgtatcagaagtcttcaaaattagcactttggcccaaggaattccatttgcaggaagttatcttaagaaactgaataatgataatgacaatacagcttaagtttattgaatgtgccaggcaccaatctaagtgctttacatgcatttgttcatttaggcctcctaatgactcatgtggcagatgctttaaccttctctttgtcagaagaggaaactgaggtagggagaggctgggtaggtaacttgctcagggtcttaggttagcttgcagagctgttattgaatccaggcagcttgcttgagagctagacccttaattactctgttttgaagatttgcatgaagtgtattcactccagtgttattcactgtggtgaaaaaggggagatgcctcatccttctaatggcaggggaattaataccttgtgagatatccatgaaatggatattgaatagccgttagaactcatattcttgaaagaaatttaaaggcatgaaaaatacccaggacaaatttttgaatacaaatgtatgttatgaaaccataatttgattatgtttttaaaaattaaccaagtataaaaacgtgtgcgtgtgtgttatttatacatggatatagatatctttggcagagggctggtgagattttaggtatttagatttttattcttcatgcttttgatcattgttctaatattcacattggacttttatttctcctatataatttaaaatattgattaaaaaacgttgcttcattcgtgtcctggtgctgaatgatgcttgaatctctgagtagacatgagaactgctctctgtaaaaagtggacactaggtggcgctcgtggaatgggaccttcccgtggaaagccaccgcagaaggcacccaagggaccacacaaggcattggtgtttccatgcacttccacttgagcgctaactgtaactccatggcttagacgtatttttaattgaccttttacacaaagcagtaagcacagattcagaagctgatactgagggagtttcgagtggcagaaccgatcatctgtcaaactgagttgatctttcccccacgttatactgggggtgagactgctcccagccaataaggtcttcttcgtgcatgggtgctccatgcttctgctgcaatttctgacttaatgtgttcttactctttcagaaactcgtttccagatcgaccctaagacgacagggaaaggagagcagcaaagaaggaaatgggattggggttaattcttccaaccgacttggtatcgacaactttgagttcatccgagtgttggggaaggggagttttgggaaggtgagtcttggctttaactgtttgggttgaagtaagtgtgctctgtgtatggggggtgtgtgtgtgtgtgcacgcatgcgcacatactcacatttctcatgtgccattctttcttcctgttgtgtgcacacaccctaagacccccaagaggactccctcatgctccctccttttgctttgccataggtgatgcttgcaagagtaaaagaaacaggagacctctatgctgtgaaggtgctgaagaaggacgtgattctgcaggatgatgatgtggaatgcaccatgaccgagaaaaggatcctgtctctggcccgcaatcaccccttcctcactcagttgttctgctgctttcagacccccgtaagtatgaatcacattcactgcaccaacagcctcttttcttacagagctgagacagtaagatactggagatttcataaagttgagtatcagaaattttgggggatgggctcccaaggcagggtcatatggaggtattggtgacttctgccaacctctggaggaaaaatggaagcttatgaattcaccccttccccttgtgctgtcctggggaaggagcaccgtgagggttgtgtgtcccaggattgaaaaggtcatcatacataaatcttctgtaggtgaagtgaaggtgaatgaaagttacagcagtctaagaacagagaaaatgccatgggctcattgtcttgatgaatttggctgtgactctgctcctaaatgctgggctggttctgacctgggcacaggagtttagaagaggggtctgagaggacccagagaggtaccttgatttggaacatcccagggcattgcttctggccttagcaacgaccctttgcctgaaaccatggactggtcttaagaattgaatctgtctgccctgtaatggcgtgcgtttaggaatgcttctggctgcagttaatggaacacttgaccagcggtggattaaacaataaatagaaaggatgagtttggttcattagtacaaactttttacttgttgtttcacataatgagaagtctggtggtaacagtgctacggttggatttgttgctcagtgacattggacatgtctttcccttggggtcccaaggtatttgctgcagccctagatgccacaccttggcatgatagcatccaaagcagaaagcataagggcaggggctgaagggcttcatggccagtccccttttctcagggagggaagatctttcctagacttgggtcaactgccctgtatgtctgttctcacattgctacaaagaactacctgagacggggtaatttatgaagaaaaaatgtttaattgactcacagttccacaggccgtacaggaggcatggctggggaggcctcaagaaacttacagtcatagcggaagggtgaaggagaagcaagcagggtttcacgtgacagcaggagagagagagagtgaagggggaaaagctacacactttcaaacagccagatctcatgagagctcactatcatgagaagatcaaggggggaaatctgcctcatgatccaatcacctcccaccaggcccttccccaacattagagattccaattcaacatgagatctgggtggggacacacagccaaaccatatcatgcccttagcaagggaggctggaagagcaaatatcaggccaagataagctggcagaccatgattggttcacaccaaccttgattcagcccctggagcagggcattctgcttttatttagaatcaggctcaaaattgggcttttggtatcaatgaagagggaaaatggccctggagaaggcaccaagagggagggtctgccacagatgtgtcagaatcttgctgtcaaaaggcattctgggagagtgaattctgggcatgttcctggcctgtgccaccatcaaggaagtaggggattccacgagagcgggataggggcatctgactggtcagggaggctggcaatacacctctagggaaggtaaagccatgctgggaccaaacgagtgaattagaattagttaggcagagaatgaaggtgaatatgtcatacagggtcaacagcacactatgggtctgctggtgagacagagcatcaggtgtgaaggaggctcagcacacacagcatatcgaggttgaacaggggccttgagaactgaggcagccgaggtgggccagggccaggccatgaagagccttgtaggctgtgttaaggggtttgggtttagtcctgagagcaatggaaagtcatggaagaagtggaagcagaggaatgacgtatcaggttgtatgttttaattatgaaaatctaatttcttgcgataggtaatataagtatcctccaaatattcatatcctttctccgaaagaaacaagtattgctcgttttatttatagcctttgagagaattttctgtacataaaagcatatatatacagcacatatatgcatacatgtttatcttttaaaaacacaaataatagtatactttgcccatgattctgtatttttaaaatttaattaatctattttttgagacagggtctcactttgtcaccaggctggagtgcagtgacggttatggctcactgcagcctcaagctcccaggctaaagctatcctcccacctcagccccctgagaaactgggacttacaggcacatgccaccacacctggctaatttttgttatttttttgtagagatggggttttgccatgtttcccaggctggtctcaaactcctgggctaaagcaatccacccacctcagtcactcaaagttctgggattacaggcgtgagccactgtacctggccactttatttttttaagacataatattaatagtatatattggaaattgttttgtatttctatataccaagctgcctcatttttaatggctgcatagtactccattttagaacatgacataattaatttaacttttcctcctctgttgctatgtattgtgttcctagtcttttatttcagtcattgttgcagtgagtccccttgtacttttgtctttgtgtgcttatgctggtgtttctgtatgacacattattagtagtagtattgcttggtcaaagggtatatgcatttcacattttgattgagttcaattgccccctcccccattgtggttaaagatagtgaacgttttattgtccatgtaccacacattgttctaagtgctttatgtctgttatttaatccttatagtaaccctctgaaataagttctgttattatcaccccatttcacagaggagaaaagttaggtacaaagaagcttagttcctaagtggcagagagcgtttgaagccaggtggtctgacggcagagtccaggcaggctcttcctatttgctgggctgcgtctctaaattcacactcccaaagcttgagtgagaaggtgcatttccccgccttcccatcatggcatgctctctgtcatttggatcatctcagagttagaaaaatggtatatttgaagttccaacttgcattcctctaactgtgagtgaagctgggcgttttcatacatttcagggtcactagtattcaaatctgtgttgggagggccacatgcagcctggagaatgggtggagcagagaagtctgggagcttggtgacaggacgcactgaaggagatacatggcttggactcccacgccaccccagcgttatcaagagaaaaaaaaatggggaggcagctaatggcttcgtgggcctcggctcccctgcgcgcctctcccttcccctctgctcctcattggtcctcagtggctcttgatggccctggagcccatgctgctcctgcccccatcctctgctttccctctgctggattggtccttggttcccctggagctccagttctccctgccccacactgctccctggagtagccttgaaacgacttccttggctgggcactttgccaacgtaaagcccctcccctgccctcacccccttgttgggctcttttcttggaatagaggagtggagttacagcttgaggcttgagagggatagctggtagctgatactggttgcccgccggggtttcaagcgccagcctcctgaacaccaccatgaacaccatgagtcaagccgcagagccaggtgacctctcagatcttcttcctagggcctgagccaaacatcacatggtttcctatgacatgaggttagatcatcttcaccaggtggtaacctttgagtcaagcagtcaactgatatttagtgagcttacctggtcttgcagcagcaatggttccaagttactactgctgaagcaaagaaagaagtatctcatacctaacaggggaatttgatctgtaaaaggaaattctggtgattataagcaaagatctcatcaacagatcatgttatggtagagtaaaatgaggaagtaacctggaacagattgggaacccaatcaatatagcatatgcaatcaaaatgttccaggaaaatgtataaattctacagtataccaaaattttagcggccacatgagattcccagtgtttagtgggtgaagcctttactagcattgaagtctatttttcctagtggaagttttatgtttgaccacaaggtggcagtcattaccgcaaagttacttttatttctccaccagagaaaccaaaggcatggaactgccattgcggtttaagatgtgtgtgttgtagtagatgtctccaaagcaaaggataaagggaatgtacccctgctttaggctaaatgataaagaagtagtgggaacccacttcaaagaagcaagtgaggctgggcgcaggcagcactttgggaggccgaggtggggcaatcacttgagcctaagtgtttgagaccagcctggccaacgtggtgaaaccccgtctactaaaaatacaaaaagtagcttggtgtggtggtgcatgcctgcaattccacctactccagaggctgagacaggaggatcgctggaacccgggaggcagaggttgcagtgagccaagatcgtgccattgcactccaacctgggtgacagagcacgactttttctcaaaaaagaaaagaaaaaaaagcagcaaaggagattttctaatgctgagatagaggtatacaattttagttttgctgtcgttgttgccttctttgaggtttgcaaggtatcggactccacagtacacatagcatttgctcgctgtgcattcgtctgttggaagtccttatttactgttgtaaaactatccccatttactgttggaaaactaactataaagaacagaaagtgttacttgctatgagaagttacagcagaaagatgcttactttttagctgcccagtgttgcaggccctggcttctcaagtaagatgctaatcagtgtgtaatgctgaactgtgacagtctcccctggagaatttaggagtgagagggggtgttggagccactctacctgcaaagcatttcattgtgagggcctttgtaaaaacagcactctcctcatgcagccctgtggaatcagtctctctgaagcccagagcagggcagtaactagccgaggatcccttagattgtaagtggcagagacaaagttcaagtttaggtcaggctgactctgactccatatgttgtcttattccgcatttagttggttgattcagcctcggcttaagcacctcctatctgtcttggctcccctccaacctgacaatatggaaatggcattgcccttgggaacagaaaacctgaactgtgttttagctttgttttccaccatctaaatggcttgagtctgtcctaaaattcctttgacccttaaagttcttatcattaaaatgaaaacaggatcgctgtgagggtcaaatgagagaatgcagaatattttgtacagtgcgaagtgctgttcaaaagtgagtcagttcaactgtggtgatgaccccctccatcctcacaccaaaggaaaaatgtcccagggcttcccaatggccagcccagacacccacatggaggggacagcaaacattgagaaggtaagtattttataggtccttttgttcttggcaaaaaggaaatagtaagcaaatttcaaatatttgataatttatcaagattgctacaaaggtttatgaacccatagattatttcccctttttttttataatcatatacactgcttacaagtatataaatgggcatccttatctgtttctgatagcactgcaaatcctaatagtttgtgactaatttgataacctgtactaaaactcacaaaatgtatactcctggatctgtttttctgataatctggactacaaaaataatataaagaataaaatgttattcataaagatgtttaattttcagtgctttgtttatatatatttttttggtcaacatctctctaatcctccagcctctggtaaccactattctattctgtttctatgagtttggctttatttgattacacatatcagtgagattaagctgcatttgtctttctgtgactggcttatttcaaataatacaatgtcctccaggttgatccatgttgcaaatgacaggatgtctttctttttaaaagctgaacagtattctattgtgtatatattccgttgtttctttgtccattcatccactggtgaacacttaggttgattccatatcttggctattgtgagtaatgctgcagtgaacatgagagtgcaggcatcttttcgacatgctgatttcattttctttggatgtatatccagtattggaattgctggagcatatggtagttctattttaattttttgaggagcctccataccgttttccaaatgagtatactaatttacattcccaccaacagtgtacaagggttcccttttctccacatcctcttcaacacctgttatttttcatcttttcaataatagctgttctgacaggtatgacataatatctcattgtggttttaatctgcatatccccaatgattagtgatattgagcattttttctatacctgttggccatttgtgtgtgtggttttgttttgtttttttttttacttttcatttatttttactttaagttctgggatacatgtgctgaacttgcaggtttgttacataggtgtacatgtgccatggtggtctgctgcacctatcaactggtcatccaggttttaagccccgaatgcattaggtatttgtcctaatgctctccttgcccttgccctcccacccaacaggctctggcatgtgatgttcccctccctgtgttctcattgttcaacttatgagtgagaacatgtggtgtttgattttctgttcctgtgttagtttgctgagaatgatggtttccagcttcatccatgtccctgcaaaggacatgagctcattcttttttatggctgcatagtattccatggtgtatatgtgctacattttctttatccagtctgtcattgatgggcatttgtgttggttccaagtctttggtattgtaaatagtgctgcaataaacatacatgtgcatgtgtctttatagtagaatgatttattaatcctttgggtatatacccagtaatgggattgctgggtcaaatggtatttctggttctagatccttgaggagtcgccacatggttttccacaatggttgaactaatttacattcccaccacctgttggccatttgtatgtcttcttttgagaaatgtctattcaggtccattaaccattttaaaattgggttgtttcttactattaagttgtttgagtttcctctgtatttggatattaactccttatccagatatgtgttttacaaatattttctcccattccacaggttgtctcttcactctgttgattgcagaaactttgcagaagctttttagtttgatatagttccatttgtctatttttgcttttgttgcctgtgcttttacggtcatattttaaaaagccattgcctatgcctgtgtcacagatttcttcccctaagtcatcatttggtaattttacagttttaggtcttacatttaagtctttagtccatttttcagtgatttttttatgtgacgtgagatgagggtctgtttttattcttctgcatgtagatatccagttttttcaacaccatttattgaagggacagtcctttctttgttgtgtgtccttgggatctttgttgataattaattgattgtaaaggtgtggatttatttctgagctctgtattctgtcccattggtctatatatctgtttttatgccagtaccataatgttttgattactgtagctttgtagtagattttgaaatcaggtagtataatgcctccagctttgttctttttggtcaagattgctttgatgtcgtgcattatttatagtaggaaaatattagaaacaacttcagcatttagcaatgggggtacacttcaataatatactatttgatgagaattttcagccagtcaagagataattttatgttacgtagttaaattgtcaggtaccgtcgtaagcacctaacatgtattgtttattgcacgtgattcctataacagccttataagataggtgcaattattatcctcattatgtcaaatggaaaagtgagacactgagcagttaagtcatttgcctaaaatctatcacagaaaatggtaaaaataggaggttagaatccaggaaataatacagaggttatgatgatgaacagtctgtatccaagtcaggttctgcccaagatagtgaattggcagacccgttcccctccccagcctcaaaaaagactgccaggaaattatgccagtaagctatcagggactggattaaggtagtggaattatgaaggatttttttcctatttaccaaatttggtaataggattacatttcttcaacaatgggggtaggaggaataagccattattaagtcagggaccagacccctggtctctcagaacctgggatacggcctagtacatgctgtctccctgactcatttatggcgaaattcacatcacactccttttatctgaatcagtgagggttgtgtcagcagctgagtccctggcatggatgtgctggaatccaggcctcccatcccccatccccggctcgcaggggcctccccttcggttctcctcctggagagagaggaacaaagactagggggtgagggatggaggggggctatcacaggaagggggagcagcaaagtaaatattggtctgaggctttgttcggagacccagtttatggaagcaaaggtttccatttggaaaacagaagacacttgtggccagtgcccgaaacagcctctcactagcaggccctgggactgggtgagagggctgagcgaagtactcctcattgttttgggaatgccttaagtttgagtttaggatccaccgctttcaggtgggccacaggacgctccgatgccggtcgcttttgcctcctcccgctcctctgcctgctggggcgtctttacgatgtcagcctgcaaggatggagttcctggcaggaccatagctttccgtaagaaaccacttctcaaatggccaacaaagttgaaatcctggtttttgaaggtcagaatatcttggaatcttgtgaaataaatgccatcgctgcatacatgctggcacaaaatgatctcacgtgtgctcagtgtacctgcagcttgagtgcaaaggaacattctttcctttgtattactttctccattgttgttgtattaaatattagcgaagaagtgggagaggcagagagcagccaggcagcaaggtagtcatgtggccctggaattcatctttcagaattttcccaggcagggaactgcactgtgaccttcagcacaagagctctgagttctaattctggcttagctggcccacttgtctttaagaagttcccctgccttttattttgagataagcctctccacctaaatgtgtgttccttacttctggacctgttggccaacgtacctttagggagtgcccctccagctggcttcaacatgtggggtcgtagcggggctgcagcctgccctgtccagctctgatgcttcccagcgcctccagctcccctttcccttcctgaggcctcactgagcatttggctgagcccctgccagccgctggatgtggggaggagaaggcatgctcagaccctacagtggcattactgcttgtgctctgtgttgctgtgacacccagcgcaaggggaagaggccctgtgacagagggttagacaaggagaaggggaagtatagccgaaaggctgggagactttcccctttacaggcatgggaattctgaagaagcctctgttcatgccttggggtagttggttgattcctggggactgaatggtgcccactttgcaggaggggatgagcgaagaaggaaagggcagttcatgagaccctagcatatgtcctaggtgctcatattcattaccaatgtctatgaaactaaactgcacattggcattccgtaaatatccattgaacaaaatatctcatttaatacccaaagcatccagggaagagtatcatcctcattttaaaggcctagaaactgagatccagagaaattaaacaacttgccccagatcacacagtaagccagagtcagtgcatggattcctgacctgactgtcctgccaaataatatacaagccatttccattatatcacaaatagaagttttgagtctctaatatgtacaactgagaatactgtcattagacgggaatagaggcagttttccacaataggtggtttttggccttttgggaaatacctaaggatatttcctctgatctgtttggaaccaggtttccccctttccctgagcagtggctgctacaaatacaatctggtttgctgcgggtcatttgttgcagtttgcctttggctcagatcaaaatagaaaatgagccagaacatggtgtttcctgtctctgcatgttcctaccttattcatagtgttccatcttttttttaggttattaattgtggatttatttccatagtaagacaggatctgactttggggccaacatatgttggacccgcctccttctctgctgaagattctctgaagacaaaatgtgcataggccttctccaggatctggatttttgggggattatttgggacctttgcttctgtcctctccggctaccaggctttctgaccttctgtagctaaatagggccacaaaaatgaataggaataagagaacctgaagttcaagctgtgtctttggctctgtctctttggacgcgactgccaagatatttggtgtgacaaaaggatttgaaatagttggcctgtttttttcagtttgaggttgttctgttgtaactggtcttgtttttggtagccatcattatcactgacaacctagagactgagtagaccgccacctccatgtctgctaagatagtataatttaaatctttgatgtaaccagaacacaacccaggtcacactttgcccctgtgtctcatgatgactgaaagcttttggggtgatgattgcttggttatggttttcctggacccaacttggaacccttaaagcctgccaagttatagatactctggcttcagtagggccctgtattgaaacaccaagtgccaggactgctcaaaaggtgaaaatgagatgggtgttctgccaggtggtttagtgaaaaatcagtcaatccaggatcccactgactgaatgctaggcaaaagaaacagttcttttatttgttttcattttttgcagagttggaggtcttgctatgttgcccacactggtcttgaactcctgacctcaagcaatcctactgccttggcctcccaaagtattaggattacaggcatgagccaccgtgcctggctagaaacagttttttaaaaactcattctaattaggaagagaagaccaatacacatggaagaaaagtgtacagtagatagcatgtagtaaatatagtccttatgctacaaaaggaaaggtagttgaaaatgaacattgggttctaggggaaactcttaagtatgtgccagtctgtgctcagaagtacaacctgattggctgtgagactaaagccaactgattgctttaagcagcccctcgggtgtgggctgcagatggttccctaggaagctgggttgagtcctgctgtgatgagctgggactgccaggtaggacctgaagcggctgccaaggggctgccaccccaccctgaagacaaaatcatacagtctaaaaacctgactgctttggatcaaaatcataccctgtaatatgttctagtttttaaataacttgggttttccttaattacttaatctctgctgagaacccagctgcatccatctcttctaggtccggaattatccacagcatctcatgagcagaaacagccatgtcaacataaagttagcagactcgtcgtgtagagagttgtgcactggcaggttaggggggaaggtgttcattcattcagcagatatgcactgagtgcttgcaaggcatagggctcgccttcaagccttgaggacgcagcagtgagcaaaacagaaaagagtccctgccctcatgaaactgacattctttgggaagcttataagcctgaagctcatgaacagattgttcatcttatagtctcagctggtgaccctccatggcagctgctccctgtggcaattcctggattttcctttcagacaggctcagagccg

SEQ ID NO: 3 - DNA, 3847 bp, Homo sapiens
  CDS (436)..(1578)
caaaaccacagataatgttcagcccagcacagtaggggtcaatttggtccacttgctcagtgacaaaaagaaaaaaaaagtgggctgtcactaaagattttgactcacaagagaggggctggtctggaggtgggaggagggagtgacgagtcaaggaggagacagggacgcaggagggtgcaaggaagtgtcttaactgagacgggggtaaggcaagagagggtggaggaaattctgcaggagacaggcttcctccagggtctggagaacccagaggcagctcctcctgagtgctgggaaggactctgggcatcttcagcccttcttactctctgaggctcaagccagaaattcaggctgcttgcagagtgggtgacagagccacggagctggtgtccctgggaccctctgcccgtcttctctccactccccagcatggaggaaggtggtgattttgacaactactatggggcagacaaccagtctgagtgtgagtacacagactggaaatcctcgggggccctcatccctgccatctacatgttggtcttcctcctgggcaccacgggcaacggtctggtgctctggaccgtgtttcggagcagccgggagaagaggcgctcagctgatatcttcattgctagcctggcggtggctgacctgaccttcgtggtgacgctgcccctgtgggctacctacacgtaccgggactatgactggccctttgggaccttcttctgcaagctcagcagctacctcatcttcgtcaacatgtacgccagcgtcttctgcctcaccggcctcagcttcgaccgctacctggccatcgtgaggccagtggccaatgctcggctgaggctgcgggtcagcggggccgtggccacggcagttctttgggtgctggccgccctcctggccatgcctgtcatggtgttacgcaccaccggggacttggagaacaccactaaggtgcagtgctacatggactactccatggtggccactgtgagctcagagtgggcctgggaggtgggccttggggtctcgtccaccaccgtgggctttgtggtgcccttcaccatcatgctgacctgttacttcttcatcgcccaaaccatcgctggccacttccgcaaggaacgcatcgagggcctgcggaagcggcgccggctgctcagcatcatcgtggtgctggtggtgacctttgccctgtgctggatgccctaccacctggtgaagacgctgtacatgctgggcagcctgctgcactggccctgtgactttgacctcttcctcatgaacatcttcccctactgcacctgcatcagctacgtcaacagctgcctcaaccccttcctctatgcctttttcgacccccgcttccgccaggcctgcacctccatgctctgctgtggccagagcaggtgcgcaggcacctcccacagcagcagtggggagaagtcagccagctactcttcggggcacagccaggggcccggccccaacatgggcaagggtggagaacagatgcacgagaaatccatcccctacagccaggagacccttgtggttgactagggctgggagcagagagaagcctggcgccctcggccctccccggcctttgcccttgctttctgaaaatcaggtagtgtggctactccttgtcctatgcacatcctttaactgtcccctgattctgccccgccctgtcctcctctactgctttattctttctcagaggtttgtggtttaggggaaagagactgggctctacagacctgaccctgcacaagccatttaatctcactcagcctcagtttctccattggtatgaaatgggggaaagtcatattgatcctaaaatgttgaagcctgagtctggacgcagtaaaagcttgtttccctctgctgctttcttagatctgcaatcgtctttcctcccttctttccttgtagtttttcccccaccactctctgcagctgccgctccttatccctgccttctggcaccaatcccctcctacagctcgtccccctccctccatccatccttctcccctgtctactttcttgttctgaagggctactaagggttaaggatcccaaagcttgcagagactgaccctgtttaagctttctatcctgttttctgagtgtgaggcagggaatgggctggggccgggggtgggctgtgtgtcagcagataattagtgctccagcccttagatctgggagctccagagcttgccctaaaattggatcacttccctgtcattttgggcattggggctagtgtgattcctgcagttcccccatggcaccatgacactgactagatatgctttctccaaattgtccgcagaccctttcatccttcctctattttctatgagaattggaaggcagcagggctgatgaatggatgtactccttggtttcattatgtgagtggggagttgggaagggcaactagagagagaggatggaggggtgtctgcatttagtccagacactgcttggctcgctccccgagtcctcctgtttctgacttcctgcataactgtgagctgaagggtttcctcatctccccatcttaccccatcatactgatttctttcttgggcactggtgctacttggtgccaagaatcatgttgtttgggatggagatgcctgcctcttgtctgtgtgtgttgtacttatatgtctatatggatgagcctggcatgaacagcagtgtgcctgggtcatttggacaaatctcctcccaccccccaatccactgcaactctgctgttcacacattacccttggcagggggtggtggggggcagggacacactgaggcaatgaaaaatgtagaataaaaatgagtccaccccctactggatttgggggctccaacggctggtccgtgctttaggagcgaagttaatgtttgcaccaggcttcctgtagggagatccctccccaaagcagctggcgccaaggcttgggggcgtcctactgagctgggttcctgctccttcttgggctccatgaaggaagtaagaggctagttgagagcctcccttggcccctttccggtgcctccccgcctggcttcaaatttatgagcattgccctcatcgtcctttcttgttccagggtcagtggccctcttcctaaggaggcctcctgcttgccatgggccaaaaggcacggggtgggttttttctctccctaccctcaggattggacctcttggcttctgctggattggggatctgggaatagggactggagcaagtgtgcagatagcatgatgtctacactgccagagagaccgtgaggatgaaattaatagtggggcctttgtgagctagaggctgggagtgtctattccgggttttgttcttggaggactatgaaagtgaaggacaagacatgagcgatggagataagaaaagcccagcttgatgtgaatggacatcttgaccctccctggaatgacgccagctctgggggcagagggaggaggagaggggaaggggctcctcacagcctagtctccccatcttaagatagcatctttcacagagtcacctcctctgcccagagctgtcctcaaagcatccagtgaacactggaagaggcttctagaagggaagaaattgtccctctgaggccgccgtgggtgacctgcagagacttcctgcctggaactcatctgtgaactgggacagaagcagaggaggctgcctgctgtgatacccccttacctcccccagtgccttcttcagaatatctgcactgtcttctgatcctgttagtcactgtggttcatcaaataaaactgtttgtgc

SEQ ID NO: 4 - protein, 380 aa, Homo sapiens
MEEGGDFDNYYGADNQSECEYTDWKSSGALIPAIYMLVFLLGTTGNGLVLWTVFRSSREKRRSADIFIASLAVADLTFVVTLPLWATYTYRDYDWPFGTFFCKLSSYLIFVNMYASVFCLTGLSFDRYLAIVRPVANARLRLRVSGAVATAVLWVLAALLAMPVMVLRTTGDLENTTKVQCYMDYSMVATVSSEWAWEVGLGVSSTTVGFVVPFTIMLTCYFFIAQTIAGHFRKERIEGLRKRRRLLSIIVVLVVTFALCWMPYHLVKTLYMLGSLLHWPCDFDLFLMNIFPYCTCISYVNSCLNPFLYAFFDPRFRQACTSMLCCGQSRCAGTSHSSSGEKSASYSSGHSQGPGPNMGKGGEQMHEKSIPYSQETLVVD

SEQ ID NO: 5 - DNA, 3522 bp, Homo sapiens
  CDS (306)..(2357)
aggggcgagtcctgcgcgagtccccgggaggcgccgcgcgcttggaagggacggtcgggcttccccggcccgctgagggctcggcggcgggctcccctcctttccacctcgggagggagggaaggaggggagggaaaagtcccacggaggaggcagaatggccagtcgaggggcgcttaggcgctgcctttccccagggctgcctcgactcctgcacctgtcccgagggctggcctgagacgggactcccggttctcccgctgcgaagcagcgcggccccccggggccggggcagcggcgccggcatgtcgtctggcaccatgaagttcaatggctatttgagggtccgcatcggtgaggcagtggggctgcagcccacccgctggtccctgcgccactcgctcttcaagaagggccaccagctgctggacccctatctgacggtgagcgtggaccaggtgcgcgtgggccagaccagcaccaagcagaagaccaacaaacccacgtacaacgaggagttttgcgctaacgtcaccgacggcggccacctcgagttggccgtcttccacgagacgcccctgggctacgaccacttcgtggccaactgcaccctgcagttccaggagctgctgcgcacgaccggcgcctcggacaccttcgagggttgggtggatctcgagccagaggggaaagtatttgtggtaataacccttaccgggagtttcactgaagctactctccagagagaccggatcttcaaacattttaccaggaagcgccaaagggctatgcgaaggcgagtccaccagatcaatggacacaagttcatggccacgtatctgaggcagcccacctactgctctcactgcagggagtttatctggggagtgtttgggaaacagggttatcagtgccaagtgtgcacctgtgtcgtccataaacgctgccatcatctaattgttacagcctgtacttgccaaaacaatattaacaaagtggattcaaagattgcagaacagaggttcgggatcaacatcccacacaagttcagcatccacaactacaaagtgccaacattctgcgatcactgtggctcactgctctggggaataatgcgacaaggacttcagtgtaaaatatgtaaaatgaatgtgcatattcgatgtcaagcgaacgtggcccctaactgtggggtaaatgcggtggaacttgccaagaccctggcagggatgggtctccaacccggaaatatttctccaacctcgaaactcgtttccagatcgaccctaagacgacagggaaaggagagcagcaaagaaggaaatgggattggggttaattcttccaaccgacttggtatcgacaactttgagttcatccgagtgttggggaaggggagttttgggaaggtgatgcttgcaagagtaaaagaaacaggagacctctatgctgtgaaggtgctgaagaaggacgtgattctgcaggatgatgatgtggaatgcaccatgaccgagaaaaggatcctgtctctggcccgcaatcaccccttcctcactcagttgttctgctgctttcagacccccgatcgtctgttttttgtgatggagtttgtgaatgggggtgacttgatgttccacattcagaagtctcgtcgttttgatgaagcacgagctcgcttctatgctgcagaaatcatttcggctctcatgttcctccatgataaaggaatcatctatagagatctgaaactggacaatgtcctgttggaccacgagggtcactgtaaactggcagacttcggaatgtgcaaggaggggatttgcaatggtgtcaccacggccacattctgtggcacgccagactatatcgctccagagatcctccaggaaatgctgtacgggcctgcagtagactggtgggcaatgggcgtgttgctctatgagatgctctgtggtcacgcgccttttgaggcagagaacgaagatgacctctttgaggccatactgaatgatgaggtggtctaccctacctggctccatgaagatgccacagggatcctaaaatctttcatgaccaagaaccccaccatgcgcttgggcagcctgactcagggaggcgagcacgccatcttgagacatcctttttttaaggaaatcgactgggcccagctgaaccatcgccaaatagaaccgcctttcagacccagaatcaaatcccgagaagatgtcagtaattttgaccctgacttcataaaggaagagccagttttaactccaattgatgagggacatcttccaatgattaaccaggatgagtttagaaacttttcctatgtgtctccagaattgcaaccatagccttatggggagtgagagagagggcacgagaacccaaagggaatagagattctccaggaatttcctctatgggaccttcccagcatcagccttagaacaagaaccttaccttcaaggagcaagtgaagaactctgtgaaggatggaactttcagatatcaactatttagagtccagagggagccatggcactagaaatagttgataatgaaatgagattttatgaagtataccgctccacctatgagcgtctgtctctgtgggcttgggatgttaacaggagccaaaaggagggaaagtgtgaagaataaagtagatctgagaaattctgagccaatcaggcttcttaattcaagagacaaaccaagacgttctgtcaactgtgctgtgctcttctttaagccaatgaaccccaattcctggcagtctacaagaagtctcttaatgctaatgaagaatttaaaggtctttttaaggaaatgaagggctttccaaatagaatgatttactctgaagaaacaaacaatggtatctctgaaactcacaacctaaagcccaatcttgaaaatatgttgtgcaccaagacgactgcttcagcttcttctcttatccttactttctttaatagatatttattaaactgtccagtgaaaaggtgccacaatgcccagtattgtaaacaacaggtttgcattcatgaagctttcattcattctggagtctactaatttacctgaatggtgtttgcattctgtgaaatgcctctccacgttgcatatgtcacacttttgtctgcacataactcttttttcacaagaagggtcactgccacaacagcacagtcagcgggtgaattacaggtgcctgctgcctgcctacctgggtaatctgatcttgtctgtatcgccgtgtgctcatcactgaagaattgcaggccactcatgtcagtgaccagatttgtggcttataaacattagcagtttatttatgttttaagatgcaaagatgtgtgtttgatattcactttaataattagaaatggatcttgtaaacagggcatatatcaaagatgaccttataatatgtacccgaatatacagttcaagaattttgtctgactggaaataaatgcattttgtagcaaaaggaaaaaaaaaaaaaaaaaa

SEQ ID NO: 6 - protein, 683 aa, Homo sapiens
MSSGTMKFNGYLRVRIGEAVGLQPTRWSLRHSLFKKGHQLLDPYLTVSVDQVRVGQTSTKQKTNKPTYNEEFCANVTDGGHLELAVFHETPLGYDHFVANCTLQFQELLRTTGASDTFEGWVDLEPEGKVFVVITLTGSFTEATLQRDRIFKHFTRKRQRAMRRRVHQINGHKFMATYLRQPTYCSHCREFIWGVFGKQGYQCQVCTCVVHKRCHHLIVTACTCQNNINKVDSKIAEQRFGINIPHKFSIHNYKVPTFCDHCGSLLWGIMRQGLQCKICKMNVHIRCQANVAPNCGVNAVELAKTLAGMGLQPGNISPTSKLVSRSTLRRQGKESSKEGNGIGVNSSNRLGIDNFEFIRVLGKGSFGKVMLARVKETGDLYAVKVLKKDVILQDDDVECTMTEKRILSLARNHPFLTQLFCCFQTPDRLFFVMEFVNGGDLMFHIQKSRRFDEARARFYAAEIISALMFLHDKGIIYRDLKLDNVLLDHEGHCKLADFGMCKEGICNGVTTATFCGTPDYIAPEILQEMLYGPAVDWWAMGVLLYEMLCGHAPFEAENEDDLFEAILNDEVVYPTWLHEDATGILKSFMTKNPTMRLGSLTQGGEHAILRHPFFKEIDWAQLNHRQIEPPFRPRIKSREDVSNFDPDFIKEEPVLTPIDEGHLPMINQDEFRNFSYVSPELQP

SEQ ID NO: 7 - DNA, 21 bp, Artificial
  an artificially synthesized primer sequence
ctgtgggctacctacacgtac

SEQ ID NO: 8 - DNA, 20 bp, Artificial
  an artificially synthesized primer sequence
taggggatggatttctcgtg

SEQ ID NO: 9 - DNA, 21 bp, Artificial
  an artificially synthesized primer sequence
cacccccactgaaaaagatga

SEQ ID NO: 10 - DNA, 19 bp, Artificial
  an artificially synthesized primer sequence
tacctgtggagcaacctgc

SEQ ID NO: 11 - DNA, 22 bp, Artificial
  an artificially synthesized primer sequence
tgccatctacatgttggtcttc

SEQ ID NO: 12 - DNA, 20 bp, Artificial
  an artificially synthesized primer sequence
gtcaccacgaaggtcaggtc

SEQ ID NO: 13 - DNA, 20 bp, Artificial
  an artificially synthesized primer sequence
tctctctttctggcctggag

SEQ ID NO: 14 - DNA, 20 bp, Artificial
  an artificially synthesized primer sequence
aatgtcggatggatgaaacc

SEQ ID NO: 15 - protein, 60 aa, Homo sapiens
FEFIRVLGKGSFGKVMLARVKETGDLYAVKVLKKDVILQDDDVECTMTEKRILSLARNHP

SEQ ID NO: 16 - protein, 60 aa, Homo sapiens
FNFIKVLGKGSFGKVMLAELKGKDEVYAVKVLKKDVILQDDDVDCTMTEKRILALARKHP

SEQ ID NO: 17 - protein, 60 aa, Homo sapiens
FIFHKVLGKGSFGKVLLGELKGRGEYFAIKALKKDVVLIDDDVECTMVEKRVLTLAAENP

SEQ ID NO: 18 - protein, 56 aa, Homo sapiens
KMLGKGSFGKVFLAEFKKTNQFFAIKALKKDVVLMDDDVECTMVEKRVLSLAWEHP

SEQ ID NO: 19 - protein, 60 aa, Homo sapiens
FNFLMVLGKGSFGKVMLADRKGTEELYAIKILKKDVVIQDDDVECTMVEKRVLALLDKPP

SEQ ID NO: 20 - protein, 60 aa, Homo sapiens
FNFLMVLGKGSFGKVMLSERKGTDELYAVKILKKDVVIQDDDVECTMVEKRVLALPGKPP

SEQ ID NO: 21 - protein, 60 aa, Homo sapiens; identical to SEQ ID NO: 20

SEQ ID NO: 22 - protein, 60 aa, Homo sapiens
FSFLMVLGKGSFGKVMLAERRGSDELYAIKILKKDVIVQDDDVDCTLVEKRVLALGGRGP

SEQ ID NO: 23 - protein, 60 aa, Homo sapiens
FDLLRVIGRGSYAKVLLVRLKKTDRIYAMKVVKKELVNDDEDIDWVQTEKHVFEQASNHP

SEQ ID NO: 24 - protein, 60 aa, Homo sapiens
FDLIRVIGRGSYAKVLLVRLKKNDQIYAMKVVKKELVHDDEDIDWVQTEKHVFEQASSNP

SEQ ID NO: 25 - DNA, 25 bp, Artificial
  an artificially synthesized probe sequence
aattcttacactcgttcttccatct

SEQ ID NO: 26 - DNA, 25 bp, Artificial
  an artificially synthesized probe sequence
gccaggtactgtgttatgttcgtgt

SEQ ID NO: 27 - DNA, 25 bp, Artificial
  an artificially synthesized probe sequence
tagattggatgggagggggtgagaa

SEQ ID NO: 28 - DNA, 25 bp, Artificial
  an artificially synthesized probe sequence
cttactctctgaggctcaagccaga

SEQ ID NO: 29 - DNA, 25 bp, Artificial
  an artificially synthesized probe sequence
ggcaccacgggaaacggtctggtgc

SEQ ID NO: 30 - DNA, 25 bp, Artificial
  an artificially synthesized probe sequence
tcttgtctgtgttgtacttatatgt

SEQ ID NO: 31 - DNA, 25 bp, Artificial
  an artificially synthesized probe sequence
ctctgctgttcatacattacccttg

SEQ ID NO: 32 - DNA, 25 bp, Artificial
  an artificially synthesized probe sequence
aggcttgggggcgtcctactgagct

SEQ ID NO: 33 - DNA, 25 bp, Artificial
  an artificially synthesized probe sequence
tggcccctttccagtgcctccccgc

SEQ ID NO: 34 - DNA, 25 bp, Artificial
  an artificially synthesized probe sequence
aattcttacacttgttcttccatct

SEQ ID NO: 35 - DNA, 25 bp, Artificial
  an artificially synthesized probe sequence
gccaggtactgtattatgttcgtgt

SEQ ID NO: 36 - DNA, 25 bp, Artificial
  an artificially synthesized probe sequence
tagattggatggaagggggtgagaa

SEQ ID NO: 37 - DNA, 25 bp, Artificial
  an artificially synthesized probe sequence
cttactctctgaagctcaagccaga

SEQ ID NO: 38 - DNA, 25 bp, Artificial
  an artificially synthesized probe sequence
ggcaccacgggcaacggtctggtgc

SEQ ID NO: 39 - DNA, 25 bp, Artificial
  an artificially synthesized probe sequence
ttgtctgtgtgtgttgtacttatat

SEQ ID NO: 40 - DNA, 25 bp, Artificial
  an artificially synthesized probe sequence
ctctgctgttcacacattacccttg

SEQ ID NO: 41 - DNA, 25 bp, Artificial
  an artificially synthesized probe sequence
aggcttgggggcatcctactgagct

SEQ ID NO: 42 - DNA, 25 bp, Artificial
  an artificially synthesized probe sequence
tggcccctttccggtgcctccccgc

SEQ ID NO: 43 - DNA, 25 bp, Artificial
  an artificially synthesized probe sequence
tagattggatgggagggggtgagaa

SEQ ID NO: 44 - DNA, 25 bp, Artificial
  an artificially synthesized probe sequence
tagattggatggaagggggtgagaa

SEQ ID NO: 45 - DNA, 25 bp, Artificial
  an artificially synthesized probe sequence
aggcttgggggcgtcctactgagct

SEQ ID NO: 46 - DNA, 25 bp, Artificial
  an artificially synthesized probe sequence
aggcttgggggcatcctactgagct

SEQ ID NO: 47 - DNA, 22 bp, Artificial
  an artificially synthesized probe sequence
attcgatcggggcggggcgagc

(19) United States
(12) Patent Application Publication
(10) Pub. No.: US 2009/0324610 A1
(43) Pub. Date: Dec. 31, 2009

(54) GENE ASSOCIATED WITH ARTERIOSCLEROTIC DISEASE, AND USE THEREOF

(75) Inventors: Yutaka Kiyohara, Fukuoka (JP); Mitsuo Iida, Fukuoka (JP); Setsuro Ibayashi, Fukuoka (JP); Michiaki Kubo, Fukuoka (JP); Jun Hata, Fukuoka (JP); Yusuke Nakamura, Tokyo (JP); Teruo Omae, Fukuoka (JP); Yasuhiro Tanaka, Tokyo (JP); Go Ichien, Tokyo (JP)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignees: KYUSHU UNIVERISTY, NATIONAL UNIVERSITY CORPORATION, FUKUOKA-SHI (JP); THE UNIVERSITY TOKYO, BUNKYO-KU (JP); HISAYAMA RESEARCH INSTITUTE FOR LIFESTYLE DISEASES, KASUYA-GUN (JP); NTT DATA CORPORATION, KOTO-KU (JP); HUBIT GENOMIX, INC, CHIYODA-KU (JP)

(21) Appl. No.: 12/298,484

(22) PCT Filed: Apr. 24, 2007
(86) PCT No.: PCT/JP2007/058780

§ 371 Date: Mar. 23, 2009

(30) Foreign Application Priority Data
Apr. 25, 2006 (JP) 2006-121284

(51) Int. Cl. A61K 39/395; C12Q 1/68; C12Q 1/48; C07H 21/00; A61K 31/7088; G01N 33/566

(52) U.S. Cl. 424/158.1; 435/6; 435/15; 536/24.31; 514/44 A; 436/501

(57) ABSTRACT

Two genes implicated in arteriosclerotic diseases such as cerebral infarction were successfully identified by performing genome-wide correlation studies using SNPs by targeting the entire genome. Polymorphic mutations that can be used to examine the presence or absence of risk factors for arteriosclerotic diseases were successfully found on the genes. Subjects can be efficiently examined for the presence or absence of risk factors for arteriosclerotic diseases using the presence or absence of the polymorphic mutations as indicators. Furthermore, methods of screening for therapeutic agents for arteriosclerotic diseases are enabled by using expression or function of the genes as index.

### CROSS REFERENCE TO RELATED APPLICATIONS

This application is a U.S. National Phase of PCT/JP2007/058780, filed Apr. 24, 2007, which claims the benefit of Japanese Application Serial No. 2006-121284 filed Apr. 25, 2006, the contents of which are hereby incorporated by reference in their entirety.

### TECHNICAL FIELD

The present invention relates to methods of testing for the presence or absence of risk factors of arteriosclerotic diseases using the expression or polymorphic mutation of the AGTRL1 gene or PRKCH gene as an index, and also relates to methods of screening for agents for treating arteriosclerotic diseases using these genes.

### BACKGROUND ART

From the 1950s to 1960s, the mortality rate of cerebral apoplexy in Japan was the highest in the world. Since then, the mortality rate has declined steadily from the early 1970s, and in the 1990s the rate has become comparable to those of Europe and the United States (Non-Patent Document 1).

Nevertheless, it has not been changed and cerebral apoplexy is still the third major cause of death among Japanese. The incidence of cerebral apoplexy showed a decreasing trend during this period, but this trend has slowed down and leveled off in recent years (Non-Patent Document 2). Once cerebral apoplexy occurs, patients are often left with physical disability or cognitive dysfunction; therefore, cerebral apoplexy is a serious public health problem (Non-Patent Document 3). The incidence rate of cerebral apoplexy increases linearly with age. Since the elderly population is increasing rapidly in Japan, primary prevention of cerebral apoplexy is an important social objective worldwide.

Cerebral apoplexy can be broadly divided into cerebral infarction, intracerebral hemorrhage, and subarachnoid hemorrhage (Non-Patent Document 4). Of them, cerebral infarction has the highest incidence, and accounts for approximately 70% of all cerebral apoplexy (Non-Patent Document 5). Cerebral infarction can be further subdivided based on the size of the responsible blood vessel and mechanism of development, into the following subtypes: lacunar infarction (LA) caused by arteriosclerosis of thin perforating arteries, atherothrombotic cerebral infarction (AT) caused by atherosclerosis that affects the extracranial and major intracranial arteries, and cardiogenic embolic infarction (CE) which occurs when a thrombus produced in the cardiac cavity travels to the brain. LA and AT occur mainly due to atherosclerosis in the thin arteries or large arteries that perfuse the brain (Non-patent Document 4).

Multifactorial diseases including cerebral apoplexy implicate a plurality of genes and environmental factors, and afflict many patients, thus, elucidating those genetic factors is considered to have a major impact on medical economics and to contribute greatly to development of diagnostic and therapeutic techniques and prevention of the diseases. High blood pressure, diabetes, lipid metabolism disorders, smoking and such are known to be risk factors for cerebral infarction from past epidemiological studies (Non-Patent Documents 1 and 6). Family history of cerebral apoplexy is also a risk factor, and the risk of cerebral apoplexy is higher in monozygotic twins than in dizygotic twins (Non-Patent Document 7). While the presence of genetic factors implicated in cerebral infarction is expected from these twin studies and family history studies (Non-Patent Document 7), genetic determinants for cerebral infarction are still mostly unknown.

Several research styles have been proposed for the objective of discovering genes that provide susceptibility to common diseases that are not genetic diseases (Non-Patent Document 8). The candidate gene approach is widely used, but the lack of reproducibility of results is a problem (Non-Patent Document 9). For example, cerebral infarction-related genes have been examined using this method, and several known candidate genes implicated in atherosclerosis have been reported. However, their reproducibility has not necessarily been confirmed in other subject groups (Non-Patent Document 10). On the other hand, genome-wide study has recently been attracting attention as a highly reliable research technique for searching genes implicated in diseases that have complex mechanisms of development such as cerebral apoplexy (Non-Patent Document 14). In Japan, genome-wide correlation analyses have identified genes implicated in myocardial infarction (Non-Patent Document 11), rheumatoid arthritis (Non-Patent Document 12), or Crohn's disease (Non-Patent Document 13), and reproducibility of the results has been confirmed in different groups. Furthermore, in genome-wide linkage analyses and correlation analyses targeting Icelandic groups, phosphodiesterase 4D (PDE4D) (Non-Patent Document 15) and 5-lipoxygenase activating protein (ALOX5AP) (Non-Patent Document 16) have been reported as novel cerebral apoplexy-related genes. However, the research method for PDE4D has been criticized for having serious problems (Non-Patent Document 17).

Information on prior art literature related to the present invention is shown below.
[Patent Document 1] WO/2004/022592
[Patent Document 2] U.S. Pat. No. 6,987,110
[Patent Document 3] U.S. Pat. No. 6,492,324
[Patent Document 4] Japanese Patent Application No. 2003-518582
[Non-Patent Document 1] Sacco R L, et al., Stroke 1997; 28: 1507-1517.
[Non-Patent Document 2] Kubo, M. et al., Stroke 34, 2349-2354 (2003).
[Non-Patent Document 3] Kiyohara, Stroke. 34, 2343-2348 (2003).
[Non-Patent Document 4] Whisnant J P, et al., Stroke 1990; 21: 637-676.
[Non-Patent Document 5] Hata J, et al., J Neurol Neurosurg Psychiatry 2005; 76: 368-372.
[Non-Patent Document 6] Tanizaki Y, et al., Stroke 2000; 31: 2616-2622.
[Non-Patent Document 7] Flobmann E, et al., Stroke 2004; 35: 212-227.
[Non-Patent Document 8] Hirschhorn, J N. & Daly, M J. Nat. Rev. Genet. 6, 95-108 (2005).
[Non-Patent Document 9] Tabor, H K. et al., Nat. Rev. Genet. 3, 1-7 (2003).
[Non-Patent Document 10] Hassan A, Markus H S. Brain 2000; 123: 1784-1812.
[Non-Patent Document 11] Ozaki, K. et al., Nat. Genet. 32, 650-654 (2002).
[Non-Patent Document 12] Tokuhiro, S. et al., Nat. Genet. 35, 341-348 (2003).
[Non-Patent Document 13] Yamazaki, K. et al., Hum. Mol. Genet. 14, 3499-3506 (2005).
[Non-Patent Document 14] Glazier A M, et al., Science 2002; 298: 2345-2349.
[Non-Patent Document 15] Gretarsdottir S, et al., Nat Genet 2003; 35: 131-138.
[Non-Patent Document 16] Helgadottir A, et al., Nat Genet 2004; 36: 233-239.
[Non-Patent Document 17] Funalot, B. et al., Nat. Genet. 36, 3 (2004).
[Non-Patent Document 18] Bright R. et al., J Neurosci. Aug. 4, 2004;24(31):6880-8.
[Non-Patent Document 19] Chintalgattu v. et al., J Pharmacol Exp Ther. November 2004;311(2):691-9.
[Non-Patent Document 20] Hanlon P R. et al., FASEB J. August 2005;19(10):1323-5.
[Non-Patent Document 21] Aronowski J. et al., J Cereb Blood Flow Metab. February 2000;20(2):343-9.
[Non-Patent Document 22] Kleinz M J, et al., Regul Pept 2005; 126: 233-240.
[Non-Patent Document 23] O'Carroll A M, et al., J Neuroendocrinol 2003; 15: 661-666.
[Non-Patent Document 24] Kagiyama S, et al., Regl Pept 2005; 125: 55-59.
[Non-Patent Document 25] Seyedabadi M, et al., Auton Neurosci. 2002; 101: 32-38.
[Non-Patent Document 26] Katugampola S D, et al., Br J Pharmacol 2001; 132: 1255-1260.
[Non-Patent Document 27] Tatemoto K, et al., Regul Pept 2001; 99: 87-92.
[Non-Patent Document 28] Masri B, et al., FASEB J 2004; 18: 1909-1911.
[Non-Patent Document 29] Hashimoto Y, et al., Int J Mol Med 2005; 16: 787-792.

### DISCLOSURE OF THE INVENTION

### [Problems to be Solved by the Invention]

Cerebral infarction-related genes that have been previously reported are abnormal NOTCH3 gene in CADASIL and abnormal genes of mitochondrial DNA in MELAS, but these are causative genes of clearly hereditary cerebral infarction, and are not genes related to commonly found cerebral infarction. Research has been carried out for candidate genes related to common cerebral infarction mostly by using polymorphisms of genes predicted from the mechanism of cerebral infarction development (for example, coagulation system genes, ACE genes, and MTHFR genes), however, no definite observation has been made. Therefore, to identify new cerebral infarction-related genes, a genome-wide association study targeting the entire human genome is necessary. To date, only one genome-wide association study targeting cerebral apoplexy has been reported, and it was the PDE4D gene and ALOX5AP gene by the deCODE group in Iceland. Since the PDE4D gene showed significant relevance only in analyses when atherothrombotic cerebral infarction and cardiogenic embolism were combined, the results are questionable. In addition, there are no reports on reproducibility of the results by other groups. It has been confirmed by several groups that the ALOX5AP gene is implicated in cerebral infarction, but its implication is very weak in groups other than the Icelandic group.

The present invention was achieved in view of the above circumstances. An objective of the present invention is to provide genes implicated in arteriosclerotic diseases such as cerebral infarction, and uses that apply the characteristics of these genes. More specifically, an objective of the present invention is to provide genes implicated in arteriosclerotic diseases, methods of testing for the presence or absence of risk factors of arteriosclerotic diseases using polymorphisms of these genes, as well as methods of screening for pharmaceutical agents for treating arteriosclerotic diseases.

### [Means for Solving the Problems]

The present inventors conducted dedicated research to achieve the above-mentioned objectives. The present inventors performed large-scale case-control studies using gene-based tag-SNP markers to investigate genetic contribution to arteriosclerotic diseases such as cerebral infarction.

The present inventors discovered that PRKCH, which is a protein kinase C (PKC) family gene, is highly associated with lacunar infarction and atherothrombotic infarction (p=4.7×10⁻⁶). In a 14-year prospective follow-up study, an SNP (1425G>A) in PRKCH affected the PKC activity and increased the risk of development of cerebral infarction (p=0.043, relative risk 2.58). The present inventors also discovered that PKCη is expressed in the vascular endothelial cells and foamy macrophages of human atherosclerotic lesions, and that it correlates with severity of the illness. The above-mentioned results indicate that SNPs in PRKCH change the kinase activity and thereby become a novel genetic risk factor implicated in cerebral infarction.

This time, the present inventors performed a large-scale gene-based case control study using 1,112 cerebral infarction cases and the same number of age- and sex-matched control cases, and discovered that PRKCH is a gene implicated in cerebral infarction. The SNP of 1425G>A in exon 9 of PRKCH was highly relevant to the lacunar infarction group and to the group in which the lacunar infarction group and the atherothrombotic infarction group were combined. PKCη was expressed in atherosclerotic lesions and the expression level increased with the severity of atherosclerosis. Functional analysis revealed that this amino acid substitution (V374I) induces the PKC activity to 1.6-times of the original level. Furthermore, in the prospective follow-up study, the incidence of cerebral infarction was 2.58 times higher in subjects who have the AA genotype than in subjects who have the GA genotype or GG genotype. These results showed that PRKCH is a gene implication in cerebral infarction and the polymorphic mutation (SNP) of 1425G>A in PRKCH, in particular, is a site responsible for it.

Furthermore, the present inventors newly discovered that SNPs in the AGTRL1 gene are implicated in cerebral infarction. It has been shown that the APJ receptor (a receptor protein that is homologous to the angiotensin type 1 receptor) encoded by the AGTRL1 gene is expressed in the cardiovascular system (Non-Patent Document 22) and in the central nervous system (Non-Patent Document 23), and it functions in regulation of blood pressure (Non-Patent Documents 24 to 27), and migration (Non-Patent Document 28) and growth (Non-Patent Document 29) of endothelial cells. Through in vitro functional analysis, the present inventors discovered that the Sp1 transcription factor binds to the promoter and SNP in the intron of AGTRL1 and affects the mRNA expression level. These results indicate that a haplotype of AGTRL1 is a novel genetic determinant for susceptibility to arteriosclerotic diseases such as cerebral infarction and is involved in the pathogenic mechanism of atherosclerosis.

Of the two genes discovered this time, the AGTRL1 gene encodes AGTRL1 which is a seven-transmembrane G-protein-coupled receptor, and is known to show 30% homology with Angiotensin II receptor type 1 which is deeply connected with hypertension and arteriosclerosis. Apelin has been reported as a ligand of AGTRL1, and it has also been reported to be involved in the regulation of blood pressure in experiments using rats. There are reports that in humans, the expressions of Apelin and AGTRL1 are increased in heart failure patients, and that the administration of Apelin causes constriction of veins. Therefore, based on previous reports, AGTRL1 is predicted to be involved in blood pressure regulation. However, there have been no reports on the connection between AGTRL1 and cerebral infarction in humans. On the other hand, the PRKCH gene encodes PKC-eta, which belongs to the PKC family, and has been reported to be highly expressed in mouse skin and suggested to be implicated in cell growth and apoptosis in cancer. However, its function in human is completely unknown, and the substrates of PKC-eta and signal transduction pathways have been hardly elucidated. Accordingly, the connection between PKC-eta and cerebral infarction is not suggested from the previous findings.

As described above, the present inventors successfully identified two genes implicated in arteriosclerotic diseases such as cerebral infarction, and thereby completed the present invention.

The present invention relates to genes implicated in arteriosclerotic diseases such as cerebral infarction, methods of testing for the presence or absence of risk factors for arteriosclerotic diseases by using polymorphisms of these genes, as well as methods of screening for pharmaceutical agents for treating arteriosclerotic diseases. More specifically, the present invention relates to:
[1] a method for testing whether or not a subject has a risk factor for arteriosclerotic disease, which uses the subject's AGTRL1 gene expression as an index;
[2] a method for testing whether or not a subject has a risk factor for arteriosclerotic disease, which comprises detecting DNA mutation in the subject's AGTRL1 gene;
[3] the method of [2], wherein said mutation changes the binding of said gene with an Sp1 transcription factor;
[4] a method for testing whether or not a subject has a risk factor for arteriosclerotic disease, which uses the subject's PRKCH gene expression as an index;
[5] a method for testing whether or not a subject has a risk factor for arteriosclerotic disease, which comprises detecting DNA mutation in the subject's PRKCH gene;
[6] the method of any one of [1] to [5], wherein the mutation is a polymorphic mutation;
[7] a method for testing whether or not a subject has a risk factor for arteriosclerotic disease, wherein the method comprises determining type of nucleotide at a polymorphic site in the subject's AGTRL 1 gene;
[8] the method of [7], wherein the polymorphic site is in the AGTRL1 gene located at (1a) position 1, (2a) position 12541, (3a) position 21545, (4a) position 33051, (5a) position 35365, (6a) position 39268, (7a) position 39353, (8a) position 39370, (9a) position 39474, (10a) position 39553, (11a) position 39665, (12a) position 41786, (13a) position 42019, (14a) position 42509, (15a) position 43029, (16a) position 43406, (17a) position 43663, (18a) position 46786, (19a) position 49764, (20a) position 64276, (21a) position 74482, (22a) position 78162, (23a) position 93492, or (24a) position 102938 of the nucleotide sequence of SEQ ID NO: 1;
[9] the method of [8], wherein the subject is determined to have a risk factor for arteriosclerotic disease when the nucleotides at the polymorphic sites of (1a) to (24a) of [8] are (1b) to (24b) below, respectively:
(1b) the type of nucleotide in the complementary strand of the AGTRL1 gene located at position 1 of the nucleotide sequence of SEQ ID NO: 1 is T;
(2b) the type of nucleotide in the complementary strand of the AGTRL1 gene located at position 12541 of the nucleotide sequence of SEQ ID NO: 1 is T;
(3b) the type of nucleotide in the complementary strand of the AGTRL1 gene located at position 21545 of the nucleotide sequence of SEQ ID NO: 1 is A;
(4b) the type of nucleotide in the complementary strand of the AGTRL1 gene located at position 33051 of the nucleotide sequence of SEQ ID NO: 1 is C;
(5b) the type of nucleotide in the complementary strand of the AGTRL1 gene located at position 35365 of the nucleotide sequence of SEQ ID NO: 1 is T;
(6b) the type of nucleotide in the complementary strand of the AGTRL1 gene located at position 39268 of the nucleotide sequence of SEQ ID NO: 1 is A;
(7b) the type of nucleotide in the complementary strand of the AGTRL1 gene located at position 39353 of the nucleotide sequence of SEQ ID NO: 1 is G;
(8b) the type of nucleotide in the complementary strand of the AGTRL1 gene located at position 39370 of the nucleotide sequence of SEQ ID NO: 1 is C;
(9b) the type of nucleotide in the complementary strand of the AGTRL1 gene located at position 39474 of the nucleotide sequence of SEQ ID NO: 1 is T;
(10b) the type of nucleotide in the complementary strand of the AGTRL1 gene located at position 39553 of the nucleotide sequence of SEQ ID NO: 1 is T;
(11b) the nucleotide in the AGTRL1 gene located at position 39665 of the nucleotide sequence of SEQ ID NO: 1 has been deleted;
(12b) the type of nucleotide in the complementary strand of the AGTRL1 gene located at position 41786 of the nucleotide sequence of SEQ ID NO: 1 is A;
(13b) the type of nucleotide in the complementary strand of the AGTRL1 gene located at position 42019 of the nucleotide sequence of SEQ ID NO: 1 is G;
(14b) the type of nucleotide in the complementary strand of the AGTRL1 gene located at position 42509 of the nucleotide sequence of SEQ ID NO: 1 is G;
(15b) the type of nucleotide in the complementary strand of the AGTRL1 gene located at position 43029 of the nucleotide sequence of SEQ ID NO: 1 is G;
(16b) the type of nucleotide in the complementary strand of the AGTRL1 gene located at position 43406 of the nucleotide sequence of SEQ ID NO: 1 is C;
(17b) the type of nucleotide in the complementary strand of the AGTRL1 gene located at position 43663 of the nucleotide sequence of SEQ ID NO: 1 is T;
(18b) the type of nucleotide in the complementary strand of the AGTRL1 gene located at position 46786 of the nucleotide sequence of SEQ ID NO: 1 is C;
(19b) the type of nucleotide in the complementary strand of the AGTRL1 gene located at position 49764 of the nucleotide sequence of SEQ ID NO: 1 is T;
(20b) the type of nucleotide in the complementary strand of the AGTRL1 gene located at position 64276 of the nucleotide sequence of SEQ ID NO: 1 is T;
(21b) the type of nucleotide in the complementary strand of the AGTRL1 gene located at position 74482 of the nucleotide sequence of SEQ ID NO: 1 is C;
(22b) the type of nucleotide in the complementary strand of the AGTRL1 gene located at position 78162 of the nucleotide sequence of SEQ ID NO: 1 is G;
(23b) the type of nucleotide in the complementary strand of the AGTRL1 gene located at position 93492 of the nucleotide sequence of SEQ ID NO: 1 is G; and
(24b) the type of nucleotide in the complementary strand of the AGTRL1 gene located at position 102938 of the nucleotide sequence of SEQ ID NO: 1 is C;
[10] a method for testing whether or not a subject has a risk factor for arteriosclerotic disease, wherein the subject is determined to have a risk factor for arteriosclerotic disease when a DNA block showing the following haplotype is detected:
(A) a haplotype in which the nucleotides in the complementary strand of the AGTRL1 gene at polymorphic sites located at positions 39268, 39353, 41786, 42019, and 43406 of the nucleotide sequence of SEQ ID NO: 1 are A, G, A, G, and C, respectively;
[11] a method for testing whether or not a subject has a risk factor for arteriosclerotic disease, which comprises the step of determining the type of nucleotide of a linked polymorphic site present within a DNA block showing the following haplotype:
(A) a haplotype in which the nucleotides of the complementary strand at polymorphic sites on the AGTRL1 gene located at positions 39268, 39353, 41786, 42019, and 43406 of the nucleotide sequence of SEQ ID NO: 1 are A, G, A, G, and C, respectively;
[12] a method for testing whether or not a subject has a risk factor for arteriosclerotic disease, which comprises the steps of:
(a) determining the type of nucleotide at a polymorphic site in the AGTRL1 gene of the subject; and
(b) determining that the subject has a risk factor for arteriosclerotic disease when the nucleotide determined in (a) is the same as the nucleotide at said polymorphic site in the AGTRL1 gene showing the haplotype of (A):
(A) a haplotype in which the nucleotides of the complementary strand at polymorphic sites in the AGTRL1 gene located at positions 39268, 39353, 41786, 42019, and 43406 of the nucleotide sequence of SEQ ID NO: 1 are A, G, A, G, and C, respectively;
[13] the method of [12], wherein said polymorphic site of (a) is in the AGTRL1 gene located at any one of positions 1, 12541, 21545, 33051, 35365, 39268, 39353, 39370, 39474, 39553, 39665, 41786, 42019, 42509, 43029, 43406, 43663, 46786, 49764, 64276, 74482, 78162, 93492, or 102938 of the nucleotide sequence of SEQ ID NO: 1;
[14] a method for testing whether or not a subject has a risk factor for arteriosclerotic disease, wherein the subject is determined to have a risk factor for arteriosclerotic disease when the expression level of the subject's AGTRL1 gene is elevated compared to that of a control;
[15] a method for testing whether or not a subject has a risk factor for arteriosclerotic disease, wherein the method comprises determining the type of nucleotide at a polymorphic site in the subject's PRKCH gene;
[16] the method of [15], wherein the polymorphic site is in the PRKCH gene located at (1a) position 1, (2a) position 16212, (3a) position 30981, (4a) position 32408, (5a) position 33463, (6a) position 34446, (7a) position 39322, (8a) position 39469, (9a) position 39471, (10a) position 49248, (11a) position 49367, or (12a) position 52030 of the nucleotide sequence of SEQ ID NO: 2;
[17] the method of [16], wherein the subject is determined to have a risk factor for arteriosclerotic disease when the nucleotides in the polymorphic sites of (1a) to (12a) of [16] are the following (1b) to (12b), respectively:
(1b) the nucleotide in the PRKCH gene located at position 1 of the nucleotide sequence of SEQ ID NO: 2 is A;
(2b) the nucleotide in the PRKCH gene located at position 16212 of the nucleotide sequence of SEQ ID NO: 2 is G;
(3b) the nucleotide in the PRKCH gene located at position 30981 of the nucleotide sequence of SEQ ID NO: 2 is A;
(4b) the nucleotide in the PRKCH gene located at position 32408 of the nucleotide sequence of SEQ ID NO: 2 is G;
(5b) the nucleotide in the PRKCH gene located at position 33463 of the nucleotide sequence of SEQ ID NO: 2 is G;
(6b) the nucleotide in the PRKCH gene located at position 34446 of the nucleotide sequence of SEQ ID NO: 2 is T;
(7b) the nucleotide in the PRKCH gene located at position 39322 of the nucleotide sequence of SEQ ID NO: 2 is T;
(8b) the nucleotide in the PRKCH gene located at position 39469 of the nucleotide sequence of SEQ ID NO: 2 is A;
(9b) the nucleotide in the PRKCH gene located at position 39471 of the nucleotide sequence of SEQ ID NO: 2 is C;
(10b) the nucleotide in the PRKCH gene located at position 49248 of the nucleotide sequence of SEQ ID NO: 2 is C;
(11b) the nucleotide in the PRKCH gene located at position 49367 of the nucleotide sequence of SEQ ID NO: 2 is G; and
(12b) the nucleotide in the PRKCH gene located at position 52030 of the nucleotide sequence of SEQ ID NO: 2 is A;
[18] a method for testing whether or not a subject has a risk factor for arteriosclerotic disease, wherein the subject is determined to have a risk factor for arteriosclerotic disease when the autophosphorylation activity or kinase activity of the subject's PRKCH protein is elevated compared to that of a control;
[19] a method for testing whether or not a subject has a risk factor for arteriosclerotic disease, wherein the subject is determined to have a risk factor for arteriosclerotic disease when the subject carries a mutant protein in which valine at position 374 in the amino acid sequence of the PRKCH protein is substituted with isoleucine;
[20] the method of any one of [1] to [19], wherein a biological sample derived from the subject is subjected to the test as a test sample;
[21] a reagent for testing for the presence or absence of a risk factor for arteriosclerotic disease, which comprises an oligonucleotide that hybridizes with a DNA comprising the polymorphic sites of (1a) to (24a) of [8] or (1a) to (12a) of [16] and has a length of at least 15 nucleotides;
[22] a reagent for testing for the presence or absence of a risk factor for arteriosclerotic disease, which comprises a solid phase to which a nucleotide probe is immobilized, wherein the nucleotide probe hybridizes with a DNA comprising the polymorphic sites of (1a) to (24a) of [8] or (1a) to (12a) of [16];
[23] a reagent for testing for the presence or absence of a risk factor for arteriosclerotic disease, which comprises a primer oligonucleotide for amplifying a DNA comprising the polymorphic sites of (1a) to (24a) of [8] or (1a) to (12a) of [16];
[24] a reagent for testing for the presence or absence of a risk factor for arteriosclerotic disease, which comprises (a) or (b) as an active ingredient:
(a) an oligonucleotide that hybridizes with a transcript of an AGTRL1 or PRKCH gene; and
(b) an antibody that recognizes an AGTRL1 or PRKCH protein;
[25] a reagent for screening for a pharmaceutical agent for treating or preventing arteriosclerotic disease, which comprises any one of (a) to (c) as an active ingredient:
(a) an oligonucleotide that hybridizes with a transcript of an AGTRL1 gene;
(b) an antibody that recognizes an AGTRL1 protein; and
(c) a polynucleotide comprising a DNA region which comprises a nucleotide site in a AGTRL1 gene located at position 39353 or 42509 of the nucleotide sequence of SEQ ID NO: 1;
[26] a reagent for screening for a pharmaceutical agent for treating or preventing arteriosclerotic disease, which comprises any one of (a) to (c) as an active ingredient:
(a) an oligonucleotide that hybridizes with a transcript of a PRKCH gene;
(b) an antibody that recognizes a PRKCH protein; and
(c) a mutant PRKCH protein which has an amino acid sequence in which valine at position 374 of the amino acid sequence of a PRKCH protein is substituted with isoleucine;
[27] a pharmaceutical agent for treating or preventing arteriosclerotic disease, which comprises as an active ingredient a substance that suppresses the expression of an AGTRL1 or PRKCH gene or suppresses the function of a protein encoded by said gene;
[28] the pharmaceutical agent of [27], wherein the substance that suppresses the expression of the AGTRL1 or PRKCH gene is a compound selected from the group consisting of (a) to (c):
(a) an antisense nucleic acid against a transcript of the AGTRL1 or PRKCH gene or a portion thereof,
(b) a nucleic acid having a ribozyme activity of specifically cleaving a transcript of the AGTRL1 or PRKCH gene; and
(c) a nucleic acid having an effect of inhibiting the expression of the AGTRL1 or PRKCH gene through an RNAi effect;
[29] the pharmaceutical agent of [27], wherein the substance that suppresses the function of the AGTRL1 or PRKCH protein is the compound of (a) or (b):
(a) an antibody that binds to an AGTRL1 or PRKCH protein; or
(b) a low-molecular-weight compound that binds to an AGTRL1 or PRKCH protein;
[30] a pharmaceutical agent for treating or preventing arteriosclerotic disease, which comprises as an active ingredient a substance that inhibits the binding of an Sp1 transcription factor with a DNA region that comprises a nucleotide site in the AGTRL1 gene located at position 39353 or 42509 of the nucleotide sequence of SEQ ID NO: 1;
[31] a pharmaceutical agent for treating or preventing arteriosclerotic disease, which comprises as an active ingredient a substance that inhibits the autophosphorylation activity of a PRKCH protein;
[32] a method of screening for a pharmaceutical agent for treating or preventing arteriosclerotic disease, which comprises selecting a compound that reduces the expression level of an AGTRL1 or PRKCH gene or reduces the activity of a protein encoded by said gene;
[33] a method of screening for a pharmaceutical agent for treating or preventing arteriosclerotic disease, which comprises the steps of:
(a) contacting a test compound with a cell that expresses an AGTRL1 or PRKCH gene;
(b) measuring the expression level of said AGTRL1 or PRKCH gene; and
(c) selecting the compound that reduces the expression level as compared with that measured in the absence of the test compound;
[34] a method of screening for a pharmaceutical agent for treating or preventing arteriosclerotic disease, which comprises the steps of:
(a) contacting a test compound with a cell or cell extract that comprises a DNA having a structure in which a transcriptional regulatory region of an AGTRL1 or PRKCH gene and a reporter gene are operably linked with each other;
(b) measuring the expression level of said reporter gene; and
(c) selecting a compound that reduces said expression level as compared with that measured in the absence of the test compound;
[35] the method of any one of [32] to [34], wherein the AGTRL1 gene is a mutant AGTRL1 gene of (a) or (b) whose expression is enhanced:
(a) a mutant AGTRL1 gene in which the nucleotide in the complementary strand of the AGTRL1 gene located at position 42509 of the nucleotide sequence of SEQ ID NO: 1 is G; or
(b) a mutant AGTRL1 gene in which the nucleotide in the complementary strand of the AGTRL1 gene located at position 39353 of the nucleotide sequence of SEQ ID NO: 1 is G;
[36] a method of screening for a pharmaceutical agent for treating or preventing arteriosclerotic disease, which comprises the steps of:
(a) contacting a test compound with an Sp1 transcription factor and a polynucleotide comprising a DNA region that comprises a nucleotide site in an AGTRL1 gene located at position 39353 or 42509 of the nucleotide sequence of SEQ ID NO: 1;
(b) measuring the binding activity between said polynucleotide and the Sp1 transcription factor; and
(c) selecting a compound that reduces said binding activity as compared with that measured in the absence of the test compound;
[37] a method of screening for a pharmaceutical agent for treating or preventing arteriosclerotic disease, which comprises the steps of:
(a) contacting a test compound with a PRKCH protein;
(b) measuring the autophosphorylation activity of the PRKCH protein; and
(c) selecting a compound that reduces the autophosphorylation activity as compared with that measured in the absence of the test compound;
[38] the method of [37], wherein said PRKCH protein is a mutant protein in which valine of position 374 in the amino acid sequence of the PRKCH protein is substituted with isoleucine;
[39] a method of screening for a pharmaceutical agent for treating or preventing arteriosclerotic disease, which comprises the steps of:
(a) contacting a test compound with a PRKCH protein;
(b) measuring the protein kinase activity of the PRKCH protein; and
(c) selecting a compound that reduces the protein kinase activity as compared with that measured in the absence of the test compound;
[40] Use of any one of the substances of (a) to (e) in the preparation of a reagent for testing for the presence or absence of a risk factor for arteriosclerotic disease:
(a) an oligonucleotide that hybridizes with a DNA comprising the polymorphic sites of (1a) to (24a) of [8] or (1a) to (12a) of [16], and has a length of at least 15 nucleotides;
(b) a solid phase to which a nucleotide probe that hybridizes with a DNA comprising the polymorphic sites of (1a) to (24a) of [8] or (1a) to (12a) of [16] is immobilized;
(c) a primer oligonucleotide for amplifying a DNA comprising the polymorphic sites of (1a) to (24a) of [8] or (1a) to (12a) of [16];
(d) an oligonucleotide that hybridizes with a transcript of an AGTRL1 or PRKCH gene;
(e) an antibody that recognizes an AGTRL1 or PRKCH protein;

Use of any one of the substances of (a) to (f) in the preparation of a reagent for screening for a pharmaceutical agent for treating or preventing arteriosclerotic disease:
(a) an oligonucleotide that hybridizes with a transcript of an AGTRL1 gene;
(b) an antibody that recognizes an AGTRL1 protein;
(c) a polynucleotide comprising a DNA region that comprises a nucleotide site in an AGTRL1 gene located at position 39353 or 42509 in the nucleotide sequence of SEQ ID NO: 1;
(d) an oligonucleotide that hybridizes with a transcript of a PRKCH gene;
(e) an antibody that recognizes a PRKCH protein;
(f) a PRKCH protein mutant that comprises an amino acid sequence in which valine of position 374 in the amino acid sequence of a PRKCH protein is substituted with isoleucine;
[42] Use of a substance that suppresses the expression of an AGTRL1 or PRKCH gene or suppresses the function of a protein encoded by said gene, in the preparation of a pharmaceutical agent for treating or preventing arteriosclerotic disease;
[43] Use of a substance of (a) or (b) in the preparation of a pharmaceutical agent for treating or preventing arteriosclerotic disease:
(a) a substance that inhibits the binding of an Sp1 transcription factor with a DNA region that comprises a nucleotide site in an AGTRL1 gene located at position 39353 or 42509 in the nucleotide sequence of SEQ ID NO: 1;
(b) a substance that inhibits the autophosphorylation activity of a PRKCH protein; and
[44] A method of treating or preventing arteriosclerotic disease, comprising the step of administering an agent of the present invention to a subject (human, non-human mammal, or such; preferably a patient with arteriosclerotic disease).

### BRIEF DESCRIPTION OF THE DRAWINGS

FIG. 1 depicts the genetic structure in PRKCH, case-control association, and linkage disequilibrium. a. Genome structure around PRKCH. b. Exon-intron structure of PRKCH. Genotyped SNPs in PRKCH are indicated below the gene (vertical lines). c. Case-control association analysis of lacunar infarction and atherothrombotic infarction. The log-transformed P values for allelic frequency are plotted on the y axis. d. Pair-wise linkage disequilibrium between SNPs measured by D′ (lower left) and Δ (upper right).

FIG. 2 presents photographs and diagrams showing a comparison of the PKC activities of 374V and 374I. a. Sequencing results of rs2230500 (1425G>A) and rs17098388 (1427A>C) are shown. b. Domain structure of PRKCH. The arrow indicates the position of rs2230500. c. A photograph showing the immunoprecipitates of mock, PRKCH-374V, and PRKCH-374I stained with Coomassie Brilliant Blue. d. A photograph showing the result of Western blotting using equal amounts of immunoprecipitates of mock, PRKCH-374V and PRKCH-374I. e. A photograph showing an autophosphorylation assay of mock, PRKCH-374V and PRKCH-374I after stimulation with 10 nM PS and 100 μM PDBu. f. The PKC activities of PRKCH-374V and PRKCH-374I after three-minute stimulation with 10 nM PS and 100 μM PDBu are shown.

FIG. 3 shows the positions of amino acid substitutions of PRKCH. The amino acid substitution V374I of PRKCH is at a position inside the conserved ATP binding site in the PKC family. The asterisks indicate the conserved ATP binding site, and # indicates the V374I amino acid substitution.

FIG. 4 shows the relative expression levels of PRKCH mRNA in various human tissues. The relative mRNA expression levels were quantified by real-time PCR and standardized by ACTB expression.

FIG. 5-1 presents photographs showing the expression of PKCη in atherosclerotic arteries. a. Low-magnification image (Masson's trichrome staining) of a case with coronary atherosclerosis (AHA type IV lesion). b. Low-magnification image of PKCη expression. c. c-1 and c-2 show lesions from serial sections of the boxed region in panel b. Each of the serial panels showed immunoreactivity towards CD31 (c-1) and PKCη (c-2). CD31-positive endothelial cells were also positive towards PKCη. d. d-1 and d-2 are lesions from serial sections of the boxed region in panel b. Each of the serial panels showed immunoreactivity towards CD68 (d-1) and PKCη (d-2). Most of the CD68-positive macrophages were also positive towards PKCη. e. e-1 and e-2 are lesions from serial sections of the boxed region in panel b. Each of the serial panels showed immunoreactivity towards α-SMA (e-1) and PKCη (e-2). A portion of the α-SMA-positive smooth muscle cells were also positive towards PKCη. The sections in panels b to e have been counterstained with hematoxylin. Scale bar: a and b=500 μm, and c to e=50 μm.

FIG. 5-2 presents a bar graph showing the relationship between the grade of atherosclerosis as defined by the AHA classification and the PKCη expression in the atherosclerotic intima. Each bar represents the mean±s.e.m. of the positive area in all sections examined in each group. PKCη expression increased linearly with the severity of coronary atherosclerosis.

FIG. 6 shows the age- and sex-adjusted incidence rates of cerebral infarction observed according to PKCη genotypes (1425G>A corresponds to the amino acid substitution V374I) during a 14-year follow-up period in the Hisayama study.

FIG. 7 shows the age- and sex-adjusted incidence rates of coronary artery diseases observed according to PKCη genotypes (1425G>A corresponds to the amino acid substitution V374I) during a 14-year follow-up period in the Hisayama study.

FIG. 8 shows the correlation analysis around the AGTRL1 gene. a. Pair-wise linkage disequilibrium (LD) map between SNPs around the AGTRL1 gene evaluated by D′ (lower left triangle) and Δ2 (upper right triangle) in each case group. SNP6 (arrow) is a marker SNP detected in the second screening. b. Case-control plot [−log₁₀ (P value)] around the AGTRL1 gene. 860 test cases with LA and AT were compared with 860 control subject cases. SNP6 (arrow) is a marker SNP detected in the second screening. c. Gene structure and polymorphisms in the AGTRL1 gene. The exact positions of SNPs are shown in Tables 1-1 to 1-6. ATG: start codon; TAG: stop codon; I/D: insertion/deletion polymorphism.

FIG. 9 presents photographs and diagrams showing the results of EMSA with AGTRL1 polymorphisms. a. EMSA using a 25-bp probe around the respective alleles of nine types of polymorphisms. Nuclear extract from SBC-3 cells was used. b. Sequences of the probes used for EMSA. Capital letters indicate the polymorphisms. 1: risk allele; 2: non-risk allele. c. Binding affinity predicted by the MATCH program between the DNA sequences around the respective alleles of SNP4 and SNP9, and Sp1. Capital letters in the sequences indicate the polymorphisms. d. Super shift assay of the SNP4 region and SNP9 region using an Sp1 antibody. Nuclear extract of SBC-3 was used. The arrows indicate the bands of the probe-Sp1 complex and supershifted bands. 1: risk allele; 2: non-risk allele; C: Sp1 consensus oligonucleotide (positive control bound to Sp1).

FIG. 10 presents photographs and diagram showing the results of RT-PCR with AGTRL1 mRNA in SP1-overexpressing cells. Mock pCAGGS vector or pCAGGS-Sp1 vector was transfected into 293T cells. The AGTRL1 mRNA increased in a time-dependent manner due to Sp1 overexpression. B2M was used as an internal control. a. Semi-quantitative RT-PCR. b. Quantitative real time RT-PCR.

FIG. 11 shows the result of transcriptional regulatory activity affected by SNPs. a. A 44-bp fragment around each allele of SNP4 (−279G/A) in the 5′ flanking region is inserted into the pGL3-basic vector. −279G and −-279A represent a risk allele and non-risk allele of SNP4, respectively. b. A 44-bp fragment around SNP4 and a 53-bp fragment around SNP9 (+1355G/A) are inserted into pGL3-basic. Luciferase assay was performed using SBC-3 cells under conditions of cotransfection with mock pCAGGS and pCAGGS-Sp1. Hap1, Hap2, and Hap 3 represent a risk haplotype (−279G and +1355G), non-risk haplotype (−279G and +1355A), and intermediate haplotype (−279G and +1355G), respectively. The data shown is mean±s.d. (n=3, *P<0.05, **P<0.01). Each sample was tested three times.

### BEST MODE FOR CARRYING OUT THE INVENTION

The present inventors identified the AGTRL1 and PRKCH genes as being implicated in arteriosclerotic diseases such as cerebral infarction, and also identified polymorphic mutations (SNPs) involved in the expression or function of these genes. Enhancement of the expression of the AGTRL1 or PRKCH gene, or enhancement of the function of a protein encoded by the gene was found to be deeply associated with the onset of arteriosclerotic diseases. A subject with elevated expression of the AGTRL1 or PRKCH gene can be determined to have a risk factor for arteriosclerotic diseases (a constitution that is prone to arteriosclerotic diseases).

In the present invention, the term “arteriosclerotic disease” normally refers to a disease caused by arteriosclerosis. Specific examples include cerebral infarction (including, for example, lacunar infarction and atherothrombotic infarction), myocardial infarction, arteriosclerosis (including, for example, atherosclerosis), arteriosclerosis obliterans, aortic aneurysm, and renal artery stenosis. Furthermore, lacunar infarction mentioned above is a disease caused by arteriolosclerosis, and cerebral vascular dementia (Binswanger's disease in particular), asymptomatic cerebral infarction, micromyocaridal infarction, and such are, for example, also included in the arteriosclerotic diseases of the present invention.

The present inventors were the first to discover that the presence or absence of a risk factor for arteriosclerosis can be examined using polymorphic mutations, expression, or such of the AGTRL1 or PRKCH gene as an index.

The present invention initially provides methods of testing whether or not a subject has a risk factor for arteriosclerotic diseases, wherein the expression of AGTRL1 or PRKCH gene in the subject (a biological sample derived from the subject) is used as an index.

Accordingly, it is possible to determine whether or not the subject has a risk factor for arteriosclerotic diseases by using, as an index, the expression of AGTRL1 or PRKCH gene or the activity (function) of the protein encoded by the gene.

Information on the nucleotide sequence of the AGTRL1 or PRKCH gene in the present invention and the amino acid sequence of the protein encoded by the gene is easily accessible through the above-mentioned GenBank Accession Number. One skilled in the art can readily obtain information on the nucleotide sequence of the gene, and the amino acid sequence of the protein encoded by the gene from a public gene database or document database based on the gene notation (gene name).

The accession numbers of public databases such as GenBank and SEQ ID NOs in the Sequence Listing for the nucleotide and amino acid sequences of the AGTRL1 gene and the PRKCH gene are shown below.
AGTRL1 mRNA: NM_{—}005161 (RefSeq) (SEQ ID NO: 3), amino acid: NP_{—}005152 (SEQ ID NO: 4)
PRKCH mRNA: NM_{—}006255 (RefSeq) (SEQ ID NO: 5), amino acid: NP_{—}006246 (SEQ ID NO: 6)

The nucleotide sequence of the genomic DNA region including the AGTRL1 gene is shown in SEQ ID NO: 1. The nucleotide sequence of the genomic DNA region comprising the PRKCH gene is shown in SEQ ID NO: 2. For both the AGTRL1 and PRKCH genes, the Sequence Listing shows plus strands.

The term “subject” as used in the present invention generally refers to a human; however, the testing method of the present invention is not necessarily limited to methods which utilize only a human subjects for examination. When a subject is a non-human organism (preferably a vertebrate, and more preferably a mammal such as a mouse, rat, monkey, dog, or cat), the subject may be tested using, as an index, the expression level of an endogenous gene of the subject organism corresponding to the AGTRL1 or PRKCH gene. Accordingly, the “AGTRL1 or PRKCH gene” in the present invention includes, for example, endogenous DNAs (e.g., AGTRL1 or PRKCH gene homologs) of other organisms, corresponding to the DNA having the nucleotide sequence of SEQ ID NO: 1 or 2.

Such endogenous DNAs of other organisms corresponding to the DNA having the nucleotide sequence of SEQ ID NO: 1 or 2 generally show a high homology to DNA of SEQ ID NO: 1 or 2. The term “high homology” means a homology of 50% or more, preferably 70% or more, more preferably 80% or more, furthermore preferably 90% or more (for example, 95% or more, particularly preferably 96%, 97%, 98%, or 99% or more). The homology can be determined using mBLAST algorithm (Altschul et al. (1990) Proc. Natl. Acad. Sci. USA 87:2264-8; Karlin and Altschul (1993) Proc. Natl. Acad. Sci. USA 90: 5873-7). When such DNAs are isolated from a living body, they are considered to hybridize with the DNA of SEQ ID NO: 1 or 2 under stringent conditions. The “stringent conditions” herein include, for example, “2×SSC, 0.1% SDS, 50° C.”, “2×SSC, 0.1% SDS, 42° C.”, and “1×SSC, 0.1% SDS, 37° C.”; and more stringent conditions include “2×SSC, 0.1% SDS, 65° C.”, “0.5×SSC, 0.1% SDS, 42° C.”, and “0.2×SSC, 0.1% SDS, 65° C.”. One skilled in the art can suitably obtain endogenous genes in other organisms corresponding to the AGTRL1 or PRKCH gene, based on the nucleotide sequence of the AGTRL1 or PRKCH gene.

The present invention further provides methods of testing whether or not a subject has a risk factor for arteriosclerotic diseases, wherein the subject is determined to have a risk factor for arteriosclerotic diseases when the expression level of AGTRL1 or PRKCH gene in the subject is elevated as compared with a control.

In the above method, a biological sample derived from the subject is generally used as a test sample. The expression level of the AGTRL1 or PRKCH gene in the test sample can be suitably measured using procedures known to one skilled in the art.

The term “expression” in the context of the “gene” includes “transcription” from the gene and “translation” into a polypeptide.

When the expression level of the gene is measured using as an index the amount of a translated product (protein) of the gene, for example, a protein sample can be prepared from a test sample, and the amount of AGTRL1 or PRKCH in the protein sample can be measured. Examples of such procedures include those known to one skilled in the art, such as enzyme-linked immunosorbent assay (ELISA), double monoclonal antibody sandwich immunoassay, monoclonal polyclonal antibody sandwich assay, immunofluorescence, Western blotting, dot blotting, immunoprecipitation, protein chip analysis (Tanpakushitsu Kakusan Koso (Protein, Nucleic Acid and Enzyme) Vol. 47 No. 5 (2002); Tanpakushitsu Kakusan Koso (in Japanese; Protein, Nucleic Acid and Enzyme) Vol. 47 No. 8 (2002)), two-dimensional electrophoresis, and SDS-polyacrylamide electrophoresis, but are not limited thereto.

When the expression level of the gene is measured using the amount of a transcript (mRNA) of the gene as an index, for example, an RNA sample can be prepared from a test sample, and the amount of AGTRL1- or PRKCH-encoding RNA contained in the RNA sample can be measured. In addition, the expression level can be evaluated by preparing a cDNA sample from the test sample and measuring the amount of AGTRL1- or PRKCH-encoding cDNA contained in the cDNA sample. The RNA sample and cDNA sample from the test sample can be prepared from a subject-derived biological sample using procedures known to one skilled in the art. Examples of such procedures include those known to one skilled in the art, such as Northern blotting, RT-PCR, and DNA array techniques.

The term “control” typically refers to the expression level of an AGTRL1 or PRKCH gene in a biological sample derived from a healthy individual. The term “expression” of the AGTRL1 or PRKCH gene as used in the present invention means both the expression of mRNA transcribed from the AGTRL1 or PRKCH gene, and the expression of a protein encoded by the AGTRL1 or PRKCH gene.

The present invention further provides methods of testing whether or not a subject has a risk factor for arteriosclerotic diseases, which include the step of detecting a mutation in the AGTRL1 or PRKCH gene of the subject.

In the present invention, “testing whether or not a subject has a risk factor for arteriosclerotic diseases” includes testing whether or not a subject has a non-risk factor for arteriosclerotic diseases, or testing to determine whether a subject is likely or less likely to develop arteriosclerotic diseases. In the method of the present invention, when a mutation is detected in the AGTRL1 or PRKCH gene, a subject is determined to have a risk factor for arteriosclerotic diseases, not to have a non-risk factor for arteriosclerotic diseases, or to have a constitution that is prone to arteriosclerotic diseases.

On the other hand, when no mutation is detected in the AGTRL1 or PRKCH gene, the subject is determined to have a non-risk factor for arteriosclerotic diseases, or have no risk factor for arteriosclerotic diseases.

The method of the present invention can determine whether a subject who is not affected by arteriosclerotic diseases is more or less likely to suffer from arteriosclerotic diseases.

The term “treatment” as used herein generally means achieving a pharmacological and/or physiological effect. The effect may be preventive in that a disease or symptom is fully or partially prevented, or may be therapeutic in that a disease or symptom is fully or partially treated. The “treatment” as used herein includes all the disease treatments in mammals, particularly in humans. Furthermore, the “treatment” also includes preventing the onset of a disease in a subject who has a risk factor for the disease but has not yet been diagnosed as being affected by the disease, suppressing the progression of the disease, alleviating the disease, and delaying its onset.

Patients who have been determined to have a risk factor for arteriosclerotic diseases by the present invention's method of testing for the presence of absence of a risk factor for arteriosclerotic diseases, can select appropriate treatments before onset and may be able to prevent the onset of arteriosclerotic diseases in advance.

Specifically, the DNA sequence of the AGTRL1 or PRKCH gene of the present invention comprises the sequence of SEQ ID NO: 1 or 2, respectively.

The “mutations” in the testing method of the present invention are generally present in ORF of the above-mentioned AGTRL1 or PRKCH gene, or in regions for regulating the expression of this gene (for example, promoter regions, enhancer regions, or introns), but the position is not limited thereto. Generally, the “mutation” preferably enhances the expression level of the above-mentioned gene, improves the stability of mRNA, or increases the activity of a protein encoded by the gene. Examples of types of mutations in the present invention include addition, deletion, substitution, and insertion of nucleotides.

In the AGTRL1 gene, the “mutation” preferably changes the binding activity of the Sp1 transcription factor. Preferred examples of the mutation include SNP4 polymorphic mutation (the type of nucleotide at the polymorphic site located at position 42509 of the nucleotide sequence of SEQ ID NO: 1 is C (and the type of nucleotide in its complementary strand is G)) and SNP9 polymorphic mutation (the type of nucleotide at the polymorphic site located at position 39353 of the nucleotide sequence of SEQ ID NO: 1 is C (and the nucleotide type of in its complementary strand is G)), which are indicated in the Examples described later.

Meanwhile, preferred embodiments in the PRKCH gene include a DNA mutation that changes the valine at position 374 to isoleucine in the amino acid sequence of the PRKCH protein encoded by the gene.

The present inventors successfully discovered polymorphic mutations in the AGTRL1 or PRKCH gene of subjects that are significantly associated with arteriosclerotic diseases. Accordingly, it is possible to test whether or not a subject has a risk factor for arteriosclerotic diseases by using, as an index, the presence or absence of a mutation (by determining the nucleotide type) at a polymorphic site on the AGTRL1 or PRKCH gene.

A preferred embodiment of the present invention relates to a method of testing whether or not a subject has a risk factor for arteriosclerotic diseases, including the step of detecting a polymorphic mutation in the AGTRL1 or PRKCH gene of the present invention.

The term “polymorphism” in genetics is generally defined as a variation of a certain nucleotide in one gene, where the variation occurs at a frequency of 1% or more in a population. However, the “polymorphism” in the present invention is not restricted by this definition. Examples of the polymorphism in the present invention include single nucleotide polymorphisms, and polymorphisms in which one to several tens of nucleotides (occasionally several thousands of nucleotides) are deleted or inserted. In addition, the number of polymorphic site is not limited to one, and two or more polymorphisms may be present.

The present invention further provides methods for testing whether or not a subject has a risk factor for arteriosclerotic diseases, including the step of determining the nucleotide type at a polymorphic site in the AGTRL1 or PRKCH gene of the present invention.

The “polymorphic site” in the present invention's method of testing for the presence or absence of a risk factor for arteriosclerotic diseases is not particularly limited so long as it is a polymorphism present in the AGTRL1 or PRKCH gene of the present invention, or in the region surrounding the gene. Information on polymorphic sites found in AGTRL1 is shown in Tables 1-1 to 1-6, and information on polymorphic sites found in the PRKCH gene is shown in Tables 2-1 to 2-6. In the “Strand” column of the Tables, “+” refers to plus strand and “−” refers to minus strand. In the “Type of polymorphism” column, “snp” refers to a single nucleotide polymorphic mutation, and “in-del” refers to an insertion/deletion mutation.

| | | | | | | |
|---|---|---|---|---|---|---|
| Chromosome | Chromosome | Position in the | | | | Polymorphism |
| No. | position | Sequence Listing | SNP ID | Strand | Nucleotide | type |

Table 1-1

| | | | | | | |
|---|---|---|---|---|---|---|
| chr11 | 56719135 | 1 | rs717211 | − | C/T | snp |
| chr11 | 56719135 | 1 | SNP_A-1682036 | + | A/G | snp |
| chr11 | 56719430 | 296 | rs717210 | − | C/T | snp |
| chr11 | 56719430 | 296 | SNP_A-1713522 | + | A/G | snp |
| chr11 | 56719958 | 824 | rs10501364 | + | A/C | snp |
| chr11 | 56719958 | 824 | SNP_A-1673288 | − | G/T | snp |
| chr11 | 56720066 | 932 | rs11228941 | + | C/T | snp |
| chr11 | 56720176 | 1042 | rs12271407 | + | C/T | snp |
| chr11 | 56720206 | 1072 | rs948849 | − | C/T | snp |
| chr11 | 56720800 | 1666 | rs10896581 | + | A/G | snp |
| chr11 | 56720974 | 1840 | rs10896582 | + | C/T | snp |
| chr11 | 56722230 | 3096 | rs11228944 | + | G/T | snp |
| chr11 | 56722315 | 3181 | rs948848 | − | A/G | snp |
| chr11 | 56722359 | 3225 | rs7103566 | + | A/G | snp |
| chr11 | 56722515 | 3381 | rs7103802 | + | C/G | snp |
| chr11 | 56722515 | 3381 | SNP_A-1751467 | + | C/G | snp |
| chr11 | 56722550 | 3416 | rs10501365 | + | A/G | snp |
| chr11 | 56722550 | 3416 | SNP_A-1673178 | + | A/G | snp |
| chr11 | 56722912 | 3778 | rs1815860 | − | C/T | snp |
| chr11 | 56723045 | 3911 | rs2511033 | + | A/C | snp |
| chr11 | 56723377 | 4243 | rs4939111 | + | C/T | snp |
| chr11 | 56723377 | 4243 | SNP_A-1669987 | + | C/T | snp |
| chr11 | 56723391 | 4257 | rs10501366 | + | A/G | snp |
| chr11 | 56723799 | 4665 | rs11228949 | + | A/C | snp |
| chr11 | 56724191 | 5057 | rs12295419 | + | G/T | snp |
| chr11 | 56724313 | 5179 | rs11605468 | + | C/T | snp |
| chr11 | 56724551 | 5417 | rs2156459 | − | C/T | snp |
| chr11 | 56724582 | 5448 | rs17145552 | + | C/T | snp |
| chr11 | 56724664 | 5530 | rs1893937 | + | A/G | snp |
| chr11 | 56725136 | 6002 | rs17145556 | + | C/T | snp |
| chr11 | 56725535 | 6401 | rs7124474 | + | C/G | snp |
| chr11 | 56725697 | 6563 | rs10607820 | + | —/AGTG | in-del |
| chr11 | 56726012 | 6878 | rs11606597 | + | A/C | snp |
| chr11 | 56726031 | 6897 | rs17145573 | + | A/G | snp |
| chr11 | 56726095 | 6961 | rs7114596 | + | G/T | snp |
| chr11 | 56726630 | 7496 | rs12575327 | + | A/G | snp |
| chr11 | 56727824 | 8690 | rs7102922 | + | A/G | snp |
| chr11 | 56728174 | 9040 | rs2511028 | + | A/C | snp |
| chr11 | 56728293 | 9159 | rs11603531 | + | A/G | snp |
| chr11 | 56728574 | 9440 | rs7123500 | + | A/T | snp |
| chr11 | 56729484 | 10350 | rs12223141 | + | G/T | snp |
| chr11 | 56729633 | 10499 | rs11228950 | + | C/T | snp |
| chr11 | 56729669 | 10535 | rs4939112 | + | C/T | snp |
| chr11 | 56730369 | 11235 | rs1943477 | + | C/T | snp |
| chr11 | 56730424 | 11290 | rs1943478 | + | A/G | snp |
| chr11 | 56731675 | 12541 | rs2156456 | + | A/G | snp |
| chr11 | 56732238 | 13104 | rs5792032 | + | —/TAA | in-del |
| chr11 | 56732869 | 13735 | rs11602335 | + | C/T | snp |
| chr11 | 56732927 | 13793 | rs1943479 | + | C/T | snp |
| chr11 | 56732989 | 13855 | rs11602357 | + | C/G | snp |
| chr11 | 56733159 | 14025 | rs12285798 | + | A/T | snp |
| chr11 | 56734089 | 14955 | rs12806994 | + | A/G | snp |
| chr11 | 56734205 | 15071 | rs11228951 | + | C/G | snp |
| chr11 | 56736043 | 16909 | rs11603194 | + | A/G | snp |
| chr11 | 56737415 | 18281 | rs12225155 | + | A/G | snp |
| chr11 | 56738240 | 19106 | rs12283943 | + | A/T | snp |

Table 1-2
Table 1-2 is the continuation of Table 1-1.

| | | | | | | |
|---|---|---|---|---|---|---|
| chr11 | 56738608 | 19474 | rs2156457 | + | G/T | snp |
| chr11 | 56738788 | 19654 | rs2156458 | + | C/T | snp |
| chr11 | 56740224 | 21090 | rs7101929 | + | C/G | snp |
| chr11 | 56740679 | 21545 | rs10896586 | + | C/T | snp |
| chr11 | 56740910 | 21776 | rs11828733 | + | A/G | snp |
| chr11 | 56741024 | 21890 | rs12099289 | + | A/G | snp |
| chr11 | 56741471 | 22337 | rs12295604 | + | C/G | snp |
| chr11 | 56741508 | 22374 | rs12807301 | + | A/G | snp |
| chr11 | 56742126 | 22992 | rs11228952 | + | C/T | snp |
| chr11 | 56742382 | 23248 | rs10160780 | + | C/T | snp |
| chr11 | 56742431 | 23297 | rs7926117 | + | C/T | snp |
| chr11 | 56742777 | 23643 | rs10896587 | + | A/G | snp |
| chr11 | 56742828 | 23694 | rs4939113 | + | A/G | snp |
| chr11 | 56742979 | 23845 | rs12286248 | + | C/T | snp |
| chr11 | 56743060 | 23926 | rs10896588 | + | A/G | snp |
| chr11 | 56743194 | 24060 | rs11228954 | + | C/T | snp |
| chr11 | 56744253 | 25119 | rs11602646 | + | A/C | snp |
| chr11 | 56744582 | 25448 | rs11228955 | + | C/G | snp |
| chr11 | 56744597 | 25463 | rs957922 | − | A/G | snp |
| chr11 | 56744872 | 25738 | rs10792077 | + | A/G | snp |
| chr11 | 56744928 | 25794 | rs4939114 | + | A/G | snp |
| chr11 | 56745027 | 25893 | rs4442567 | + | A/G | snp |
| chr11 | 56745093 | 25959 | rs11228956 | + | C/T | snp |
| chr11 | 56745703 | 26569 | rs12290010 | + | C/T | snp |
| chr11 | 56745960 | 26826 | rs4939115 | + | C/T | snp |
| chr11 | 56746204 | 27070 | rs12288876 | + | A/G | snp |
| chr11 | 56746265 | 27131 | rs10736681 | + | C/T | snp |
| chr11 | 56747026 | 27892 | rs11228957 | + | C/G | snp |
| chr11 | 56747173 | 28039 | rs11604352 | + | C/T | snp |
| chr11 | 56747255 | 28121 | rs10605080 | + | —/GTTTGTTT | in-del |
| chr11 | 56747768 | 28634 | rs10896593 | + | C/T | snp |
| chr11 | 56748250 | 29116 | rs11228958 | + | A/G | snp |
| chr11 | 56748263 | 29129 | rs10896594 | + | A/G | snp |
| chr11 | 56748324 | 29190 | rs2510357 | + | C/T | snp |
| chr11 | 56748439 | 29305 | rs7928962 | + | C/T | snp |
| chr11 | 56748523 | 29389 | rs12802382 | + | G/T | snp |
| chr11 | 56748861 | 29727 | rs12225016 | + | C/T | snp |
| chr11 | 56748988 | 29854 | rs4939117 | + | A/G | snp |
| chr11 | 56749355 | 30221 | rs4939118 | + | A/T | snp |
| chr11 | 56749423 | 30289 | rs10652895 | + | —/AT | in-del |
| chr11 | 56749424 | 30290 | rs10652894 | + | —/TA | in-del |
| chr11 | 56750418 | 31284 | rs10431125 | + | A/T | snp |
| chr11 | 56750715 | 31581 | rs9667550 | + | C/T | snp |
| chr11 | 56751156 | 32022 | rs11422182 | + | —/A | in-del |
| chr11 | 56751611 | 32477 | rs7130787 | + | C/T | snp |
| chr11 | 56751745 | 32611 | rs17651297 | + | C/G | snp |
| chr11 | 56751841 | 32707 | rs1893676 | + | A/G | snp |
| chr11 | 56751910 | 32776 | rs1893677 | + | C/G | snp |
| chr11 | 56752185 | 33051 | rs1943482 | + | A/G | snp |
| chr11 | 56752529 | 33395 | rs10792078 | + | A/C | snp |
| chr11 | 56752655 | 33521 | rs17151761 | + | A/G | snp |
| chr11 | 56752790 | 33656 | rs7926885 | + | A/G | snp |
| chr11 | 56752915 | 33781 | rs7927969 | + | C/G | snp |
| chr11 | 56753784 | 34650 | rs4938853 | + | C/T | snp |
| chr11 | 56754010 | 34876 | rs4938854 | + | C/T | snp |
| chr11 | 56754225 | 35091 | rs7109894 | + | C/T | snp |
| chr11 | 56754408 | 35274 | rs721607 | + | C/T | snp |
| chr11 | 56754499 | 35365 | rs721608 | + | A/G | snp |
| chr11 | 56755401 | 36267 | rs17151767 | + | A/G | snp |
| chr11 | 56755459 | 36325 | rs1943483 | + | A/G | snp |

Table 1-3
Table 1-3 is the continuation of Table 1-2.

| | | | | | | | |
|---|---|---|---|---|---|---|---|
| | chr11 | 56755730 | 36596 | rs1943484 | + | C/T | snp |
| | chr11 | 56755753 | 36619 | rs5792033 | + | —/TCTC | in-del |
| | chr11 | 56755815 | 36681 | rs11228960 | + | C/T | snp |
| | chr11 | 56755816 | 36682 | rs11228961 | + | C/T | snp |
| | chr11 | 56755823 | 36689 | rs11228962 | + | C/T | snp |
| | chr11 | 56756234 | 37100 | rs10543434 | + | —/TC | in-del |
| | chr11 | 56756244 | 37110 | rs4939119 | + | —/C/CT/T | mixed |
| | chr11 | 56756246 | 37112 | rs4939120 | + | C/T | snp |
| | chr11 | 56756253 | 37119 | rs4939121 | + | C/T | snp |
| | chr11 | 56756257 | 37123 | rs4939122 | + | C/T | snp |
| | chr11 | 56757293 | 38159 | rs10667795 | + | —/TGGATGGA | in-del |
| | chr11 | 56757711 | 38577 | rs3177149 | − | C/T | snp |
| | chr11 | 56758186 | 39052 | rs1044235 | − | A/C | snp |
| SNP10 | chr11 | 56758402 | 39268 | rs2282623 | + | [symbol] | snp |
| SNP9 | chr11 | 56758487 | 39353 | rs2282624 | + | [symbol] | snp |
| SNP8 | chr11 | 56758504 | 39370 | rs2282625 | + | [symbol] | snp |
| | chr11 | 56758608 | 39474 | rs746885 | + | A/G | snp |
| SNP7 | chr11 | 56758687 | 39553 | rs746886 | + | [symbol] | snp |
| | chr11 | 56758727 | 39593 | rs948846 | + | A/G | snp |
| | chr11 | 56758823 | 39689 | rs746887 | + | A/G | snp |
| | chr11 | 56759081 | 39947 | rs12270028 | + | A/C | snp |
| | chr11 | 56760157 | 41023 | rs7943508 | + | C/T | snp |
| SNP6 | chr11 | 56760920 | 41786 | rs948847 | + | [symbol] | snp |
| SNP5 | chr11 | 56761153 | 42019 | rs11544374 | − | [symbol] | snp |
| | chr11 | 56761425 | 42291 | rs2510358 | + | G/T | snp |
| SNP4 | chr11 | 56761643 | 42509 | rs9943582 | + | [symbol] | snp |
| SNP3 | chr11 | 56762163 | 43029 | rs10501367 | + | [symbol] | snp |
| | chr11 | 56762163 | 43029 | SNP_A-1660323 | + | C/T | snp |
| | chr11 | 56762184 | 43050 | rs11605847 | + | G/T | snp |
| SNP2 | chr11 | 56762540 | 43406 | rs7119375 | + | [symbol] | snp |
| | chr11 | 56762540 | 43406 | SNP_A-1701491 | + | A/G | snp |
| SNP1 | chr11 | 56762797 | 43663 | rs4939123 | + | [symbol] | snp |
| | chr11 | 56763122 | 43988 | rs7120078 | + | A/G | snp |
| | chr11 | 56763174 | 44040 | rs10571462 | + | —/AAAA | in-del |
| | chr11 | 56763177 | 44043 | rs7120095 | + | A/T | snp |
| | chr11 | 56763292 | 44158 | rs12365036 | + | A/G | snp |
| | chr11 | 56763489 | 44355 | rs12365057 | + | A/G | snp |
| | chr11 | 56763502 | 44368 | rs12365058 | + | A/G | snp |
| | chr11 | 56764233 | 45099 | rs17651573 | + | A/G | snp |
| | chr11 | 56764379 | 45245 | rs7130847 | + | A/T | snp |
| | chr11 | 56764758 | 45624 | rs7128178 | + | A/G | snp |
| | chr11 | 56765112 | 45978 | rs10736682 | + | A/G | snp |
| | chr11 | 56765920 | 46786 | rs1893675 | + | G/T | snp |
| | chr11 | 56766152 | 47018 | rs12576521 | + | G/T | snp |
| | chr11 | 56766174 | 47040 | rs948844 | + | A/C | snp |
| | chr11 | 56766220 | 47086 | rs12576524 | + | A/G | snp |
| | chr11 | 56766667 | 47533 | rs11228966 | + | C/T | snp |
| | chr11 | 56766678 | 47544 | rs11228967 | + | A/G | snp |
| | chr11 | 56766990 | 47856 | rs10792079 | + | A/C | snp |
| | chr11 | 56767735 | 48601 | rs4939124 | + | G/T | snp |
| | chr11 | 56768620 | 49486 | rs17651610 | + | A/C | snp |
| | chr11 | 56768746 | 49612 | rs17151782 | + | A/C | snp |
| | chr11 | 56768851 | 49717 | rs7928905 | + | G/T | snp |
| | chr11 | 56768898 | 49764 | rs499318 | + | A/G | snp |
| | chr11 | 56769089 | 49955 | rs501160 | + | G/T | snp |
| | chr11 | 56769318 | 50184 | rs11606862 | + | A/G | snp |
| | chr11 | 56769824 | 50690 | rs599947 | − | C/T | snp |
| | chr11 | 56770404 | 51270 | rs534130 | + | A/T | snp |
| | chr11 | 56770411 | 51277 | rs694902 | − | A/G | snp |
| | chr11 | 56770479 | 51345 | rs12280902 | + | C/T | snp |

Table 1-4
Table 1-4 is the continuation of Table 1-3.

| | | | | | | |
|---|---|---|---|---|---|---|
| chr11 | 56771061 | 51927 | rs11228968 | + | A/T | snp |
| chr11 | 56771238 | 52104 | rs2511032 | − | A/G | snp |
| chr11 | 56771239 | 52105 | rs2511031 | − | A/T | snp |
| chr11 | 56771245 | 52111 | rs4938855 | + | G/T | snp |
| chr11 | 56771333 | 52199 | rs2511030 | − | C/T | snp |
| chr11 | 56771699 | 52565 | rs12789789 | + | C/G | snp |
| chr11 | 56771939 | 52805 | rs652016 | + | C/T | snp |
| chr11 | 56772312 | 53178 | rs7108880 | + | A/G | snp |
| chr11 | 56772338 | 53204 | rs10534300 | + | —/CAAAA | in-del |
| chr11 | 56772428 | 53294 | rs481516 | + | A/T | snp |
| chr11 | 56772478 | 53344 | rs654635 | + | C/T | snp |
| chr11 | 56772508 | 53374 | rs17151797 | + | A/G | snp |
| chr11 | 56772937 | 53803 | rs11825586 | + | C/G | snp |
| chr11 | 56773084 | 53950 | rs11228969 | + | A/C | snp |
| chr11 | 56773085 | 53951 | rs11228970 | + | A/T | snp |
| chr11 | 56773307 | 54173 | rs10896595 | + | C/T | snp |
| chr11 | 56773310 | 54176 | rs668408 | + | C/T | snp |
| chr11 | 56773637 | 54503 | rs1943469 | + | C/T | snp |
| chr11 | 56773809 | 54675 | rs1788969 | + | A/G | snp |
| chr11 | 56773893 | 54759 | rs1262323 | + | A/G | snp |
| chr11 | 56774012 | 54878 | rs1943470 | + | A/C | snp |
| chr11 | 56774133 | 54999 | rs518272 | + | C/T | snp |
| chr11 | 56774260 | 55126 | rs519277 | + | A/G | snp |
| chr11 | 56774875 | 55741 | rs2226845 | + | C/T | snp |
| chr11 | 56774978 | 55844 | rs4938856 | + | C/T | snp |
| chr11 | 56775445 | 56311 | rs12418878 | + | A/C | snp |
| chr11 | 56775819 | 56685 | rs11228971 | + | A/G | snp |
| chr11 | 56776168 | 57034 | rs578831 | + | C/T | snp |
| chr11 | 56776615 | 57481 | rs2846039 | + | G/T | snp |
| chr11 | 56776666 | 57532 | rs625565 | + | A/G | snp |
| chr11 | 56777092 | 57958 | rs10896596 | + | C/G | snp |
| chr11 | 56777280 | 58146 | rs499885 | + | A/G | snp |
| chr11 | 56777440 | 58306 | rs639248 | + | C/T | snp |
| chr11 | 56777483 | 58349 | rs522656 | + | A/G | snp |
| chr11 | 56777953 | 58819 | rs12576471 | + | C/T | snp |
| chr11 | 56777991 | 58857 | rs641550 | + | C/T | snp |
| chr11 | 56778031 | 58897 | rs1939491 | + | A/G | snp |
| chr11 | 56778039 | 58905 | rs528101 | + | A/C | snp |
| chr11 | 56778056 | 58922 | rs652353 | + | A/C | snp |
| chr11 | 56778177 | 59043 | rs4938857 | + | A/C | snp |
| chr11 | 56778375 | 59241 | rs4384399 | + | A/G | snp |
| chr11 | 56778725 | 59591 | rs655402 | + | C/G | snp |
| chr11 | 56778973 | 59839 | rs656366 | + | C/T | snp |
| chr11 | 56779036 | 59902 | rs558266 | + | C/T | snp |
| chr11 | 56779119 | 59985 | rs559168 | + | A/G | snp |
| chr11 | 56779228 | 60094 | rs1939492 | + | G/T | snp |
| chr11 | 56779548 | 60414 | rs7130252 | + | A/G | snp |
| chr11 | 56779741 | 60607 | rs12574780 | + | C/G | snp |
| chr11 | 56779921 | 60787 | rs670919 | + | A/C | snp |
| chr11 | 56780249 | 61115 | rs479949 | + | A/T | snp |
| chr11 | 56781406 | 62272 | rs1257753 | + | C/T | snp |
| chr11 | 56783090 | 63956 | rs2510359 | + | A/C | snp |
| chr11 | 56783134 | 64000 | rs7936545 | + | C/T | snp |
| chr11 | 56783410 | 64276 | rs7102963 | + | A/C | snp |
| chr11 | 56783480 | 64346 | rs2508770 | + | A/G | snp |
| chr11 | 56783481 | 64347 | rs2508771 | + | A/G | snp |
| chr11 | 56783956 | 64822 | rs11604560 | + | A/C | snp |
| chr11 | 56784334 | 65200 | rs546403 | + | A/G | snp |
| chr11 | 56784862 | 65728 | rs4939126 | + | A/G | snp |
| chr11 | 56785231 | 66097 | rs2846040 | + | G/T | snp |

Table 1-5
Table 1-5 is the continuation of Table 1-4.

| | | | | | | |
|---|---|---|---|---|---|---|
| chr11 | 56786530 | 67396 | rs2846041 | + | G/T | snp |
| chr11 | 56786573 | 67439 | rs2851727 | − | C/T | snp |
| chr11 | 56786755 | 67621 | rs7930138 | + | G/T | snp |
| chr11 | 56786820 | 67686 | rs11228973 | + | A/T | snp |
| chr11 | 56786869 | 67735 | rs2846042 | + | C/T | snp |
| chr11 | 56787211 | 68077 | rs11228974 | + | A/C | snp |
| chr11 | 56787499 | 68365 | rs11228975 | + | A/C | snp |
| chr11 | 56787538 | 68404 | rs11228976 | + | A/C | snp |
| chr11 | 56787703 | 68569 | rs12271448 | + | A/G | snp |
| chr11 | 56787842 | 68708 | rs12421633 | + | C/T | snp |
| chr11 | 56788241 | 69107 | rs12800196 | + | C/T | snp |
| chr11 | 56788734 | 69600 | rs11228978 | + | A/G | snp |
| chr11 | 56789364 | 70230 | rs12295518 | + | C/T | snp |
| chr11 | 56789457 | 70323 | rs7106133 | + | A/T | snp |
| chr11 | 56789982 | 70848 | rs7120644 | + | A/G | snp |
| chr11 | 56790549 | 71415 | rs2846044 | + | A/C | snp |
| chr11 | 56790795 | 71661 | rs6591413 | + | C/T | snp |
| chr11 | 56791039 | 71905 | rs6591414 | + | G/T | snp |
| chr11 | 56791325 | 72191 | rs10896597 | + | A/C | snp |
| chr11 | 56791604 | 72470 | rs2155230 | + | A/G | snp |
| chr11 | 56792179 | 73045 | rs12279206 | + | A/G | snp |
| chr11 | 56792336 | 73202 | rs12292875 | + | C/T | snp |
| chr11 | 56792454 | 73320 | rs4939127 | + | A/G | snp |
| chr11 | 56792721 | 73587 | rs12575637 | + | C/G | snp |
| chr11 | 56792930 | 73796 | rs12803405 | + | C/T | snp |
| chr11 | 56792962 | 73828 | rs2511027 | + | A/T | snp |
| chr11 | 56793014 | 73880 | rs17151809 | + | C/G | snp |
| chr11 | 56793030 | 73896 | rs17151811 | + | A/T | snp |
| chr11 | 56793141 | 74007 | rs2846045 | + | A/G | snp |
| chr11 | 56793147 | 74013 | rs2846046 | + | A/G | snp |
| chr11 | 56793206 | 74072 | rs12785770 | + | C/T | snp |
| chr11 | 56793251 | 74117 | rs12804848 | + | A/C | snp |
| chr11 | 56793280 | 74146 | rs12804170 | + | C/T | snp |
| chr11 | 56793310 | 74176 | rs2851724 | + | A/C | snp |
| chr11 | 56793337 | 74203 | rs2846047 | + | C/T | snp |
| chr11 | 56793616 | 74482 | rs1892963 | + | A/G | snp |
| chr11 | 56793824 | 74690 | rs2155231 | + | G/T | snp |
| chr11 | 56793970 | 74836 | rs11607817 | + | C/T | snp |
| chr11 | 56794028 | 74894 | rs17652078 | + | C/T | snp |
| chr11 | 56794775 | 75641 | rs1939493 | + | C/T | snp |
| chr11 | 56794826 | 75692 | rs17652103 | + | A/G | snp |
| chr11 | 56794948 | 75814 | rs11228979 | + | C/G | snp |
| chr11 | 56794989 | 75855 | rs11228980 | + | C/T | snp |
| chr11 | 56795297 | 76163 | rs10688423 | + | —/CACA | in-del |
| chr11 | 56795524 | 76390 | rs2846048 | + | C/T | snp |
| chr11 | 56795714 | 76580 | rs11410686 | + | —/C | in-del |
| chr11 | 56795939 | 76805 | rs6591415 | + | A/G | snp |
| chr11 | 56796245 | 77111 | rs11228982 | + | A/G | snp |
| chr11 | 56796246 | 77112 | rs2846049 | + | G/T | snp |
| chr11 | 56796411 | 77277 | rs4938859 | + | G/T | snp |
| chr11 | 56796446 | 77312 | rs2846050 | + | C/T | snp |
| chr11 | 56796965 | 77831 | rs1939494 | + | A/C | snp |
| chr11 | 56797296 | 78162 | rs1892964 | + | A/C | snp |
| chr11 | 56797722 | 78588 | rs12421755 | + | C/T | snp |
| chr11 | 56797954 | 78820 | rs2155232 | + | A/G | snp |
| chr11 | 56798288 | 79154 | rs4939128 | + | C/T | snp |
| chr11 | 56798341 | 79207 | rs2186679 | + | A/C | snp |
| chr11 | 56798499 | 79365 | rs12577794 | + | A/C | snp |
| chr11 | 56798568 | 79434 | rs2155233 | + | C/T | snp |
| chr11 | 56799293 | 80159 | rs10717194 | + | —/A | in-del |

Table 1-6
Table 1-6 is the continuation of Table 1-5.

| | | | | | | |
|---|---|---|---|---|---|---|
| chr11 | 56799584 | 80450 | rs11366848 | + | —/G | in-del |
| chr11 | 56799586 | 80452 | rs11353075 | + | —/G | in-del |
| chr11 | 56800019 | 80885 | rs1939495 | + | G/T | snp |
| chr11 | 56800122 | 80988 | rs2846051 | + | A/G | snp |
| chr11 | 56802391 | 83257 | rs7931298 | + | A/C | snp |
| chr11 | 56802859 | 83725 | rs2508769 | + | C/T | snp |
| chr11 | 56803125 | 83991 | rs12362074 | + | C/G | snp |
| chr11 | 56803412 | 84278 | rs11228984 | + | C/T | snp |
| chr11 | 56803873 | 84739 | rs12788046 | + | G/T | snp |
| chr11 | 56803971 | 84837 | rs12806978 | + | A/G | snp |
| chr11 | 56804174 | 85040 | rs12806656 | + | C/T | snp |
| chr11 | 56804760 | 85626 | rs11228985 | + | A/G | snp |
| chr11 | 56805355 | 86221 | rs6650184 | + | A/C | snp |
| chr11 | 56805680 | 86546 | rs2226613 | + | A/C | snp |
| chr11 | 56805802 | 86668 | rs4565915 | + | C/T | snp |
| chr11 | 56805827 | 86693 | rs2846052 | + | A/G | snp |
| chr11 | 56805987 | 86853 | rs1939496 | + | A/G | snp |
| chr11 | 56806163 | 87029 | rs11600878 | + | C/T | snp |
| chr11 | 56806367 | 87233 | rs12797544 | + | A/G | snp |
| chr11 | 56806481 | 87347 | rs12797769 | + | A/G | snp |
| chr11 | 56806529 | 87395 | rs12419065 | + | C/T | snp |
| chr11 | 56807458 | 88324 | rs7396193 | + | A/G | snp |
| chr11 | 56808360 | 89226 | rs7395614 | + | C/T | snp |
| chr11 | 56808411 | 89277 | rs7396611 | + | C/T | snp |
| chr11 | 56808583 | 89449 | rs10896598 | + | C/T | snp |
| chr11 | 56809073 | 89939 | rs7948723 | + | A/C | snp |
| chr11 | 56810681 | 91547 | rs4939132 | + | A/C | snp |
| chr11 | 56811694 | 92560 | rs7128371 | + | G/T | snp |
| chr11 | 56812626 | 93492 | rs4938861 | + | A/C | snp |
| chr11 | 56813068 | 93934 | rs5792034 | + | —/TG | in-del |
| chr11 | 56813428 | 94294 | rs11605053 | + | C/T | snp |
| chr11 | 56813469 | 94335 | rs10896599 | + | A/G | snp |
| chr11 | 56813606 | 94472 | rs17151820 | + | C/G | snp |
| chr11 | 56814224 | 95090 | rs5792035 | + | —/TGGA | in-del |
| chr11 | 56814626 | 95492 | rs17573746 | + | A/G | snp |
| chr11 | 56815008 | 95874 | rs6591416 | + | C/G | snp |
| chr11 | 56815075 | 95941 | rs6591417 | + | A/C | snp |
| chr11 | 56815125 | 95991 | rs12797375 | + | C/T | snp |
| chr11 | 56815329 | 96195 | rs10631051 | + | —/AG | in-del |
| chr11 | 56816391 | 97257 | rs5012051 | + | A/G | snp |
| chr11 | 56816872 | 97738 | rs12786396 | + | C/T | snp |
| chr11 | 56816887 | 97753 | rs12808788 | + | A/G | snp |
| chr11 | 56816926 | 97792 | rs6591418 | + | A/T | snp |
| chr11 | 56816946 | 97812 | rs12296071 | + | A/G | snp |
| chr11 | 56817138 | 98004 | rs12792781 | + | C/T | snp |
| chr11 | 56817985 | 98851 | rs11438573 | + | —/T | in-del |
| chr11 | 56818021 | 98887 | rs7934304 | + | C/G | snp |
| chr11 | 56818207 | 99073 | rs7935236 | + | C/G | snp |
| chr11 | 56818941 | 99807 | rs5792036 | + | —/G | in-del |
| chr11 | 56819085 | 99951 | rs1939488 | + | G/T | snp |
| chr11 | 56819147 | 100013 | rs11601123 | + | C/G | snp |
| chr11 | 56819313 | 100179 | rs11228987 | + | A/G | snp |
| chr11 | 56820462 | 101328 | rs10792083 | + | C/T | snp |
| chr11 | 56820705 | 101571 | rs12365072 | + | C/T | snp |
| chr11 | 56821452 | 102318 | rs6591419 | + | C/T | snp |
| chr11 | 56821557 | 102423 | rs6591420 | + | C/G | snp |
| chr11 | 56821796 | 102662 | rs12802096 | + | C/T | snp |
| chr11 | 56822072 | 102938 | rs1939489 | + | G/T | snp |

TABLE 2
The shaded region in the following Table 2 indicates the LD block.

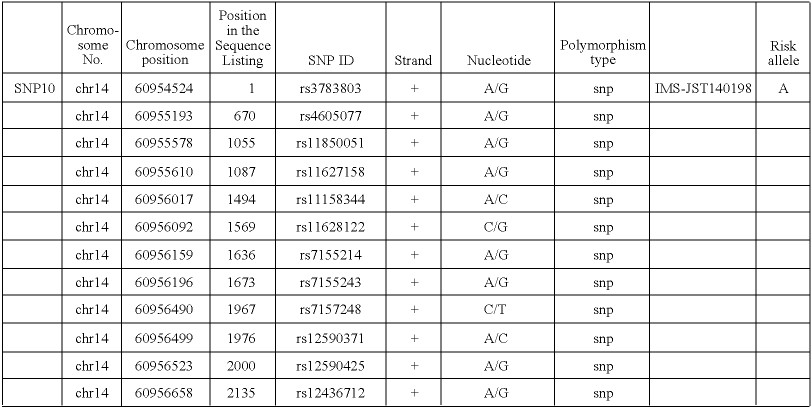

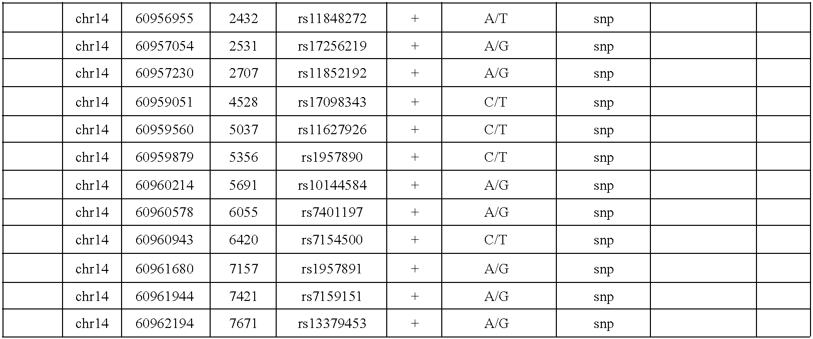

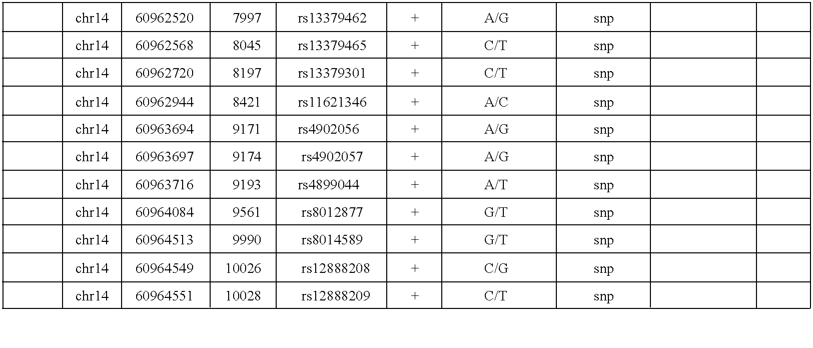

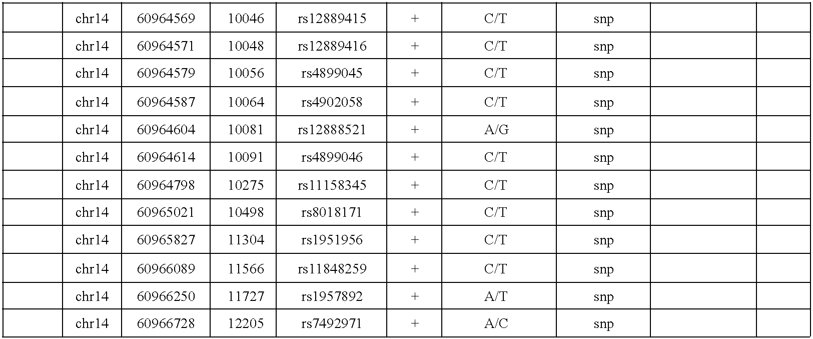

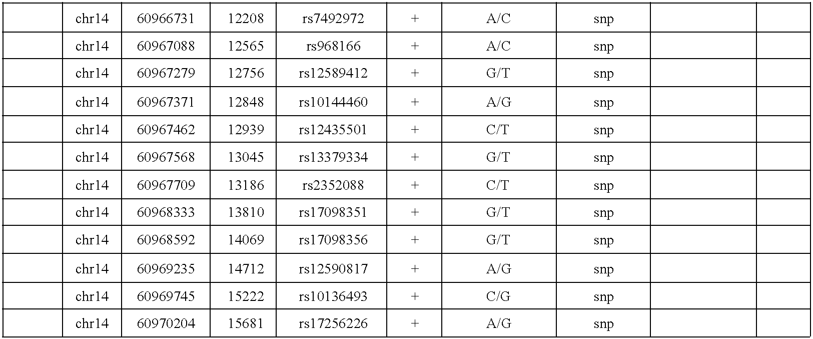

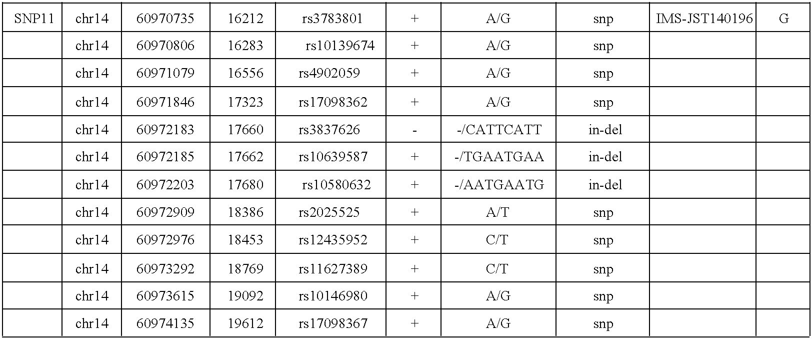

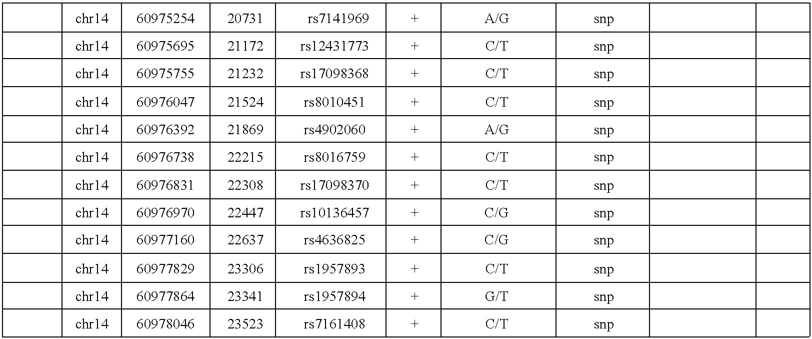

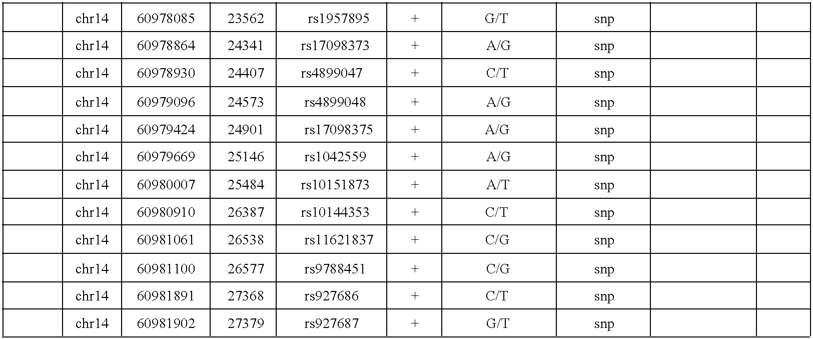

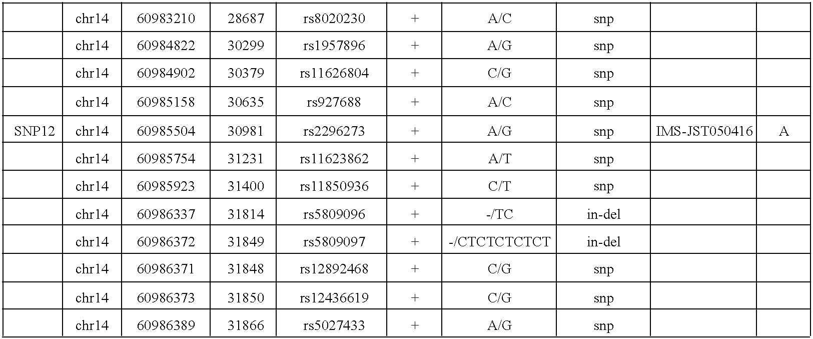

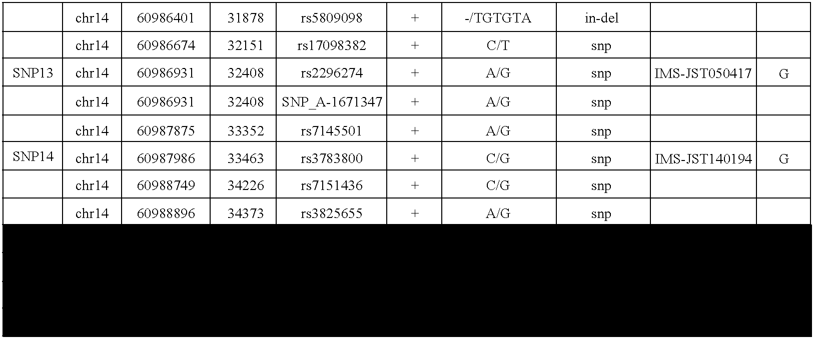

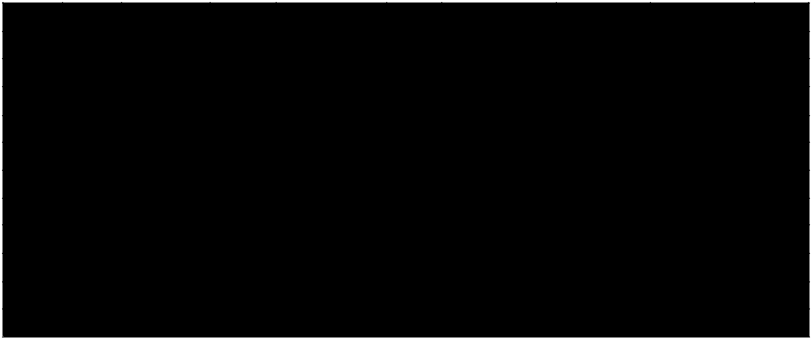

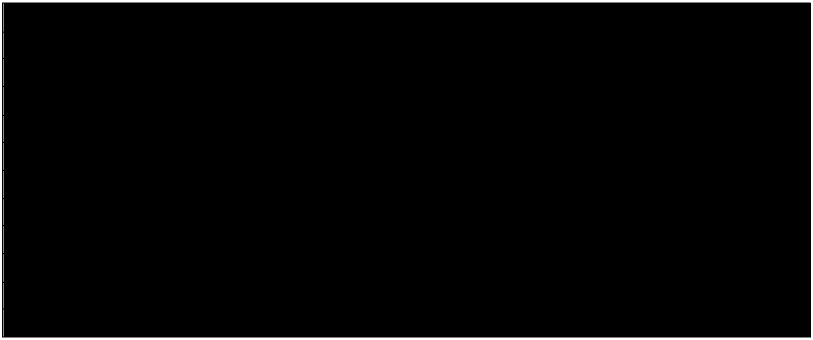

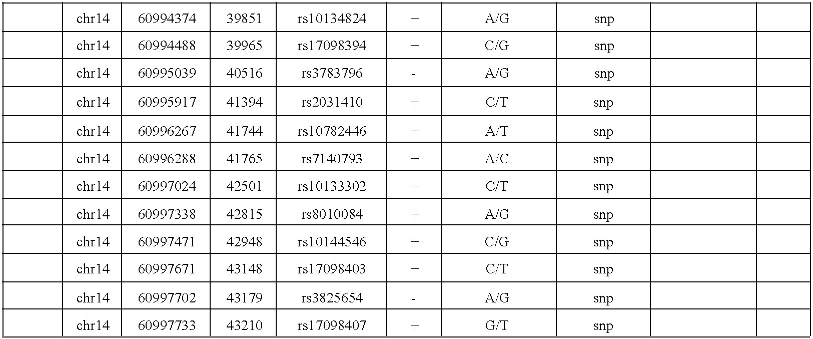

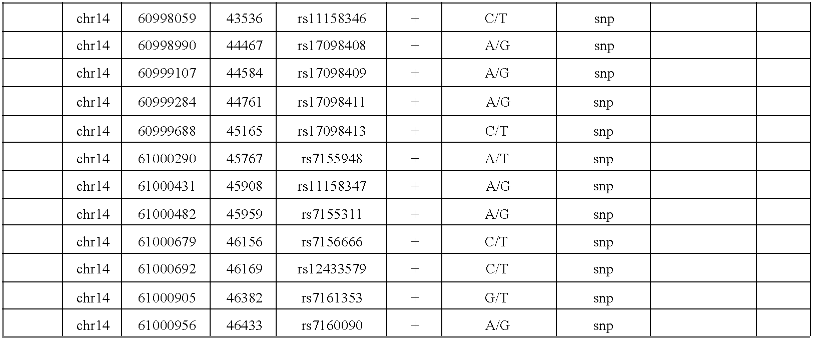

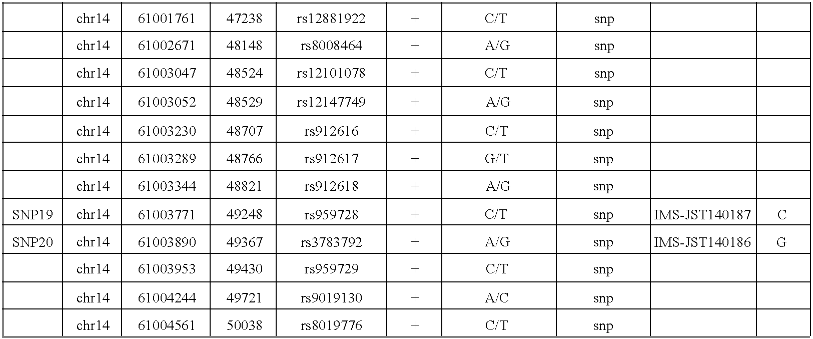

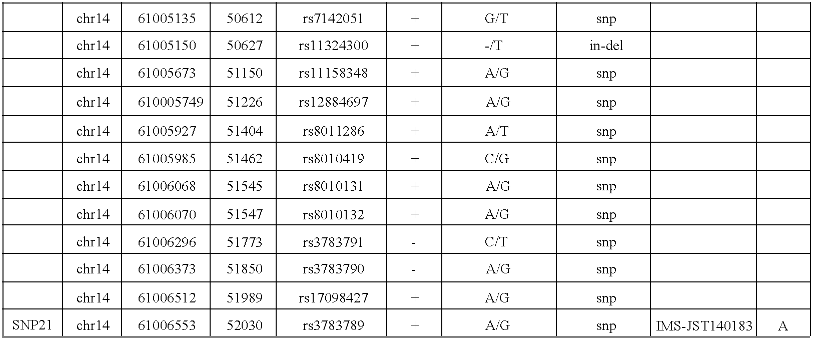

More specifically, a preferred embodiment of the present invention is a method of determining the type of nucleotides at the polymorphic sites listed in the above-mentioned Tables.

Of the above-mentioned polymorphic sites, polymorphic sites that can be used in the present invention's method of testing for the presence or absence of a risk factor for arteriosclerotic diseases are preferably those located in the AGTRL1 or PRKCH gene or in the region surrounding the gene. For example, preferred polymorphic sites in the AGTRL1 gene are located at positions 1, 296, 824, 932, 1042, 1072, 1666, 1840, 3096, 3181, 3225, 3381, 3416, 3778, 3911, 4243, 4257, 4665, 5057, 5179, 5417, 5448, 5530, 6002, 6401, 6563, 6878, 6897, 6961, 7496, 8690, 9040, 9159, 9440, 10350, 10499, 10535, 11235, 11290, 12541, 13104, 13735, 13793, 13855, 14025, 14955, 15071, 16909, 18281, 19106, 19474, 19654, 21090, 21545, 21776, 21890, 22337, 22374, 22992, 23248, 23297, 23643, 23694, 23845, 23926, 24060, 25119, 25448, 25463, 25738, 25794, 25893, 25959, 26569, 26826, 27070, 27131, 27892, 28039, 28121, 28634, 29116, 29129, 29190, 29305, 29389, 29727, 29854, 30221, 30289, 30290, 31284, 31581, 32022, 32477, 32611, 32707, 32776, 33051, 33395, 33521, 33656, 33781, 34650, 34876, 35091, 35274, 35365, 36267, 36325, 36596, 36619, 36681, 36682, 36689, 37100, 37110, 37112, 37119, 37123, 38159, 38577, 39052, 39268, 39353, 39370, 39474, 39553, 39593, 39689, 39947, 41023, 41786, 42019, 42291, 42509, 43029, 43050, 43406, 43663, 43988, 44040, 44043, 44158, 44355, 44368, 45099, 45245, 45624, 45978, 46786, 47018, 47040, 47086, 47533, 47544, 47856, 48601, 49486, 49612, 49717, 49764, 49955, 50184, 50690, 51270, 51277, 51345, 51927, 52104, 52105, 52111, 52199, 52565, 52805, 53178, 53204, 53294, 53344, 53374, 53803, 53950, 53951, 54173, 54176, 54503, 54675, 54759, 54878, 54999, 55126, 55741, 55844, 56311, 56685, 57034, 57481, 57532, 57958, 58146, 58306, 58349, 58819, 58857, 58897, 58905, 58922, 59043, 59241, 59591, 59839, 59902, 59985, 60094, 60414, 60607, 60787, 61115, 62272, 63956, 64000, 64276, 64346, 64347, 64822, 65200, 65728, 66097, 67396, 67439, 67621, 67686, 67735, 68077, 68365, 68404, 68569, 68708, 69107, 69600, 70230, 70323, 70848, 71415, 71661, 71905, 72191, 72470, 73045, 73202, 73320, 73587, 73796, 73828, 73880, 73896, 74007, 74013, 74072, 74117, 74146, 74176, 74203, 74482, 74690, 74836, 74894, 75641, 75692, 75814, 75855, 76163, 76390, 76580, 76805, 77111, 77112, 77277, 77312, 77831, 78162, 78588, 78820, 79154, 79207, 79365, 79434, 80159, 80450, 80452, 80885, 80988, 83257, 83725, 83991, 84278, 84739, 84837, 85040, 85626, 86221, 86546, 86668, 86693, 86853, 87029, 87233, 87347, 87395, 88324, 89226, 89277, 89449, 89939, 91547, 92560, 93492, 93934, 94294, 94335, 94472, 95090, 95492, 95874, 95941, 95991, 96195, 97257, 97738, 97753, 97792, 97812, 98004, 98851, 98887, 99073, 99807, 99951, 100013, 100179, 101328, 101571, 102318, 102423, 102662, and 102938 in the nucleotide sequence of SEQ ID NO: 1.

In addition, polymorphic sites in the PRKCH gene located at positions 1, 670, 1055, 1087, 1494, 1569, 1636, 1673, 1967, 1976, 2000, 2135, 2432, 2531, 2707, 4528, 5037, 5356, 5691, 6055, 6420, 7157, 7421, 7671, 7997, 8045, 8197, 8421, 9171, 9174, 9193, 9561, 9990, 10026, 10028, 10046, 10048, 10056, 10064, 10081, 10091, 10275, 10498, 11304, 11566, 11727, 12205, 12208, 12565, 12756, 12848, 12939, 13045, 13186, 13810, 14069, 14712, 15222, 15681, 16212, 16283, 16556, 17323, 17660, 17662, 17680, 18386, 18453, 18769, 19092, 19612, 20731, 21172, 21232, 21524, 21869, 22215, 22308, 22447, 22637, 23306, 23341, 23523, 23562, 24341, 24407, 24573, 24901, 25146, 25484, 26387, 26538, 26577, 27368, 27379, 28687, 30299, 30379, 30635, 30981, 31231, 31400, 31814, 31848, 31849, 31850, 31866, 31878, 32151, 32408, 33352, 33463, 34226, 34373, 34446, 34826, 34932, 35303, 35431, 35443, 35552, 35706, 35940, 36119, 36475, 36491, 36572, 36631, 36635, 36771, 37157, 37691, 37707, 38017, 38079, 38109, 39236, 39322, 39370, 39445, 39469, 39471, 39851, 39965, 40516, 41394, 41744, 41765, 42501, 42815, 42948, 43148, 43179, 43210, 43536, 44467, 44584, 44761, 45165, 45767, 45908, 45959, 46156, 46169, 46382, 46433, 47238, 48148, 48524, 48529, 48707, 48766, 48821, 49248, 49367, 49430, 49721, 50038, 50612, 50627, 51150, 51226, 51404, 51462, 51545, 51547, 51773, 51850, 51989, and 52030 are also preferred (the above polymorphic sites may be herein simply referred to as “the polymorphic sites of the present invention”).

One skilled in the art can suitably obtain information regarding the particular nucleotides at the above sites based on the above-listed rs numbers for the dbSNP database. Regarding SNP IDs, those with “rs” at the head are registration IDs in the dbSNP database that have been uniquely assigned to respective single sequences by NCBI. The dbSNP database is publicly available on a web site (http://www.ncbi.nlm.nih.gov/SNP/index.html), and detailed information on SNPs in a nucleotide sequence (for example, position on a chromosome, nucleotide type at polymorphic site, and adjacent sequences) can be obtained by conducting search on the web site using a registration ID number stated in an SNP ID. By using the information, one skilled in the art can easily perform the test of the present invention.

Generally, one skilled in the art can easily find the actual genomic position, adjacent sequences and such of the polymorphic sites of the present invention using the registration IDs given to the polymorphisms disclosed herein, such as the rs numbers in the dbSNP database. If this information cannot be found by such means, one skilled in the art can readily find the actual genomic position corresponding to the polymorphic site based on the sequence of SEQ ID NO: 1 and information on the polymorphic site or such. For example, the genomic position of the polymorphic site of the present invention can be determined by consulting a public genome database or such. Specifically, even when the nucleotide sequences are slightly different between the nucleotide sequence in the sequence listing and the actual genomic nucleotide sequence, the actual genomic position of the polymorphic site of the present invention can be precisely identified by, for example, conducting a homology search of the genomic sequence based on the nucleotide sequence in the sequence listing. Even when the genomic position cannot be identified, the test of the present invention can be easily conducted based on the sequence listing and the information on the polymorphic sites disclosed herein.

Genomic DNA usually has a mutually complementary double-stranded DNA structure. Accordingly, even when the DNA sequence of one strand is disclosed herein for the sake of convenience, it will be naturally understood that the other sequence complementary to the above sequence (nucleotides) is also disclosed. When a DNA sequence (nucleotides) in one strand is known, the other sequence (nucleotides) complementary to the above sequence (nucleotides) is obvious to one skilled in the art. Regarding the human genome sequence, the International Human Genome Project build35, which is said to be an almost final version, has been published, and the sequences and the like described herein are based on the results of the International Human Genome Project build35.

In the present invention's method of testing for the presence or absence of a risk factor for arteriosclerotic diseases, the following polymorphic sites are preferably tested.

In a more preferred embodiment of the present invention, methods of testing for the presence of absence of a risk factor for arteriosclerotic diseases involve testing the polymorphic sites comprised in the region between the polymorphic sites of (1a) and (24a) listed below, or preferably the polymorphic site in the AGTRL1 gene located at (1a) position 1, (2a) position 12541, (3a) position 21545, (4a) position 33051, (5a) position 35365, (6a) position 39268, (7a) position 39353, (8a) position 39370, (9a) position 39474, (10a) position 39553, (11a) position 39665, (12a) position 41786, (13a) position 42019, (14a) position 42509, (15a) position 43029, (16a) position 43406, (17a) position 43663, (18a) position 46786, (19a) position 49764, (20a) position 64276, (21a) position 74482, (22a) position 78162, (23a) position 93492, or (24a) position 102938 of the nucleotide sequence of SLQ ID NO: 1.

In a preferred embodiment of the present invention, a subject is determined to have a risk factor for arteriosclerotic diseases when the type of nucleotide at the polymorphic sites of (1a) to (24a) described above are (1b) to (24b) listed below, respectively. The presence or absence of a risk factor for arteriosclerotic diseases can be determined regardless of whether or not a subject is suffering from an arteriosclerotic disease.
(1b) The nucleotide type of in the complementary strand of the AGTRL1 gene located at position 1 of the nucleotide sequence of SLQ ID NO: 1 is T.
(2b) The type of nucleotide in the complementary strand of the AGTRL1 gene located at position 12541 of the nucleotide sequence of SEQ ID NO: 1 is T.
(3b) The type of nucleotide in the complementary strand of the AGTRL1 gene located at position 21545 of the nucleotide sequence of SEQ ID NO: 1 is A.
(4b) The type of nucleotide in the complementary strand of the AGTRL1 gene located at position 33051 of the nucleotide sequence of SEQ ID NO: 1 is C.
(5b) The type of nucleotide in the complementary strand of the AGTRL1 gene located at position 35365 of the nucleotide sequence of SEQ ID NO: 1 is T.
(6b) The type of nucleotide in the complementary strand of the AGTRL1 gene located at position 39268 of the nucleotide sequence of SEQ ID NO: 1 is A.
(7b) The type of nucleotide in the complementary strand of the AGTRL1 gene located at position 39353 of the nucleotide sequence of SEQ ID NO: 1 is G.
(8b) The type of nucleotide in the complementary strand of the AGTRL1 gene located at position 39370 of the nucleotide sequence of SEQ ID NO: 1 is C.
(9b) The type of nucleotide in the complementary strand of the AGTRL1 gene located at position 39474 of the nucleotide sequence of SEQ ID NO: 1 is T.
(10b) The type of nucleotide in the complementary strand of the AGTRL1 gene located at position 39553 of the nucleotide sequence of SEQ ID NO: 1 is T.
(11b) A site in the AGTRL1 gene located at position 39665 of the nucleotide sequence of SEQ ID NO: 1 has been deleted.
(12b) The type of nucleotide in the complementary strand of the AGTRL1 gene located at position 41786 of the nucleotide sequence of SEQ ID NO: 1 is A.
(13b) The type of nucleotide in the complementary strand of the AGTRL1 gene located at position 42019 of the nucleotide sequence of SEQ ID NO: 1 is G.
(14b) The type of nucleotide in the complementary strand of the AGTRL1 gene located at position 42509 of the nucleotide sequence of SEQ ID NO: 1 is G.
(15b) The type of nucleotide in the complementary strand of the AGTRL1 gene located at position 43029 of the nucleotide sequence of SEQ ID NO: 1 is G.
(16b) The type of nucleotide in the complementary strand of the AGTRL1 gene located at position 43406 of the nucleotide sequence of SEQ ID NO: 1 is C.
(17b) The type of nucleotide in the complementary strand of the AGTRL1 gene located at position 43663 of the nucleotide sequence of SEQ ID NO: 1 is T.
(18b) The type of nucleotide in the complementary strand of the AGTRL1 gene located at position 46786 of the nucleotide sequence of SEQ ID NO: 1 is C.
(19b) The type of nucleotide in the complementary strand of the AGTRL1 gene located at position 49764 of the nucleotide sequence of SEQ ID NO: 1 is T.
(20b) The type of nucleotide in the complementary strand of the AGTRL1 gene located at position 64276 of the nucleotide sequence of SEQ ID NO: 1 is T.
(21b) The type of nucleotide in the complementary strand of the AGTRL1 gene located at position 74482 of the nucleotide sequence of SEQ ID NO: 1 is C.
(22b) The type of nucleotide in the complementary strand at a site on the AGTRL1 gene located at position 78162 of the nucleotide sequence of SEQ ID NO: 1 is G.
(23b) The type of nucleotide in the complementary strand of the AGTRL1 gene located at position 93492 of the nucleotide sequence of SEQ ID NO: 1 is G.
(24b) The type of nucleotide in the complementary strand of the AGTRL1 gene located at position 102938 of the nucleotide sequence of SEQ ID NO: 1 is C.

The above-mentioned nucleotides represent those either in the plus strand or the minus strand of the nucleotide sequence of a gene of the present invention. Those skilled in the art can appropriately determine the type of nucleotide that should be detected in the polymorphic sites based on the information disclosed herein.

In another embodiment, and in a more preferred embodiment of the present invention, methods of testing for the presence or absence of a risk factor for arteriosclerotic diseases involve testing the polymorphic sites comprised in the region between the polymorphic sites of (1a) and (12a) listed below, or preferably the polymorphic site on the PRKCH gene located at (1a) position 1, (2a) position 16212, (3a) position 30981, (4a) position 32408, (5a) position 33463, (6a) position 34446, (7a) position 39322, (8a) position 39469, (9a) position 39471, (10a) position 49248, (11a) position 49367, or (12a) position 52030 of the nucleotide sequence of SEQ ID NO: 2.

In a preferred embodiment of the present invention, a subject is determined to have a risk factor for arteriosclerotic diseases when the type of nucleotide at the polymorphic sites of (1a) to (12a) mentioned above are (1b) to (12b) listed below, respectively. The presence or absence of a risk factor for arteriosclerotic diseases can be determined regardless of whether or not a subject is suffering from an arteriosclerotic disease.
(1b) The type of nucleotide in the PRKCH gene located at position 1 of the nucleotide sequence of SEQ ID NO: 2 is A.
(2b) The type of nucleotide in the PRKCH gene located at position 16212 of the nucleotide sequence of SEQ ID NO: 2 is G.
(3b) The type of nucleotide in the PRKCH gene located at position 30981 of the nucleotide sequence of SEQ ID NO: 2 is A.
(4b) The type of nucleotide in the PRKCH gene located at position 32408 of the nucleotide sequence of SEQ ID NO: 2 is G.
(5b) The type of nucleotide in the PRKCH gene located at position 33463 of the nucleotide sequence of SEQ ID NO: 2 is G.
(6b) The type of nucleotide in the PRKCH gene located at position 34446 of the nucleotide sequence of SEQ ID NO: 2 is T.
(7b) The type of nucleotide in the PRKCH gene located at position 39322 of the nucleotide sequence of SEQ ID NO: 2 is T.
(8b) The type of nucleotide in the PRKCH gene located at position 39469 of the nucleotide sequence of SEQ ID NO: 2 is A.
(9b) The type of nucleotide in the PRKCH gene located at position 39471 of the nucleotide sequence of SEQ ID NO: 2 is C.
(10b) The type of nucleotide in the PRKCH gene located at position 49248 of the nucleotide sequence of SEQ ID NO: 2 is C.
(11b) The type of nucleotide in the PRKCH gene located at position 49367 of the nucleotide sequence of SEQ ID NO: 2 is G.
(12b) The type of nucleotide in the PRKCH gene located at position 52030 of the nucleotide sequence of SEQ ID NO: 2 is A.

In the methods of the present invention, the above-mentioned polymorphic mutations may be detected in one of the genomes (heterozygous); however, without particular limitation, they are preferably detected in both of the genomes (homozygous).

For example, when a subject is suitably determined to have a risk factor for arteriosclerotic diseases, the genotype in the PRKCH gene located at position 39469 of the nucleotide sequence of SEQ ID NO: 2 is AA (homozygous).

In the present invention, since the polymorphic sites may be strongly linked to their surrounding DNA regions, the testing method of the present invention can be carried out by detecting polymorphic mutations other than those described above that are present in such a strongly linked DNA block.

For the AGTRL1 gene, for example, the nucleotide of an “adjacent polymorphic site” (for example, a polymorphic site listed in Tables 1-1 to 1-6 above) is determined beforehand in a human subgroup that includes arteriosclerotic disease patients in which the nucleotide at the polymorphic sites are (1b) and (24b) as above.

Next, whether or not a subject has a risk factor for arteriosclerotic diseases can be tested by determining the nucleotides of the “adjacent polymorphic site” in the subject and comparing it with the previously-determined nucleotides. When the nucleotide of the subject is identical to the previously-determined nucleotide, the subject is determined to have a risk factor for arteriosclerotic diseases. The testing method of the present invention can determine whether or not a subject has a risk factor for arteriosclerotic diseases, and can be utilized, for example, in deciding courses of treatment and agent doses.

For example, in a human subgroups including persons suffering from arteriosclerotic diseases in which the nucleotide at a polymorphic site on the AGTRL1 gene at position 39268 in the nucleotide sequence of SEQ ID NO: 1 is A, the nucleotide at an adjacent polymorphic site, such as position 296, is determined. When the frequency that the nucleotide of this site is T is higher in the persons suffering from arteriosclerotic diseases than in persons who do not suffer from the diseases, a subject is tested for the nucleotide at the polymorphic site of position 1, and determined to have a risk factor of arteriosclerotic diseases if the nucleotide type of this site is also T.

As described above, the discovery of genetic regions associated with arteriosclerotic diseases by the present invention allows those skilled in the art to test the presence or absence of a risk factor for arteriosclerotic diseases without undue burden.

As the human genome analysis progresses, information on whole nucleotide sequences and polymorphisms such as SNPs, microsatellites, VNTRs, RFLPs has been enriched. While the genomic nucleotide sequences are now being revealed in detail, the current top concern is to analyze a relationship between a gene or a specific sequence and a function (phenotype such as disease or disease progression). One of promising solutions to this is a genetic statistical analysis using haplotypes.

Human chromosomes are present in pairs and each pair is derived from mother and father. The term “haplotype” refers to a combination of the genotypes in either one of the pair of an individual, and shows how gene loci are arranged on a paternally- or maternally-derived chromosome. A child inherits one chromosome each from the father and mother. Thus, if no recombination occurred at the time of gametogenesis, the genes on each chromosome would inevitably be transferred together to the child, namely, they would be linked to each other. However, since recombination does in fact occur upon meiosis, genes carried on one chromosome are not necessarily linked. Inversely, however, even when genetic recombination occurs, gene loci closely located in one chromosome are strongly linked.

When allele dependency is found by observing this phenomenon in a population, this is referred to as “linkage disequilibrium”. For example, when three gene loci are observed and no linkage disequilibrium is seen among them, there expected to be 2³ different haplotypes, and the frequency of each haplotype is predicted from the frequencies of the gene loci. However, when a linkage disequilibrium is seen, there are only less than 2³ haplotypes, and their frequencies are different from those predicted.

It has recently been demonstrated that haplotypes are useful for analyzing linkage disequilibrium (Genetic Epidemiology 23:221-233), and further studies have been made. As a result, it has been revealed that the genome has portions susceptible or insusceptible to recombination, and that the portions that are inherited from ancestry to progeny as single regions (regions specified by haplotypes) are common to human races (Science 226, 5576:2225-2229). Specifically, there are tightly linked DNA regions, and these regions are generally called DNA blocks. In the present invention, the presence or absence of a risk factor for arteriosclerotic diseases can also be tested by detecting the presence or absence of a DNA block that includes a polymorphic site of the present invention.

Namely, a preferred embodiment of the present invention provides a method of testing whether or not a subject has a risk factor for arteriosclerotic diseases, wherein the detection of the presence of a DNA block showing the following haplotype indicates that a subject has a risk factor for arteriosclerotic diseases:

(A) a haplotype in which nucleotides of the complementary strand at polymorphic sites of positions 39268, 39353, 41786, 42019, and 43406 in the nucleotide sequence of SEQ ID NO: 1 are A, G, A, G, and C, respectively, wherein the polymorphic sites are located in the AGTRL1 gene.

In the present invention, the term “DNA block” refers to a portion (region) which shows an intense linkage disequilibrium among gene loci. If a DNA block associated with arteriosclerotic diseases is found, the presence or absence of a risk factor for arteriosclerotic diseases can be tested by detecting the DNA block.

If (a DNA block showing) a haplotype associated with arteriosclerotic diseases is found, the presence or absence of a risk factor for arteriosclerotic diseases can be tested by detecting the haplotype. As a result of dedicated research, the present inventors successfully discovered haplotypes associated with the susceptibility to arteriosclerotic diseases.

Accordingly, the present invention provides methods of testing whether or not a subject has a risk factor for arteriosclerotic diseases, which includes the step of detecting (a DNA block showing) a haplotype which is associated with arteriosclerotic diseases and is present on the AGTRL1 gene.

The present method can determine whether or not a subject has a risk factor for arteriosclerotic diseases by detecting a “haplotype associated with arteriosclerotic diseases” in the subject. These determinations can be used, for example, in deciding courses of treatment.

The above-mentioned “(DNA block showing) haplotype associated with arteriosclerotic diseases” specifically includes the following (DNA block showing) haplotype: (A) a haplotype in which the nucleotides of the complementary strand at polymorphic sites on the AGTRL1 gene located at positions 39268, 39353, 41786, 42019, and 43406 of the nucleotide sequence of SEQ ID NO: 1 are A, G, A, G, and C, respectively.

A preferred embodiment of the above-mentioned method of the present invention is a method of testing whether or not a subject has a risk factor for arteriosclerotic diseases, which comprises the step of determining the type of nucleotides of linked polymorphic sites present in a DNA block showing the following haplotype:
(A) a haplotype in which the nucleotides in the complementary strand of the AGTRL1 gene located at polymorphic sites at positions 39268, 39353, 41786, 42019, and 43406 of the nucleotide sequence of SEQ ID NO: 1 are A, G, A, G, and C, respectively.

Specifically, even polymorphic sites other than those specifically described herein that are included in the above-mentioned DNA block and linked with the polymorphic sites of the present invention may be used for the testing method of the present invention.

A preferred embodiment of the above-mentioned method is a method for testing whether or not a subject has a risk factor for arteriosclerotic diseases, which comprises the steps of:
(a) determining the type of nucleotide at a polymorphic site in the AGTRL1 gene of the subject; and
(b) determining that the subject has a risk factor for arteriosclerotic diseases when the nucleotide determined in (a) is the same as the nucleotide at the aforementioned polymorphic site in an AGTRL1 gene that shows the haplotype of (A) described below:
(A) a haplotype in which the nucleotides in the complementary strand of the AGTRL1 gene at polymorphic sites located at positions 39268, 39353, 41786, 42019, and 43406 of the nucleotide sequence of SEQ ID NO: 1 are A, G, A, G, and C, respectively.

Examples of the polymorphic site in step (a) include the polymorphic sites listed in Tables 1-1 to 1-6 above. Preferred polymorphic sites are those on the AGTRL1 gene located at positions 1, 12541, 21545, 33051, 35365, 39268, 39353, 39370, 39474, 39553, 39665, 41786, 42019, 42509, 43029, 43406, 43663, 46786, 49764, 64276, 74482, 78162, 93492, and 102938 in the nucleotide sequence of SEQ ID NO: 1.

Preferably, the polymorphic site in step (a) above includes, for example, the following polymorphic site (1) or (2):

(1) a polymorphic site on the GRK5 gene located at position 34799 in the nucleotide sequence of SEQ ID NO: 1; and

(2) a polymorphic site on the GRK5 gene located at position 60001 in the nucleotide sequence of SEQ ID NO: 1.

The nucleotide at a polymorphic site of the present invention can be determined by one skilled in the art using various methods. For example, it can be determined by directly determining the nucleotide sequence of a DNA having a polymorphic site of the present invention.

Typically, a test sample to be subjected to the testing methods of the present invention is preferably a biological sample collected from a subject beforehand. The biological sample may include, for example, a DNA sample. In the present invention, such DNA samples can be prepared based on chromosomal DNA or RNA extracted from, for example, the blood, skin, oral mucosa, a tissue or cell collected or excised by surgery, or a body fluid collected for testing or such, of the subject.

Thus, in the methods of the present invention, a subject-derived biological sample (a biological sample obtained from the subject beforehand) is subjected as a test sample to the test.

One skilled in the art can prepare a biological sample suitably using known techniques. For example, DNA samples can be prepared, for example, by PCR using chromosomal DNA or RNA as a template and primers that hybridize to a DNA having a polymorphic site of the present invention.

Next, the nucleotide sequence of isolated DNA is determined. The nucleotide sequence of the isolated DNA can be easily determined by one skilled in the art using, for example, a DNA sequencer.

Variations in the nucleotides at the polymorphic sites of the present invention are normally already known. The phrase “determining the nucleotide” in the present invention does not necessarily mean determining whether the nucleotide at a certain polymorphic site is A, G, T, or C. For example, when the nucleotide variations at a certain polymorphic site are known to be A or G, it is only necessary to find that the nucleotide of the site is “not A” or “not G”.

There are a variety of known methods for determining the nucleotide of a polymorphic site whose nucleotide variations are known. The method for determining the nucleotide in the present invention is not particularly limited. For example, TaqMan PCR, AcycloPrime, and MALDI-TOF/MS are in practical use as analysis methods using PCR. In addition, the Invader method and RCA method are known as non-PCR-dependent methods for determining nucleotide types. The nucleotide can also be determined using DNA arrays. These methods will be briefly illustrated below. Any of these processes can be applied to the determination of the nucleotide of a polymorphic site in the present invention.

### [TaqMan PCR]

The principle of TaqMan PCR is as follows. TaqMan PCR is an analysis method using a TaqMan probe and a set of primers that can amplify a region containing an allele. The TaqMan probe is designed to hybridize with the region containing the allele, which is amplified by the set of primers.

When the TaqMan probe is allowed to hybridize with a target nucleotide sequence under a condition near the Tm of the TaqMan probe, the hybridization efficiency of the TaqMan probe is significantly lowered due to the difference in a single nucleotide. When PCR is conducted in the presence of the TaqMan probe, elongation from the primers reaches the hybridized TaqMan probe in due course. Then, the TaqMan probe is decomposed from its 5′ end by the 5′-3′ exonuclease activity of DNA polymerase. By labeling the TaqMan probe with a reporter dye and a quencher, the decomposition of the TaqMan probe can be traced as a change in fluorescent signal. Specifically, when the TaqMan probe is decomposed, the reporter dye is released away from the quencher and thereby generates a fluorescence signal. When the hybridization of the TaqMan probe is reduced due to the difference in a single nucleotide, the decomposition of the TaqMan probe does not proceed, and a fluorescence signal is not generated.

Multiple nucleotide types can be determined simultaneously by designing TaqMan probes corresponding to a polymorphism and further modifying them so that the decomposition of each probe produces a different signal. For example, 6-carboxy-fluorescein (FAM) and VIC are used as reporter dyes for TaqMan probes for allele A and allele B, respectively, in a given allele. The generation of fluorescence signals by the reporter dyes are inhibited by a quencher when the probes are not decomposed. When each probe hybridizes with the corresponding allele, a fluorescence signal is observed upon the hybridization. Specifically, when the signal of either FAM or VIC is more intense than the other, the allele is found to be a homozygote of allele A or allele B. On the other hand, when the allele is a heterozygote of allele A and allele B, the two signals are to be detected at substantially identical levels. By using the TaqMan PCR, PCR and the determination of nucleotide type can be simultaneously conducted using a genome as an analysis target without a time-consuming step like separation on a gel. Accordingly, the TaqMan PCR is useful as a method capable of determining nucleotide types of many subjects.

### [AcycloPrime]

AcycloPrime is also in practice use as a method of determining a nucleotide using PCR. AcycloPrime uses one pair of primers for genome amplification, and one primer for polymorphism detection. Initially, a genomic region containing a polymorphic site is amplified by PCR. This step is the same as a regular genomic PCR. Next, the resultant PCR product is annealed with a primer for detecting SNPs, and an elongation reaction is conducted. The primer for detecting SNPs is so designed as to be annealed with a region adjacent to the polymorphic site to be detected.

In this step, a nucleotide derivative (terminator), which is labeled with a fluorescence polarization dye and is blocked at its 3′-OH, is used as a nucleotide substrate for elongation. As a result, only one complementary nucleotide is incorporated at the nucleotide at a position corresponding to the polymorphic site, and the elongation reaction is terminated. The incorporation of the nucleotide derivative to the primer can be detected by an increase of fluorescence polarization (FP) due to the increase of molecular weight. When two labels having different wavelengths are used as fluorescence polarization dyes, it is possible to determine whether a particular SNP is either of two nucleotides. Since the level of fluorescence polarization can be quantified, a single analysis can also determine whether an allele is a homozygote or heterozygote.

### [MALDI-TOF/MS]

The nucleotide type can also be determined by analyzing a PCR product through MALDI-TOF/MS. The MALDI-TOF/MS can quantify molecular weights very accurately. Thus, it is used in a variety of fields as an analysis method that can distinguish a slight difference in the amino acid sequence of a protein and the nucleotide sequence of a DNA. To determine a nucleotide through MALDI-TOF/MS, a region containing an allele to be analyzed is initially amplified by PCR. Next, an amplified product is isolated, and the molecular weight thereof is measured using MALDI-TOF/MS. Since the nucleotide sequence of the allele is already known, the nucleotide sequence of the amplified product is uniquely determined based on the molecular weight.

The determination of a nucleotide using MALDI-TOF/MS requires the separation of a PCR product and such. However, this technique is expected to enable accurate determination of a nucleotide without using labeled primers and probes. This technique can also be applied to simultaneous detection of polymorphisms at multiple sites.

### [SNP-Specific Labeling Method Using Type IIs Restriction Enzymes]

Methods that can determine a nucleotide type more rapidly using PCR have been reported. For example, the nucleotide at a polymorphic site can be determined using a type IIs restriction enzyme. This method uses a primer having a type IIs restriction enzyme-recognition sequence in PCR. Common restriction enzymes (type II), which are used in gene recombination, recognize a specific nucleotide sequence and cleaves a specific site in the nucleotide sequence. In contrast, type IIs restriction enzymes recognize a specific nucleotide sequence and cleaves a site away from the recognized nucleotide sequence. The number of nucleotides between the recognized sequence and the cleaving site depends on each enzyme. Accordingly, the amplified product can be cleaved exactly at the polymorphic site by a type IIs restriction enzyme when a primer that contains a recognition sequence of the type IIs restriction enzyme is allowed to anneal at a position away from the polymorphic site by the specific number of nucleotides.

A cohesive end containing a SNP nucleotide is formed at an end of the amplified product cleaved by the type IIs restriction enzyme. Then, adaptors having a nucleotide sequence corresponding to the cohesive end of the amplified product are ligated. The adaptors include different nucleotide sequences containing nucleotides corresponding to polymorphic mutations, and they can be labeled with different fluorescent dyes in advance. Finally, the amplified product is labeled with a fluorescent dye corresponding to the nucleotide of the polymorphic site.

When PCR is conducted using a primer having a type IIs restriction enzyme-recognition sequence in combination with a capture primer, the amplified product can be fluorescently labeled and then immobilized to a solid phase using the capture primer. For example, when a biotin-labeled primer is used as the capture primer, the amplified product can be captured by avidin-linked beads. The nucleotide can be determined by tracing the fluorescence dye of the amplified product thus captured.

### [Determination of Nucleotide Type at Polymorphic Site Using Magnetic Fluorescence Beads]

There are also known techniques capable of analyzing plural alleles in parallel in a single reaction system. Analyzing multiple alleles in parallel is called “multiplexing”. In typing methods using fluorescent signals, fluorescent elements having different fluorescence wavelengths are necessary for multiplexing. However, not so many fluorescent elements are available in actual analyses. In contrast, when multiple fluorescent elements are mixed with resins or such, even limited kinds of fluorescent elements can yield various fluorescence signals distinguishable from each other. In addition, it is possible to prepare magnetically-separable beads that emit fluorescence by adding a magnetically-adsorbable component to the resins. Multiplex polymorphism typing using such magnetic fluorescent beads has been developed (Baiosaiensu To Baioindasutorii (Bioscience & Bioindustry), Vol. 60 No. 12, 821-824).

In the multiplex polymorphism typing using magnetic fluorescent beads, probes having at their end a nucleotide complementary to a polymorphic site of each allele are immobilized to the magnetic fluorescent beads. The two components are combined so that each allele corresponds to each magnetic fluorescent bead with a unique fluorescence signal. On the other hand, fluorescently-labeled oligoDNA having a nucleotide sequence complementary to an region on the allele that is adjacent to the complementary sequence hybridized by the probe immobilized on the magnetic fluorescent bead, is prepared.

A region containing the allele is amplified by asymmetric PCR, and hybridized with the magnetic fluorescent bead-immobilized probe and the fluorescently-labeled oligoDNA, and the two are then ligated. When the end of the magnetic fluorescent bead-immobilized probe has a nucleotide sequence complementary to the nucleotide at the polymorphic site, they are efficiently ligated. Inversely, when the terminal nucleotide is different due to a polymorphism, The efficiency of the ligation between the two is lowered. As a result, the fluorescently-labeled oligoDNA binds with each magnetic fluorescent bead only when the sample has a nucleotide complementary to that of the magnetic fluorescent bead.

The nucleotide is determined by magnetically recovering the magnetic fluorescent beads, and detecting the presence of fluorescently-labeled oligoDNA on the magnetic fluorescent beads. The fluorescence signal can be analyzed for each one of the magnetic fluorescent beads using a flow cytometer. Thus, when a number of different magnetic fluorescent beads are mixed, the signals can be easily separated. Namely, the “multiplexing”, in which a number of different polymorphic sites are analyzed in parallel in a single reaction vessel, is achieved.

### [Invader Method]

Non-PCR-dependent genotyping methods have also been in practical use. For example, the invader method achieves the determination of nucleotide types using only a special nuclease called “cleavase” and three different oligonucleotides: allele probe, invader probe, and FRET probe. Of these probes, only the FRET probe needs to be labeled.

The allele probe is designed to hybridize with a region adjacent to an allele to be detected. The 5′-side of the allele probe is linked with a flap having a nucleotide sequence not involved in hybridization. The allele probe has a structure such that it is hybridized with the 3′-side of a polymorphic site and is linked to the flap on the polymorphic site.

On the other hand, the invader probe includes a nucleotide sequence which hybridizes with the 5′-side of the polymorphic site. The nucleotide sequence of the invader probe is designed so that its 3′-end corresponds to the polymorphic site as a result of hybridization. The invader probe may have an arbitrary nucleotide at the position corresponding to the polymorphic site. Namely, the nucleotide sequences of the invader probe and the allele probe are designed so that they become adjacent to each other across the polymorphic site upon hybridization.

When the polymorphic site has a nucleotide complementary to the nucleotide sequence of the allele probe, both the invader probe and the allele probe hybridize with the allele and then form a structure in which the invader probe invades the nucleotide of the allele probe corresponding to the polymorphic site. In the oligonucleotides which have formed the invasion structure as above, the cleavase cleaves the strand that has been invaded. Since the cleavage occurs on the invasion structure, it results in removal of the flap of the allele probe. On the other hand, when the nucleotide of the polymorphic site is not complementary to the nucleotide of the allele probe, no competition occurs between the invader probe and the allele probe, and no invasion structure is formed. Accordingly, the flap is not cleaved by the cleavase.

The FRET probe is for detecting the flap thus separated. The FRET probe has a self-complementary sequence on its 5′-end side and forms a hairpin loop in which a single-stranded portion is present in its 3′-end side. The single-stranded portion in the 3′-end side of the FRET probe has a nucleotide sequence complementary to the flap, and the flap can hybridize with this portion. The nucleotide sequences of the flap and the FRET probe are designed so that the flap hybridizes with the FRET probe and forms a structure in which the 3′-end of the flap invades the 5′-end portion of the self-complementary sequence of the FRET probe. The cleavase recognizes and cleaves the invasion structure. By labeling the FRET probe with a reporter dye and a quencher, which are similar to those in the TaqMan PCR, at the positions that sandwich the region to be cleaved by the cleavase, the cleavage of the FRET probe can be detected as a change in fluorescence signal.

Theoretically, uncleaved flaps should also hybridize with the FRET probe. However, in fact, the FRET-binding efficiency is largely different between cleaved flaps and flaps in the form of allele probes. Accordingly, cleaved flaps can be specifically detected by using FRET probes.

To determine nucleotides based on the invader method, two different allele probes having nucleotide sequences complementary to allele A and allele B, respectively, may be prepared. In this case, the flaps of the two have different nucleotide sequences. By preparing two different FRET probes for detecting the flaps with distinguishable reporter dyes, nucleotides can be determined in the same manner as in TaqMan PCR.

An advantage of the invader process is that the labeling is necessary only to the oligonucleotide of the FRET probe. The oligonucleotide of the FRET probe may be the same regardless of nucleotide sequences to be detected. Accordingly, mass production is possible. On the other hand, the allele probe and the invader probe do not need to be labeled. After all, the reagents for genotyping can be produced at low cost.

### [RCA]

Non-PCR-dependent methods for determining nucleotide sequences include rolling circle amplification (RCA). The rolling circle amplification (RCA) is a process of amplifying DNA based on a reaction in which a DNA polymerase having a strand displacing activity synthesizes a long complementary strand using a cyclic single-stranded DNA as a template (Lizardri P M et al., Nature Genetics 19, 225, 1998). In the RCA, an amplification reaction is constituted using a primer which anneals to a cyclic DNA to initiate complementary strand synthesis, and a second primer which anneals to a long complementary strand formed by the former primer.

The RCA uses a DNA polymerase having a strand displacing activity. Accordingly, a double-stranded portion formed by the complementary strand synthesis is displaced as a result of a complementary strand synthesis reaction initiated by another primer annealed to a further 5′ region. For example, a complementary strand synthesis reaction using cyclic DNA as a template does not complete by one cycle. The complementary strand synthesis continues while displacing a previously synthesized complementary strand, and produces a long single-stranded DNA. Meanwhile, the second primer anneals to the long single-stranded DNA produced from the template cyclic DNA, and initiates a complementary strand synthesis. In the RCA method, since the single-stranded DNA is produced using a cyclic DNA as a template, it has repeated nucleotide sequence of the identical nucleotide sequence. Accordingly, the continuous production of a long single strand leads to continuous annealing of the second primer. As a result, single-stranded portions to which the primer can anneal are continuously produced without a degeneration step. DNA amplification is thus achieved.

When cyclic single-stranded DNAs necessary for RCA are prepared depending on the nucleotide types of polymorphic sites, the nucleotide types can be determined using RCA. To this end, a padlock probe, which is a single linear strand, is used. The padlock probe has at its 5′- and 3′-ends nucleotide sequences that are complementary to both sides of a polymorphic site to be detected. These nucleotide sequences are linked by a portion called “backbone”, which is composed of a special nucleotide sequence. If the polymorphic site has a nucleotide sequence complementary to the ends of the padlock probe, the ends that have hybridized to the allele can be ligated by a DNA ligase. As a result, the linear padlock probe is cyclized, and an RCA reaction is triggered. The efficiency of the DNA ligase reaction is significantly reduced when the ends to be ligated is not completely complementary. Accordingly, the nucleotide can be determined by detecting the presence or absence of the ligation using the RCA method.

The RCA method can amplify DNA, but does not yield a signal as it is. In addition, when only the presence or absence of amplification is used as an index, the reaction must be conducted for every allele to determine the nucleotide type. There are known methods in which these points are improved for nucleotide determination. For example, molecular beacons can be used to determine nucleotide types in a single tube based on the RCA method. The molecular beacon is a signal-generating probe using a fluorescent dye and a quencher as in the TaqMan method. The molecular beacon includes complementary nucleotide sequences at the 5′-end and 3′-end and forms a hairpin structure by itself. When the vicinities of the two ends are labeled with a fluorescent dye and a quencher, a fluorescence signal is not detectable from the molecular beacon forming a hairpin structure. The molecular beacon in which a part thereof is designed as a nucleotide sequence complementary to an RCA amplified product hybridizes to the RCA amplified product. The hairpin structure is resolved as a result of hybridization, and a fluorescence signal is produced.

An advantage of such molecular beacons is that a common nucleotide sequence can be used in the molecular beacons, regardless of subjects to be detected, by using the nucleotide sequence of the backbone portion of the padlock probe. When different backbone nucleotide sequences are used for different alleles, and two molecular beacons having different fluorescence wavelengths are used in combination, nucleotide types can be determined in a single tube. Since the cost for synthesizing fluorescently-labeled probes is high, it is an economical advantage that a common probe can be used regardless of subjects to be assayed.

These methods have been developed for rapid genotyping of a large quantity of samples. All methods but MALDI-TOF/MS generally require the preparation of labeled probes and such in any way. In contrast, nucleotide typing methods that do not depend on labeled probes and such have long been performed. Examples of such methods include methods based on restriction fragment length polymorphisms (RFLP) and the PCR-RFLP method.

The RFLP method is based on the fact that a mutation in a recognition site of a restriction enzyme, or an insertion or deletion of nucleotides in a DNA fragment yielded by restriction enzyme treatment, can be detected as a change in the size of the fragment formed after the restriction enzyme treatment. If there is a restriction enzyme that recognizes a nucleotide sequence having a polymorphism to be detected, the nucleotide at the polymorphic site can be identified according to the principle of RFLP.

Methods of detecting a difference in nucleotides using a change in the secondary structure of a DNA as an index are also known as methods requiring no labeled probe. PCR-SSCP is based on the fact that the secondary structures of single-stranded DNAs reflect differences in their nucleotide sequence (Cloning and polymerase chain reaction-single-strand conformation polymorphism analysis of anonymous Alu repeats on chromosome 11. Genomics. Jan. 1, 1992; 12(1): 139-146., Detection of p53 gene mutations in human brain tumors by single-strand conformation polymorphism analysis of polymerase chain reaction products. Oncogene. Aug. 1, 1991; 6(8): 1313-1318., Multiple fluorescence-based PCR-SSCP analysis with postlabeling., PCR Methods Appl. Apr. 1, 1995; 4(5): 275-282.). The PCR-SSCP method is conducted by the steps of dissociating a PCR product into single-stranded DNAs, and separating them on a non-denaturing gel. Since the mobility of DNA on the gel varies depending on the secondary structure of single-stranded DNA, a nucleotide difference at the polymorphic site can be detected as a difference in mobility.

Another example of the methods requiring no labeled probe is denaturant gradient gel electrophoresis (DGGE). DGGE is a method in which a mixture of DNA fragments is electrophoresed in a polyacrylamide gel with a gradient of denaturant concentration, and the DNA fragments are separated depending on the difference in their instability. When an unstable DNA fragment having a mismatch moves to a position at a certain denaturant concentration in the gel, a DNA sequence around the mismatch is partially dissociated to single strands due to its instability. The mobility of the partially denatured DNA fragment is much slower and differs from that of a complete double-stranded DNA having no dissociated portion. Thus, the two fragments can be separated from each other.

Specifically, a region having a polymorphic site is initially amplified by PCR or such. A probe DNA with a known nucleotide sequence is allowed to hybridize with the amplified product to form a double-strand. This is electrophoresed in a polyacrylamide gel with a gradually increasing concentration of a denaturant such as urea, and is compared with a control. A DNA fragment that has a mismatch as a result of hybridization with the probe DNA is dissociated to single strands in a portion at a lower concentration of the denaturant and then shows a markedly slow mobility. The presence or absence of the mismatch can be detected by detecting the difference in mobility thus occurred.

In addition, nucleotides can also be determined by using DNA arrays (Saibo Kogaku Bessatsu “DNA Maikuroarei To Saishin PCR-ho” (Cell Technology Suppl., “DNA Microarray and Latest PCR Techniques”), Shujunsha Co., Ltd., published on Apr. 20, 2000, pp. 97-103 “OligoDNA Chippu Niyoru SNP No Bunseki (SNP Analyses with OligoDNA Chips)”, Shin-ichi Kajie). In a DNA array, a sample DNA (or RNA) is allowed to hybridize with many probes arrayed in one plate, and the plate is then scanned to detect the hybridizations with the probes. Since reactions on many probes can be observed simultaneously, such DNA arrays are useful, for example, to analyze many polymorphic sites simultaneously.

DNA arrays generally are generally composed of thousands of nucleotides densely printed on a substrate. Normally, these DNAs are printed on a surface layer of a non-porous substrate. The surface layer of the substrate is generally made of glass, but a porous membrane such as a nitrocellulose membrane can also be used.

In the present invention, an example of techniques for immobilizing (arraying) nucleotides is oligonucleotide-based arrays developed by Affymetrix, Inc. In oligonucleotide arrays, the oligonucleotides are generally synthesized in vitro. For example, in situ oligonucleotide synthesis methods are known, such as photolithographic techniques (Affymetrix, Inc.), and inkjet techniques for immobilizing chemical compounds (Rosetta Inpharmatics LLC). Any of these techniques can be used for preparing substrates used in the present invention.

The oligonucleotides are composed of nucleotide sequences complementary to regions containing SNPs to be detected. The length of nucleotide probes to bind with the substrate is, when they are oligonucleotides, generally 10 to 100 bases, preferably 10 to 50 bases, and more preferably 15 to 25 bases. In addition, the DNA array method generally uses a mismatch (MM) probe to avoid errors due to cross-hybridization (non-specific hybridization). The mismatch probe constitutes a pair with an oligonucleotide having a nucleotide sequence completely complementary to a target nucleotide sequence. The oligonucleotide that is composed of a nucleotide sequence completely complementary to a target nucleotide sequence is called a perfect match (PM) probe. The influence of cross-hybridization can be reduced by erasing signals observed with the mismatch probe in data analyzing processes.

Samples for genotyping by the DNA array method can be prepared based on biological samples collected from subjects, according to known methods to one skilled in the art. The biological samples are not particularly limited. For example, DNA samples can be prepared from chromosomal DNA extracted from tissues or cells such as blood, peripheral blood leucocytes, skin, or oral mucosa; tears, saliva, urine, faeces, or hair, of the subjects. A particular region of the chromosomal DNA is amplified using primers for amplifying a region having a polymorphic site to be determined. In this step, multiple regions can be amplified simultaneously by using multiplex PCR. The multiplex PCR is a PCR method in which multiple sets of primers are used in one reaction solution. The multiplex PCR is useful to analyze multiple polymorphic sites.

In the DNA array method, a DNA sample is amplified by PCR and the amplified product is labeled. Labeled primers are used for labeling the amplified product. For example, initially, a genomic DNA is amplified by PCR with a set of primers specific to a region containing a polymorphic site. Next, a biotin-labeled DNA is synthesized by labeling PCR using a biotin-labeled primer. The biotin-labeled DNA thus synthesized is allowed to hybridize with oligonucleotide probes on a chip. The reaction solution and conditions of hybridization can be suitably adjusted according to conditions such as the length of nucleotide probes immobilized on the substrate, and reaction temperature. One skilled in the art can appropriately design hybridization conditions. Fluorescent dye-labeled avidin is added to detect a hybridized DNA. The array is analyzed with a scanner, and the presence or absence of hybridization is detected using fluorescence as an index.

The above-mentioned process will be illustrated more specifically. Initially, DNA containing a polymorphic site of the present invention is prepared, and a solid phase to which nucleotide probes are immobilized is obtained. Next, the DNA is contacted with the solid phase. Further, DNA hybridized with the nucleotide probes immobilized on the solid phase is detected to determine the nucleotide type at the polymorphic site of the present invention.

The term “solid phase” as used in the context of the present invention refers to a material to which a nucleotide can be immobilized. The solid phase used in the present invention is not particularly limited, so long as a nucleotide can be immobilized thereto. Specific examples thereof include solid phases including microplate wells, plastic beads, magnetic particles, and substrates. In the present invention, a substrate generally used in DNA array techniques is preferably used as a solid phase. The term “substrate” in the present invention means a plate-like material to which a nucleotide can be immobilized. In the present invention, the nucleotide includes oligonucleotides and polynucleotides.

In addition to the above-mentioned methods, the allele-specific oligonucleotide (ASO) hybridization method can be used to detect a nucleotide at a specific site. The allele-specific oligonucleotide (ASO) is composed of a nucleotide sequence that hybridizes with a region containing a polymorphic site to be detected. When the ASO is hybridized with a sample DNA and a mismatch occurs at the polymorphic site due to a polymorphism, the hybridization efficiency is lowered. The mismatch can be detected by Southern blotting or a method using a special fluorescent reagent which is quenched when intercalating to a gap in a hybrid. The mismatch can also be detected by the ribonuclease A mismatch cleavage method.

Oligonucleotides that hybridize with DNA containing a polymorphic site of the present invention and have at least a 15-nucleotide chain length are usable as a reagent (testing agent) for testing whether a subject has a risk factor for arteriosclerotic diseases. These are used in tests in which the gene expression is used as an index, or tests using gene polymorphism as an index.

The oligonucleotides specifically hybridize with DNA having a polymorphic site of the present invention. The phrase “specifically hybridizes” as used herein means that significant cross-hybridizations do not occur with DNAs encoding other proteins under usual hybridization conditions, preferably under stringent hybridization conditions (for example, conditions stated by Sambrook et al., Molecular Cloning, Cold Spring Harbour Laboratory Press, New York, USA, 2nd Ed. 1989). When specific hybridization is possible, the oligonucleotide does not need to be completely complementary to the nucleotide sequence containing a polymorphic sites of the present invention in a gene to be detected or in an adjacent DNA region of the gene.

Examples of hybridization conditions in the present invention include “2×SSC, 0.1% SDS, 50° C.”, “2×SSC, 0.1% SDS, 42° C.”, and “1×SSC, 0.1% SDS, 37° C.”; and as more stringent conditions, “2×SSC, 0.1% SDS, 65° C.”, “0.5×SSC, 0.1% SDS, 42° C.”, and “0.2×SSC, 0.1% SDS, 65° C.”. More specifically, as a process using the Rapid-hyb Buffer (Amersham Life Science), hybridization can be carried out by conducting prehybridization at 68° C. for 30 minutes or more; adding probes to form hybrids while maintaining at 68° C. for one hour or more; thereafter carrying out three times of washing in 2×SSC, 0.1% SDS at room temperature for 20 minutes; subsequently carrying out three times of washing in 1×SSC, 0.1% SDS at 37° C. for 20 minutes; and finally carrying out two times of washing in 1×SSC, 0.1% SDS at 50° C. for 20 minutes. Hybridization can also be conducted, for example, by carrying out prehybridization in the Expresshyb Hybridization Solution (CLONTECH) at 55° C. for 30 minutes or more; adding labeled probes and incubating at 37° C. to 55° C. for one hour or more; carrying out three times of washing in 2×SSC, 0.1% SDS at room temperature for 20 minutes; and carrying out washing once in 1×SSC, 0.1% SDS at 37° C. for 20 minutes. More stringent conditions are available, for example, by setting the temperatures of prehybridization, hybridization and/or the second washing at higher levels. For example, the temperatures of prehybridization and hybridization can be set to 60° C., and, as more stringent conditions, to 68° C. One skilled in the art can set the conditions in consideration of, in addition to these conditions such as salt concentrations of buffers and temperatures, other conditions such as concentrations, lengths, and nucleotide sequence structures of probes, and reaction times.

The oligonucleotide can be used as a probe or primer in the above testing method according to the present invention. The length of the oligonucleotide, if used as a primer, is generally 15 bp to 100 bp, and preferably 17 bp to 30 bp. The primer is not particularly limited, so long as it can amplify at least a part of a DNA having a polymorphic site of the present invention.

The present invention provides a primer for amplifying a region having a polymorphic site of the present invention, and a probe that hybridizes with a DNA region containing the polymorphic site.

Such primers for amplifying a region having the polymorphic site of the present invention also include primers that can initiate complementary strand synthesis toward the polymorphic site using as a template a DNA containing the polymorphic site. The primers can be described as primers for imparting an origin of replication to the 3′ side of a polymorphic site in a DNA containing the polymorphic site. The distance between the polymorphic site and the region with which the primer hybridizes is arbitrary. As the distance between them, a suitable number of nucleotides can be selected according to the technique for analyzing the nucleotide at the polymorphic site. For example, when the primer is a primer for analysis using DNA chips, it is possible to design the primer to yield an amplification product having a length of 20 to 500, generally 50 to 200 nucleotides as a region that includes the polymorphic site. One skilled in the art can design a primer according to the analysis technique based on nucleotide sequence information on an adjacent DNA region containing the polymorphic site. The nucleotide sequence constituting the primer according to the present invention can be not only a nucleotide sequence completely complementary to a genomic nucleotide sequence but also suitable modifications thereof.

The primer according to the present invention can be added with arbitrary nucleotide sequences, in addition to nucleotide sequences complementary to a genomic nucleotide sequence. For example, primers added with a type IIs restriction enzyme-recognition sequence are used in primers for a method of analyzing polymorphisms using a type IIs restriction enzyme. Such primers with modified nucleotide sequences are also included in the primers for use in the present invention. In addition, the primers for use in the present invention can be modified. For example, primers labeled with fluorescent substance or substance with binding affinity, such as biotin or digoxin, are used in various genotyping methods. These modified primers are also included within the present invention.

On the other hand, the phrase “probe that hybridizes with a region containing a polymorphic site” in the present invention refers to probes that can hybridize with a polynucleotide that has a nucleotide sequence of a region containing a polymorphic site. More specifically, a probe having a polymorphic site in its nucleotide sequence is a preferably probe for use in the present invention. Alternatively, a probe may be designed to have an end corresponding to a nucleotide (base) adjacent to a polymorphic site in some methods of analyzing the nucleotide at the polymorphic site. Accordingly, a preferred probe in the present invention includes a probe which does not contain a polymorphic site in its nucleotide sequence but contains a nucleotide sequence complementary to a neighboring region to the polymorphic site.

In other words, a probe that can hybridize with a polymorphic site of the present invention on a genomic DNA or with a neighboring site to the polymorphic site is preferable as a probe for use in the present invention. Such probes according to the present invention can be subjected to alternations in nucleotide sequence, addition of nucleotide sequence, or modification, as in the primers. For example, probes for use in the Invader process are added with a nucleotide sequence which constitutes a flap and does not relate to the genome. Such probes are also included in the probe of the present invention, so long as they hybridize with a region containing a polymorphic site. The nucleotide sequence constituting a probe according to the present invention can be designed according to the analysis method based on nucleotide sequence of a DNA region neighboring the present invention's polymorphic site on the genome.

Primers or probes according to the present invention can be synthesized by arbitrary methods based on the nucleotide sequence constituting the same. In a primer or probe according to the present invention, the length of a nucleotide sequence complementary to a genomic DNA is usually 15 to 100, generally 15 to 50, and usually 15 to 30. Procedures of synthesizing oligonucleotides having a given nucleotide sequence, based on the given nucleotide sequence, have been known. In addition, it is possible in the synthesis of oligonucleotides to introduce arbitrary modifications to the oligonucleotides using nucleotide derivatives modified with, for example, fluorescent dye or biotin. Procedures of binding synthesized oligonucleotides with, for example, fluorescent dye has also been known.

The oligonucleotides according to the present invention, when used as a probe, may be suitably labeled with, for example, a radioisotope or a nonradioactive compound. When used as a primer, it is possible, for example, to design its structure so that the 3′-end region of the oligonucleotide is complementary to a target sequence and so that a restriction enzyme-recognition sequence, a tag, or the like is added to the 5′-end of the oligonucleotide. Such a polynucleotide having a nucleotide sequence composed of at least 15 successive nucleotides can hybridize with an mRNA of the AGTRL1 or PRKCH gene.

The oligonucleotides according to the present invention may contain a nucleotide (base) other than naturally-occurring nucleotides, according to necessity, the examples of which include 4-acetylcytidine, 5-(carboxyhydroxymethyl)uridine, 2′-O-methylcytidine, 5-carboxymethylaminomethyl-2-thiouridine, 5-carboxymethylaminomethyluridine, dihydrouridine, 2′-O-methylpseudouridine, β-D-galactosylqueuosine, 2′-O-methylguanosine, inosine, N6-isopentenyladenosine, 1-methyladenosine, 1-methylpseudouridine, 1-methylguanosine, 1-methylinosine, 2,2-dimethylguanosine, 2-methyladenosine, 2-methylguanosine, 3-methylcytidine, 5-methylcytidine, N6-methyladenosine, 7-methylguanosine, 5-methylaminomethyluridine, 5-methoxyaminomethyl-2-thiouridine, βP-D-mannosylqueuosine, 5-methoxycarbonylmethyl-2-thiouridine, 5-methoxycarbonylmethyluridine, 5-methoxyuridine, 2-methylthio-N6-isopentenyladenosine, N-((9-β-D-ribofuranosyl-2-methylriopurine-6-yl)carbamoyl)threonine, N-((9-β-D-ribofuranosylpurine-6-yl)N-methylcarbamoyl)threonine, uridine-5-oxyacetic acid-methylester, uridine-5-oxyacetic acid, wybutoxosine, pseudouridine, queuosine, 2-thiocytidine, 5-methyl-2-thiouridine, 2-thiouridine, 4-thiouridine, 5-methyluridine, N-((9-β-D-ribofuranosylpurine-6-yl)carbamoyl)threonine, 2′-O-methyl-5-methyluridine, 2′-O-methyluridine, wybutosine, and 3-(3-amino-3-carboxypropyl)uridine.

The present invention also provides a reagent (testing agent) for use in a method of testing whether the subject has a risk factor for arteriosclerotic diseases. The reagent according to the present invention includes the primer and/or probe according to the present invention as described above. In testing whether the subject has a risk factor for arteriosclerotic diseases, a primer and/or a probe for amplifying a DNA containing a polymorphic site of the invention is used.

The reagent according to the present invention can be combined with, for example, various enzymes, enzyme substrates, and buffers according to the nucleotide typing process. Examples of the enzymes include enzymes necessary for the various analysis processes exemplified as the nucleotide typing process, such as DNA polymerase, DNA ligase, or IIs restriction enzyme. As the buffer, suitable buffers for maintaining the activity of the enzymes used in the analysis is suitably selected. As the enzyme substrate, for example, a substrate for complementary strand synthesis is used.

A control composed of a known nucleotide at the polymorphic site may be attached to the reagent of the present invention. The control can be a genome or a genomic fragment whose nucleotide type at the polymorphic site is already identified. The genome may be an extract from a cell; and a cell or cell fraction can also be used. When cells are used as the control, it is possible to prove that the extraction procedure of a genomic DNA is conducted suitably, based on the result of the control. Alternatively, the DNA having a nucleotide sequence that includes the polymorphic site may be used as the control. Specifically, a YAC vector and a BAC vector containing a DNA derived from a genome and having a known nucleotide type at the polymorphic site of the present invention are useful as the control. It is also possible to use, as the control, a vector prepared by splicing only several hundreds of bases corresponding to the polymorphic site and inserting the bases into a vector.

Another embodiment of the reagent of the present invention is a reagent for testing whether the subject has a risk factor for arteriosclerotic diseases, which includes a nucleotide probe immobilized on the solid phase, in which the nucleotide probe hybridizes with a DNA comprising a polymorphic site of the present invention.

These reagents find utility in the context of tests that use an index the polymorphic site of the present invention. Methods for preparing these are as mentioned above.

A preferred embodiment of the present invention relates to a method for testing whether the subject has a risk factor for arteriosclerotic diseases, which includes the step of detecting a transcription or translation product of the AGTRL1 or PRKCH gene.

Accordingly, oligonucleotides usable as a probe in the detection of a transcription product of the AGTRL1 or PRKCH gene in the testing method, such as oligonucleotides that hybridize with the transcription product of the AGTRL1 or PRKCH gene, is one example of testing reagents according to the present invention.

In addition, antibodies that recognize an AGTRL1 or PRKCH protein (anti-AGTRL1 protein antibody or anti-PRKCH protein antibody) and is usable in the detection of a translation product of the AGTRL1 or PRKCH gene in the testing method is also a preferred example of testing reagents according to the present invention.

The present inventors elucidated that enhanced expression of the AGTRL1 gene is associated with arteriosclerotic diseases. They also showed that autophosphorylation of the PRKCH protein is associated with arteriosclerotic diseases such as cerebral infarction. Therefore, substances that suppress the expression of the AGTRL1 or PRKCH gene or the function (activity) of a protein encoded by the gene may become therapeutic or preventive agents for arteriosclerotic diseases.

The present invention provides agents for treating arteriosclerotic diseases that include, as an active ingredient, substances that inhibit the expression of AGTRL1 or PRKCH protein, or substances that inhibit the function (activity) of a protein encoded by the gene (AGTRL1 or PRKCH protein).

In a preferred embodiment, the present invention initially provides agents for treating arteriosclerotic diseases (agent and/or pharmaceutical composition for treating or preventing arteriosclerotic diseases), which includes, as an active ingredient, an expression inhibitor for the expression of AGTRL1 or PRKCH gene.

The AGTRL1 or PRKCH gene expression inhibitor in the present invention includes, for example, substances that inhibit the transcription of AGTRL1 or PRKCH or the translation of the transcription product. Examples of preferred embodiments of the above expression inhibitors in the present invention include compounds (nucleic acids) selected from the group consisting of (a) to (c) below:

(a) an antisense nucleic acid to a transcription product of the AGTRL1 or PRKCH gene or a part thereof;

(b) a nucleic acid having ribozyme activity of specifically cleaving a transcription product of the AGTRL1 or PRKCH gene; and

(c) a nucleic acid having an action of inhibiting expression of the AGTRL1 or PRKCH gene through an RNA interference effect.

The term “nucleic acid” in the present invention means an RNA or a DNA. The “nucleic acid” in the present invention also includes chemically synthesized nucleic acid analogues such as a so-called PNA (peptide nucleic acid). PNA has a three-dimensional structure closely resembling that of a nucleic acid, and has a polyamide skeleton having glycine units instead of the pentose-phosphate skeleton of the nucleic acid as a basic skeleton structure.

As methods for inhibiting the expression of specific endogenous genes, a method using an antisense technique is well known to one skilled in the art. As actions for an antisense nucleic acid to inhibit the expression of a target gene, there are multiple factors as mentioned below. Specifically, examples of such actions include: inhibition of transcription initiation due to triplex formation; transcription inhibition due to hybridization with a site in which a local open loop structure is formed by RNA polymerase; transcription inhibition due to hybridization with RNA which is under synthesis; inhibition of splicing due to hybridization with an intron-exon junction; inhibition of splicing due to hybridization with a spliceosome-forming site; inhibition of translocation from the nucleus to the cytoplasm due to hybridization with mRNA; inhibition of splicing due to hybridization with a capping site or a poly(A) addition site; inhibition of translation initiation due to hybridization with translation initiation factor-binding site; inhibition of translation due to hybridization with a ribosome-binding site in the vicinity of initiation codon; inhibition of peptide chain elongation due to hybridization with a coding region or polysome-binding site of mRNA; and inhibition of gene expression due to hybridization with an interaction site between nucleic acid and protein. Thus, antisense nucleic acid inhibits the expression of a target gene by inhibiting various processes, such as transcription, splicing, or translation (Hirashima and Inoue, Shin Seikagaku Jikken Koza 2 Kakusan IV Idenshi No Fukusei To Hatsugen (Experimental Biochemistry, New Ed., 2, Nucleic Acid IV, Replication and Expression of Genes), edited by The Japanese Biochemical Society, TOKYO KAGAKU DOJIN CO., LTD., 1993, 319-347.).

The antisense nucleic acid for use in the present invention may inhibit the expression of AGTRL1 or PRKCH gene by any of the above-mentioned actions. As one embodiment, it may be effective for inhibiting gene translation to design an antisense sequence to be complementary to a noncoding region adjacent to the 5′-end of the mRNA of the AGTRL1 or PRKCH gene. In addition, sequences complementary to a coding region or to a noncoding region at the 3′ side can also be used. Thus, antisense nucleic acids for use in the present invention also include nucleic acids having an antisense sequence to not only a sequence of a coding region of the AGTRL1 or PRKCH gene, but also to a sequence of a noncoding region of the AGTRL1 or PRKCH gene. The antisense nucleic acid to be used is linked downstream of a suitable promoter, and a sequence containing a transcription terminator signal is preferably linked to the 3′ side. Nucleic acids thus prepared can be used to transform a desired animal according to known methods. The sequence of the antisense nucleic acid is preferably a sequence complementary to endogenous AGTRL1 or PRKCH gene of the animal to be transformed, or a part thereof. However, the sequence may not be completely complementary, so long as the gene expression can be effectively inhibited. A transcribed RNA has a complementarity of preferably 90% or more, and most preferably 95% or more to a transcription product of the target gene. To effectively inhibit the expression of the target gene (AGTRL1 or PRKCH) using an antisense nucleic acid, the length of the antisense nucleic acid is preferably at least 15 nucleotides or more and less than 25 nucleotides, but the length of the antisense nucleic acid for use in the present invention is not necessarily limited thereto.

The antisense for use in the present invention is not particularly limited; however, it can be prepared, for example, based on the nucleotide sequence of SEQ ID NO: 1 or 2.

Expression of AGTRL1 or PRKCH gene may be inhibited by using ribozymes, or the DNA encoding ribozymes. The term “ribozyme” refers to RNA molecules having catalytic activity. There are ribozymes having a variety of activities. As a result of studies focusing, of such ribozymes, on ribozymes as enzymes that cleave RNA, it becomes possible to design ribozymes that site-specifically cleave RNA. Of ribozymes, some have a size of 400 nucleotides or more, such as Group I intron-type ribozymes and M1 RNA belonging to RNase P, others have an active domain of about 40 nucleotides, called hammerhead or hairpin ribozymes (Makoto Koizumi and Eiko Otsuka, Tanpakushitsu, Kakusan Koso (PROTEIN, NUCLEIC ACID, AND ENZYME), 1990, 35, 2191.).

For example, self-cleaving domain of the hammerhead ribozyme cleaves the 3′-side of C15 in a sequence of G13U14C15; the formation of a base pair of U14 and A9 is considered to be important to its activity; and cleavage can occur at A15 or U15 instead of C15 (Koizumi, M. et al., FEBS Lett, 1988, 228, 228.). By designing a ribozyme having a substrate-binding site complementary to an RNA sequence adjacent to a target site, an RNA-cleaving ribozyme that recognizes UC, UU, or UA in the target RNA and acts in a restriction enzyme-like manner can be prepared (Koizumi, M. et al., FEBS Lett, 1988, 239, 285., Makoto Koizumi and Eiko Otsuka, Tanpakushitsu, Kakusan Koso (PROTEIN, NUCLEIC ACID, AND ENZYME), 1990, 35, 2191., Koizumi, M. et al., Nucl Acids Res, 1989, 17, 7059.).

The hairpin ribozyme is also useful for the objects of the present invention. This ribozyme is found, for example, in the minus strand of satellite RNA of tobacco ringspot virus (Buzayan, J M., Nature, 1986, 323, 349.). There has been shown that target-specific RNA-cleaving ribozyme can also be prepared from hairpin ribozyme (Kikuchi, Y. & Sasaki, N., Nucl Acids Res, 1991, 19, 6751., Yo Kikuchi, Kagaku To Seibutsu (Chemistry and Biology), 1992, 30, 112.). Thus, by specifically cleaving transcription products of the AGTRL1 or PRKCH gene in the present invention using ribozymes, expression of the gene can be suppressed.

In addition, suppression of the expression of endogenous gene can also be conducted through RNA interference (RNAi) using double-stranded RNA containing a sequence identical or similar to the sequence of a target gene. Nucleic acids having inhibitory action due to RNAi effect for use in the present invention is generally also called as siRNA. RNAi is a phenomenon in which a double-stranded RNA is introduced into, for example, a cell to induce the destruction of a mRNA of the target gene to thereby suppress the expression of the target gene, in which the double-stranded RNA contains a sense RNA having a sequence identical to the mRNA of the target gene and an antisense RNA having a sequence complementary thereto. Since RNAi can thus suppress the expression of the target gene, it receives attention as an easy and convenient gene knock-out method as an alternative to conventional complicated, inefficient gene destroying techniques through homologous recombination; or as a method applicable to gene therapies. RNA for use in RNAi does not necessarily need to be completely identical to AGTRL1 or PRKCH gene or a partial region of the gene, but preferably has complete homology.

A preferred embodiment of the above nucleic acid (c) in the present invention includes double-stranded RNA (siRNA) having an RNAi (RNA interference) effect to AGTRL1 or PRKCH gene. More specifically, it includes double-stranded RNA (siRNA) composed of a sense RNA and an antisense RNA to a partial sequence of the nucleotide sequence of SEQ ID NO: 1.

Although the details of the RNAi mechanism remains unrevealed, it is considered that an enzyme called DICER (one type of the RNase III nuclease family) comes in contact with the double-stranded RNA, and the double-stranded RNA is decomposed into small fragments called small interfering RNAs or siRNAs. Double-stranded RNAs having RNAi effect in the present invention also include double-stranded RNA before the decomposition by DICER. Namely, even RNAs with such a long strand as not to exhibit RNAi effect in its original length is expected to be decomposed in a cell into siRNAs having RNAi effect, and thus, the length of the double-stranded RNA in the present invention is not particularly limited.

For example, a long double-stranded RNA corresponding to a region of the full-length or substantially full-length of the mRNA of AGTRL1 or PRKCH gene of the present invention may be decomposed beforehand with DICER, and the decomposition product thereof may be used as a therapeutic agent for arteriosclerotic diseases. The decomposition product is expected to contain double-stranded RNA molecules having RNAi effect (siRNA). Following to this method, there is no need to particularly select a region on mRNA expected to have RNAi effect. Thus, it is not always necessary to precisely specify a region on the mRNA of AGTRL1 or PRKCH gene of the present invention which has RNAi effect.

Of the above described RNA molecules, those that have a conformation in which one end is closed, such as an siRNA having hairpin structure (shRNA), is also included within the present invention. Namely, single stranded RNA molecules that can intramolecularly form a double-stranded RNA structure is also included within the present invention.

The above “double-stranded RNA that can be suppressed through RNAi effect” for use in the present invention can be appropriately produced by one skilled in the art based on the nucleotide sequence of the AGTRL1 or PRKCH gene of the present invention as a target for the double-stranded RNA. For example, the double-stranded RNA for use in the present invention can be produced based on the nucleotide sequence of SEQ ID NO: 1 or 2. Namely, based on the nucleotide sequence of SEQ ID NO: 1 or 2, it is within the range of usual trials for one skilled in the art to suitably select an arbitrary consecutive RNA region of the mRNA, which is the transcription product of the sequence, and prepare a double-stranded RNA corresponding to the selected region. In addition, the selection of siRNA sequence having stronger RNAi effect from among the mRNA sequences, which are the transcription products of the sequence, can be suitably conducted by one skilled in the art according to known methods. If one of the two strands (for example, the nucleotide sequence of SEQ ID NO: 1 or 2) has been identified, one skilled in the art can easily know the nucleotide sequence of the other strand (complementary strand). The siRNA can be suitably produced by one skilled in the art using commercially available nucleic acid synthesizers. In addition, a general contract synthesis for customers can be used for the synthesis of desired RNA.

A DNA (vector) that can express the above-mentioned RNA of the present invention is also included in the preferred embodiments of the compounds that can suppress the expression of AGTRL1 or PRKCH gene of the present invention. For example, a DNA (vector) that can express the above double-stranded RNA according to the present invention is a DNA having a structure in which a DNA encoding one strand of the double-stranded RNA, and a DNA encoding the other strand of the double-stranded RNA are linked with promoters so that they can each be expressed. The above mentioned DNA of the present invention can be suitably produced by one skilled in the art according to general genetic engineering techniques. More specifically, an expression vector for use in the present invention can be prepared by suitably inserting a DNA encoding the RNA of the present invention into various known expression vectors.

The expression inhibitors of present invention further include compounds that suppress expression of the AGTRL1 or PRKCH gene by binding with, for example, the expression regulatory region (for example, a promoter region) of the AGTRL1 or PRKCH gene. The compound may be obtained, for example, by a screening method using a promoter DNA fragment of the AGTRL1 or PRKCH gene and using the binding activity with the DNA fragment as an index. One skilled in the art can suitably determine whether a desired compound suppresses expression of the AGTRL1 or PRKCH gene of the present invention according to known methods, such as a reporter assay.

In addition, the present inventors demonstrated that expression of the AGTRL1 gene is enhanced by polymorphic mutations “SNP4” and “SNP9” in the AGTRL1 gene.

These polymorphic mutations exist in the DNA region that binds to Sp1, which is a transcriptional regulator. These polymorphic mutations change the binding activity of the Sp1 transcription factor towards the DNA region and thereby enhance the expression of the AGTRL1 gene.

Therefore, substances that lower the binding activity between the DNA region of the AGTRL1 gene containing the above-mentioned polymorphism and the Sp1 transcription factor are considered to suppress the transcription of the AGTRL1 gene, and may be one of the preferred embodiments of the expression-suppressing substances of the present invention mentioned above. Examples of the above-mentioned DNA regions include DNA regions comprising the polymorphic site “SNP4” or “SNP9”.

Furthermore, polynucleotides comprising a DNA sequence that contains a polymorphic mutation of the present invention and has an altered binding activity with the Sp1 transcription factor are useful and can be used suitably, for example, for methods of screening for pharmaceutical agents for treating or preventing atherosclerotic diseases to be described later. For example, the polynucleotides of (a) and (b) below can be used for the purpose of screening for therapeutic agents for arteriosclerotic diseases:
(a) partial polynucleotide fragments of the DNA sequence of SEQ ID NO: 1 which comprises the polymorphic sites located at position 42509 or 39353; and
(b) polynucleotides comprising a nucleotide sequence having one or more nucleotide additions, deletions, or substitutions in the sequence of the polynucleotide of (a), in which the binding ability with Sp1 transcription factor is enhanced.

Furthermore, polypeptides comprising a protein encoded by a DNA carrying a polymorphic mutation of the present invention, in which the autophosphorylation activity of the PRKCH protein has been enhanced can be suitably used, for example, in methods of screening for pharmaceutical agents for treating or preventing arteriosclerotic diseases to be described later. For example, the polypeptides of (a) and (b) below can be used for the purpose of screening for therapeutic agents for arteriosclerotic diseases:
(a) a full-length polypeptide or fragments of the PRKCH protein, in which valine at position 374 in the amino acid sequence of the PRKCH protein is substituted with isoleucine; and
(b) a polypeptide comprising an amino acid having one or more amino acid additions, deletions, or substitutions in the sequence of the polypeptide of (a), in which the autophosphorylation activity is enhanced.

The present invention also provides therapeutic agents for arteriosclerotic diseases which comprise as an active ingredient a substance that suppresses the function of the AGTRL1 or PRKCH protein.

Substances that suppress the function of the AGTRL1 or PRKCH protein of the present invention include, for example, the compounds of (a) and (b) below:
(a) antibodies that bind to the AGTRL1 or PRKCH protein; and
(b) low-molecular weight compounds that bind to the AGTRL1 or PRKCH protein.

The present inventors discovered a relationship between the enhanced autophosphorylation activity of the PRKCH protein and arteriosclerotic diseases. Therefore, preferred examples of the function-suppressing substances of the present invention include antibodies or low-molecular compounds that inhibit the increase of the autophosphorylation activity of the PRKCH protein.

An antibody that binds with an AGTRL1 or PRKCH protein (anti-AGTRL1 antibody or anti-PRKCH antibody) can be prepared according to methods known to one skilled in the art. When the antibody is a polyclonal antibody, it can be obtained, for example, in the following manner. Small animals such as rabbit are immunized with natural AGTRL1 or PRKCH protein, or recombinant (recombination) AGTRL1 or PRKCH protein expressed as fusion protein with GST in microorganisms such as Escherichia coli, or partial peptides thereof, and the serum is collected from the small animal. Serum is purified, for example, through precipitation with ammonium sulfate, a protein A or protein G column, DEAE ion exchange chromatography, or an affinity column coupled with AGTRL1 or PRKCH protein or synthetic peptide to yield the antibody. When the antibody is a monoclonal antibody, it can be prepared, for example, in the following manner. Small animals such as mice are immunized with AGTRL1 or PRKCH protein or partial peptides thereof, the spleen is extirpated from the mice, and is ground to separate cells; the cells and mouse myeloma cells are fused using reagents such as polyethylene glycol; and clones that produce antibodies that bind with AGTRL1 or PRKCH protein are selected from the resulting fused cells (hybridomas). Next, the obtained hybridomas are intraperitoneally transplanted to a mouse; the ascites is recovered from the mouse; and the obtained monoclonal antibodies are purified, for example, through precipitation with ammonium sulfate, a protein A or protein G column, DEAE ion exchange chromatography, or an affinity column coupled with AGTRL1 or PRKCH protein or synthetic peptides to yield the antibody.

The antibody of the present invention is not particularly limited in its form and includes, in addition to the above-mentioned polyclonal antibody and monoclonal antibody, human antibody, humanized antibody obtained by gene recombination, antibody fragment, and modified antibody thereof, so long as it binds with AGTRL1 or PRKCH protein of the present invention.

The AGTRL1 or PRKCH protein for use as sensitizing antigen for obtaining the antibody in the present invention is not limited in animal species as its origin; however, it is preferably a protein derived from mammals such as mice or humans and is particularly preferably a human-derived protein. Such a human-derived protein can be suitably obtained by one skilled in the art using the gene sequence or amino acid sequence disclosed in the present specification.

The protein for use as sensitizing antigen in the present invention may be an entire protein or a partial peptide of the protein. Examples of the partial peptide of proteins include amino (N) terminal fragment, carboxy (C) terminal fragment, or kinase activity site at a center part, of a protein. The term “antibody” in the present specification means refers to antibodies that react with a full-length protein or a fragment of the protein.

Examples of the antibodies for use in the present invention include polyclonal antibodies, monoclonal antibodies, chimeric antibodies, single stranded antibodies (scFv) (Huston et al. (1988) Proc. Natl. Acad. Sci. USA 85: 5879-83; The Pharmacology of Monoclonal Antibody, Vol. 113, Rosenburg and Moore ed., Springer Verlag (1994) pp. 269-315), humanized antibodies, polyspecific antibodies (LeDoussal et al. (1992) Int. J. Cancer Suppl. 7: 58-62; Paulus (1985) Behring Inst. Mitt. 78: 118-32; Millstein and Cuello (1983) Nature 305: 537-9; Zimmermann (1986) Rev. Physiol. Biochem. Pharmacol. 105: 176-260; VanDijk et al. (1989) Int. J. Cancer 43: 944-9), and antibody fragments such as Fab, Fab′, F(ab′)2, Fc, and Fv. These antibodies may be modified, for example, with PEG according to necessity. Antibodies can be configured to be detectable without using a secondary antibody, by preparing a fusion protein with, for example, β-galactosidase, maltose binding protein, glutathione S-transferase (GST), or a green fluorescent protein (GFP). Antibodies can be altered to be detectable and recoverable using, for example, avidin or streptavidin by labeling the antibody with, for example, biotin.

Besides obtaining the above described hybridomas by immunizing animals other than humans with antigens, hybridomas that produce desired human antibodies having binding activity with a protein can be obtained by sensitizing human lymphocytes, such as human lymphocytes infected by EB virus, in vitro with the protein, cells expressing the protein, or the lysate thereof, and fusing the sensitized lymphocytes with human-derived myeloma cells having a permanent division potential, such as U266.

Antibodies against the AGTRL1 or PRKCH protein of the present invention is expected to suppress the function of AGTRL1 or PRKCH protein by binding with said protein and to have, for example, a therapeutic or improving effect on arteriosclerotic diseases such as brain infarction. When the obtained antibody is used for administration to humans (antibody therapy), it is preferably a human antibody or a human-type antibody for lowering immunogenicity.

Substances that can inhibit the function of AGTRL1 or PRKCH protein in the present invention further include low molecular weight substances (low molecular weight compounds) that bind with the AGTRL1 or PRKCH protein. The low molecular weight substances that bind with the AGTRL1 or PRKCH protein in the present invention may be natural or artificial compounds. The compounds can be generally prepared or obtained according to methods known to one skilled in the art. The compound of the present invention can also be obtained by screening methods mentioned below.

The above-mentioned low-molecular weight compound that binds with an AGTRL1 or PRKCH protein of (b) includes, for example, compounds having a high affinity for AGTRL1 or PRKCH.

Substances that can inhibit the function of AGTRL1 or PRKCH protein of the present invention include mutant AGTRL1 or PRKCH proteins having dominant-negative property to the AGTRL1 or PRKCH protein. The “mutant AGTRL1 or PRKCH proteins having dominant-negative property to the AGTRL1 or PRKCH protein” refer to proteins having the function of causing the activity of endogenous wildtype protein to disappear or reducing the activity of endogenous wildtype protein by expressing the gene encoding the protein.

Substances (compounds) known to inhibit the function of AGTRL1 or PRKCH protein can be a suitable and specific example of “substances that can suppress the function of GRK5 protein” in the present invention.

The function inhibitor according to the present invention can be suitably obtained by screening methods according to the present invention using the AGTRL1 or PRKCH activity as an index.

Substances that inhibit (suppress) the autophosphorylation activity or protein kinase activity of the PRKCH protein are also useful as a therapeutic agent for arteriosclerotic diseases according to the present invention. Accordingly, the present invention provides a therapeutic agent for arteriosclerotic diseases, such an agent containing as an active ingredient a substance that inhibits the autophosphorylation activity or protein kinase activity of the PRKCH protein.

The present invention further provides methods of screening for an agent (candidate compound) for treating or preventing arteriosclerotic diseases, including the step of selecting compounds that lower the expression level of AGTRL1 or PRKCH gene or the activity of the protein encoded by the gene.

One embodiment of the screening methods of the present invention is a method using the expression level of AGTRL1 or PRKCH gene as an index. Compounds that lower the expression level of AGTRL1 or PRKCH gene are expected to serve as agents for the prevention and treatment of arteriosclerotic diseases.

The above described method of the present invention is, for example, a method of screening for an agent for treating or preventing arteriosclerotic diseases, which includes the following steps (a) to (c):

(a) contacting a test compound with cells that express AGTRL1 or PRKCH gene;

(b) measuring the expression level of AGTRL1 or PRKCH gene; and

(c) selecting the compound that lowers the expression level as compared to the expression level measured in the absence of the test compound.

In the present method, initially a test compound is contacted with cells that express AGTRL1 or PRKCH gene. The “cells” used herein can be derived from humans, mice, cats, dogs, cattle, sheep, birds, or other pets or livestock, but are not limited thereto. Cells expressing an endogenous AGTRL1 or PRKCH gene, or cells expressing an exogenous AGTRL1 or PRKCH gene which has been introduced thereto can be used as the “cells that express AGTRL1 or PRKCH gene”. Cells expressing an exogenous AGTRL1 or PRKCH gene can be generally produced by introducing to a host cell, expression vectors into which AGTRL1 or PRKCH gene has been inserted. The expression vector can be produced according to general genetic engineering techniques.

Test compounds for use in the method are not particularly limited. Examples thereof include single compounds such as natural compounds, organic compounds, inorganic compounds, proteins, and peptides; and compound libraries, expression products of gene libraries, cell extracts, cell culture supernatants, products of fermenting microorganisms, marine organism extracts, and vegetable extracts, but are not limited thereto.

Test compounds may be “contacted” with cells that expresses AGTRL1 or PRKCH gene generally by adding the test compounds to the culture medium of the cells that express the AGTRL1 or PRKCH gene, but is not limited thereto. When the test compound is, for example, proteins, “contacting” can be carried out by introducing DNA vectors that express the protein into the cells.

In the present method, next, the expression level of the AGTRL1 or PRKCH gene is measured. The term “gene expression” as used herein includes both transcription and translation. Gene expression levels can be measured according to methods known to one skilled in the art. The transcription level of the gene can be measured, for example, by extracting mRNA from cells that express the AGTRL1 or PRKCH gene according to common procedures, and carrying out northern hybridization or RT-PCR using the extracted mRNA as the template. The gene translation level can be measured by recovering protein fractions from cells that expresses the AGTRL1 or PRKCH gene, and detecting the expression of AGTRL1 or PRKCH protein by electrophoresis such as sodium dodecyl sulfate-polyacrylamide gel electrophoresis (SDS-PAGE). In addition, the gene translation level can also be measured by carrying out Western blotting using antibodies against AGTRL1 or PRKCH protein to detect the expression of the protein. Antibodies for use in the detection of AGTRL1 or PRKCH protein is not particularly limited, so long as they are detectable antibodies, and for example, both monoclonal antibodies and polyclonal antibodies can be used.

In the present method, next, compounds that lower the expression level as compared to the expression level when the test compound is not contacted (control) is selected. Such compounds that lower the expression level can be an agent for treating or preventing arteriosclerotic diseases.

Another embodiment of the screening methods according to the present invention is a method of identifying compounds that lower the expression level of the AGTRL1 or PRKCH gene of the present invention, using the expression of a reporter gene as an index.

The above-mentioned method of the present invention is, for example, a method of screening for an agent for treating or preventing arteriosclerotic diseases, which includes the following steps (a) to (c):

(a) contacting a test compound with cells containing DNA having a structure in which a transcriptional regulatory region of AGTRL1 or PRKCH gene and a reporter gene are operably linked with each other;

(b) measuring the expression level of the reporter gene; and

(c) selecting the compound that lowers the expression level as compared to the expression level measured in the absence of the test compound.

In the present method, initially a test compound is contacted with cells or cell extracts that include a DNA having a structure in which a transcriptional regulatory region of the AGTRL1 or PRKCH gene and a reporter gene are operably linked with each other. The phrase “operably linked” herein means that a transcriptional regulatory region of the AGTRL1 or PRKCH gene and a reporter gene bind with each other so that the expression of the reporter gene is induced as a result that a transcription factor binds with the transcriptional regulatory region of the AGTRL1 or PRKCH gene. Accordingly, even when the reporter gene is linked with another gene and forms a fused protein with another gene product, one is included within the meaning of “operably linked”, so long as the expression of the fused protein is induced as a result of binding of a transcription factor with a transcriptional regulatory region of the AGTRL1 or PRKCH gene. A transcriptional regulatory region of the AGTRL1 or PRKCH gene in the genome can be obtained according to known methods based on the cDNA nucleotide sequence of the AGTRL1 or PRKCH gene by one skilled in the art.

The reporter gene for use in the present method is not particularly limited, so long as its expression is detectable, and includes, for example, the CAT gene, lacZ gene, luciferase gene, and GFP gene. The “cells containing DNA having a structure in which a transcriptional regulatory region of an AGTRL1 or PRKCH gene and a reporter gene are operably linked with each other” can be, for example, cells introduced with vectors into which such structure is inserted. Such vectors can be prepared according to methods well known to one skilled in the art. The vectors can be introduced into cells according to general methods such as calcium phosphate precipitation, electroporation, a Lipofectamine method, or microinjection. The “cells containing DNA that has a structure in which a transcriptional regulatory region of an AGTRL1 or PRKCH gene and a reporter gene are operably linked with each other” further include cells in which the structure has been inserted into their chromosome. The DNA structure can be inserted to the chromosome according to methods generally used by one skilled in the art, such as gene transfer technique using homologous recombination.

The “cell extracts containing DNA that has a structure in which a transcriptional regulatory region of an AGTRL1 or PRKCH gene and a reporter gene are operably linked with each other” can be, for example, cell extracts that are contained in commercially available in vitro transcription/translation kit and are added with DNA that has a structure in which a transcriptional regulatory region of an AGTRL1 or PRKCH gene and a reporter gene are operably linked with each other.

The “contact” in the present method can be conducted by adding a test compound to a culture medium of the “cells containing DNA having a structure in which a transcriptional regulatory region of an AGTRL1 or PRKCH gene and a reporter gene are operably linked with each other”, or by adding a test compound to the above described commercially available cell extracts containing the DNA. When the test compound is a protein, the contact can also be conducted by introducing DNA vectors that express the protein into the cells.

Next, the expression level of the reporter gene is measured in the present method. The expression level of the reporter gene can be measured by methods known to one skilled in the art according to the type of the reporter gene. When the reporter gene is, for example, the CAT gene, the expression level of the reporter gene can be measured by detecting acetylation of chloramphenicol by the gene product. The expression level of the reporter gene can be measured by detecting coloring of a dye compound catalyzed by an expression product of the lacZ gene when the reporter gene is the lacZ gene; by detecting fluorescence of a fluorescent compound catalyzed by an expression product of the luciferase gene when the reporter gene is the luciferase gene; or by detecting fluorescence of the GFP protein when the reporter gene is the GFP gene.

Next, in the present method, compounds that lower (suppress) the measured expression level of the reporter gene as compared to the expression level measured in the absence of the test compound is selected. The compounds that lower (suppress) the expression level can be an agent for treating or preventing arteriosclerotic diseases.

As the AGTRL1 or PRKCH gene for use in the above-mentioned screening method of the present invention, a wildtype gene can be generally used, and an AGTRL1 gene (mutant AGTRL1 gene) containing a polymorphic mutation (“SNP4” and/or “SNP9”) which is involved in elevated expression and discovered herein can also be suitably used.

This mutant AGTRL1 gene originally shows enhanced gene expression and is suitable for screening a substance that suppresses (lowers) the expression of the gene. In addition, a substance that suppresses (lowers) the enhanced gene expression of a mutant AGTRL gene found in patients actually suffering from brain infarction is expected to be a suitable agent for preventing or treating arteriosclerotic diseases such as brain infarction.

Another embodiment of the screening method of the present invention is a method that uses as an index the activity of interaction (binding activity) between the Sp1 transcription factor and the DNA region in the AGTRL1 gene that binds to the Sp1 transcription factor.

The above-described method of the present invention is, for example, a method of screening for pharmaceutical agents for treating or preventing arteriosclerotic diseases, which comprises the steps of:
(a) contacting a test compound with the Sp1 transcription factor and a polynucleotide comprising a DNA region which comprises a nucleotide site in an AGTRL1 gene located at position 42509 or 39353 of the nucleotide sequence of SEQ ID NO: 1;
(b) measuring the binding activity between the polynucleotide and the Sp1 transcription factor; and
(c) selecting a compound that reduces the binding activity as compared with that measured in the absence of the test compound.

In the present method, a test compound is first contacted with the Sp1 transcription factor and a polynucleotide comprising a DNA region which comprises a nucleotide site in an AGTRL1 gene located at position 42509 or 39353 of the nucleotide sequence of SEQ ID NO: 1.

Next, the activity of interaction (binding activity) between the polynucleotide and the Sp1 transcription factor is measured. This interaction activity can be evaluated using various methods well known to those skilled in the art.

For example, the interaction activity can be evaluated by a shift assay. More specifically, it can be carried out appropriately by the method to be described in Example 9.

Subsequently, compounds that reduce (suppress) the interaction activity as compared with that measured in the absence of the test compound are selected. Compounds that reduce (suppress) the activity will become pharmaceutical agents for treating arteriosclerotic diseases.

A further embodiment of the screening method of the present invention is a method that uses the autophosphorylation activity or protein kinase activity of the PRKCH protein as an index. Compounds that reduce these activities are expected to show therapeutic effects for arteriosclerotic diseases.

The above-described method of the present invention is, for example, a method of screening for pharmaceutical agents for treating or preventing arteriosclerotic diseases, which comprises the steps of:
(a) contacting a test compound with a PRKCH protein;
(b) measuring the autophosphorylation activity or protein kinase activity of the PRKCH protein; and
(c) selecting a compound that reduces the activity as compared with that measured in the absence of the test compound.

In the present method, the PRKCH protein is first contacted with a test compound. This “contact” can also be carried out, for example, by contacting a test compound with cells expressing the PRKCH protein.

Next, the autophosphorylation activity or protein kinase activity of the PRKCH protein is measured. The PRKCH protein used in the above-mentioned method is preferably a mutant protein whose autophosphorylation activity is enhanced. A preferred example of such a mutant protein is a mutant protein in which valine at position 374 in the amino acid sequence of the PRKCH protein is substituted with isoleucine. Furthermore, a wild-type protein that has no mutations can also be used. Furthermore, it may be a partial polypeptide containing the part to be phosphorylated in the PRKCH protein, or a mutant polypeptide containing this part.

The amino acid site to be autophosphorylated in the PRKCH protein includes, for example, T510 (threonine at position 510), T650 (threonine at position 650), and S672 (serine at position 672). It is known that one of these positions (T510) is phosphorylated by PKD1 and the remaining two positions (T650 and S672) are autophosphorylated (B. D. Gomperts et al., translation supervised by Y. Kajiro, “Signal Transduction”, Medical Science International, p 206). A preferred embodiment of the screening method of the present invention is a method that uses the autophosphorylation states of the above-mentioned amino acid sites as index.

The measurement of phosphorylation activity can be carried out by means well known to those skilled in the art. For example, it can be measured by Western blotting using phosphorylation-specific antibodies, or such. More specifically, the measurement of phosphorylation activity can be carried out appropriately by methods to be described in the Examples.

Furthermore, in the above-mentioned method, compounds that reduce the aforementioned phosphorylation activity as compared with that measured in the absence of the test compound are selected. Compounds selected in this manner are expected to exhibit therapeutic or preventive effects on arteriosclerotic diseases as a result of suppressing the autophosphorylation activity or protein kinase activity of the PRKCH protein.

Compounds used in the various screening methods described above are also useful as reagents for screening for pharmaceutical agents for treating or preventing arteriosclerotic diseases.

Specific examples of the above-mentioned reagents of the present invention include reagents for screening for pharmaceutical agents for treating or preventing arteriosclerotic diseases, which comprise any one of the following (a) to (d) as an active ingredient:
(a) an oligonucleotide that hybridizes with a transcript of the AGTRL1 or PRKCH gene;
(b) an antibody that recognizes the AGTRL1 or PRKCH protein;
(c) a polynucleotide comprising a DNA region which comprises a nucleotide site in the AGTRL1 gene located at position 39353 or 42509 of the nucleotide sequence of SEQ ID NO: 1; and
(d) a mutant PRKCH protein having an amino acid sequence in which valine at position 374 in the amino acid sequence of the PRKCH protein is substituted with isoleucine.

The present invention further provides kits which include, for example, various agents and/or reagents used for carrying out the testing methods or screening methods of the present invention.

The kit of the present invention can include, for example, a reagent suitably selected from among the reagents of the present invention, according to the testing method or screening method to be conducted. For example, the kit of the present invention may include, as a constitutional component, the genes, proteins, oligonucleotides and antibodies of the present invention. More specific examples include (1) primer oligonucleotides for use in the present invention, and PCR reaction reagents (such as Taq polymerase and buffer); (2) probe oligonucleotides for use in the present invention, and hybridization buffer; and (3) anti-AGTRL1 antibody or anti-PRKCH antibody and ELISA reagent.

The kit of the present invention may further suitably include, for example, control samples, buffer, and directions for use.

All the prior art documents cited in the present specification are incorporated herein by reference.

Furthermore, the present invention relates to methods for treating or preventing arteriosclerotic diseases comprising the step of administering a pharmaceutical agent of the present invention to a subject. A required amount of the pharmaceutical agent of the present invention is administered to a mammal, including human, within a dose range that is considered to be safe. The dose of the pharmaceutical agent of the present invention can be appropriately determined by considering the type of dosage form, method of administration, age and body weight of the patient, symptoms of the patient, and such, and ultimately by the decision of a physician or veterinarian.

### Examples

Herein below, the present invention will be specifically described with reference to the Examples, but it is not to be construed as being limited thereto.

### <Materials, Methods, and Such of Various Experiments Relating to the PRKCH Gene>

### Test Groups

In a genome-wide case-control study, cerebral infarction cases from seven affiliated hospitals of Kyushu University were registered. All cases were diagnosed and classified by physicians who were experts in cerebral apoplexy using clinical information and brain imaging tests. The subtypes of cerebral infarction were determined according to the diagnostic criteria of Classification of Cerebrovascular Diseases III proposed by the National Institute of Neurological Disorders and Stroke (Whisnant J P, et al., Stroke; 21:637-676 (1990)), the Trial of Org 10172 in Acute Stroke Treatment (TOAST) (Adams, H P Jr. et al., Stroke 24, 35-41 (1993)), and Cerebral Embolism Task Force (No authors listed, Arch. Neurol. 43, 71-84 (1986)).

Control subjects were incorporated from the participants in the Hisayama Study. The Hisayama study is an ongoing prospective epidemiologic study on cardiovascular diseases using a regional population established in 1961. Details of this study have been described previously (Kubo, M. et al., Stroke 34, 2349-2354 (2003); and Kiyohara, Stroke. 34, 2343-2348 (2003)). Screening survey was performed for this study from 2002 to 2003. In short, a total of 3,328 participants who were aged 40 or older (78% participation rate) agreed to participate in a health examination and underwent a comprehensive assessment. After excluding subjects with a past medical history of cerebral apoplexy or coronary artery disease, the present inventors selected age—(within five years) and sex-matched control subjects by 1:1 matching using random numbers.

To examine the risk of rs2230500 on the onset of cerebral infarction, the present inventors used a cohort of the Hisayama study established in 1988 (Kubo, M. et al., Stroke 34, 2349-2354 (2003)). In short, 2,742 Hisayama residents aged 40 years or older participated in a health examination in 1988 (80% participation rate). After excluding subjects with a past medical history of cerebral apoplexy or coronary artery disease, a continuous follow-up was performed on 2,637 participants for the onset of cardiovascular diseases or death. Of the cohort subjects, 1,683 subjects participated in the examination in 2002.

The present inventors obtained informed consent statements signed by all the test subjects as approved by the ethics committees of the Faculty of Medical Sciences in Kyushu University, the Institute of Medical Science in the University of Tokyo, and each participating hospital.

### SNP Genotyping

Genomic DNA was extracted from peripheral blood leukocytes by a standard protocol. SNPs were genotyped using the multiplex PCR-based Invader assay (Third Wave Technologies, Madison, Wis.) as described by Ohnishi et al. (Ohnishi Y. et al., J. Hum. Genet. 46, 471-477 (2001)), or by direct sequencing of PCR products using the ABI3700 capillary sequencer (Applied Biosystems) following a standard protocol.

### Cell Culturing, Transfection, and Immunoprecipitation

293T cells were maintained at 37° C. under 5% CO₂ in DMEM supplemented with 10% fetal calf serum and 1% antibiotics. A plasmid expressing full-length PKCη-374V was constructed by cloning human thymus cDNA into p3xFLAG-CMV-14 expression vector (Sigma). A plasmid expressing full-length PKCη-374I was constructed from p3xFLAG-CMV-14-PKCη-374V vector using the QuikChange XL Site-Directed Mutagenesis Kit (Stratagene) according to the manufacturer's instructions. For transient transfection, 293T cells were plated in 15-cm culture dishes, and when nearly confluent, p3xFLAG-CMV-14-PKCη-374V or p3xFLAG-CMV-14-PKCη-374I was transfected using FuGENE6 (Roche) according to the manufacturer's instructions. Forty-eight hours later, the cells were collected and lysed at 4° C. in lysis buffer containing 1% Nonidet P-40, 150 mM NaCl, 50 mM Tris-HCl, pH8.0, 1 mM phenylmethylsulfonyl fluoride, 1 mM dithiothreitol, and 0.1% Protease Inhibitor Cocktail Set III (Calbiochem). After a 30-minute incubation on ice, the lysates were centrifuged at 15,000 rpm at 4° C. for 15 minutes. The supernatant was pretreated with rec-protein G-Sepharose 4B conjugate (Zymed) and normal mouse IgG for 30 minutes, and then incubated with anti-Flag M2 affinity gel (Sigma) for three to four hours at 4° C. After incubation, the gel was washed twice with the lysis buffer and once with 1× TBS buffer, and the Flag-labeled protein was eluted by adding 15 μg of 3× Flag peptide (Sigma). The purity and amount of the immunoprecipitates were evaluated by Coomassie Brilliant Blue staining. Protein concentration was determined by the Bradford method.

### PKC Activity and Autophosphorylation Assay

PKC autophosphorylation activity and kinase activity were measured according to the method described by Ikuta et al. (Ikuta, T. et al., Cell Growth Diff. 5, 943-947 (1994)). For the PKC autophosphorylation assay, immunoprecipitates for mock, PKCη-374V, and PKCη-374I were incubated at 30° C. for the indicated duration in a reaction mixture of a total volume of 50 μL containing 20 mM Tris-HCl (pH7.5), 5 mM MgSO₄, 1 mM EGTA, and 5 μCi of [γ-³²P]ATP together with 10 μM phosphatidyl serine (PS, Sigma) and 100 nM phorbol-12,13-dibutyrate (PDBu, Sigma), and were then subjected to SDS-PAGE and autoradiography. Protein kinase activity was determined by the incorporation of ³²P from [γ-³²P]ATP into the myelin basic protein (MBP) peptide (Sigma). The incubation mixture (total volume of 50 μL) contained 20 mM Tris-HCl (pH7.5), 5 mM MgSO₄, 1 mM EGTA, 100 μM ATP, 1 μCi of [γ-³²P]ATP, 10 μg MBP peptide, and PKCη immunoprecipitates, as well as 10 μM PS and 100 nM PDBu. After incubation at 30° C. for three minutes, the reaction was stopped by direct application to P81 phosphocellulose square (Upstate). After washing with 75 mM phosphoric acid, radioactivity was quantified. The unit activity was defined as incorporation of 1 nmol/minute of radioactive phosphoric acid from ATP to MBP.

### Quantification of PRKCH Expression Using Real-Time PCR

The present inventors performed real-time quantitative PCR using ABI 7700 (Applied Biosystems) together with SYBR Premix ExTag (TaKaRa) according to the manufacturers' instructions. Human total RNAs derived from various tissues (Clonetech) were purchased, and single-chain cDNAs were synthesized from 1 μg of total RNA using oligo d(T)₁₂₋₁₈ primer and Superscript III reverse transcriptase (Invitrogen). Relative expression of PRKCH mRNA was normalized to the beta actin expression level in the same cDNA using the standard curve method described by the manufacturer.

### Immunohistochemistry Test and Morphometrical Analysis of the Coronary Artery

From 16 (eight males and eight females) Japanese patients of ages 68 to 91 (81.1±6.2) who resided in Hisayama, the hearts were obtained by autopsy within 16 hours of death at Kyushu University. Cannula was inserted into the coronary artery and after washing with 0.1 mol/L phosphate buffered saline (pH7.4), perfusion with 1 L of 4% (wt/vol) paraformaldehyde (in 0.1 mol/L sodium phosphate, pH7.4) was carried out at 100 mmHg. Next, the hearts were soaked for at least 24 hours at 4° C. in 4% paraformaldehyde. The right coronary artery and left anterior descending coronary artery were dissected from the surface of the heart, cut perpendicularly to the long axis at 3-mm intervals and then embedded in paraffin. Sixty blocks were obtained and 3-μm-thick continuous sections were prepared at once. The sections from each block were successively subjected to hematoxylin and eosin staining, Elastica-van Gieson staining, and Masson trichrome staining, and immunohistochemistry tests. In accordance with the definitions proposed by the Committee on Vascular Lesions of the Council on Arteriosclerosis of AHA (Stary, H C. et al., Circulation. 92, 1355-74 (1995)), each section was carefully classified into types of atherosclerotic lesion.

Immunohistochemistry tests were performed as described by Nakano et al. (Nakano, T. et al., Hum Pathol. 36, 330-40 (2005)). In brief, deparaffinized sections were incubated with 3% nonfat milk, and incubated with primary antibodies against human PKCη (Santa Cruz), endothelial cells (anti-human CD31, Dako), monocytes/macrophages (anti-human CD68, Dako), and smooth muscle cells (anti-human α-SMA, Sigma), and then with peroxidase-labeled secondary antibodies (Dako). The slides were incubated with 3,3α-diaminobenzidine tetrachloride (DAB), and then counterstained with hematoxylin. As a negative control, nonimmune rabbit IgG or nonimmune mouse IgG of each isotype was substituted instead of the respective primary antibodies. Tissue blocks collected from the human mammary glands were used as positive control for PKCη (Masso-Welch, P A. et al., Breast Can. Res. Treat. 68, 211-223 (2001)). A single observer who was unaware of the types of atherosclerotic lesion quantified PKCη-positive lesions, and analysis was preformed by determining the positive area in the atherosclerotic intima. All images were captured and analyzed by the image software of US National Institutes of Health.

### Statistical Analysis

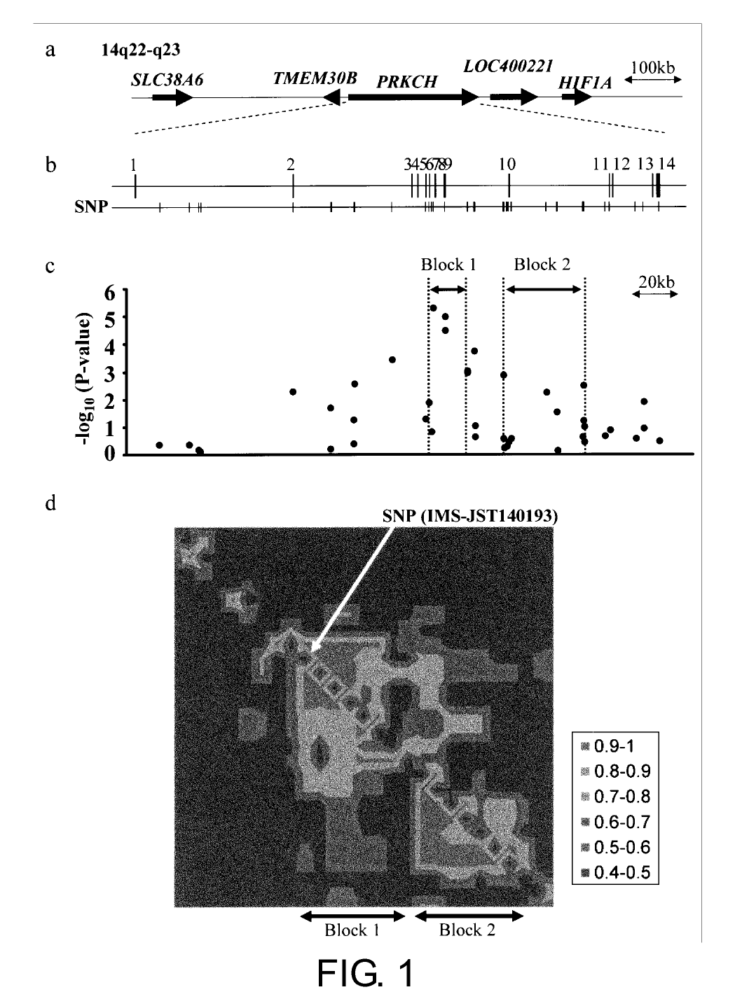

Data are presented as mean±s.d. unless otherwise stated. Association and Hardy-Weinberg equilibrium were evaluated by chi-square test and Fisher's exact test. Calculations of linkage disequilibrium index and Δ index, and creation of FIG. 1d were carried out as described by Tokuhiro et al. (Tokuhiro, S. et al., Nat. Genet. 35, 341-348 (2003)). For adjustment of multiple testing, the present inventors repeated a random permutation test 10,000 times using the MULTTEST procedure in SAS 9.12 software. The difference in the incidence rate of cerebral infarction due to the rs2230500 genotype over 14 years was evaluated by using the Cox proportional hazard model after making adjustments for age and sex. For adjustment of clinical risk factors, a logistic regression model was used for the case-control study and Cox proportional hazards regression model was used for the prospective cohort study, using the SAS software. PKCη-positive areas were compared across grades of artery atherosclerosis using Spearman's rank correlation with Bonferroni correction.

### Example 1

### Large-Scale Case-Control Study Using Gene-Based SNP Markers

To identify genes that are susceptible and implicated in cerebral infarction, the present inventors used 1,112 cerebral infarction patients and 1,112 age- and sex-matched control subjects to perform a large-scale genome-wide case-control study. The clinical characteristics of the subject groups in this case-control study are shown in Table 3.

TABLE 3

| | Case | Control | P value |
|---|---|---|---|
| N | 1112 | 1112 | |
| Age | 70.2 ± 10.0 | 70.1 ± 10.1 | 0.87 |
| Male (%) | 60.7 | 60.7 | |
| Cerebral infarction subtypes | | | |
| Lacunar (LA) | 491 | | |
| Atherothrombotic (AT) | 369 | | |
| Combined LA + AT | 860 | | |
| Cardiogenic embolism | 136 | | |
| Others | 116 | | |
| Hypertension (%) | 78.1 | 53.7 | <0.0001 |
| Hyperlipidemia (%) | 48.3 | 41.8 | 0.0024 |
| Diabetes (%) | 30.4 | 21.1 | <0.0001 |

First, the present inventors genotyped 188 Japanese cases with cerebral infarction and 188 age- and sex-matched control cases using 52,608 gene-based tag-SNPs selected from the JSNP database (Tsunoda, T. et al., Hum. Mol. Genet. 13, 1623-1632 (2004)). Allele frequencies of 48,083 successfully genotyped SNPs (overall success rate of 91.4%) were compared, and 1,098 SNPs showing p<0.01 between the cases and controls were identified.

The present inventors subsequently genotyped the remaining cases and controls for these SNPs in the second screening. When the subjects were stratified according to the subtypes of cerebral infarction, several SNPs that highly correlated with the combined lacunar infarction and atherothrombotic infarction group were identified. Of them, SNP_{—}15 (IMS-JST140193, rs3783799) in intron 6 of PRKCH on chromosome 14q22-23 highly correlated with the lacunar infarction group (p=4.73×10⁻⁶ for allele frequency) and the combined lacunar infarction and atherothrombotic infarction group (p=7.91×10⁻⁶ in the dominant model, Table 5). Even after permutation tests using all SNPs that were genotyped in the second screening, SNP_{—}15 still showed correlation with lacunar infarction (p=0.0036) and lacunar-atherothrombotic combined infarction (p=0.0063). Accordingly, the present inventors considered that the susceptibility locus for lacunar infarction and atherothrombotic infarction might be located around SNP_{—}15.

Table 4 below shows the genotyped SNP positions in the PRKCH gene (NCBI Build 35).

TABLE 4

| SNP Name | Position | jsnp_jd | dbSNP rs# | ContigAcc | ContigPos |
|---|---|---|---|---|---|
| SNP_01 | Intron 1 | IMS-JST140210 | rs3783814 | NT_026437.11 | 42799673 |
| SNP_02 | Intron 1 | IMS-JST140207 | rs3783812 | NT_026437.11 | 42812443 |
| SNP_03 | Intron 1 | IMS-JST140206 | rs1033910 | NT_026437.11 | 42816593 |
| SNP_04 | Intron 1 | IMS-JST140205 | rs3783810 | NT_026437.11 | 42817442 |
| SNP_05 | Exon 2 | IMS-JST093801 | rs3742633 | NT_026437.11 | 42857722 |
| SNP_06 | Intron 2 | IMS-JST140204 | rs767757 | NT_026437.11 | 42874127 |
| SNP_07 | Intron 2 | IMS-JST140203 | rs767755 | NT_026437.11 | 42874201 |
| SNP_08 | Intron 2 | IMS-JST140202 | rs2209386 | NT_026437.11 | 42884237 |
| SNP_09 | Intron 2 | IMS-JST140201 | rs3783806 | NT_026437.11 | 42884263 |
| SNP_10 | Intron 2 | IMS-JST140198 | rs3783803 | NT_026437.11 | 42884524 |
| SNP_11 | Intron 2 | IMS-JST140196 | rs3783801 | NT_026437.11 | 42900735 |
| SNP_12 | Intron 4 | IMS-JST050416 | rs2296273 | NT_026437.11 | 42915504 |
| SNP_13 | Intron 5 | IMS-JST050417 | rs2296274 | NT_026437.11 | 42916931 |
| SNP_14 | Intron 6 | IMS-JST140194 | rs3783800 | NT_026437.11 | 42917986 |
| SNP_15 | Intron 6 | IMS-JST140193 | rs3783799 | NT_026437.11 | 42918969 |
| SNP_16 | Intron 8 | IMS-JST050419 | rs2296276 | NT_026437.11 | 42923845 |
| SNP_17 | Exon 9 | IMS-JST050420 | rs2230500 | NT_026437.11 | 42923992 |
| SNP_18 | Exon 9 | na | rs17098388 | NT_026437.11 | 42923994 |
| SNP_19 | Intron 9 | IMS-JST140187 | rs959728 | NT_026437.11 | 42933771 |
| SNP_20 | Intron 9 | IMS-JST140186 | rs3783792 | NT_026437.11 | 42933890 |
| SNP_21 | Intron 9 | IMS-JST140183 | rs3783789 | NT_026437.11 | 42936553 |
| SNP_22 | Intron 9 | IMS-JST140179 | rs3783786 | NT_026437.11 | 42936977 |
| SNP_23 | Intron 9 | IMS-JST140178 | rs3783785 | NT_026437.11 | 42937045 |
| SNP_24 | Intron 9 | IMS-JST140171 | rs3783778 | NT_026437.11 | 42949333 |
| SNP_25 | Intron 9 | IMS-JST140170 | rs3783777 | NT_026437.11 | 42949441 |
| SNP_26 | Intron 9 | IMS-JST140169 | rs3783776 | NT_026437.11 | 42949496 |
| SNP_27 | Intron 9 | IMS-JST140168 | rs912619 | NT_026437.11 | 42949686 |
| SNP_28 | Intron 9 | IMS-JST140167 | rs3783774 | NT_026437.11 | 42950854 |
| SNP_29 | Intron 9 | IMS-JST140166 | rs1536015 | NT_026437.11 | 42951558 |
| SNP_30 | Intron 10 | IMS-JST140165 | rs3783772 | NT_026437.11 | 42952680 |
| SNP_31 | Intron 10 | IMS-JST140163 | rs3783771 | NT_026437.11 | 42952898 |
| SNP_32 | Intron 10 | IMS-JST140154 | rs3783762 | NT_026437.11 | 42967956 |
| SNP_33 | Intron 10 | IMS-JST140153 | rs2245448 | NT_026437.11 | 42972568 |
| SNP_34 | Intron 10 | IMS-JST140152 | rs3783760 | NT_026437.11 | 42972779 |
| SNP_35 | Intron 10 | IMS-JST103326 | rs1091680 | NT_026437.11 | 42983840 |
| SNP_36 | Intron 10 | IMS-JST103327 | rs3751292 | NT_026437.11 | 42984053 |
| SNP_37 | Intron 10 | IMS-JST103328 | rs3751293 | NT_026437.11 | 42984152 |
| SNP_38 | Intron 10 | IMS-JST103329 | rs2252267 | NT_026437.11 | 42984437 |
| SNP_39 | Intron 10 | IMS-JST103330 | rs3751295 | NT_026437.11 | 42984490 |
| SNP_40 | Intron 10 | IMS-JST140150 | rs3783758 | NT_026437.11 | 42993489 |
| SNP_41 | Intron 10 | IMS-JST093800 | rs2463117 | NT_026437.11 | 42995426 |
| SNP_42 | Intron 12 | IMS-JST140146 | rs3783755 | NT_026437.11 | 43006640 |
| SNP_43 | Intron 12 | IMS-JST140145 | rs3783754 | NT_026437.11 | 43009990 |
| SNP_44 | Intron 12 | IMS-JST140144 | rs3783753 | NT_026437.11 | 43010112 |
| SNP_45 | Exon 14 | IMS-JST173548 | rs3813410 | NT_026437.11 | 43016888 |

Table 5 below is a summary of the case-control correlation analysis of PRKCH_SNP_{—}15

TABLE 5

| | | | | | | | | | P-value | | Odds ratio | |
|---|---|---|---|---|---|---|---|---|---|---|---|---|
| | Case | | | | Control | | | | (Adjusted_P) | | (95% c.i.) | |
| Phenotype | 11 | 12 | 22 | Total | 11 | 12 | 22 | Total | 1/2 | 11 + 12/22 | 1/2 | 11 + 12/22 |
| Cerebral infarction | 59 | 394 | 655 | 1108 | 41 | 336 | 731 | 1108 | 5.25E−04 | 8.51E−04 | 1.29 | 1.34 |
| | | | | | | | | | (0.34) | (0.54) | (1.12~1.49) | (1.13~1.59) |
| Lacunar infarction | 28 | 178 | 282 | 488 | 11 | 131 | 345 | 487 | 4.73E−06 | 2.10E−05 | 1.69 | 1.77 |
| group | | | | | | | | | (0.0036) | (0.015) | (1.35~2.12) | (1.36~2.31) |
| Atherothrombotic | 15 | 136 | 217 | 368 | 17 | 109 | 243 | 369 | 0.134 | 0.0536 | 1.21 | 1.34 |
| infarction group | | | | | | | | | (ND) | (ND) | (0.94~1.56) | (1.00~1.81) |
| Combined lacunar | 43 | 314 | 499 | 856 | 28 | 240 | 588 | 856 | 1.00E−05 | 7.91E−06 | 1.46 | 1.57 |
| infarction and | | | | | | | | | (0.0097) | (0.0063) | (1.23~1.73) | (1.29~1.91) |
| atherothrombotic | | | | | | | | | | | | |
| infarction group | | | | | | | | | | | | |

In Table 5 above, allele 1 is defined as a risk allele. P-values were adjusted by performing 10⁴ permutation tests on all SNPs examined in the screening. “c.i.” refers to confidence interval.

Next, the present inventors performed high-precision mapping of PRKCH using 45 SNPs, and the extent of LD in SNP_{—}15 was evaluated using lacunar infarction and atherothrombotic infarction cases as well as age- and sex-matched controls. These SNPs were genotyped and D′ and Δ indices were calculated for 27 types of SNPs with minor allele frequency greater than 0.2 (FIG. 1d). Two LD blocks were identified in PRKCH, and SNP_{—}15 was found to be positioned in block 1 (FIG. 1d). The present inventors also compared the allele frequencies between the cases and the controls, and discovered that the association was greatest in block 1 of PRKCH and gradually decreased in the 5′ and 3′ regions. From these results, the present inventors concluded that PRKCH is a susceptibility gene for lacunar and atherothrombotic cerebral infarctions.

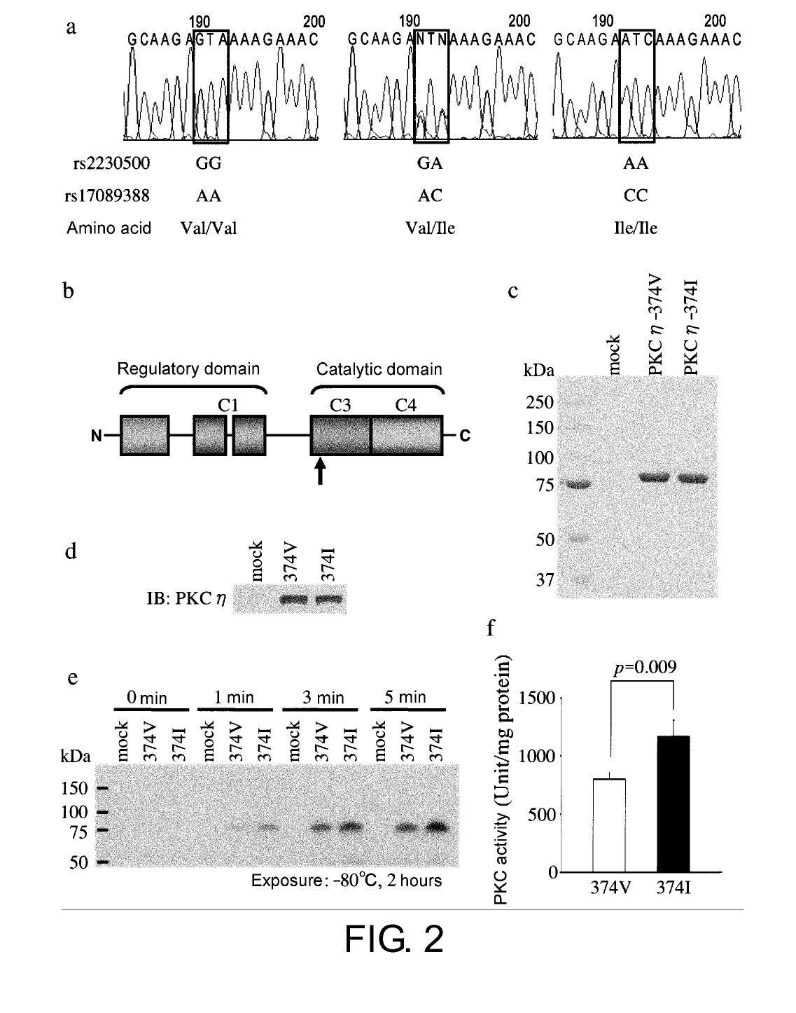

Next, the present inventors sequenced all of the exons in PRKCH including the 5′ and 3′ untranslated regions from 48 cases and 48 controls. As a result, four SNPs were identified: rs3742633 (695A>G, in exon 2), rs2230500 (1425G>A, in exon 9), rs17098388 (1427A>C, in exon 9), and rs1088680 (1979C>T, in exon 12). Of them, rs3742633 and rs1088680 were positioned outside of block 1 and did not show association. rs2230500 and rs17098388 had a single-nucleotide gap between each other and were positioned in block 1. Furthermore, rs2230500 induced an amino acid substitution of isoleucine for valine (V374T, FIG. 2a), but on the other hand, rs17098388 was a synonymous SNP. The sequencing result revealed these two SNPs were completely linked.

Next, the present inventors genotyped rs2230500 by direct sequencing in all lacunar and atherothrombotic infarction cases as well as in age- and sex-matched controls. The A allele of rs2230500 which induces amino acid change into Ile was observed at high frequency in the cases, and this was highly associated with both lacunar infarction (p=9.84×10⁻⁶, odds ratio (OR) of 1.66, 95% confidence interval (c.i.) of 1.33 to 2.09, allele frequency model, Table 7), and in the combined lacunar infarction and atherothrombotic infarction group (p=4.92×10⁻⁵, OR of 1.42, 95% c.i. of 1.20 to 1.68). Permutation tests were applied to genotyped 45 SNPs in PRKCH, and the results obtained were p=0.0004 for lacunar infarction and p=0.0014 for the combined lacunar infarction and atherothrombotic infarction group. These results were both statistically significant.

To elucidate the confounding effects of the difference between the cases and the controls, the present inventors adjusted clinical risk factors using a logistic regression model. Genotype risk for cerebral infarction was substantially unchanged after adjustments of age, sex, hypertension, hyperlipidemia, and diabetes (Table 8). Therefore, the present inventors established a hypothesis that the V374I amino acid substitution in PRKCH might bring about the onset of cerebral infarction by affecting the signal transduction of PKCη.

Table 6 below shows the results of case-control correlation analysis of 45 types of SNPs in the PRKCH gene for the subgroups of the combined lacunar infarction and atherothrombotic infarction group.

TABLE 6

| SNP | MAF | | Allele model | | Dominant model | | Allele model | | Dominant model | |
|---|---|---|---|---|---|---|---|---|---|---|
| Name | Case | Control | P-Value | Adjusted_P | P-Value | Adjusted_P | OR | 95% CI | OR | 95% CI |
| SNP_01 | 0.452 | 0.462 | 0.562 | 1 | 0.467 | 1 | 1.04 | (0.91~1.19) | 1.09 | (0.86~1.38) |
| SNP_02 | 0.389 | 0.382 | 0.654 | 1 | 0.896 | 1 | 1.03 | (0.90~1.18) | 1.01 | (0.83~1.23) |
| SNP_03 | 0.448 | 0.449 | 0.958 | 1 | 0.765 | 1 | 1.00 | (0.88~1.15) | 0.97 | (0.76~1.22) |
| SNP_04 | 0.009 | 0.008 | 0.691 | 1 | 0.690 | 1 | 1.17 | (0.54~2.54) | 1.17 | (0.54~2.55) |
| SNP_05 | 0.199 | 0.237 | 0.00608 | 0.16 | 0.0108 | 0.24915 | 1.26 | (1.07~1.48) | 1.77 | (1.14~2.77) |
| SNP_06 | 0.452 | 0.491 | 0.0234 | 0.46 | 0.0609 | 0.7696 | 1.17 | (1.02~1.34) | 1.24 | (0.99~1.55) |
| SNP_07 | 0.216 | 0.212 | 0.798 | 1 | 0.912 | 1 | 1.02 | (0.87~1.20) | 1.01 | (0.83~1.23) |
| SNP_08 | 0.362 | 0.363 | 0.940 | 1 | 0.413 | 1 | 1.01 | (0.87~1.16) | 1.12 | (0.85~1.49) |
| SNP_09 | 0.190 | 0.169 | 0.102 | 0.94 | 0.0734 | 0.85115 | 1.16 | (0.97~1.38) | 1.20 | (0.98~1.47) |
| SNP_10 | 0.138 | 0.107 | 0.00472 | 0.13 | 0.00400 | 0.1048 | 1.34 | (1.09~1.65) | 1.39 | (1.11~1.75) |
| SNP_11 | 0.141 | 0.102 | 0.000567 | 0.020 | 0.00100 | 0.03215 | 1.44 | (1.17~1.77) | 1.47 | (1.17~1.84) |
| SNP_12 | 0.264 | 0.290 | 0.0891 | 0.91 | 0.0530 | 0.72955 | 1.14 | (0.98~1.32) | 1.41 | (0.99~1.99) |
| SNP_13 | 0.480 | 0.437 | 0.0130 | 0.31 | 0.00691 | 0.1831 | 1.19 | (1.04~1.37) | 1.33 | (1.08~1.64) |
| SNP_14 | 0.024 | 0.027 | 0.595 | 1 | 0.157 | 0.99925 | 1.12 | (0.74~1.71) | 0.00 | |
| SNP_15 | 0.234 | 0.173 | 1.00E−05 | 0.0002 | 7.90E−06 | 0.00015 | 1.46 | (1.23~1.73) | 1.57 | (1.29~1.91) |
| SNP_16 | 0.235 | 0.176 | 1.63E−05 | 0.00045 | 1.73E−05 | 0.00045 | 1.44 | (1.22~1.71) | 1.54 | (1.27~1.88) |
| SNP_17 | 0.228 | 0.173 | 4.92E−05 | 0.0016 | 4.12E−05 | 0.0014 | 1.42 | (1.20~1.68) | 1.51 | (1.24~1.85) |
| SNP_18 | 0.228 | 0.173 | 4.92E−05 | 0.0016 | 4.12E−05 | 0.0014 | 1.42 | (1.20~1.68) | 1.51 | (1.24~1.85) |
| SNP_19 | 0.266 | 0.219 | 0.00143 | 0.047 | 0.00303 | 0.087 | 1.29 | (1.10~1.51) | 1.34 | (1.10~1.62) |
| SNP_20 | 0.267 | 0.220 | 0.00127 | 0.042 | 0.00307 | 0.08775 | 1.29 | (1.11~1.51) | 1.34 | (1.10~1.62) |
| SNP_21 | 0.208 | 0.160 | 0.000272 | 0.0096 | 0.000645 | 0.01965 | 1.38 | (1.16~1.64) | 1.42 | (1.16~1.74) |
| SNP_22 | 0.498 | 0.478 | 0.162 | 0.99 | 0.550 | 1 | 1.10 | (0.96~1.26) | 1.07 | (0.86~1.32) |
| SNP_23 | 0.438 | 0.459 | 0.233 | 1 | 0.361 | 1 | 1.09 | (0.95~1.24) | 1.11 | (0.88~1.40) |
| SNP_24 | 0.142 | 0.153 | 0.365 | 1 | 0.275 | 1 | 1.09 | (0.90~1.32) | 1.42 | (0.76~2.66) |
| SNP_25 | 0.233 | 0.190 | 0.00210 | 0.063 | 0.00268 | 0.0755 | 1.29 | (1.10~1.53) | 1.35 | (1.11~1.64) |
| SNP_26 | 0.233 | 0.190 | 0.00207 | 0.064 | 0.00267 | 0.0783 | 1.29 | (1.10~1.53) | 1.35 | (1.11~1.64) |
| SNP_27 | 0.461 | 0.456 | 0.758 | 1 | 0.596 | 1 | 1.02 | (0.89~1.17) | 1.06 | (0.86~1.30) |
| SNP_28 | 0.463 | 0.455 | 0.662 | 1 | 0.520 | 1 | 1.03 | (0.90~1.18) | 1.07 | (0.87~1.32) |
| SNP_29 | 0.142 | 0.152 | 0.425 | 1 | 0.358 | 1 | 1.08 | (0.89~1.30) | 1.33 | (0.72~2.48) |
| SNP_30 | 0.141 | 0.152 | 0.363 | 1 | 0.275 | 1 | 1.09 | (0.90~1.32) | 1.42 | (0.76~2.66) |
| SNP_31 | 0.141 | 0.152 | 0.352 | 1 | 0.267 | 0.9997 | 1.09 | (0.91~1.32) | 1.43 | (0.76~2.67) |
| SNP_32 | 0.156 | 0.189 | 0.00952 | 0.24 | 0.323 | 1 | 1.26 | (1.06~1.51) | 1.39 | (0.72~2.66) |
| SNP_33 | 0.461 | 0.426 | 0.0392 | 0.66 | 0.0672 | 0.83935 | 1.15 | (1.01~1.32) | 1.21 | (0.99~1.49) |
| SNP_34 | 0.126 | 0.128 | 0.818 | 1 | 0.856 | 1 | 1.02 | (0.84~1.25) | 1.07 | (0.51~2.23) |
| SNP_35 | 0.407 | 0.424 | 0.324 | 1 | 0.685 | 1 | 1.07 | (0.93~1.23) | 1.05 | (0.82~1.35) |
| SNP_36 | 0.124 | 0.094 | 0.00456 | 0.136 | 0.00857 | 0.22565 | 1.37 | (1.10~1.70) | 1.37 | (1.08~1.74) |
| SNP_37 | 0.170 | 0.148 | 0.0736 | 0.859 | 0.146 | 0.97665 | 1.18 | (0.98~1.42) | 1.17 | (0.95~1.44) |
| SNP_38 | 0.414 | 0.427 | 0.414 | 1 | 0.698 | 1 | 1.06 | (0.92~1.21) | 1.05 | (0.82~1.34) |
| SNP_39 | 0.153 | 0.134 | 0.120 | 0.968 | 0.153 | 0.98655 | 1.16 | (0.96~1.41) | 1.17 | (0.94~1.45) |
| SNP_40 | 0.089 | 0.079 | 0.272 | 1 | 0.299 | 1 | 1.14 | (0.90~1.46) | 1.15 | (0.89~1.48) |
| SNP_41 | 0.403 | 0.422 | 0.255 | 1 | 0.147 | 0.9751 | 1.08 | (0.94~1.24) | 1.20 | (0.94~1.53) |
| SNP_42 | 0.463 | 0.446 | 0.320 | 1 | 0.372 | 1 | 1.07 | (0.94~1.22) | 1.10 | (0.89~1.35) |
| SNP_43 | 0.344 | 0.381 | 0.0219 | 0.449 | 0.0229 | 0.47195 | 1.18 | (1.02~1.35) | 1.39 | (1.05~1.84) |
| SNP_44 | 0.143 | 0.126 | 0.150 | 0.987 | 0.135 | 0.9722 | 1.16 | (0.95~1.41) | 1.18 | (0.95~1.47) |
| SNP_45 | 0.131 | 0.123 | 0.464 | 1 | 0.358 | 1 | 1.08 | (0.88~1.32) | 1.11 | (0.89~1.39) |

TABLE 7

| | | | | | | | | | | | P-value | | Odds ratio | |
|---|---|---|---|---|---|---|---|---|---|---|---|---|---|---|
| | | | | | | | | | Minor allele | | (Adjusted_P) | | (95% c.i.) | |
| | Case | | | | Control | | | | frequency | | | A + AG | | AA + AG |
| Phenotype | AA | AG | GG | Total | AA | AG | GG | Total | Case | Control | A vs. G | vs. GG | A vs. G | vs. GG |
| Lacunar infarction | 27 | 178 | 286 | 491 | 11 | 130 | 344 | 485 | 0.236 | 0.157 | 9.84E−06 | 3.47E−05 | 1.66 | 1.75 |
| group | | | | | | | | | | | (0.0004) | (0.0009) | (1.33~2.09) | (1.34~2.28) |
| Combined lacunar | 41 | 310 | 507 | 858 | 27 | 239 | 582 | 848 | 0.228 | 0.173 | 4.92E−05 | 4.12E−05 | 1.42 | 1.51 |
| infarction and | | | | | | | | | | | (0.00155) | (0.0014) | (1.20~1.68) | (1.24~1.85) |
| atherothrombotic | | | | | | | | | | | | | | |
| infarction group | | | | | | | | | | | | | | |

TABLE 8

| | Age- and sex-adjusted | | | Multivariable-adjusted | | |
|---|---|---|---|---|---|---|
| Genotype | OR | (95% c.i.) | p-Value | HR | (95% c.i.) | p-Value |
| GG | 1.00 | | | 1.00 | | |
| GA | 1.52 | (1.22-1.88) | 0.0001 | 1.61 | (1.26-2.06) | 0.0001 |
| AA | 1.82 | (1.09-3.03) | 0.022 | 2.10 | (1.18-3.73) | 0.012 |
| GG | 1.00 | | | 1.00 | | |
| GA + AA | 1.55 | (1.26-1.91) | <0.0001 | 1.66 | (1.31-2.11) | <0.0001 |

stratification between the cases and controls, but in both analyses, significant group stratification was not indicated in the subjects of the present inventors.

### Example 2

### Effect of V374I on PKCη Activity

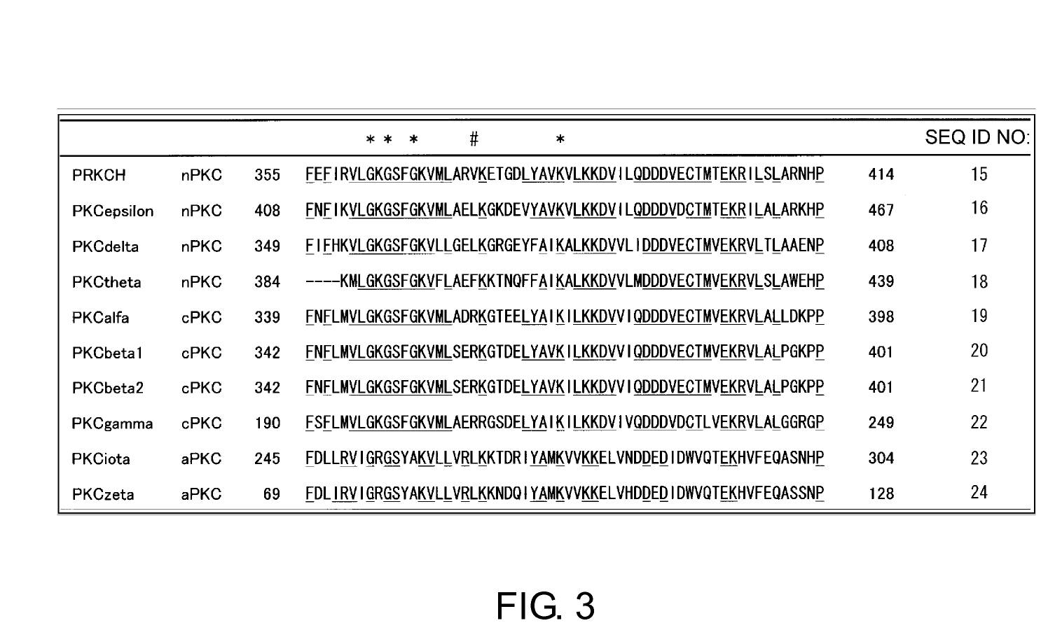

The V374I amino acid substitution in PKCη exists inside the ATP binding site which is conserved in the PKC family (FIG. 2b, FIG. 3) (Osada, S. et al., Cell Growth Diff. 4, 167-175 (1993)). Therefore, the present inventors investigated the effect of V374I on PKCη kinase activity. The present inventors constructed Flag-PKCη-374V and Flag-PKCη-374I expression vectors, and transfected them into 293T cells. After transient transfection, both proteins were immunoprecipitated and purified using an anti-Flag M2 agarose gel. The quality and concentration of the immunoprecipitates were evaluated by Coomassie Brilliant Blue staining and Western blotting (FIG. 2c, d). Since PKCη has been reported to be activated by autophosphorylation (Nishizuka, Y. Science. 258, 607-614 (1992)), the present inventors examined the kinase activity of each protein by autophosphorylation assay. After stimulation with 10 nM phosphatidyl serine (PS) and 100 nM phorbol 12,13-dibutyrate (PDBu), autophosphorylation of PKCη was observed for at least one minute, and the degree of autophosphorylation was higher in PKCη-374I than in PKCη-374V (FIG. 2d). To confirm these results, PKC activity was examined using myelin basic protein as a substrate. The activity in PKCη-374I was 1.6-times greater than that of PKCη-374V (p=0.009, FIG. 2e). These results suggest the possibility that the amino acid change from 374V to 374I in PKCη induces a higher level of autophosphorylation and kinase activation after stimulation and leads to activation of the downstream signal transduction pathway.

### Example 3

### Expression of PKCη in Atherosclerosis

In mice, PKCη is expressed mainly in the epithelial tissues including the skin, digestive tract, and airway (Osada, S. et al., J. Biol. Chem. 265, 22434-22440 (1990)). The expression pattern of PKCη in humans is undetermined, and the relationship between PKCη and cerebral infarction cannot be explained from this distribution pattern in mice.

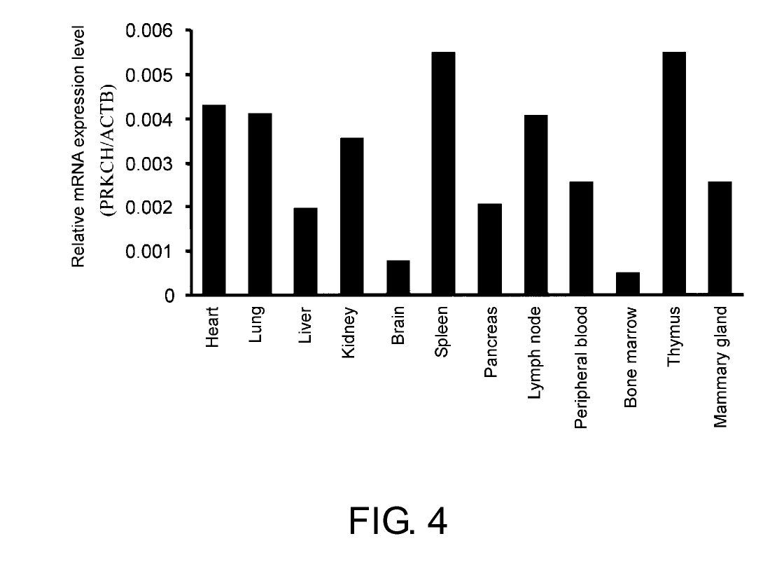

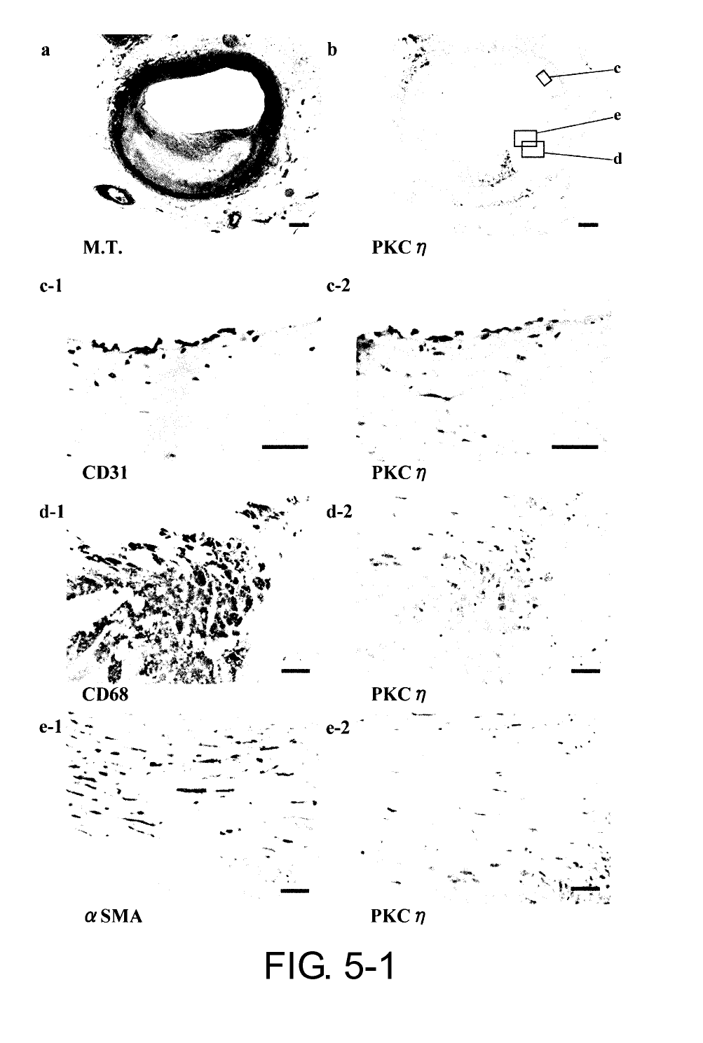

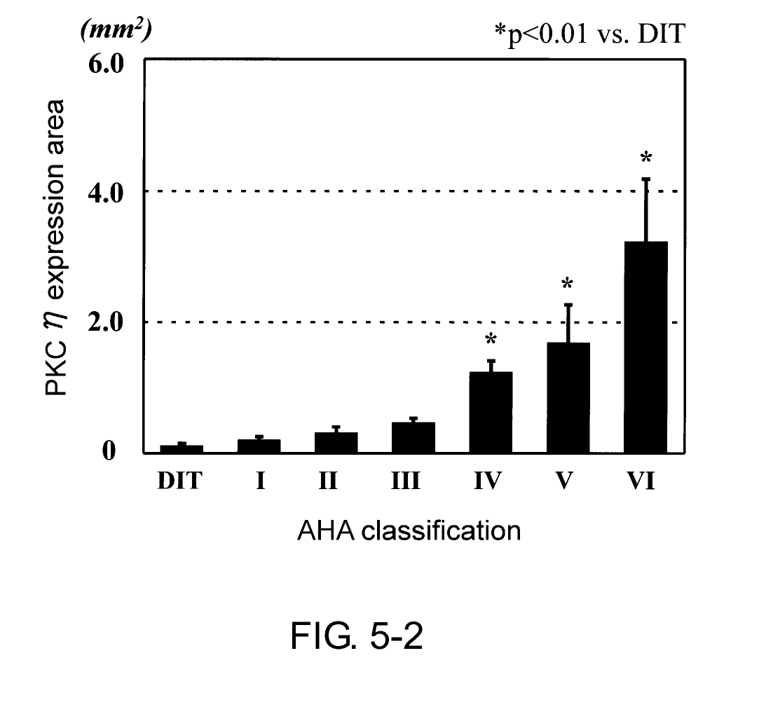

To examine the expression pattern of PKCη in human tissues, the present inventors performed quantitative real-time PCR using human cDNA in various tissues. PKCη was expressed universally in the various human tissues, and expression in the thymus and spleen was somewhat high (FIG. 4). Based on these results and the association to cerebral infarction, the present inventors examined the expression of PKCη in atherosclerotic lesions. Immunohistostaining of coronary artery preparations obtained at the time of autopsy showed that PKCη was expressed in the endothelial cells in the arterial intima, a portion of foamy macrophages, and spindle smooth muscle cells in the arterial intima and media (b to e of FIG. 5-1). In the outer membrane, PKCη was constantly expressed by a portion of vascular endothelial cells (data not shown). This expression could not be observed in preparations that had been pre-absorbed using immunogenic peptides or normal rabbit IgG

To further examine the relationship with PKCη and atherosclerosis, 60 coronary artery preparations obtained at the time of autopsy were carefully classified according to the types of atherosclerotic lesion (AHA classification (Stary, H C. et al., Circulation. 92, 1355-74 (1995))). PKCη-positive cells were quantified by NIH imaging. PKCη expression matched the severity of coronary artery atherosclerosis, and it was observed at higher frequency in advanced lesions (p<0.0001, f of FIG. 5-2). These results suggest that PKCη is closely related to the onset and progress of atherosclerosis in humans.

### Example 4

### Validation Test of V374I Using a Population-Based Prospective Cohort

In case-control studies, there is a danger of eliciting false-positive results due to selection bias of subjects and especially controls. Therefore, a candidate SNP identified in a single correlation analysis should be verified in a different population. However, the minor allele frequencies of SNP_{—}15 (IMS-JST140193) used as a marker SNP in the correlation analysis by the present inventors were 0.239 in the Japanese group, 0.229 in the Chinese group, 0.022 in the African group, and 0.00 in the European group. These data suggest that the candidate SNP in PRKCH is specific to the Asian population and that this is difficult to reproduce in Caucasians.

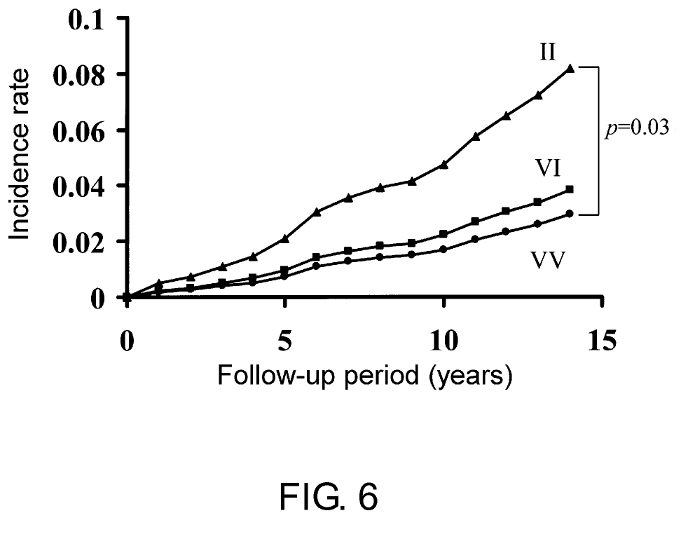

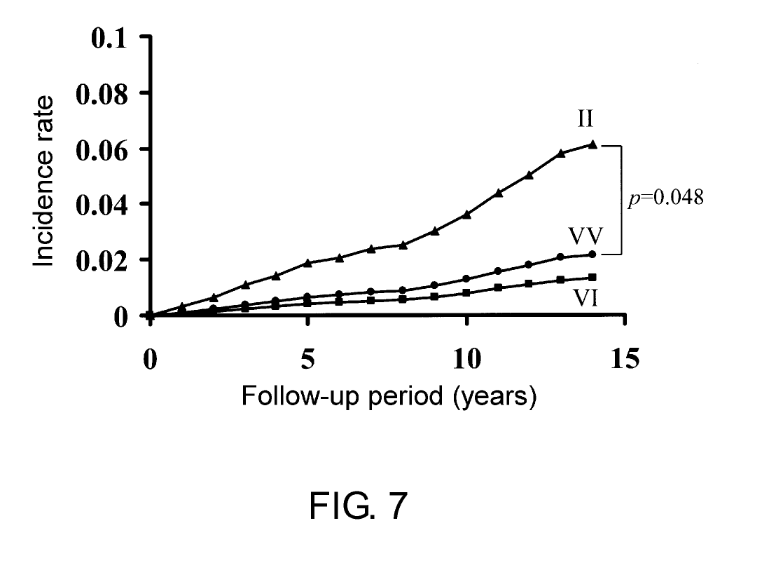

To overcome this problem, the present inventors examined the association of rs2230500 in a population-based prospective cohort constructed in 1988. During a 14-year-long follow-up study, of the 1,642 subjects who had not had a medical history of cerebral apoplexy at the time of base-line examination, cerebral infarction occurred for the first time in 67 subjects. As a result of comparing the cerebral infarction incidence rates of the rs2230500 genotype, the present inventors discovered that the incidence rate increased linearly in the order of GG, GA, and AA genotypes (this corresponds to amino acid substitutions VV, VI, and II) (FIG. 6). The age- and sex-adjusted relative risk of cerebral infarction incidence rate in comparison to the GG genotype was 1.31 (95% c.i., 0.78 to 2.19) for the GA genotype and 2.83 (95% c.i., 1.11 to 7.22) for the AA genotype (Table 9). The risk for development of cerebral infarction in the AA genotype subjects was significantly higher in comparison to that of the GA genotype or GG genotype subjects (p=0.043, relative risk 2.58, 95% c.i., 1.03 to 6.44). This relationship did not change substantially even after adjustment of baseline clinical risk factors including age, sex, hypertension, diabetes, serum cholesterol, smoking, and drinking habits. The risk of rs2230500 for development of coronary artery disease using 1,661 subjects in the same cohort yielded similar results (FIG. 7). These results indicate that rs2230500 in PRKCH is a genetic risk factor

TABLE 9

| | | | Age- and sex-adjusted | | | Multivariable-adjusted | | |
|---|---|---|---|---|---|---|---|---|
| Genotype | Number | Event | HR | (95% c.i.) | p-Value | HR | (95% c.i.) | p-Value |
| GG | 1063 | 39 | 1.00 | | | 1.00 | | |
| AG | 518 | 23 | 1.31 | (0.78-2.19) | 0.309 | 1.31 | (0.78-2.20) | 0.317 |
| AA | 61 | 5 | 2.83 | (1.11-7.22) | 0.030 | 2.91 | (1.14-7.47) | 0.026 |
| GG + AG | 1581 | 62 | 1.00 | | | 1.00 | | |
| AA | 61 | 5 | 2.58 | (1.03-6.44) | 0.043 | 2.66 | (1.06-6.68) | 0.038 |

In Table 9 above, “HR” refers to hazard ratio and “c.i.” refers to confidence interval. Adjustments for multivariable analysis were made for age, sex, hypertension, diabetes, cholesterol, smoking, and drinking.

### <Materials, Methods, and Such for Various Experiments Relating to the AGTRL1 Gene>

### Test Population

Since 1961, the present inventors have been conducting cohort studies using a regional population on cardiovascular diseases in residents of Hisayama which is a suburban area adjacent to Fukuoka city, Japan (Hata J. et al., J. Neurol. Neurosurg. Psychiatry 2005; 76: 368-372; and Tanizaki Y., et al., Stroke 2000, 31: 2616-2622). Between 2002 and 2003, the present inventors performed screening studies on residents of Hisayama, and 3,328 residents aged of 40 or more (78% of the whole population belonging to this age group) participated in this study. Of them, 3,196 participants (96%) agreed to use their own clinical data and DNA samples for the present study.

The present inventors registered cerebral infarction patients from Kyushu University Hospital and six affiliated hospitals in the Fukuoka urban area (National Hospital Organization Kyushu Medical Center, National Hospital Organization Fukuoka-Higashi Medical Center, Japanese Red Cross Fukuoka Hospital, Hakujyuji Hospital, Imazu Red Cross Hospital, and Seiai Rehabilitation Hospital). All test cases were diagnosed as cerebral apoplexy by a neurologist using clinical information and brain imaging tests including computed tomography (CT) and/or magnetic resonance imaging (MRI), and subdivided into LA, AT, CE, and other subtypes. The subjects were all Japanese and written informed consents were obtained for participation in the study.

This study has been approved by the ethics committees of the Faculty of Medical Sciences in Kyushu University, the Institute of Medical Science in the University of Tokyo, and each participating hospital.

### Correlation Analysis

The present inventors performed genome-wide correlation analysis in a stepwise manner consisting of first and second screening. In the first screening, 188 test cases affected by cerebral infarction were randomly selected. For each case, one age- and sex-matched control subject was randomly selected from Hisayama residents who had never been affected by cerebral apoplexy or coronary artery disease.

In the second screening, 860 test cases with LA and AT were incorporated as controls. Other subtypes of CE and cerebral infarction were not included in the second screening since they have mechanisms that are different from LA and AT. For each test case, one age- and sex-matched control subject was randomly selected from Hisayama residents. In both groups, the mean age±s.d. was 70±10 years old, and 60.7% of the subjects were men.

### SNP Genotyping

Genomic DNA samples were extracted from whole blood by standard methods. Many genomic fragments were amplified using each polymerase chain reaction (PCR), and SNPs were genotyped using invader assay (Ohnishi Y, et al., J. Hum. Genet. 2001, 46:471-477; Lyamichev V., et al., Nat. Biotech. 1999, 17:292-296).

### Cell Culture

Human gastric adenocarcinoma SBC-3 cells were grown in RPMI 1640 medium containing 10% fetal bovine serum (FBS) and 1% antibiotic/antifungal solution (SIGMA). Human embryonic kidney fibroblast 293T cells were grown in Dulbecco's modified Eagle medium containing 10% FBS and 1% antibiotic/antifungal solution (SIGMA). Both cells were incubated at 37° C. and humidified air containing 5% CO₂.

### Electrophoretic Mobility Shift Assay (EMSA)

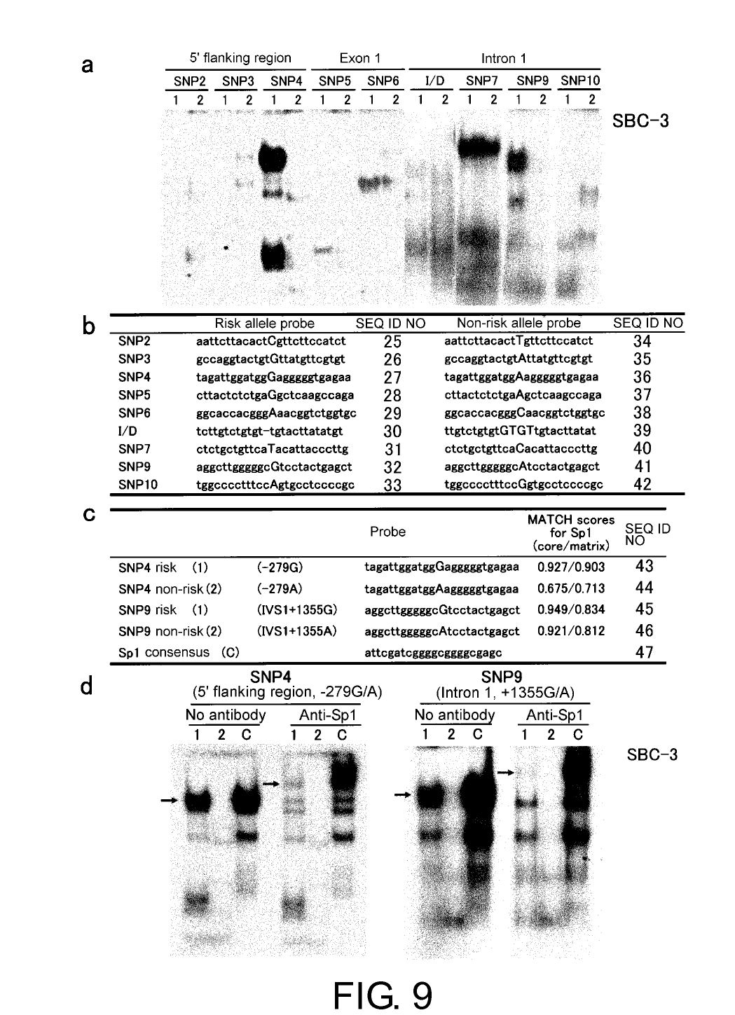

Probes used for EMSA were constructed as 25-bp double-stranded oligonucleotides around each polymorphism as shown in FIG. 9a. Sp1-concensus oligonucleotide (SantaCruz, sc-2502) was used as a positive control for Sp1 binding. Each probe was labeled with [γ-³²P]-ATP (Amersham) using T₄ polynucleotide kinase (TOYOBO). 10 μg of SBC-3 nuclear extract was incubated at room temperature for 30 minutes with a probe (>500,000 cpm) in a reaction mixture containing 15 mM Tris-HCl (pH7.5), 6.5% glycerol, 50 mM KCl, 0.7 mM EDTA (pH8.0), 0.2 mM dithiothreitol, 1 mg/mL bovine serum albumin, 1 μg of poly (dI-dC), and 0.1 μg of salmon sperm DNA. For the Sp1 supershift assay, 2 μg of anti-human Sp1 goat polyclonal antibody (SantaCruz, sc-59X) was added, and this was further incubated at room temperature for 30 minutes. This mixture was subjected to electrophoresis at 120 V for three hours on a 4% polyacrylamide gel containing 0.5× Tris-borate-EDTA buffer. After drying the gel, it was exposed to an X-ray film.

### Overexpression of Sp1 and Reverse Transcriptase Polymerase Chain Reaction (RT-PCR)

Full-length human Sp1 cDNA was subcloned into a pCAGGS expression vector (pCAGGS-Sp1). 293T cells grown until nearly confluent in a 6-well culture plate were transfected with 1 μg of pCAGGS-Sp1 or mock pCAGGS using 3 μL of FuGENE6 (Roche) per well. Total RNA was collected at various times before or after transfection, and purified using the RNeasy Mini Kit and RNase-free DNase Set (QIAGEN). cDNA was synthesized using Superscript II reverse transcriptase (Invitrogen). Expression levels of human AGTRL1 and housekeeping gene B2M were determined using semi-quantitative RT PCR, and were quantitatively confirmed by Real Time RT-PCR.

For semi-quantitative RT-PCR, ExTaq DNA polymerase (TaKaRa) and each of the following primer pairs were used to amplify the cDNA

AGTRL1
(5′-CTGTGGGCTACCTACACGTAC-3′ (SEQ ID NO: 7)
and
5′-TAGGGGATGGATTTCTCGTG-3′ (SEQ ID NO: 8)),
and B2M
(5′-CACCCCCACTGAAAAAGATGA-3′ (SEQ ID NO: 9)
and
5′-TACCTGTGGAGCAACCTGC-3′ (SEQ ID NO: 10)).

For real-time PCR, cDNA was amplified using SYBR Premix ExTaq (TaKaRa) and analyzed using ABI PRISM 7700 (Applied Biosystems) and each of the following primer pairs:

AGTRL1
(5′-TGCCATCTACATGTTGGTCTTC-3′ (SEQ ID NO: 11)
and
5′-GTCACCACGAAGGTCAGGTC-3′ (SEQ ID NO: 12)),
and B2M
(5′-TCTCTCTTTCTGGCCTGGAG-3′ (SEQ ID NO: 13)
and
5′-AATGTCGGATGGATGAAACC-3′ (SEQ ID NO: 14)).

mRNA was quantified by correlating the obtained PCR threshold cycle to a cDNA standard curve. Standardized AGTRL1 expression level was obtained by dividing the AGTRL1 value by the B2M value.

### Luciferase Assay

A DNA fragment corresponding to nt-291 to -248 of the 5′ flanking region of AGTRL1 containing any one of the alleles of SNP4 (rs9943582), and/or to nt+1329 to +1381 of the intron containing any one of the alleles of SNP9 (rs2282624) was synthesized, and cloned into the multicloning site of the pGL3-basic reporter plasmid (Promega). SBC-3 cells grown to be nearly confluent in a 24-well culture plate were transfected with 90 ng of each reporter construct, 10 ng of pRL-CMV vector used as an internal control of transfection efficiency, and 100 ng of either the pCAGGS-Sp1 or mock pCAGGS vector, using 0.6 μL of FuGENE6 (Roche) per well. The cells were collected 48 hours later and luciferase activity was measured using a Dual-Luciferase Reporter Assay System (Promega).

### Statistical Analyses

Statistical analyses for correlation analysis and Hardy-Weinberg equilibrium were performed as described by Yamada et al (Yamada, R., et al., Am. J. Hum. Genet. 2001; 68: 674-685). For adjustment of multiple tests, the present inventors performed 10,000 permutation tests using the MULTITEST method of SAS software (SAS Institute). Haplotype frequency and linkage disequilibrium index (D′ and Δ2) were evaluated using an expectation-maximization algorithm. Relative luciferase activities were compared by Student's t-test.

### URL

The JSNP database is available at http://snp.ims.u-tokyo.ac.jp/index.html. The International HapMap Project database is available at http://www.hapmap.org. The dbSNP database provided by the National Center of Biotechnology Information in the U.S. is available at http://www.ncbi.nlm.nih.gov/SNP/index.html. The GENSCAN program is available at http://genes.mit.edu/GENSCAN.html. The MATCH program is available at http://www.gene-regulation.com/.

### Example 5

### Genome-Wide Correlation Analysis

The present inventors performed genome-wide correlation analysis in a stepwise manner. For the first screening, 188 Japanese patients with cerebral infarction and 188 age- and sex-matched control subjects were used, and 52,608 gene-based SNPs were selected from the JSNP database (Haga H., et al, J. Hum. Genet. 2002; 47: 605-610) by a high-throughput multiplex PCR-invader assay method (Ohnishi Y, et al, J. Hum. Genet. 2001; 46: 471-477). Genotypes and allele frequencies were compared between the cases and controls, and 1,098 types of SNPs whose p-values were shown to be less than 0.01 were identified.

Next, for second screening, the present inventors used 860 patients with LA or AT and 860 age- and sex-matched control subjects to genotype these 1,098 types of SNPs. The present inventors discovered that of them, an SNP in the AGTRL1 gene (SNP6, rs948847) showed low p-values in the allele frequency model (p=0.000028) and the recessive model (p=0.000057). The adjusted p-value in the recessive model after performing permutation tests for multiple tests was 0.0498. The present inventors considered that SNP6 was a candidate marker SNP associated with cerebral infarction.

### Example 6

### High Precision Mapping Around Marker SNP6

To examine the chromosome 11q12 region where SNP6 is positioned, the present inventors used all subjects to genotype 48 types of SNPs in the 240-kb region around AGTRL1. This region covered seven genes: PRG2, PRG3, P2RX3, SSRP1, TNKS1BP1, AGTRL1, and LRRC55. Of them, ten types of SNPs were in AGTRL1, and 38 types of other SNPs whose minor allele frequency was more than 20% were selected from the database of International HapMap Project (The International HapMap Consortium. Nature 2005; 437: 1299-1320) and the JSNP database (Haga H., et al, J. Hum. Genet. 2002; 47: 605-610).

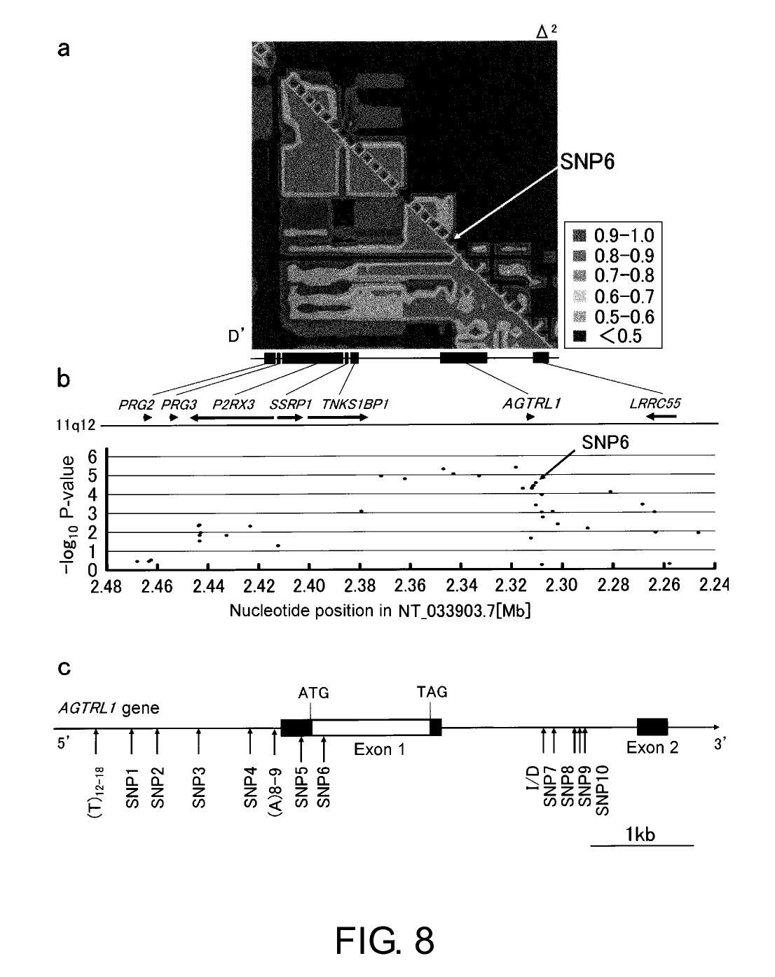

The present inventors constructed a linkage disequilibrium (LD) map of this region (FIG. 8a). These D′-values showed that marker SNP6 is associated with the region covered by five genes (P2RX3, SSRP1, TNKS1BP1, AGTRL1, and LRRC55). Therefore, it was difficult to determine a single candidate gene implicated in cerebral infarction from the LD mapping alone.

Subsequently, the present inventors evaluated the P-values in correlation analyses within this region (FIG. 8b). SNP showing the most significant association was positioned between the TNKS1BP1 gene and the AGTRL1 gene, but there were no reports of genes or expression sequence tags in this region, and open reading frames could not be predicted by the GENSCAN program either. SNPs in P2RX3, SSRP1, TNKSBP1, and LRRC55 had lower degrees of significance of association compared to that of the marker SNP6 in the AGTRL1 gene. Furthermore, the present inventors selected AGTRL1 as a candidate gene implicated in cerebral infarction since the APJ receptor, which is an AGTRL1 product, has been reported to be expressed in the cardiovascular system (Kleinz M J, et al., Regul. Pept. 2005; 126: 233-240) and the central nervous system (O'Carroll A M, et al., J. Neuroendocrinol. 2003; 15: 661-666), and has a function associated with the cardiovascular system (Kagiyama S, et al., Regl. Pept. 2005; 125: 55-59; Seyedabadi M, et al., Auton. Neurosci. 2002, 101: 32-38; Katugampola S D, et al., Br. J. Pharmacol. 2001, 132: 1255-1260; Tatemoto K, et al, Regul. Pept. 2001, 99: 87-92; Masri B, et al., FASEB J. 2004, 18: 1909-1911; Hashimoto Y, et al., Int. J. Mol. Med. 2005, 16: 787-792).

### Example 7

### SNP Analysis of the AGTRL1 Gene

The AGTRL1 gene consists of two exons and one intron (FIG. 8c). The protein-coding region exists only in exon 1. By the direct sequencing described in a previous report (Saito S., et al., J. Hum. Genet. 2003; 48: 461-468), the region ranging from 2 kb upstream of the transcription start site to the last exon has been screened for genetic mutations in the AGTRL1 gene. In that report, nine SNPs, two simple-repeat polymorphisms, and one insertion/deletion (I/D) polymorphism were found. The present inventors discovered another SNP (SNP5) in the 5′ untranslated region (UTR) of exon 1 that had not been discovered in previous reports, but was already registered as rs11544374 in the dbSNP database. The present inventors genotyped these ten types of SNPs using 860 test cases having LA and AT, and 860 control subjects. The I/D polymorphism in the intron was also genotyped by direct sequencing, and was found to have absolute linkage with SNP7 and SNP10.

In this case-control study, there were nine polymorphisms showing significant association with cerebral infarction (SNP2, 3, 4, 5, 6, 7, 9, 10, and I/D) (Table 10).

Table 10 below shows case-control correlation analysis of the AGTRL1 gene.

TABLE 10

| | | |
|---|---|---|
| SNP | Case (n = 860) | Control (n = 860) |

| SNP | dbSNP ID | position (1/2) | 11 | 12 | 22 | Total | 11 | 12 | 22 | Total |
|---|---|---|---|---|---|---|---|---|---|---|
| SNP1 | rs4939123 | −1433T/A | 4 | 105 | 745 | 854 | 1 | 81 | 778 | 860 |
| SNP2 | rs7119375 | −1176C/T | 450 | 347 | 59 | 856 | 373 | 391 | 91 | 855 |
| SNP3 | rs10501367 | −799G/A | 451 | 345 | 59 | 855 | 373 | 391 | 91 | 855 |
| SNP4 | rs9943582 | −279G/A | 450 | 346 | 59 | 855 | 376 | 389 | 94 | 859 |
| SNP5 | rs11544374 | +212G/A (5′UTR) | 495 | 314 | 47 | 856 | 427 | 362 | 69 | 858 |
| SNP6 | rs948847 | +445A/C (Gly45Gly) | 431 | 357 | 70 | 858 | 345 | 405 | 101 | 851 |
| SNP7 | rs746886 | IVS1 + 1155T/C | 159 | 398 | 299 | 856 | 110 | 412 | 337 | 859 |
| SNP8 | rs2282625 | IVS1 + 1338C/T | 375 | 382 | 98 | 855 | 368 | 400 | 92 | 860 |
| SNP9 | rs2282624 | IVS1 + 1355G/A | 182 | 410 | 264 | 856 | 124 | 418 | 318 | 860 |
| SNP10 | rs2282623 | IVS1 + 1440A/G | 160 | 399 | 300 | 859 | 112 | 411 | 331 | 854 |

| | Allele frequency (1/2) | | Recessive model (11/12 + 22) | | Dominant model (11/12 + 22) | |
|---|---|---|---|---|---|---|
| SNP | OR (95% CI) | P-value | OR (95% CI) | P-value | OR (95% CI) | P-value |
| SNP1 | 1.40 (1.04~1.87) | 0.024 | 4.04 (0.45~36.2) | 0.18 | 1.39 (1.02~1.88) | 0.034 |
| SNP2 | 1.35 (1.17~1.56) | 0.000054 | 1.43 (1.18~1.73) | 0.00022 | 1.61 (1.14~2.27) | 0.0061 |
| SNP3 | 1.36 (1.17~1.57) | 0.000043 | 1.44 (1.19~1.75) | 0.00016 | 1.61 (1.14~2.26) | 0.0062 |
| SNP4 | 1.36 (1.17~1.57) | 0.000040 | 1.43 (1.18~1.73) | 0.00024 | 1.66 (1.18~2.33) | 0.0033 |
| SNP5 | 1.31 (1.13~1.53) | 0.00043 | 1.38 (1.14~1.67) | 0.00082 | 1.51 (1.03~2.21) | 0.036 |
| SNP6 | 1.36 (1.18~1.57) | 0.000028 | 1.48 (1.22~1.79) | 0.000057 | 1.52 (1.10~2.09) | 0.011 |
| SNP7 | 1.24 (1.08~1.42) | 0.0025 | 1.55 (1.19~2.02) | 0.0010 | 1.20 (0.99~1.46) | 0.065 |
| SNP8 | 1.01 (0.87~1.16) | 0.92 | 1.04 (0.86~1.26) | 0.66 | 0.93 (0.68~1.25) | 0.61 |
| SNP9 | 1.31 (1.14~1.50) | 0.00012 | 1.60 (1.25~2.06) | 0.00021 | 1.32 (1.08~1.61) | 0.0073 |
| SNP10 | 1.22 (1.06~1.39) | 0.0052 | 1.52 (1.17~1.97) | 0.0018 | 1.18 (0.97~1.44) | 0.10 |

In Table 10 above, “1” refers to risk allele, “2” refers to non-risk allele, “OR” refers to odds ratio, and “CT” refers to confidence interval.

Furthermore, the results of correlation analyses in all cerebral infarctions are shown in Tables 11-1 and 11-2 below. The results of correlation analyses in the lacunar group and atheroma group are shown in Tables 12-1 and 12-2 below.

Table 11-1

| | Patient group | | | | Control group | | | | | | | |
|---|---|---|---|---|---|---|---|---|---|---|---|---|
| | (1: risk allele) | | | | (1: risk allele) | | | | 2 × 3 Contingency table | (1/2) | (11/12 + 22) | (11 + 12/22) |
| dbSNP ID | 11 | 12 | 22 | Total | 11 | 12 | 22 | Total | (genotype) p-Value | p-Value | p-Value | p-Value |
| rs1939489 | 583 | 445 | 78 | 1106 | 496 | 498 | 118 | 1112 | 1.15E−04 | 2.28E−05 | 1.33E−04 | 3.15E−03 |
| rs4938861 | 584 | 444 | 80 | 1108 | 496 | 498 | 118 | 1112 | 1.54E−04 | 3.01E−05 | 1.34E−04 | 5.06E−03 |
| rs1892964 | 586 | 445 | 77 | 1108 | 493 | 501 | 117 | 1111 | 5.62E−05 | 1.12E−05 | 6.02E−05 | 2.82E−03 |
| rs1892963 | 585 | 445 | 77 | 1107 | 498 | 496 | 118 | 1112 | 1.03E−04 | 2.06E−05 | 1.46E−04 | 2.36E−03 |
| rs7102963 | 581 | 447 | 78 | 1106 | 493 | 498 | 117 | 1108 | 1.39E−04 | 2.85E−05 | 1.55E−04 | 3.60E−03 |
| rs499318 | 585 | 444 | 76 | 1105 | 493 | 498 | 117 | 1108 | 5.40E−05 | 1.07E−05 | 7.04E−05 | 2.15E−03 |
| rs1893675 | 536 | 467 | 105 | 1108 | 442 | 535 | 134 | 1111 | 1.87E−04 | 7.88E−05 | 4.58E−05 | 4.96E−02 |
| rs4939123 | 9 | 133 | 964 | 1106 | 2 | 107 | 1001 | 1110 | 1.87E−02 | 9.94E−03 | 3.39E−02 | 2.49E−02 |
| rs7119375 | 576 | 447 | 85 | 1108 | 482 | 508 | 115 | 1105 | 2.31E−04 | 6.17E−05 | 8.18E−05 | 2.48E−02 |
| rs10501367 | 577 | 445 | 84 | 1106 | 482 | 510 | 115 | 1107 | 1.38E−04 | 3.73E−05 | 4.84E−05 | 2.16E−02 |
| rs9943582 | 574 | 448 | 85 | 1107 | 486 | 507 | 118 | 1111 | 2.88E−04 | 6.62E−05 | 1.32E−04 | 1.63E−02 |
| rs11544374 | 641 | 400 | 67 | 1108 | 557 | 463 | 88 | 1108 | 1.27E−03 | 3.50E−04 | 3.43E−04 | 8.03E−02 |
| rs948847 | 551 | 455 | 99 | 1105 | 452 | 523 | 128 | 1103 | 1.12E−04 | 4.27E−05 | 2.76E−05 | 4.07E−02 |
| ss49849485 | | | | | | | | | | | | |
| rs746886 | 212 | 503 | 393 | 1108 | 145 | 543 | 423 | 1111 | 5.00E−04 | 3.10E−03 | 9.66E−05 | 2.03E−01 |
| rs746885 | 0 | 22 | 1086 | 1108 | 0 | 12 | 1099 | 1111 | #DIV/0! | 8.37E−02 | #DIV/0! | 8.25E−02 |
| rs2282625 | 499 | 483 | 125 | 1107 | 481 | 511 | 119 | 1111 | 5.33E−01 | 6.71E−01 | 3.98E−01 | 6.62E−01 |
| rs2282624 | 243 | 517 | 348 | 1108 | 161 | 558 | 393 | 1112 | 2.85E−05 | 1.24E−04 | 5.35E−06 | 4.94E−02 |
| rs2282623 | 213 | 505 | 391 | 1109 | 147 | 541 | 417 | 1105 | 8.38E−04 | 4.39E−03 | 1.67E−04 | 2.25E−01 |
| rs721608 | 641 | 402 | 64 | 1107 | 585 | 433 | 93 | 1111 | 1.08E−02 | 2.89E−03 | 1.29E−02 | 1.74E−02 |
| rs1943482 | 291 | 527 | 289 | 1107 | 228 | 563 | 321 | 1112 | 5.24E−03 | 4.40E−03 | 1.29E−03 | 1.45E−01 |
| rs10896586 | 204 | 517 | 387 | 1108 | 154 | 526 | 432 | 1112 | 8.54E−03 | 3.86E−03 | 3.47E−03 | 5.56E−02 |
| rs2156456 | 225 | 505 | 375 | 1105 | 151 | 527 | 429 | 1107 | 8.88E−05 | 9.19E−05 | 2.58E−05 | 1.85E−02 |
| rs717211 | 186 | 500 | 420 | 1106 | 120 | 516 | 474 | 1110 | 1.40E−04 | 2.11E−04 | 4.17E−05 | 2.33E−02 |

Table 11-2
Table 11-2 is the continuation of Table 11-1.

| | | | | | |
|---|---|---|---|---|---|
| | Position on the gene | 1 | 2 | | |
| dbSNP ID | AGTRL1 | Risk | Non-risk | JSNP | NT_033903.7 |

| | | | | | | |
|---|---|---|---|---|---|---|
| rs1939489 | | | C | A | | 2371291 |
| rs4938861 | | | G | T | | 2361845 |
| rs1892964 | | | G | T | | 2346515 |
| rs1892963 | | | C | T | | 2342835 |
| rs7102963 | | | T | G | | 2332629 |
| rs499318 | | | T | C | | 2318117 |
| rs1893675 | | | C | A | | 2315139 |
| rs4939123 | 5′flank, −1433 | SNP1 | T | A | ssj0009877 | 2312016 |
| rs7119375 | 5′flank, −1176 | SNP2 | C | T | ssj0009878 | 2311759 |
| rs10501367 | 5′flank, −799 | SNP3 | G | A | ssj0009879 | 2311382 |
| rs9943582 | 5′flank, −279 | SNP4 | G | A | ssj0009880 | 2310862 |
| rs11544374 | ex1(5UTR). 212 | SNP5 | G | A | | 2310372 |
| rs948847 | ex1(Gly45Gly). 445 | SNP6 | A | C | IMS-JST092074 | 2310139 |
| ss49849485 | int1. 1045-1048 | I/D | del | ins | ssj0009883 | 2308013-2308016 |
| rs746886 | int1. 1155 | SNP7 | T | C | ssj0009884 | 2307906 |
| rs746885 | int1. 1234 | | T | C | | 2307827 |
| rs2282625 | int1. 1338 | SNP8 | C | T | IMS-JST031820 | 2307723 |
| rs2282624 | int1. 1355 | SNP9 | G | A | IMS-JST031819 | 2307706 |
| rs2282623 | int1. 1440 | SNP10 | A | G | IMS-JST031818 | 2307621 |
| rs721608 | | | T | C | | 2303718 |
| rs1943482 | | | C | T | | 2301404 |
| rs10896586 | | | A | G | | 2289898 |
| rs2156456 | | | T | C | | 2280894 |
| rs717211 | | | T | C | | 2268354 |

Table 12-1

| | Patient group | | | | Control group | | | | 2 × 3 | | | |
|---|---|---|---|---|---|---|---|---|---|---|---|---|
| | (1: risk allele) | | | | (1: risk allele) | | | | Contingency table | (1/2) | (11/12 + 22) | (11 + 12/22) |
| dbSNP ID | 11 | 12 | 22 | Total | 11 | 12 | 22 | Total | (genotype) p-Value | p-Value | p-Value | p-Value |
| rs1939489 | 458 | 340 | 56 | 854 | 384 | 379 | 97 | 860 | 5.59E−05 | 1.19E−05 | 2.01E−04 | 6.08E−04 |
| rs4938861 | 459 | 339 | 58 | 856 | 384 | 379 | 97 | 860 | 8.68E−05 | 1.65E−05 | 2.02E−04 | 1.14E−03 |
| rs1892964 | 462 | 338 | 56 | 856 | 381 | 382 | 96 | 859 | 2.76E−05 | 5.12E−06 | 6.79E−05 | 7.36E−04 |
| rs1892963 | 461 | 339 | 56 | 856 | 385 | 378 | 97 | 860 | 4.71E−05 | 9.58E−06 | 1.67E−04 | 5.76E−04 |
| rs7102963 | 458 | 339 | 57 | 854 | 381 | 380 | 96 | 857 | 6.31E−05 | 1.22E−05 | 1.48E−04 | 1.03E−03 |
| rs499318 | 459 | 339 | 55 | 853 | 379 | 380 | 97 | 856 | 2.07E−05 | 4.19E−06 | 8.07E−05 | 3.91E−04 |
| rs1893675 | 417 | 364 | 75 | 856 | 340 | 412 | 108 | 860 | 2.31E−04 | 5.50E−05 | 1.28E−04 | 1.08E−02 |
| rs4939123 | 4 | 105 | 745 | 854 | 1 | 81 | 778 | 860 | 6.11E−02 | 2.40E−02 | 1.77E−01 | 3.37E−02 |
| rs7119375 | 450 | 347 | 59 | 856 | 373 | 391 | 91 | 855 | 2.42E−04 | 5.37E−05 | 2.14E−04 | 6.09E−03 |
| rs10501367 | 451 | 345 | 59 | 855 | 373 | 391 | 91 | 855 | 1.95E−04 | 4.25E−05 | 1.60E−04 | 6.23E−03 |
| rs9943582 | 450 | 346 | 59 | 855 | 376 | 389 | 94 | 859 | 1.89E−04 | 4.01E−05 | 2.42E−04 | 3.34E−03 |
| rs11544374 | 495 | 314 | 47 | 856 | 427 | 362 | 69 | 858 | 1.84E−03 | 4.32E−04 | 8.18E−04 | 3.55E−02 |
| rs948847 | 431 | 357 | 70 | 858 | 345 | 405 | 101 | 851 | 1.15E−04 | 2.80E−05 | 5.72E−05 | 1.06E−02 |
| ss49849485 | | | | | | | | | | | | |
| rs746886 | 159 | 398 | 299 | 856 | 110 | 412 | 337 | 859 | 3.29E−03 | 2.54E−03 | 1.02E−03 | 6.52E−02 |
| rs746885 | 0 | 16 | 840 | 856 | 0 | 9 | 851 | 860 | #DIV/0! | 1.57E−01 | #DIV/0! | 1.55E−01 |
| rs2282625 | 375 | 382 | 98 | 855 | 368 | 400 | 92 | 860 | 7.21E−01 | 9.25E−01 | 6.55E−01 | 6.14E−01 |
| rs2282624 | 182 | 410 | 264 | 856 | 124 | 418 | 318 | 860 | 3.24E−04 | 1.17E−04 | 2.13E−04 | 7.27E−03 |
| rs2282623 | 160 | 399 | 300 | 859 | 112 | 411 | 331 | 854 | 6.23E−03 | 5.15E−03 | 1.80E−03 | 1.00E−01 |
| rs721608 | 503 | 305 | 47 | 855 | 448 | 336 | 75 | 859 | 3.89E−03 | 9.17E−04 | 5.42E−03 | 9.22E−03 |
| rs1943482 | 218 | 417 | 220 | 855 | 176 | 422 | 262 | 860 | 1.70E−02 | 4.19E−03 | 1.33E−02 | 2.92E−02 |
| rs10896586 | 151 | 412 | 293 | 856 | 121 | 398 | 341 | 860 | 2.77E−02 | 7.04E−03 | 4.29E−02 | 2.00E−02 |
| rs2156456 | 171 | 395 | 288 | 854 | 117 | 391 | 347 | 855 | 4.04E−04 | 8.12E−05 | 4.64E−04 | 3.34E−03 |
| rs717211 | 141 | 392 | 322 | 855 | 93 | 390 | 375 | 858 | 9.70E−04 | 3.77E−04 | 6.60E−04 | 1.09E−02 |

Table 12-2
Table 12-2 is the continuation of Table 12-1.

| | | | | | |
|---|---|---|---|---|---|
| | position | 1 | 2 | | |
| dbSNP ID | AGTRL1 | Risk | Non-risk | JSNP | NT_033903.7 |

| | | | | | | |
|---|---|---|---|---|---|---|
| rs1939489 | | | C | A | | 2371291 |
| rs4938861 | | | G | T | | 2361845 |
| rs1892964 | | | G | T | | 2346515 |
| rs1892963 | | | C | T | | 2342835 |
| rs7102963 | | | T | G | | 2332629 |
| rs499318 | | | T | C | | 2318117 |
| rs1893675 | | | C | A | | 2315139 |
| rs4939123 | 5′flank, −1433 | SNP1 | T | A | ssj0009877 | 2312016 |
| rs7119375 | 5′flank, −1176 | SNP2 | C | T | ssj0009878 | 2311759 |
| rs10501367 | 5′flank, −799 | SNP3 | G | A | ssj0009879 | 2311382 |
| rs9943582 | 5′flank, −279 | SNP4 | G | A | ssj0009880 | 2310862 |
| rs11544374 | ex1(5UTR). 212 | SNP5 | G | A | | 2310372 |
| rs948847 | ex1(Gly45Gly). 445 | SNP6 | A | C | IMS-JST092074 | 2310139 |
| ss49849485 | int1. 1045-1048 | I/D | del | ins | ssj0009883 | 2308013-2308016 |
| rs746886 | int1. 1155 | SNP7 | T | C | ssj0009884 | 2307906 |
| rs746885 | int1. 1234 | | T | C | | 2307827 |
| rs2282625 | int1. 1338 | SNP8 | C | T | IMS-JST031820 | 2307723 |
| rs2282624 | int1. 1355 | SNP9 | G | A | IMS-JST031819 | 2307706 |
| rs2282623 | int1. 1440 | SNP10 | A | G | IMS-JST031818 | 2307621 |
| rs721608 | | | T | C | | 2303718 |
| rs1943482 | | | C | T | | 2301404 |
| rs10896586 | | | A | G | | 2289898 |
| rs2156456 | | | T | C | | 2280894 |
| rs717211 | | | T | C | | 2268354 |

TABLE 13

| | | Haplotype frequency | | | | Recessive model | | Dominant model | |
|---|---|---|---|---|---|---|---|---|---|
| Haplotype | SNPs | Case (%) | Control (%) | OR (95% CI) | P-value | OR (95% CI) | P-value | OR (95% CI) | P-value |
| Hap1 | C-G-A-G-A | 41.6 | 36.5 | 1.23 (1.08~1.42) | 0.0024 | 1.60 (1.22~2.09) | 0.00058 | 1.20 (0.98~1.45) | 0.075 |
| Hap2 | C-G-A-A-G | 26.3 | 26.0 | 1.02 (0.87~1.18) | 0.83 | 1.00 (0.68~1.48) | 0.99 | 1.03 (0.85~1.24) | 0.79 |
| Hap3 | T-A-C-A-G | 23.4 | 28.8 | 0.76 (0.65~0.88) | 0.00032 | 0.66 (0.45~0.97) | 0.033 | 0.72 (0.59~0.87) | 0.00062 |

In Table 13 above, five SNPs (SNP2, 5, 6, 9, and 10) were used for haplotype estimation. “OR” refers to odds ratio and “CT” refers to confidence interval.

SNPs in Hap1 were all risk alleles. Risk for cerebral infarction found in Hap1 was significantly higher than those in other haplotypes (odds ratio (OR)=1.60 (95% confidence interval (CI), 1.22 to 2.09); p=0.00058 in the recessive model). On the other hand, SNPs of Hap3 were all non-risk alleles, and risk for cerebral infarction found in Hap 3 was significantly lower than those in others (OR=0.76 (95% CI, 0.65 to 0.88); p=0.00032 in an allele frequency model). As a result, Hap1, Hap2, and Hap3 were determined to be haplotypes of the risk type, intermediate type, and non-risk type, respectively.

### Example

### Sp1 Transcription Factor Binds to the Risk Allele of SNP4 and SNP9 of the AGTRL1 Gene

To select cell systems that highly express AGTRL1 mRNA, the present inventors performed RT-PCR using cDNA from 89 types of cell systems. As a result, SBC-3 cells expressed AGTRL1 mRNA at the highest level (data not shown).

EMSA was performed to evaluate the binding between the transcription factor and DNA sequences around SNPs. ³²P-labeled double-stranded DNA probes were constructed for each allele of the nine candidate polymorphisms, and electrophoresis was performed after incubation with nuclear extract from SBC-3 cells (FIG. 9a). The present inventors found that DNA-protein binding was detected in risk allele SNP4 (−279G), but the same binding was not detected in the non-risk allele (−279A) of SNP4. The present inventors also discovered that DNA-protein binding was detected in the risk allele (+1355G) of SNP9, but not in the non-risk allele (+1355A). Similar results were obtained by using nuclear extracts from 293T cells (data not shown). The sequences of the probes used in these assays are shown in FIG. 9b. These results suggest the possibility that some kind of transcription factor binds in an allele-specific manner to transactivate AGTRL1 expression.

From the MATCH program using the TRANSFAC database, transcription factor Sp1 was predicted to bind to the DNA sequences around the risk alleles of SNP4 and SNP9 (FIG. 9c). To confirm the binding between these SNPs and Sp1 in vitro, the present inventors performed Sp1 supershift assays using a specific antibody (FIG. 9d). The band for the DNA-protein complex in the risk allele of SNP4 supershifted upward in the presence of an anti-Sp1 antibody. A similar supershift was also detected for the risk allele of SNP9.

According to the above, the present inventors concluded that the Sp1 transcription factor binds to the DNA sequences around the risk alleles of SNP4 and SNP9. The present inventors established a hypothesis that the interaction between Sp1 and these SNPs may affect the promoter activity or enhancer activity of the AGTRL1 gene.

### Example 10

### Sp1 Induces the Transcription of AGTRL1 mRNA

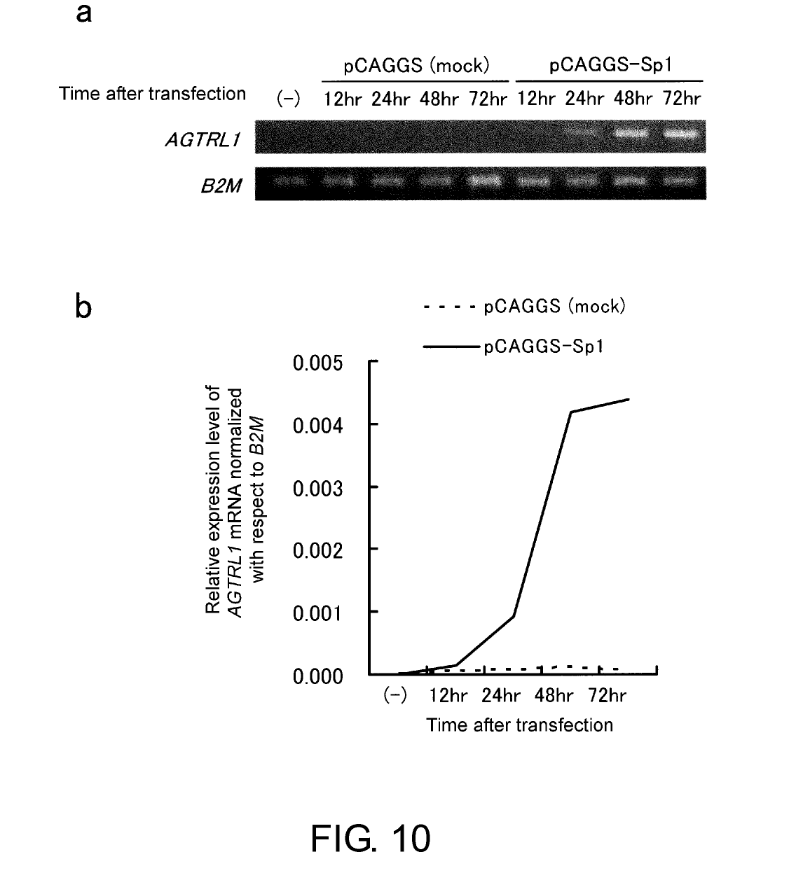

To confirm the hypothesis that Sp1 affects transcription of the AGTRL1 gene, the present inventors transfected 293 T cells with either the pCAGGS mock vector or the Sp1-expression vector (pCAGGS-Sp1), and compared the AGTRL1 mRNA levels by semi-quantitative RT-PCR at various time points (FIG. 10a). The present inventors found that AGTRL1 mRNA was not detected in endogenous 293T cells, but overexpression of Sp1 significantly induced transcription of AGTRL1 mRNA. The same results were quantitatively confirmed by real-time RT-PCR as well (FIG. 10b).

### Example 11

### Sp1 Activates the Promoter Function in the Risk Allele of SNP4

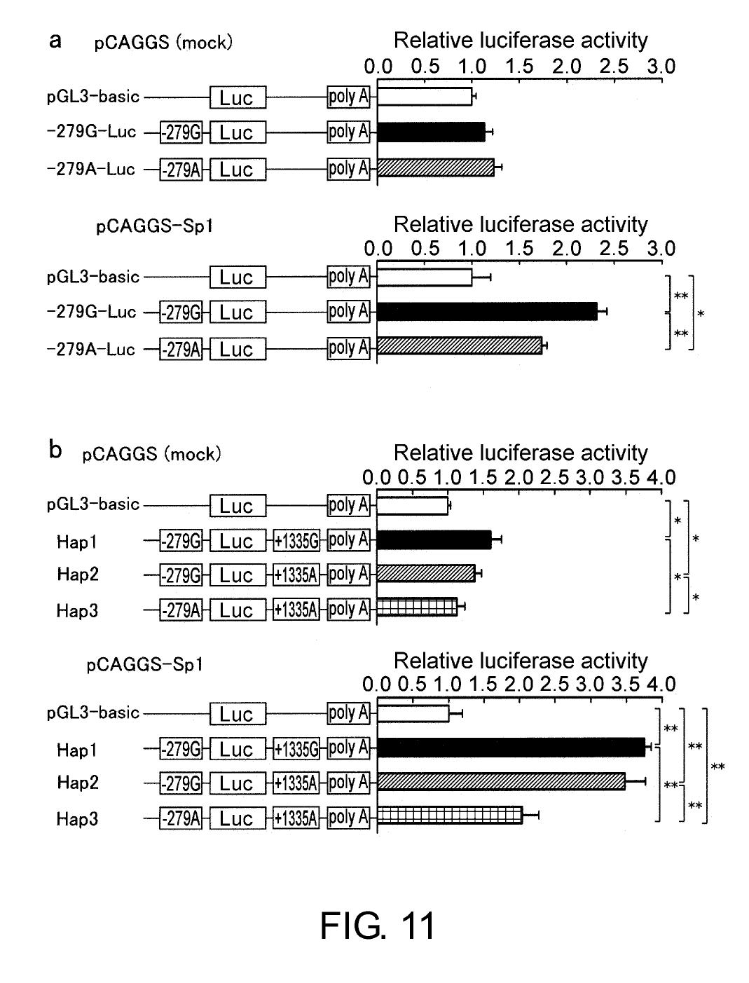

To evaluate the promoter activity in SNP4, the present inventors prepared constructs in which a 44-bp fragment around SNP4 corresponding to the risk allele (−279G-Luc) or non-risk allele (−279A-Luc) was contained in the luciferase reporter vector (pGL3-basic), and then performed luciferase assays using SBC-3 cells cotransfected with one of the constructs and mock pCAGGS vector or pCAGGS-Sp1 vector (FIG. 11a). In cells transfected with mock pCAGGS, increase of luciferase activity due to the SNP4 fragment was not observed. The luciferase activity of Sp1-overexpressing cells with the risk allele was at a level 2.3 times greater than that of the control (pGL3-basic). The activity of cells with the non-risk allele was at a level only 1.7 times greater than that of the control. That is, the function of the promoter around SNP4 was activated in the presence of the Sp1 transcription factor, and the risk allele showed a significantly stronger activity than the non-risk allele (p=0.003).

### Example 12

### The Combination of SNP4 and SNP9 Affects the Transcription of AGTRL1

The present inventors prepared constructs containing a 44-bp fragment around SNP4 and a 53-bp fragment around SNP9 in the luciferase reporter vector (pGL3-basic) for each of the three haplotypes shown in Table 13, and then performed luciferase assays using SBC-3 cells cotransfected with one of the constructs and mock pCAGGS vector or pCAGGS-Sp1 vector (FIG. 11b). In cells transfected with mock pCAGGS, the Hap1 construct (risk haplotype) showed the highest luciferase activity, and the Hap3 construct (non-risk haplotype) showed the lowest luciferase activity. The Hap2 construct (intermediate haplotype) showed intermediate activity. As a result, an intronic enhancer that enhances the promoter activity of the AGTRL1 gene was found to be present near the risk allele of SNP9 (+1355G). These data match the results of the odds ratio in haplotype analyses (Table 11). The activity was further enhanced in Sp1-overexpressing cells.

### INDUSTRIAL APPLICABILITY

The present inventors performed genome-wide association study using SNPs, which targeted the whole genome to identify cerebral infarction-related genes. The patient group consisted of 1,112 cerebral infarction patients who were making regular hospital visits to the Kyushu University Graduate School of Medical Sciences, Department of Medicine and Clinical Science and related facilities. For the control group, a sex- and age-matched control subject was randomly selected for each case in the patient group from Hisayama residents who had taken health examinations conducted from 2002 to 2003. Written informed consents were obtained after explanation by those in charge of explaining based on the “ethical guidelines for human genome/genetic analysis research” co-issued by the Ministry of Education, Culture, Sports, Science, and Technology, Ministry of Health, Labour and Welfare, and Ministry of Economy, Trade and Industry, and then blood samples and clinical information were obtained. In the first screening, 188 cases were randomly selected from each of the patient group (1,112 cases) and the control group (1,112 cases), and 52,608 SNPs distributed in the whole genome were measured. The second screening was performed using all subjects for the 1,098 SNPs which were confirmed to show association between the patient group and the control group in the first screening, and genes implicated in cerebral infarction were identified. SNPs in the whole genome were measured using a high-throughput SNP typing system that uses the invader method of the Institute of Medical Science, the University of Tokyo.

As a result, the AGTRL1 (Angiotensin II receptor-like 1) gene and PRKCH (Protein kinase C eta) gene were identified as cerebral infarction-related genes. In the AGTRL1 gene, most of the SNPs present at the 5′ side, within the gene, and at the 3′ side showed significant difference of p<1×10⁻⁴ between the patient group and the control group. This relationship was also observed when the subjects were limited to patients with arteriosclerosis-related cerebral infarction (lacunar infarction and atherothrombotic infarction). Furthermore, luciferase assay and gel shift assay showed differences in the binding of the transcription factor among the SNPs of the AGTRL1 gene, and difference in the binding of the transcription factor to the region containing the SNP at the 5′ side or the 3′ side was found to cause change in the expression level of the AGTRL1 gene mRNA. Therefore, it was considered that in the AGTRL1 gene, change in the AGTRL1 expression level due to difference in the binding of the transcription factor to the regions containing a SNP at the 5′ or 340 side is associated with the onset of arteriosclerotic diseases such as cerebral infarction.

On the other hand, in the PRKCH gene, significant difference of p<1×10⁻⁴ was observed only in the region at the central part (intron 5 to intron 10) of the gene in the group of patients with arteriosclerosis-related cerebral infarction (lacunar infarction group and atherothrombotic infarction group). Direct sequencing of this region confirmed an SNP (rs2230500) in which the amino acid at position 374 is substituted from valine (Val) to isoleucine (Ile) through change of the allele from G to A, and there were significantly more people that carry Ile (A allele) in the cerebral infarction patient group. In vitro autophosphorylation assay was conducted to examine the effect of this amino acid substitution on the activity of protein kinase η (Protein kinase C eta; PKC-eta), which is a gene product of PRKCH, and revealed amino acid substitution from Val to Ile enhanced the autophosphorylation reaction of PKC-eta. More specifically, the SNP (rs2230500) which substitutes the amino acid at position 374 of PKC-eta from Val to Ile affects the activity of PKC-eta, and this difference in activity was considered to be related to cerebral infarction. Furthermore, immunohistological staining of autopsy samples of human coronary arteries was conducted using a commercially available antibody against PKC-eta (rabbit polyclonal antibody, Santa Cruz). PKC-eta was expressed in the endothelial cells of the human coronary artery and in the arteriosclerotic plaques, and the degree of expression strongly correlated with the degree of arteriosclerosis. More specifically, PKC-eta was found to be deeply implicated with the onset/development of human arteriosclerosis. Furthermore, to examine whether the SNP (rs2230500) accompanying amino acid substitution of PKC-eta is implicated in the onset of human arteriosclerotic diseases, 1,683 cases were used to examine its relation to the onset of cerebral infarction and myocardial infarction. The cases used were the third population of the Hisayama study which had underwent a general Hisayama resident health examination in 1988 and were under long-term observation, and their genomic DNAs were collected from 2002 to 2003. During the 14-year follow-up period, onset of cerebral infarction was observed in 67 cases and onset of myocardial infarction was observed in 37 cases. Those who had the AA allele for rs2230500 had 2.83 times higher risk for cerebral infarction than those with the GG allele. Similarly, the risk for myocardial infarction was 2.89 times higher in those with AA than in those with GG. From these results, SNP (rs2230500) which substitutes the amino acid at position 374 existing in exon 9 of the PRKCH gene from Val to Ile is considered to increase in the activity of PKC-eta through amino acid substitution, promote arteriosclerosis in humans via various types of signal transduction, and ultimately increase the risk for cerebral infarction and myocardial infarction in ordinary residents.

In conventional search for candidate genes using genome-wide correlation studies in common diseases such as hypertension, myocardial infarction, cerebral apoplexy, diabetes, chronic rheumatoid arthritis, and such, case-control studies are used in which candidate regions are narrowed down by comparing the differences in SNP frequency between the disease group and control group. However, it is well known that the incidence rate of diseases such as cerebral infarction increases with aging, and the incidence rates differ between sexes. Therefore, when SNP differences are compared between the patient group and the control group, the age and male-female ratio of the two groups have to be taken into account. However, there are no reports so far in which the disease group and the control group completely matched. One of the reasons is that while collecting samples for the patient group can be accomplished relatively easily since it consists of patients visiting the hospital, it is difficult to collect samples for the control group which consists of ordinary residents who do not carry diseases, and in order to match the sex and age, it may be necessary to collect samples several times greater in number than the patient group. Since the epidemiological population of Hisayama consisted of ordinary residents who had taken health examinations and it included a number of those who did not have illnesses (such population was difficult to collect in previous studies), it was possible to set a control group in which the sex and age matched for each case in the patient group. Furthermore, it is known that in case-control studies, if there is a bias when establishing the control group, difference between the disease group and the control group may be different from it really should be (selection bias). Since the residents of Hisayama have a standard Japanese composition in terms of sex, age, and such according to results of previous epidemiological investigations, they have been proven to be a sample population of the entire Japanese population. A control group that has been established randomly from this population is considered to be unbiased. Therefore, it is considered that disease-related genes have been identified with greater accuracy and higher precision compared to reports made in the past by establishing the Hisayama population as the control group of this search for cerebral infarction-related genes.

The greatest advantage of using the Hisayama residents is that this population is a population used in a long-term prospective follow-up study targeting ordinary residents. To examine whether disease-related genes discovered by genome-wide association studies and their genetic polymorphisms are truly related to the diseases, at this time, a population completely different from the population from which the disease-related genes were selected is used to perform case-control studies and examine whether or not there is reproducibility. However, even if reproducibility is observed using a different population, it may be false positive since the possibility of bias due to selection of subject population cannot be denied in case-control studies. Generally, to examine the association of risk factors with diseases, the research method of a prospective follow-up study (cohort study) which performs a long-term follow-up on a population that has not developed the disease and examines the effect of risk factors on the onset of the disease is considered to be the most accurate. However, since collection of subjects and follow-up studies involve an enormous amount of time, effort, and money, there have been no reports so far of examining the relation of genetic polymorphisms to diseases using this method. Since Hisayama cohort is a population established for this purpose and previous populations have also been subjected to continued follow-up investigation, the data of a 14-year long-term prospective follow-up investigation made it possible to examine the association of genetic polymorphisms with cerebral infarction as in the PRKCH gene of this study. This result is the first case in the world that has proven at the level of ordinary residents that genetic polymorphisms are related to the onset of a disease. Furthermore, since the Hisayama cohort has a standard Japanese composition in terms of sex, age, and such, it can be regarded as a sample population of the entire Japanese population, and the results obtained this time may be applicable not only to Hisayama residents but to the entire Japanese population.

1. A method for testing whether or not a subject has a risk factor for arteriosclerotic disease, which uses the subject's AGTRL1 gene expression as an index.

2. A method for testing whether or not a subject has a risk factor for arteriosclerotic disease, which comprises detecting DNA mutation in the subject's AGTRL1 gene.

3. The method of claim 2, wherein said mutation changes the binding of said gene with an Sp1 transcription factor.

4. A method for testing whether or not a subject has a risk factor for arteriosclerotic disease, which uses the subject's PRKCH gene expression as an index.

5. A method for testing whether or not a subject has a risk factor for arteriosclerotic disease, which comprises detecting DNA mutation in the subject's PRKCH gene.

6. The method of any one of claims 1 to 5, wherein the mutation is a polymorphic mutation.

7. A method for testing whether or not a subject has a risk factor for arteriosclerotic disease, wherein the method comprises determining type of nucleotide at a polymorphic site in the subject's AGTRL1 gene.

8. The method of claim 7, wherein the polymorphic site is in the AGTRL1 gene located at (1a) position 1, (2a) position 12541, (3a) position 21545, (4a) position 33051, (5a) position 35365, (6a) position 39268, (7a) position 39353, (8a) position 39370, (9a) position 39474, (1a) position 39553, (11a) position 39665, (12a) position 41786, (13a) position 42019, (14a) position 42509, (15a) position 43029, (16a) position 43406, (17a) position 43663, (18a) position 46786, (19a) position 49764, (20a) position 64276, (21a) position 74482, (22a) position 78162, (23a) position 93492, or (24a) position 102938 of the nucleotide sequence of SEQ ID NO: 1.

9. The method of claim 8, wherein the subject is determined to have a risk factor for arteriosclerotic disease when the nucleotides at the polymorphic sites of (1a) to (24a) of claim 8 are (1b) to (24b) below, respectively:
(1b) the type of nucleotide in the complementary strand of the AGTRL1 gene located at position 1 of the nucleotide sequence of SEQ ID NO: 1 is T;
(2b) the type of nucleotide in the complementary strand of the AGTRL1 gene located at position 12541 of the nucleotide sequence of SEQ ID NO: 1 is T;
(3b) the type of nucleotide in the complementary strand of the AGTRL1 gene located at position 21545 of the nucleotide sequence of SEQ ID NO: 1 is A;
(4b) the type of nucleotide in the complementary strand of the AGTRL1 gene located at position 33051 of the nucleotide sequence of SEQ ID NO: 1 is C;
(5b) the type of nucleotide in the complementary strand of the AGTRL1 gene located at position 35365 of the nucleotide sequence of SEQ ID NO: 1 is T;
(6b) the type of nucleotide in the complementary strand of the AGTRL1 gene located at position 39268 of the nucleotide sequence of SEQ ID NO: 1 is A;
(7b) the type of nucleotide in the complementary strand of the AGTRL1 gene located at position 39353 of the nucleotide sequence of SEQ ID NO: 1 is G;
(8b) the type of nucleotide in the complementary strand of the AGTRL1 gene located at position 39370 of the nucleotide sequence of SEQ ID NO: 1 is C;
(9b) the type of nucleotide in the complementary strand of the AGTRL1 gene located at position 39474 of the nucleotide sequence of SEQ ID NO: 1 is T;
(10b) the type of nucleotide in the complementary strand of the AGTRL1 gene located at position 39553 of the nucleotide sequence of SEQ ID NO: 1 is T;
(11b) the nucleotide in the AGTRL1 gene located at position 39665 of the nucleotide sequence of SEQ ID NO: 1 has been deleted;
(12b) the type of nucleotide in the complementary strand of the AGTRL1 gene located at position 41786 of the nucleotide sequence of SEQ ID NO: 1 is A;
(13b) the type of nucleotide in the complementary strand of the AGTRL1 gene located at position 42019 of the nucleotide sequence of SEQ ID NO: 1 is G;
(14b) the type of nucleotide in the complementary strand of the AGTRL1 gene located at position 42509 of the nucleotide sequence of SEQ ID NO: 1 is G;
(15b) the type of nucleotide in the complementary strand of the AGTRL1 gene located at position 43029 of the nucleotide sequence of SEQ ID NO: 1 is G;
(16b) the type of nucleotide in the complementary strand of the AGTRL1 gene located at position 43406 of the nucleotide sequence of SEQ ID NO: 1 is C;
(17b) the type of nucleotide in the complementary strand of the AGTRL1 gene located at position 43663 of the nucleotide sequence of SEQ ID NO: 1 is T;
(18b) the type of nucleotide in the complementary strand of the AGTRL1 gene located at position 46786 of the nucleotide sequence of SEQ ID NO: 1 is C;
(19b) the type of nucleotide in the complementary strand of the AGTRL1 gene located at position 49764 of the nucleotide sequence of SEQ ID NO: 1 is T;
(20b) the type of nucleotide in the complementary strand of the AGTRL1 gene located at position 64276 of the nucleotide sequence of SEQ ID NO: 1 is T;
(21b) the type of nucleotide in the complementary strand of the AGTRL1 gene located at position 74482 of the nucleotide sequence of SEQ ID NO: 1 is C;
(22b) the type of nucleotide in the complementary strand of the AGTRL1 gene located at position 78162 of the nucleotide sequence of SEQ ID NO: 1 is G;
(23b) the type of nucleotide in the complementary strand of the AGTRL1 gene located at position 93492 of the nucleotide sequence of SEQ ID NO: 1 is G; and
(24b) the type of nucleotide in the complementary strand of the AGTRL1 gene located at position 102938 of the nucleotide sequence of SEQ ID NO: 1 is C.

10. A method for testing whether or not a subject has a risk factor for arteriosclerotic disease, wherein the subject is determined to have a risk factor for arteriosclerotic disease when a DNA block showing the following haplotype is detected:
(A) a haplotype in which the nucleotides in the complementary strand of the AGTRL1 gene at polymorphic sites located at positions 39268, 39353, 41786, 42019, and 43406 of the nucleotide sequence of SEQ ID NO: 1 are A, G, A, G, and C, respectively.

11. A method for testing whether or not a subject has a risk factor for arteriosclerotic disease, which comprises the step of determining the type of nucleotide of a linked polymorphic site present within a DNA block showing the following haplotype:
(A) a haplotype in which the nucleotides of the complementary strand at polymorphic sites on the AGTRL1 gene located at positions 39268, 39353, 41786, 42019, and 43406 of the nucleotide sequence of SEQ ID NO: 1 are A, G, A, G, and C, respectively.

12. A method for testing whether or not a subject has a risk factor for arteriosclerotic disease, which comprises the steps of:
(a) determining the type of nucleotide at a polymorphic site in the AGTRL1 gene of the subject; and
(b) determining that the subject has a risk factor for arteriosclerotic disease when the nucleotide determined in (a) is the same as the nucleotide at said polymorphic site in the AGTRL1 gene showing the haplotype of (A):
(A) a haplotype in which the nucleotides of the complementary strand at polymorphic sites in the AGTRL1 gene located at positions 39268, 39353, 41786, 42019, and 43406 of the nucleotide sequence of SEQ ID NO: 1 are A, G, A, G, and C, respectively.

13. The method of claim 12, wherein said polymorphic site of (a) is in the AGTRL1 gene located at any one of positions 1, 12541, 21545, 33051, 35365, 39268, 39353, 39370, 39474, 39553, 39665, 41786, 42019, 42509, 43029, 43406, 43663, 46786, 49764, 64276, 74482, 78162, 93492, or 102938 of the nucleotide sequence of SEQ ID NO: 1.

14. A method for testing whether or not a subject has a risk factor for arteriosclerotic disease, wherein the subject is determined to have a risk factor for arteriosclerotic disease when the expression level of the subject's AGTRL1 gene is elevated compared to that of a control.

15. A method for testing whether or not a subject has a risk factor for arteriosclerotic disease, wherein the method comprises determining the type of nucleotide at a polymorphic site in the subject's PRKCH gene.

16. The method of claim 15, wherein the polymorphic site is in the PRKCH gene located at (1a) position 1, (2a) position 16212, (3a) position 30981, (4a) position 32408, (5a) position 33463, (6a) position 34446, (7a) position 39322, (8a) position 39469, (9a) position 39471, (10a) position 49248, (11a) position 49367, or (12a) position 52030 of the nucleotide sequence of SEQ ID NO: 2.

17. The method of claim 16, wherein the subject is determined to have a risk factor for arteriosclerotic disease when the nucleotides in the polymorphic sites of (1a) to (12a) of claim 16 are the following (1b) to (12b), respectively:
(1b) the nucleotide in the PRKCH gene located at position 1 of the nucleotide sequence of SEQ ID NO: 2 is A;
(2b) the nucleotide in the PRKCH gene located at position 16212 of the nucleotide sequence of SEQ ID NO: 2 is G;
(3b) the nucleotide in the PRKCH gene located at position 30981 of the nucleotide sequence of SEQ ID NO: 2 is A;
(4b) the nucleotide in the PRKCH gene located at position 32408 of the nucleotide sequence of SEQ ID NO: 2 is G;
(5b) the nucleotide in the PRKCH gene located at position 33463 of the nucleotide sequence of SEQ ID NO: 2 is G;
(6b) the nucleotide in the PRKCH gene located at position 34446 of the nucleotide sequence of SEQ ID NO: 2 is T;
(7b) the nucleotide in the PRKCH gene located at position 39322 of the nucleotide sequence of SEQ ID NO: 2 is T;
(8b) the nucleotide in the PRKCH gene located at position 39469 of the nucleotide sequence of SEQ ID NO: 2 is A;
(9b) the nucleotide in the PRKCH gene located at position 39471 of the nucleotide sequence of SEQ ID NO: 2 is C;
(10b) the nucleotide in the PRKCH gene located at position 49248 of the nucleotide sequence of SEQ ID NO: 2 is C;
(11b) the nucleotide in the PRKCH gene located at position 49367 of the nucleotide sequence of SEQ ID NO: 2 is G; and
(12b) the nucleotide in the PRKCH gene located at position 52030 of the nucleotide sequence of SEQ ID NO: 2 is A.

18. A method for testing whether or not a subject has a risk factor for arteriosclerotic disease, wherein the subject is determined to have a risk factor for arteriosclerotic disease when the autophosphorylation activity or kinase activity of the subject's PRKCH protein is elevated compared to that of a control.

19. A method for testing whether or not a subject has a risk factor for arteriosclerotic disease, wherein the subject is determined to have a risk factor for arteriosclerotic disease when the subject carries a mutant protein in which valine at position 374 in the amino acid sequence of the PRKCH protein is substituted with isoleucine.

20. The method of any one of claims 1 to 19, wherein a biological sample derived from the subject is subjected to the test as a test sample.

21. A reagent for testing for the presence or absence of a risk factor for arteriosclerotic disease, which comprises an oligonucleotide that hybridizes with a DNA comprising the polymorphic sites of (1a) to (24a) of claim 8 or (1a) to (12a) of claim 16 and has a length of at least 15 nucleotides.

22. A reagent for testing for the presence or absence of a risk factor for arteriosclerotic disease, which comprises a solid phase to which a nucleotide probe is immobilized, wherein the nucleotide probe hybridizes with a DNA comprising the polymorphic sites of (1a) to (24a) of claim 8 or (1a) to (12a) of claim 16.

23. A reagent for testing for the presence or absence of a risk factor for arteriosclerotic disease, which comprises a primer oligonucleotide for amplifying a DNA comprising the polymorphic sites of (1a) to (24a) of claim 8 or (1a) to (12a) of claim 16.

24. A reagent for testing for the presence or absence of a risk factor for arteriosclerotic disease, which comprises (a) or (b) as an active ingredient:
(a) an oligonucleotide that hybridizes with a transcript of an AGTRL1 or PRKCH gene; and
(b) an antibody that recognizes an AGTRL1 or PRKCH protein.

25. A reagent for screening for a pharmaceutical agent for treating or preventing arteriosclerotic disease, which comprises any one of (a) to (c) as an active ingredient:
(a) an oligonucleotide that hybridizes with a transcript of an AGTRL1 gene;
(b) an antibody that recognizes an AGTRL1 protein; and
(c) a polynucleotide comprising a DNA region which comprises a nucleotide site in a AGTRL1 gene located at position 39353 or 42509 of the nucleotide sequence of SEQ ID NO: 1.

26. A reagent for screening for a pharmaceutical agent for treating or preventing arteriosclerotic disease, which comprises any one of (a) to (c) as an active ingredient:
(a) an oligonucleotide that hybridizes with a transcript of a PRKCH gene;
(b) an antibody that recognizes a PRKCH protein; and
(c) a mutant PRKCH protein which has an amino acid sequence in which valine at position 374 of the amino acid sequence of a PRKCH protein is substituted with isoleucine.

27. A pharmaceutical agent for treating or preventing arteriosclerotic disease, which comprises as an active ingredient a substance that suppresses the expression of an AGTRL1 or PRKCH gene or suppresses the function of a protein encoded by said gene.

28. The pharmaceutical agent of claim 27, wherein the substance that suppresses the expression of the AGTRL1 or PRKCH gene is a compound selected from the group consisting of (a) to (c):
(a) an antisense nucleic acid against a transcript of the AGTRL1 or PRKCH gene or a portion thereof;
(b) a nucleic acid having a ribozyme activity of specifically cleaving a transcript of the AGTRL1 or PRKCH gene; and
(c) a nucleic acid having an effect of inhibiting the expression of the AGTRL1 or PRKCH gene through an RNAi effect.

29. The pharmaceutical agent of claim 27, wherein the substance that suppresses the function of the AGTRL1 or PRKCH protein is the compound of (a) or (b):
(a) an antibody that binds to an AGTRL1 or PRKCH protein; or
(b) a low-molecular-weight compound that binds to an AGTRL1 or PRKCH protein.

30. A pharmaceutical agent for treating or preventing arteriosclerotic disease, which comprises as an active ingredient a substance that inhibits the binding of an Sp1 transcription factor with a DNA region that comprises a nucleotide site in the AGTRL1 gene located at position 39353 or 42509 of the nucleotide sequence of SEQ ID NO: 1.

31. A pharmaceutical agent for treating or preventing arteriosclerotic disease, which comprises as an active ingredient a substance that inhibits the autophosphorylation activity of a PRKCH protein.

32. A method of screening for a pharmaceutical agent for treating or preventing arteriosclerotic disease, which comprises selecting a compound that reduces the expression level of an AGTRL1 or PRKCH gene or reduces the activity of a protein encoded by said gene.

33. A method of screening for a pharmaceutical agent for treating or preventing arteriosclerotic disease, which comprises the steps of:
(a) contacting a test compound with a cell that expresses an AGTRL1 or PRKCH gene;
(b) measuring the expression level of said AGTRL1 or PRKCH gene; and
(c) selecting the compound that reduces the expression level as compared with that measured in the absence of the test compound.

34. A method of screening for a pharmaceutical agent for treating or preventing arteriosclerotic disease, which comprises the steps of:
(a) contacting a test compound with a cell or cell extract that comprises a DNA having a structure in which a transcriptional regulatory region of an AGTRL1 or PRKCH gene and a reporter gene are operably linked with each other;
(b) measuring the expression level of said reporter gene; and
(c) selecting a compound that reduces said expression level as compared with that measured in the absence of the test compound.

35. The method of any one of claims 32 to 34, wherein the AGTRL1 gene is a mutant AGTRL1 gene of (a) or (b) whose expression is enhanced:
(a) a mutant AGTRL1 gene in which the nucleotide in the complementary strand of the AGTRL1 gene located at position 42509 of the nucleotide sequence of SEQ ID NO: 1 is G; or
(b) a mutant AGTRL1 gene in which the nucleotide in the complementary strand of the AGTRL1 gene located at position 39353 of the nucleotide sequence of SEQ ID NO: 1 is G.

36. A method of screening for a pharmaceutical agent for treating or preventing arteriosclerotic disease, which comprises the steps of:
(a) contacting a test compound with an Sp1 transcription factor and a polynucleotide comprising a DNA region that comprises a nucleotide site in an AGTRL1 gene located at position 39353 or 42509 of the nucleotide sequence of SEQ ID NO: 1;
(b) measuring the binding activity between said polynucleotide and the Sp1 transcription factor; and
(c) selecting a compound that reduces said binding activity as compared with that measured in the absence of the test compound.

37. A method of screening for a pharmaceutical agent for treating or preventing arteriosclerotic disease, which comprises the steps of:
(a) contacting a test compound with a PRKCH protein;
(b) measuring the autophosphorylation activity of the PRKCH protein; and
(c) selecting a compound that reduces the autophosphorylation activity as compared with that measured in the absence of the test compound.

38. The method of claim 37, wherein said PRKCH protein is a mutant protein in which valine of position 374 in the amino acid sequence of the PRKCH protein is substituted with isoleucine.

39. A method of screening for a pharmaceutical agent for treating or preventing arteriosclerotic disease, which comprises the steps of:
(a) contacting a test compound with a PRKCH protein;
(b) measuring the protein kinase activity of the PRKCH protein; and
(c) selecting a compound that reduces the protein kinase activity as compared with that measured in the absence of the test compound.
